SEQUENCE LISTING (281 sequences)

SEQ ID NO: 1 - protein, 250 aa, Homo sapiens
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 2 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 3 - protein, 275 aa, Homo sapiens
MSSFGYRTLTVALFTLICCPGSDEKVFEVHVRPKKLAVEPKGSLEVNCSTTCNQPEVGGLETSLDKILLDEQAQWKHYLVSNISHDTVLQCHFTCSGKQESMNSNVSVYQPPRQVILTLQPTLVAVGKSFTIECRVPTVEPLDSLTLFLFRGNETLHYETFGKAAPAPQEATATFNSTADREDGHRNFSCLAVLDLMSRGGNIFHKHSAPKMLEIYEPVSDSQMVIIVTVVSVLLSLFVTSVLLCFIFGQHLRQQRMGTYGVRAAWRRLPQAFRP

SEQ ID NO: 4 - protein, 547 aa, Homo sapiens
MATMVPSVLWPRACWTLLVCCLLTPGVQGQEFLLRVEPQNPVLSAGGSLFVNCSTDCPSSEKIALETSLSKELVASGMGWAAFNLSNVTGNSRILCSVYCNGSQITGSSNITVYRLPERVELAPLPPWQPVGQNFTLRCQVEDGSPRTSLTVVLLRWEEELSRQPAVEEPAEVTATVLASRDDHGAPFSCRTELDMQPQGLGLFVNTSAPRQLRTFVLPVTPPRLVAPRFLEVETSWPVDCTLDGLFPASEAQVYLALGDQMLNATVMNHGDTLTATATATARADQEGAREIVCNVTLGGERREARENLTVFSFLGPIVNLSEPTAHEGSTVTVSCMAGARVQVTLDGVPAAAPGQPAQLQLNATESDDGRSFFCSATLEVDGEFLHRNSSVQLRVLYGPKIDRATCPQHLKWKDKTRHVLQCQARGNPYPELRCLKEGSSREVPVGIPFFVNVTHNGTYQCQASSSRGKYTLVVVMDIEAGSSHFVPVFVAVLLTLGVVTIVLALMYVFREHQRSGSYHVREESTYLPLTSMQPTEAMGEEPSRAE

SEQ ID NO: 5 - protein, 271 aa, Homo sapiens
MGSLFPLSLLFFLAAAYPGVGSALGRRTKRAQSPKGSPLAPSGTSVPFWVRMSPEFVAVQPGKSVQLNCSNSCPQPQNSSLRTPLRQGKTLRGPGWVSYQLLDVRAWSSLAHCLVTCAGKTRWATSRITAYKPPHSVILEPPVLKGRKYTLRCHVTQVFPVGYLVVTLRHGSRVIYSESLERFTGLDLANVTLTYEFAAGPRDFWQPVICHARLNLDGLVVRNSSAPITLMLAWSPAPTALASGSIAALVGILLTVGAAYLCKCLAMKSQA

SEQ ID NO: 6 - protein, 332 aa, Homo sapiens
MPGPSPGLRRALLGLWAALGLGLFGLSAVSQEPFWADLQPRVAFVERGGSLWLNCSTNCPRPERGGLETSLRRNGTQRGLRWLARQLVDIREPETQPVCFFRCARRTLQARGLIRTFQRPDRVELMPLPPWQPVGENFTLSCRVPGAGPRASLTLTLLRGAQELIRRSFAGEPPRARGAVLTATVLARREDHGANFSCRAELDLRPHGLGLFENSSAPRELRTFSLSPDAPRLAAPRLLEVGSERPVSCTLDGLFPASEARVYLALGDQNLSPDVTLEGDAFVATATATASAEQEGARQLVCNVTLGGENRETRENVTIYSFPAPLLTLSEP

SEQ ID NO: 7 - protein, 299 aa, Homo sapiens
MGTKAQVERKLLCLFILAILLCSLALGSVTVHSSEPEVRIPENNPVKLSCAYSGFSSPRVEWKFDQGDTTRLVCYNNKITASYEDRVTFLPTGITFKSVTREDTGTYTCMVSEEGGNSYGEVKVKLIVLVPPSKPTVNIPSSATIGNRAVLTCSEQDGSPPSEYTWFKDGIVMPTNPKSTRAFSNSSYVLNPTTGELVFDPLSASDTGEYSCEARNGYGTPMTSNAVRMEAVERNVGVIVAAVLVTLILLGILVFGIWFAYSRGHFDRTKKGTSSKKVIYSQPSARSEGEFKQTSSFLV

SEQ ID NO: 8 - protein, 417 aa, Homo sapiens
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

SEQ ID NO: 9 - protein, 538 aa, Homo sapiens
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 10 - protein, 187 aa, synthetic construct
FSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHR

SEQ ID NO: 11 - DNA, 750 bp, Homo sapiens
  source 1..750: mol_type = unassigned DNA
atggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtattctgaaatgtgacagaaaaccagacagaaccaactccaat

SEQ ID NO: 12 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 13 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 14 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 15 - protein, 177 aa, Homo sapiens
MCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 16 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 17 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 18 - protein, 302 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 19 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 20 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 21 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 22 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 23 - protein, 383 aa, Homo sapiens
MGLGPVFLLLAGIFPFAPPGAAAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 24 - protein, 383 aa, Homo sapiens
MGLGRVLLFLAVAFPFAPPAAAAEPHSLRYNLMVLSQDGSVQSGFLAEGHLDGQPFLRYDRQKRRAKPQGQWAENVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTDFPYVSAAMPCFVIIIILCVPCCKKKTSAAEGPELVSLQVLDQHPVGTGDHRDAAQLGFQPLMSATGSTGSTEGT

SEQ ID NO: 25 - protein, 101 aa, synthetic construct
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVT

SEQ ID NO: 26 - protein, 86 aa, synthetic construct
PSISDFEIPTSNIRRIICSTSGGFPEPHLSWLENGEELNAINTTVSQDPETELYAVSSKLDFNMTTNHSFMCLIKYGHLRVNQTFN

SEQ ID NO: 27 - protein, 99 aa, synthetic construct
NETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELS

SEQ ID NO: 28 - protein, 76 aa, synthetic construct
NVYINLTCSSIHGYPEPKKMSVLLRTKNSTIEYDGVMQKSQDNVTELYDVSISLSVSFPDVTSNMTIFCILETDKT

SEQ ID NO: 29 - DNA, 864 bp, Homo sapiens
  source 1..864: mol_type = unassigned DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 30 - DNA, 987 bp, Homo sapiens
  source 1..987: mol_type = unassigned DNA
atggatccccagtgcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaactctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaactcaactatcgagtatgatggtgttatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaaggtctgatgaagcccagcgtgtttttaaaagttcgaagacatcttcatgcgacaaaagtgatacatgtttt

SEQ ID NO: 31 - protein, 248 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSAAAKP

SEQ ID NO: 32 - protein, 472 aa, synthetic construct
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRGSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 33 - protein, 648 aa, synthetic construct
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLGSGDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSAGSSGGSGGGGSGGGGSGGGGSSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 34 - protein, 21 aa, synthetic construct
GSSGGSGGGGSGGGGSGGGGS

SEQ ID NO: 35 - protein, 23 aa, synthetic construct
GSSGGSGGGGSGGGGSGGGGSSG

SEQ ID NO: 36 - protein, 7 aa, synthetic construct
GFSLTSY

SEQ ID NO: 37 - protein, 5 aa, synthetic construct
WAGGS

SEQ ID NO: 38 - protein, 15 aa, synthetic construct
DKRAPGKLYYGYPDY

SEQ ID NO: 39 - protein, 15 aa, synthetic construct
RASESVEYYVTSLMQ

SEQ ID NO: 40 - protein, 7 aa, synthetic construct
AASNYES

SEQ ID NO: 41 - protein, 9 aa, synthetic construct
QQTRKVPST

SEQ ID NO: 42 - protein, 6 aa, synthetic construct
GYTFSY

SEQ ID NO: 43 - protein, 6 aa, synthetic construct
YPGNVN

SEQ ID NO: 44 - protein, 11 aa, synthetic construct
SHYGLDWNFDV

SEQ ID NO: 45 - protein, 10 aa, synthetic construct
HASQNIYVLN

SEQ ID NO: 46 - protein, 7 aa, synthetic construct
KASNLHT

SEQ ID NO: 47 - protein, 9 aa, synthetic construct
QQGQTYPYT

SEQ ID NO: 48 - protein, 7 aa, synthetic construct
GYTFTRY

SEQ ID NO: 49 - protein, 6 aa, synthetic construct
NPSRGY

SEQ ID NO: 50 - protein, 10 aa, synthetic construct
YYDDHYCLDY

SEQ ID NO: 51 - protein, 10 aa, synthetic construct
SASSSVSYMN

SEQ ID NO: 52 - protein, 7 aa, synthetic construct
DTSKLAS

SEQ ID NO: 53 - protein, 9 aa, synthetic construct
QQWSSNPFT

SEQ ID NO: 54 - protein, 5 aa, synthetic construct
RYTMH

SEQ ID NO: 55 - protein, 17 aa, synthetic construct
YINPSRGYTNYNQKVKD

SEQ ID NO: 56 - protein, 10 aa, synthetic construct
YYDDHYCLDY

SEQ ID NO: 57 - protein, 10 aa, synthetic construct
SASSSVSYMN

SEQ ID NO: 58 - protein, 8 aa, synthetic construct
DTSKLASG

SEQ ID NO: 59 - protein, 9 aa, synthetic construct
QQWSSNPFT

SEQ ID NO: 60 - protein, 275 aa, synthetic construct
MGVKVLFALICIAVAEAFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLGSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPDNLLPSWAITLISVNGIFVICCLTYCFAPRCRERRRNERLRRESVRPV

SEQ ID NO: 61 - protein, 286 aa, synthetic construct
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLGSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPDNLLPSWAITLISVNGIFVICCLTYCFAPRCRERRRNERLRRESVRPV

SEQ ID NO: 62 - protein, 266 aa, synthetic construct
MGVKVLFALICIAVAEAVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKAGSGFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSSGHSRHRYALIPIPLAVITTCIVLYMNGILKCDRKPDRTNSN

SEQ ID NO: 63 - protein, 283 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKAGSGFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSSGHSRHRYALIPIPLAVITTCIVLYMNGILKCDRKPDRTNSN

SEQ ID NO: 64 - protein, 348 aa, synthetic construct
MGVKVLFALICIAVAEAVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKAGSGFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 65 - protein, 370 aa, synthetic construct
MGVKVLFALICIAVAEAFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLGSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 66 - protein, 365 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKAGSGFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 67 - protein, 381 aa, synthetic construct
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLGSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 68 - protein, 467 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 69 - protein, 613 aa, synthetic construct
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSAGSSGGSGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKAGSGFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 70 - protein, 635 aa, synthetic construct
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSAGSSGGSGGGGSGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLGSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 71 - protein, 632 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKAGSGDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSAGSSGGSGGGGSGGGGSGGGGSSGFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 72 - protein, 648 aa, synthetic construct; identical to SEQ ID NO: 33

SEQ ID NO: 73 - protein, 726 aa, synthetic construct
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSAGSSGGSGGGGSGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRGSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 74 - protein, 511 aa, synthetic construct
NFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 75 - DNA, 1554 bp, synthetic construct
  source 1..1554: mol_type = other DNA
atggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 76 - DNA, 1560 bp, synthetic construct
  source 1..1560: mol_type = other DNA
gccaccatggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 77 - protein, 496 aa, synthetic construct
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 21 aa, synthetic construct
MALPVTALLLPLALLLHAARP

SEQ ID NO: 79 - protein, 498 aa, synthetic construct
LLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - DNA, 1296 bp, synthetic construct
  source 1..1296: mol_type = other DNA
atgatctacgacgtgtccaagaggccatctggagtgcctgatcggttctccggctctaagagcggcaattccgcctctctggacatcagcggactgcagtccgaggacgaggcagattactattgcgccgcctgggacgatagcctgtccgagtttctgttcggcaccggcacaaagctgaccgtgctgggctctacaagcggatccggcaagccaggatctggagagggcagcacaaagggacaggtgcagctggtggagagcggaggaaacctggtgcagccaggaggctccctgcgcctgtcttgtgccgccagcggctttaccttcggctcttttagcatgtcctgggtgcgccaggcacctggaggaggactggagtgggtggccggcctgagcgcccggtctagcctgacacactatgccgactccgtgaagggccgcttcaccatctcccgggataacgccaagaatagcgtgtacctgcagatgaatagcctgcgggtggaggacacagccgtgtactattgcgccaggcgctcctatgattcctctggctactggggccacttttactcttatatggacgtgtggggacagggcaccctggtgacagtgagctccaccaccacacctgctcctagaccacctacacccgctcctaccatcgccagccagcctctgtctctgagacctgaggcctgtagacctgccgctggaggcgctgtgcacaccagaggactggatttcgcctgcgacatctacatctgggctcctctggctggaacatgcggcgtgctgctcctgagcctggtgatcacactgtactgcaagcgcggccggaagaagctgctctacatctttaagcagccattcatgcgccccgtgcagaccacacaggaggaggacggctgctcctgtcggtttccagaggaggaggagggaggatgtgagctgagagtgaagttctctaggagcgccgatgcccctgcctatcagcagggacagaaccagctgtacaacgagctgaatctgggccggagagaggagtacgacgtgctggataagaggaggggaagagacccagagatgggaggcaagcctcggagaaagaacccacaggagggcctgtataatgagctgcagaaggacaagatggccgaggcctactccgagatcggcatgaagggagagaggcgccggggcaagggacacgatggcctgtatcagggcctgagcaccgccacaaaggacacatacgatgccctgcacatgcaggccctgcctccaaggtga

SEQ ID NO: 81 - DNA, 1497 bp, synthetic construct
  source 1..1497: mol_type = other DNA
ctgctgctggtgacaagcctgctgctgtgcgagctgcctcacccagcctttctgctgatcccctccgtgctgacccagcctagctccgtgtctgccgcaccaggacagaaggtgacaatcagctgttccggctctaccagcaacatcggcaacaattacgtgagctggtaccagcagcaccctggcaaggccccaaagctgatgatctacgacgtgtccaagaggccatctggagtgcctgatcggttctccggctctaagagcggcaattccgcctctctggacatcagcggactgcagtccgaggacgaggcagattactattgcgccgcctgggacgatagcctgtccgagtttctgttcggcaccggcacaaagctgaccgtgctgggctctacaagcggatccggcaagccaggatctggagagggcagcacaaagggacaggtgcagctggtggagagcggaggaaacctggtgcagccaggaggctccctgcgcctgtcttgtgccgccagcggctttaccttcggctcttttagcatgtcctgggtgcgccaggcacctggaggaggactggagtgggtggccggcctgagcgcccggtctagcctgacacactatgccgactccgtgaagggccgcttcaccatctcccgggataacgccaagaatagcgtgtacctgcagatgaatagcctgcgggtggaggacacagccgtgtactattgcgccaggcgctcctatgattcctctggctactggggccacttttactcttatatggacgtgtggggacagggcaccctggtgacagtgagctccaccaccacacctgctcctagaccacctacacccgctcctaccatcgccagccagcctctgtctctgagacctgaggcctgtagacctgccgctggaggcgctgtgcacaccagaggactggatttcgcctgcgacatctacatctgggctcctctggctggaacatgcggcgtgctgctcctgagcctggtgatcacactgtactgcaagcgcggccggaagaagctgctctacatctttaagcagccattcatgcgccccgtgcagaccacacaggaggaggacggctgctcctgtcggtttccagaggaggaggagggaggatgtgagctgagagtgaagttctctaggagcgccgatgcccctgcctatcagcagggacagaaccagctgtacaacgagctgaatctgggccggagagaggagtacgacgtgctggataagaggaggggaagagacccagagatgggaggcaagcctcggagaaagaacccacaggagggcctgtataatgagctgcagaaggacaagatggccgaggcctactccgagatcggcatgaagggagagaggcgccggggcaagggacacgatggcctgtatcagggcctgagcaccgccacaaaggacacatacgatgccctgcacatgcaggccctgcctccaaggtga

SEQ ID NO: 82 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - DNA, 336 bp, synthetic construct
  source 1..336: mol_type = other DNA
cgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacatatgatgccctgcacatgcaggccctgccacctagg

SEQ ID NO: 84 - protein, 42 aa, synthetic construct
KRGRKKLLYIFKQPFMRPVQTTQEEDGCSCRFPEEEEGGCEL

SEQ ID NO: 85 - DNA, 336 bp, synthetic construct; identical to SEQ ID NO: 83
  source 1..336: mol_type = other DNA

SEQ ID NO: 86 - protein, 194 aa, synthetic construct
ESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - DNA, 582 bp, synthetic construct
  source 1..582: mol_type = other DNA
gagtctaagtatggccctccatgccccccttgtcctatgttctgggtgctggtggtggtgggaggcgtgctggcctgttactccctgctggtgaccgtggcctttatcatcttctgggtgaagcgcggccggaagaagctgctgtatatctttaagcagcccttcatgagacctgtgcagaccacacaggaggaggacggctgcagctgtaggtttccagaggaggaggagggaggatgcgagctgcgcgtgaagttctctcggagcgccgatgcccctgcctaccagcagggacagaaccagctgtataacgagctgaatctgggccggagagaggagtacgacgtgctggataagaggaggggaagagacccagagatgggaggcaagcctcggagaaagaacccacaggagggcctgtacaatgagctgcagaaggacaagatggccgaggcctattccgagatcggcatgaagggagagaggcgccggggcaagggacacgatggcctgtaccagggcctgagcaccgccacaaaggacacctatgatgccctgcacatgcaggccctgccacccagg

SEQ ID NO: 88 - protein, 216 aa, synthetic construct
ESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 89 - DNA, 648 bp, synthetic construct
  source 1..648: mol_type = other DNA
gagtccaagtacggcccaccctgccctccatgtcccatgttttgggtgctggtggtggtgggaggcgtgctggcctgttattccctgctggtgaccgtggccttcatcatcttttgggtgaagcgcggccggaagaagctgctgtacatcttcaagcagcccttcatgagacccgtgcagaccacacaggaggaggacggctgcagctgtaggttcccagaggaggaggagggaggatgcgagctgagggtgaagttttcccggtctgccgatgcccctgcctatcagcagggccagaatcagctgtacaacgagctgaatctgggcaggcgcgaggagtacgacgtgctggataagaggagaggaagggaccctgagatgggaggcaagccaaggcgcaagaaccctcaggagggcctgtataatgagctgcagaaggacaagatggccgaggcctactccgagatcggcatgaagggagagcggagaaggggcaagggacacgatggcctgtatcagggcctgagcaccgccacaaaggacacctacgatgcactgcacatgcaggccctgccacctagaggatctggagccacaaacttcagcctgctgaagcaggccggcgatgtggaggagaatcctggacca

SEQ ID NO: 90 - protein, 40 aa, synthetic construct
ESKYGPPCPPCPMFWVLVVVGGVLACYSLLVTVAFIIFWV

SEQ ID NO: 91 - DNA, 120 bp, synthetic construct
  source 1..120: mol_type = other DNA
gagtctaagtatggcccaccctgccctccatgtccaatgttctgggtgctggtggtggtgggaggcgtgctggcctgttactccctgctggtgaccgtggcctttatcatcttctgggtg

SEQ ID NO: 92 - protein, 22 aa, synthetic construct
MLLLVTSLLLCELPHPAFLLIP

SEQ ID NO: 93 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 94 - protein, 11 aa, synthetic construct
RASQDISKYLN

SEQ ID NO: 95 - protein, 7 aa, synthetic construct
HTSRLHS

SEQ ID NO: 96 - protein, 9 aa, synthetic construct
QQGNTLPYT

SEQ ID NO: 97 - protein, 4 aa, synthetic construct
DYGV

SEQ ID NO: 98 - protein, 16 aa, synthetic construct
VIWGSETTYYNSALKS

SEQ ID NO: 99 - protein, 12 aa, synthetic construct
HYYYGGSYAMDY

SEQ ID NO: 100 - protein, 267 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 101 - DNA, 66 bp, synthetic construct
  source 1..66: mol_type = other DNA
atgctgctgctggtgacctccctgctgctgtgcgagctgcctcacccagcctttctgctgatcccc

SEQ ID NO: 102 - DNA, 735 bp, synthetic construct
  source 1..735: mol_type = other DNA
gacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatcaccggatccacatctggaagcggcaagccaggaagcggagagggatccacaaagggagaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagc

SEQ ID NO: 103 - protein, 267 aa, synthetic construct; identical to SEQ ID NO: 100

SEQ ID NO: 104 - DNA, 801 bp, synthetic construct
  source 1..801: mol_type = other DNA
atgctgctgctggtgaccagcctgctgctgtgcgagctgccacaccctgccttcctgctgatcccagatatccagatgacacagaccacatcctctctgtccgcctctctgggcgacagagtgaccatctcttgtagggccagccaggatatctccaagtacctgaactggtatcagcagaagcctgacggcacagtgaagctgctgatctaccacacctctaggctgcacagcggagtgccatcccggttcagcggatccggatctggaacagactattctctgaccatcagcaacctggagcaggaggatatcgccacatacttttgccagcagggcaataccctgccatatacattcggcggaggaaccaagctggagatcaccggaagcacatccggatctggcaagccaggatccggagagggatctacaaagggagaggtgaagctgcaggagagcggaccaggactggtggcacccagccagtccctgtctgtgacctgtacagtgtctggcgtgagcctgcccgattacggcgtgtcctggatcagacagccaccaaggaagggactggagtggctgggcgtgatctggggctctgagaccacatactataatagcgccctgaagtcccggctgaccatcatcaaggacaacagcaagtcccaggtgtttctgaagatgaatagcctgcagaccgacgatacagccatctactattgcgccaagcactactattacggcggctcctacgccatggattattggggccagggcacctccgtgacagtgagctcc

SEQ ID NO: 105 - DNA, 801 bp, synthetic construct
  source 1..801: mol_type = other DNA
atgctgctgctggtgacatccctgctgctgtgcgagctgccacacccagccttcctgctgatccccgatatccagatgacccagaccacaagctccctgagcgcctccctgggcgacagggtgacaatctcttgtcgggccagccaggatatctccaagtatctgaattggtaccagcagaagcccgacggcaccgtgaagctgctgatctatcacacatctagactgcacagcggcgtgccttccaggttttctggcagcggctccggcaccgactactctctgacaatcagcaacctggagcaggaggatatcgccacctatttctgccagcagggcaataccctgccttacacatttggcggcggcacaaagctggagatcaccggctctacaagcggatccggcaagccaggatccggagagggatctaccaagggagaggtgaagctgcaggagagcggacctggactggtggcaccatctcagagcctgtccgtgacctgtacagtgtctggcgtgagcctgccagattatggcgtgagctggatcaggcagccacctaggaagggactggagtggctgggcgtgatctggggctccgagaccacatactataacagcgccctgaagtcccgcctgaccatcatcaaggacaactctaagagccaggtgttcctgaagatgaattccctgcagaccgacgatacagccatctactattgcgccaagcactactattacggcggctcttatgccatggattactggggccagggcaccagcgtgacagtgtctagc

SEQ ID NO: 106 - DNA, 762 bp, synthetic construct
  source 1..762: mol_type = other DNA
tccgtgctgacccagcctagctccgtgtctgccgcaccaggacagaaggtgacaatcagctgttccggctctaccagcaacatcggcaacaattacgtgagctggtaccagcagcaccctggcaaggccccaaagctgatgatctacgacgtgtccaagaggccatctggagtgcctgatcggttctccggctctaagagcggcaattccgcctctctggacatcagcggactgcagtccgaggacgaggcagattactattgcgccgcctgggacgatagcctgtccgagtttctgttcggcaccggcacaaagctgaccgtgctgggctctacaagcggatccggcaagccaggatctggagagggcagcacaaagggacaggtgcagctggtggagagcggaggaaacctggtgcagccaggaggctccctgcgcctgtcttgtgccgccagcggctttaccttcggctcttttagcatgtcctgggtgcgccaggcacctggaggaggactggagtgggtggccggcctgagcgcccggtctagcctgacacactatgccgactccgtgaagggccgcttcaccatctcccgggataacgccaagaatagcgtgtacctgcagatgaatagcctgcgggtggaggacacagccgtgtactattgcgccaggcgctcctatgattcctctggctactggggccacttttactcttatatggacgtgtggggacagggcaccctggtgacagtgagctcc

SEQ ID NO: 107 - protein, 254 aa, synthetic construct
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSS

SEQ ID NO: 108 - DNA, 327 bp, synthetic construct
  source 1..327: mol_type = other DNA
tccgtgctgacccagcctagctccgtgtctgccgcaccaggacagaaggtgacaatcagctgttccggctctaccagcaacatcggcaacaattacgtgagctggtaccagcagcaccctggcaaggccccaaagctgatgatctacgacgtgtccaagaggccatctggagtgcctgatcggttctccggctctaagagcggcaattccgcctctctggacatcagcggactgcagtccgaggacgaggcagattactattgcgccgcctgggacgatagcctgtccgagtttctgttcggcaccggcacaaagctgaccgtgctg

SEQ ID NO: 109 - protein, 109 aa, synthetic construct
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVL

SEQ ID NO: 110 - DNA, 54 bp, synthetic construct
  source 1..54: mol_type = other DNA
ggctctacaagcggatccggcaagccaggatctggagagggcagcacaaaggga

SEQ ID NO: 111 - protein, 18 aa, synthetic construct
GSTSGSGKPGSGEGSTKG

SEQ ID NO: 112 - DNA, 381 bp, synthetic construct
  source 1..381: mol_type = other DNA
caggtgcagctggtggagagcggaggaaacctggtgcagccaggaggctccctgcgcctgtcttgtgccgccagcggctttaccttcggctcttttagcatgtcctgggtgcgccaggcacctggaggaggactggagtgggtggccggcctgagcgcccggtctagcctgacacactatgccgactccgtgaagggccgcttcaccatctcccgggataacgccaagaatagcgtgtacctgcagatgaatagcctgcgggtggaggacacagccgtgtactattgcgccaggcgctcctatgattcctctggctactggggccacttttactcttatatggacgtgtggggacagggcaccctggtgacagtgagctcc

SEQ ID NO: 113 - protein, 127 aa, synthetic construct
QVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSS

SEQ ID NO: 114 - DNA, 762 bp, synthetic construct; identical to SEQ ID NO: 106
  source 1..762: mol_type = other DNA

SEQ ID NO: 115 - DNA, 327 bp, synthetic construct; identical to SEQ ID NO: 108
  source 1..327: mol_type = other DNA

SEQ ID NO: 116 - DNA, 54 bp, synthetic construct; identical to SEQ ID NO: 110
  source 1..54: mol_type = other DNA

SEQ ID NO: 117 - DNA, 381 bp, synthetic construct; identical to SEQ ID NO: 112
  source 1..381: mol_type = other DNA

SEQ ID NO: 118 - DNA, 353 bp, synthetic construct
  source 1..353: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagc

SEQ ID NO: 119 - DNA, 353 bp, synthetic construct; identical to SEQ ID NO: 118
  source 1..353: mol_type = other DNA

SEQ ID NO: 120 - protein, 1296 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGPMPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 121 - DNA, 3891 bp, synthetic construct
  source 1..3891: mol_type = other DNA
atgctgctgctggtgacctccctgctgctgtgcgagctgcctcacccagcctttctgctgatccccgacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatcaccggatccacatctggaagcggcaagccaggaagcggagagggatccacaaagggagaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagcgagtctaagtatggcccaccctgccctccatgtccaatgttctgggtgctggtggtggtgggaggcgtgctggcctgttactccctgctggtgaccgtggcctttatcatcttctgggtgaagagaggcaggaagaagctgctgtatatctttaagcagcccttcatgcgccctgtgcagaccacacaggaggaggacggctgcagctgtcggtttccagaggaggaggagggaggatgcgagctgcgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacatatgatgccctgcacatgcaggccctgccacctaggggatctggagccaccaactttagcctgctgaagcaggcaggcgatgtggaggagaatccaggacctgagatgtggcacgagggactggaggaggcaagcaggctgtactttggcgagcggaatgtgaagggcatgttcgaggtgctggagccactgcacgcaatgatggagaggggaccacagaccctgaaggagacatccttcaaccaggcatacggaagggacctgatggaggcacaggagtggtgccggaagtatatgaagtctggcaatgtgaaggacctgctgcaggcctgggatctgtattaccacgtgtttagaaggatcagcaagggctccggcgccaccaacttctccctgctgaagcaggccggcgatgtggaagaaaatccaggaccaatgcctctgggactgctgtggctgggactggccctgctgggcgccctgcacgcccaggccggcgtgcaggtggagacaatcagccctggcgacggcagaacctttccaaagaggggccagacatgcgtggtgcactacaccggcatgctggaggatggcaagaagttcgactcctctcgcgatcggaacaagccctttaagttcatgctgggcaagcaggaagtgatcagaggctgggaggagggcgtggcccagatgtctgtgggccagagggccaagctgacaatcagcccagactatgcatacggagcaaccggacaccctggaatcatcccaccacacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgccaaggatccccaccctgaagaacctggaggacctggtgaccgagtaccacggcaatttcagcgcctggtccggcgtgtctaagggactggcagagtccctgcagccagattattctgagcggctgtgcctggtgagcgagatccctccaaagggaggcgccctgggagagggaccaggagccagcccctgcaaccagcactccccttactgggcccccccttgttataccctgaagccagagacaggctctggcgccaccaacttcagcctgctgaagcaagccggcgacgtggaagaaaacccaggaccaatggcactgccagtgaccgccctgctgctgcctctggccctgctgctgcacgcagccagacccatcctgtggcacgaaatgtggcatgaaggcctggaggaggcaagcagactgtactttggcgagagaaatgtgaaaggaatgtttgaggtgctggagcctctgcacgccatgatggagaggggccctcagaccctgaaggagacatcctttaaccaggcctacggcagagacctgatggaggcccaggagtggtgcaggaagtatatgaagagcggaaatgtgaaagacctgctgcaggcctgggatctgtactaccacgtgttccgccggatctctaagggcaaggatacaatcccttggctgggacacctgctggtgggactgagcggagcctttggcttcatcatcctggtgtatctgctgatcaactgcagaaatacaggcccatggctgaagaaggtgctgaagtgtaacacccctgacccatccaagttcttttctcagctgagctccgagcacggcggcgatgtgcagaagtggctgtctagcccctttccttcctctagcttcagccctggaggactggcacctgagatctccccactggaggtgctggagagggacaaggtgacccagctgctgctgcagcaggataaggtgccagagcccgcctccctgtcctctaaccacagcctgacctcctgctttacaaatcagggctacttctttttccacctgccagacgcactggagatcgaggcatgtcaggtgtatttcacatacgatccctatagcgaggaggaccctgatgagggagtggccggcgccccaaccggaagctcccctcagccactgcagccactgagcggagaggacgatgcatattgtacatttccttcccgcgacgatctgctgctgttctctccaagcctgctgggaggaccatctccacccagcaccgcacctggaggatccggggcaggggaggagcggatgcctccatctctgcaggagagagtgccaagggactgggatccacagcctctgggaccacctacccctggagtgccagacctggtggatttccagccaccccctgagctggtgctgcgggaggcaggagaggaggtgccagacgcaggacctagagagggcgtgagctttccctggtccaggccaccaggacagggagagttccgcgccctgaacgcccggctgcccctgaatacagacgcctacctgtctctgcaggagctgcagggccaggatcctacccacctggtgtga

SEQ ID NO: 122 - protein, 1297 aa, synthetic construct
MEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGPMPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGATNFSLLKQAGDVEENPGPMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - DNA, 3894 bp, synthetic construct
  source 1..3894: mol_type = other DNA
atggagatgtggcacgagggactggaggaggcaagcagactgtactttggcgagaggaacgtgaagggcatgttcgaggtgctggagccactgcacgcaatgatggagaggggaccacagaccctgaaggagacatctttcaaccaggcatacggaagggacctgatggaggcacaggagtggtgccggaagtatatgaagagcggcaatgtgaaggacctgctgcaggcctgggatctgtactatcacgtgtttcggagaatctccaagggctctggcgccaccaacttctccctgctgaagcaggccggcgatgtggaggagaatcctggaccaatgccactgggactgctgtggctgggactggccctgctgggcgccctgcacgcccaggccggcgtgcaggtggagacaatcagccctggcgacggacgcacctttccaaagaggggacagacatgcgtggtgcactacaccggcatgctggaggatggcaagaagttcgacagctccagagataggaataagccctttaagttcatgctgggcaagcaggaagtgatcaggggatgggaggagggagtggcacagatgtctgtgggacagcgggccaagctgacaatcagcccagactatgcatacggagcaaccggacaccctggaatcatcccacctcacgccacactggtgtttgatgtggagctgctgaagctgggcgagggcagcaacacctccaaggagaatccatttctgttcgccctggaggccgtggtcatctctgtgggcagcatgggcctgatcatctccctgctgtgcgtgtacttttggctggagcgcacaatgccacggatccccaccctgaagaacctggaggacctggtgaccgagtaccacggcaatttctccgcctggtctggcgtgagcaagggactggcagagtctctgcagccagattatagcgagcggctgtgcctggtgagcgagatcccacccaagggaggcgccctgggagagggaccaggagcctccccttgcaaccagcactctccttactgggcccctccatgttataccctgaagccagagacaggcagcggagctactaacttctccctgctgaagcaagcaggcgacgtggaagaaaatcctggaccaatggcactgccagtgaccgccctgctgctgcctctggccctgctgctgcacgcagccagacccatcctgtggcacgaaatgtggcatgaaggcctggaggaggcaagcaggctgtactttggcgagcggaatgtgaaaggaatgtttgaagtgctggagcctctgcacgccatgatggagaggggccctcagaccctgaaggagacatcctttaaccaggcctacggcagagacctgatggaggcccaggagtggtgcaggaagtatatgaagtctggaaatgtgaaagacctgctgcaggcctgggatctgtattatcacgtgttcaggcgcatctctaagggcaaggatacaatcccttggctgggacacctgctggtgggactgagcggagcctttggcttcatcatcctggtgtatctgctgatcaactgccgcaatacaggcccatggctgaagaaggtgctgaagtgtaacacccccgacccttccaagttcttttctcagctgtctagcgagcacggcggcgatgtgcagaagtggctgtcctctccatttcccagctcctctttcagcccaggaggactggcaccagagatctccccactggaggtgctggagagggacaaggtgacccagctgctgctgcagcaggataaggtgcctgagccagcctccctgagctccaaccactccctgacctcttgctttacaaatcagggctacttctttttccacctgccagacgcactggagatcgaggcatgtcaggtgtatttcacatacgatccctatagcgaggaggaccctgatgagggagtggccggcgccccaaccggatctagcccacagcctctgcagccactgagcggagaggacgatgcatattgtacatttccttcccgcgacgatctgctgctgttctctccaagcctgctgggaggaccaagcccaccttccaccgcaccaggcggctccggggcaggggaggagcggatgccaccctctctgcaggagagagtgccaagggactgggatccacagccactgggacctccaacccctggagtgccagacctggtggatttccagccccctccagagctggtgctgagagaggcaggagaggaggtgcctgacgcaggaccaagagagggcgtgagctttccttggtccaggccacctggacagggagagttcagagccctgaacgccaggctgcccctgaatacagacgcctacctgtctctgcaggagctgcagggccaggatcctacacacctggtcggatctggcgccaccaactttagcctgctgaagcaggcaggcgacgtggaagagaaccctggaccaatgctgctgctggtgaccagcctgctgctgtgcgagctgccacaccctgccttcctgctgatcccagatatccagatgacacagaccacatcctctctgtccgcctctctgggcgacagagtgaccatctcttgtagggccagccaggatatctccaagtacctgaactggtatcagcagaagcctgacggcacagtgaagctgctgatctaccacacctctaggctgcacagcggagtgccatcccggttcagcggatccggatctggaacagactattctctgaccatcagcaacctggagcaggaggatatcgccacatacttttgccagcagggcaataccctgccatatacattcggcggaggaaccaagctggagatcaccggaagcacatccggatctggcaagccaggatccggagagggatctacaaagggagaggtgaagctgcaggagagcggaccaggactggtggcacccagccagtccctgtctgtgacctgtacagtgtctggcgtgagcctgcccgattacggcgtgtcctggatcagacagccaccaaggaagggactggagtggctgggcgtgatctggggctctgagaccacatactataatagcgccctgaagtcccggctgaccatcatcaaggacaacagcaagtcccaggtgtttctgaagatgaatagcctgcagaccgacgatacagccatctactattgcgccaagcactactattacggcggctcctacgccatggattattggggccagggcacctccgtgacagtgagctccgagtctaagtatggccctccatgccccccttgtcctatgttctgggtgctggtggtggtgggaggcgtgctggcctgttactccctgctggtgaccgtggcctttatcatcttctgggtgaagcgcggccggaagaagctgctgtatatctttaagcagcccttcatgagacctgtgcagaccacacaggaggaggacggctgcagctgtaggtttccagaggaggaggagggaggatgcgagctgcgcgtgaagttctctcggagcgccgatgcccctgcctaccagcagggacagaaccagctgtataacgagctgaatctgggccggagagaggagtacgacgtgctggataagaggaggggaagagacccagagatgggaggcaagcctcggagaaagaacccacaggagggcctgtacaatgagctgcagaaggacaagatggccgaggcctattccgagatcggcatgaagggagagaggcgccggggcaagggacacgatggcctgtaccagggcctgagcaccgccacaaaggacacctatgatgccctgcacatgcaggccctgccacccaggtga

SEQ ID NO: 124 - protein, 794 aa, synthetic construct
MEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGPMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKRRR

SEQ ID NO: 125 - DNA, 2385 bp, synthetic construct
  source 1..2385: mol_type = other DNA
atggagatgtggcacgagggactggaggaggcatccagactgtacttcggcgagaggaacgtgaagggcatgtttgaggtgctggagccactgcacgccatgatggagagaggcccccagaccctgaaggagacatctttcaaccaggcctatggaagggacctgatggaggcacaggagtggtgccggaagtacatgaagagcggcaatgtgaaggacctgctgcaggcctgggatctgtactatcacgtgttccggagaatcagcaagggctccggcgccaccaactttagcctgctgaagcaggcaggcgacgtggaggagaatccaggacctatgctgctgctggtgacatccctgctgctgtgcgagctgccacacccagccttcctgctgatccccgatatccagatgacccagaccacaagctccctgagcgcctccctgggcgacagggtgacaatctcttgtcgggccagccaggatatctccaagtatctgaattggtaccagcagaagcccgacggcaccgtgaagctgctgatctatcacacatctagactgcacagcggcgtgccttccaggttttctggcagcggctccggcaccgactactctctgacaatcagcaacctggagcaggaggatatcgccacctatttctgccagcagggcaataccctgccttacacatttggcggcggcacaaagctggagatcaccggctctacaagcggatccggcaagccaggatccggagagggatctaccaagggagaggtgaagctgcaggagagcggacctggactggtggcaccatctcagagcctgtccgtgacctgtacagtgtctggcgtgagcctgccagattatggcgtgagctggatcaggcagccacctaggaagggactggagtggctgggcgtgatctggggctccgagaccacatactataacagcgccctgaagtcccgcctgaccatcatcaaggacaactctaagagccaggtgttcctgaagatgaattccctgcagaccgacgatacagccatctactattgcgccaagcactactattacggcggctcttatgccatggattactggggccagggcaccagcgtgacagtgtctagcgagtccaagtacggcccaccctgccctccatgtcccatgttttgggtgctggtggtggtgggaggcgtgctggcctgttattccctgctggtgaccgtggccttcatcatcttttgggtgaagcgcggccggaagaagctgctgtacatcttcaagcagcccttcatgagacccgtgcagaccacacaggaggaggacggctgcagctgtaggttcccagaggaggaggagggaggatgcgagctgagggtgaagttttcccggtctgccgatgcccctgcctatcagcagggccagaatcagctgtacaacgagctgaatctgggcaggcgcgaggagtacgacgtgctggataagaggagaggaagggaccctgagatgggaggcaagccaaggcgcaagaaccctcaggagggcctgtataatgagctgcagaaggacaagatggccgaggcctactccgagatcggcatgaagggagagcggagaaggggcaagggacacgatggcctgtatcagggcctgagcaccgccacaaaggacacctacgatgcactgcacatgcaggccctgccacctagaggatctggagccacaaacttcagcctgctgaagcaggccggcgatgtggaggagaatcctggaccaatgggaagaggactgctgaggggactgtggccactgcacatcgtgctgtggaccaggatcgcctctacaatcccaccccacgtgcagaagagcgtgaacaatgacatgatcgtgaccgataacaatggcgccgtgaagtttccccagctgtgcaagttctgtgacgtgcgcttttccacctgtgataaccagaagtcctgcatgtctaattgtagcatcacatccatctgcgagaagcctcaggaggtgtgcgtggccgtgtggcggaagaacgacgagaatatcaccctggagacagtgtgccacgatcccaagctgccttatcacgacttcatcctggaggatgccgcctctcctaagtgtatcatgaaggagaagaagaagccaggcgagaccttctttatgtgcagctgttcctctgacgagtgcaacgataatatcatcttctccgaggagtacaacacctctaatcctgacctgctgctggtcatctttcaggtgacaggcatctccctgctgcctccactgggcgtggccatctctgtgatcatcatcttttattgttacagagtgaacaggcagcagaagcgccggcgctag

SEQ ID NO: 126-127 - skipped (2 entries)

SEQ ID NO: 128 - protein, 10 aa, synthetic construct
TSNIGNNYVS

SEQ ID NO: 129 - protein, 11 aa, synthetic construct
LMIYDVSKRPS

SEQ ID NO: 130 - protein, 10 aa, synthetic construct
AAWDDSLSEF

SEQ ID NO: 131 - protein, 9 aa, synthetic construct
FTFGSFSMS

SEQ ID NO: 132 - protein, 14 aa, synthetic construct
WVAGLSARSSLTHY

SEQ ID NO: 133 - protein, 19 aa, synthetic construct
RRSYDSSGYWGHFYSYMDV

SEQ ID NO: 134 - protein, 208 aa, synthetic construct [sequence omitted]

SEQ ID NO: 135 - protein, 224 aa, synthetic construct
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 136 - protein, 92 aa, synthetic construct
FSQQIYGVVYGNVTFHVPSNVPLKEVLWKKQKDKVAELENSEFRAFSSFKNRVYLDTVSGSLTIYNLTSSDEDEYEMESPNITDTMKFFLYV

SEQ ID NO: 137 - protein, 323 aa, synthetic construct
IFPLAFFIYVPMNEQIVIGRLDEDIILPSSFERGSEVVIHWKYQDSYKVHSYYKGSDHLESQDPRYANRTSLFYNEIQNGNASLFFRRVSLLDEGIYTCYVGTAIQVITNKVVLKVGVFLTPVMKYEKRNTNSFLICSVLSVYPRPIITWKMDNTPISENNMEETGSLDSFSINSPLNITGSNSSYECTIENSLLKQTWTGRWTMKDGLHKMQSEHVSLSCQPVNDYFSPNQDFKVTWSRMKSGTFSVLAYYLSSSQNTIINESRFSWNKELINQSDFSMNLMDLNLSDSGEYLCNISSDEYTLLTIHTVHVEPSQETASHNK

SEQ ID NO: 138 - protein, 453 aa, synthetic construct
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYE

SEQ ID NO: 139 - protein, 133 aa, synthetic construct
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 140 - protein, 184 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 141 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLR

SEQ ID NO: 142 - protein, 323 aa, synthetic construct
WPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRN

SEQ ID NO: 143 - protein, 155 aa, synthetic construct
QRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 144 - protein, 161 aa, synthetic construct
LPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSS

SEQ ID NO: 145 - protein, 128 aa, synthetic construct
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 146 - protein, 172 aa, synthetic construct
QRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 147 - protein, 193 aa, synthetic construct
QGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLAR

SEQ ID NO: 148 - protein, 407 aa, synthetic construct
KDSAPTVVSGILGGSVTLPLNISVDTEIENVIWIGPKNALAFARPKENVTIMVKSYLGRLDITKWSYSLCISNLTLNDAGSYKAQINQRNFEVTTEEEFTLFVYEQLQEPQVTMKSVKVSENFSCNITLMCSVKGAEKSVLYSWTPREPHASESNGGSILTVSRTPCDPDLPYICTAQNPVSQRSSLPVHVGQFCTDPGASRGGTTGETVVGVLGEPVTLPLALPACRDTEKVVWLFNTSIISKEREEAATADPLIKSRDPYKNRVWVSSQDCSLKISQLKIEDAGPYHAYVCSEASSVTSMTHVTLLIYRRLRKPKITWSLRHSEDGICRISLTCSVEDGGNTVMYTWTPLQKEAVVSQGESHLNVSWRSSENHPNLTCTASNPVSRSSHQFLSENICSGPERNTK

SEQ ID NO: 149 - protein, 204 aa, synthetic construct
KDSEIFTVNGILGESVTFPVNIQEPRQVKIIAWTSKTSVAYVTPGDSETAPVVTVTHRNYYERIHALGPNYNLVISDLRMEDAGDYKADINTQADPYTTTKRYNLQIYRRLGKPKITQSLMASVNSTCNVTLTCSVEKEEKNVTYNWSPLGEEGNVLQIFQTPEDQELTYTCTAQNPVSNNSDSISARQLCADIAMGFRTHHTG

SEQ ID NO: 150 - protein, 204 aa, synthetic construct
QSSLTPLMVNGILGESVTLPLEFPAGEKVNFITWLFNETSLAFIVPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTK

SEQ ID NO: 151 - protein, 283 aa, synthetic construct
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSH

SEQ ID NO: 152 - protein, 243 aa, synthetic construct
EPHSLRYNLMVLSQDGSVQSGFLAEGHLDGQPFLRYDRQKRRAKPQGQWAEDVLGAETWDTETEDLTENGQDLRRTLTHIKDQKGVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTD

SEQ ID NO: 153 - protein, 244 aa, synthetic construct
MAAAASPAFLLCLPLLHLLSGWSRAGWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGTTQPKAMATTLSPWSLLIIFLCFILAGR

SEQ ID NO: 154 - protein, 246 aa, synthetic construct
MAAAAATKILLCLPLLLLLSGWSRAGRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMSSGTTQLRATATTLILCCLLIILPCFILPGI

SEQ ID NO: 155 - protein, 244 aa, synthetic construct
MAAAASPAILPRLAILPYLLFDWSGTGRADAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPGLAQPKAIATTLSPWSFLIILCFILPGI

SEQ ID NO: 156 - protein, 195 aa, synthetic construct
HSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPVNASDIHWSSSSLPD

SEQ ID NO: 157 - protein, 198 aa, synthetic construct
GLADPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAGAPPTMSSGTAQPR

SEQ ID NO: 158 - protein, 246 aa, synthetic construct
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 159 - protein, 238 aa, synthetic construct [sequence omitted]

SEQ ID NO: 160 - protein, 238 aa, synthetic construct
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFS

SEQ ID NO: 161 - protein, 162 aa, synthetic construct
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAA

SEQ ID NO: 162 - protein, 220 aa, synthetic construct
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEA

SEQ ID NO: 163 - protein, 244 aa, synthetic construct
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASTTENLYFQG

SEQ ID NO: 164 - protein, 128 aa, synthetic construct
LSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPG

SEQ ID NO: 165 - protein, 133 aa, synthetic construct [sequence omitted]

SEQ ID NO: 166 - protein, 152 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 167 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 168 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 169 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 170 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 171 - protein, 138 aa, synthetic construct
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 172 - DNA, 388 bp, synthetic construct
  source 1..388: mol_type = other DNA
aatgaaagaccccacctgtaggtttggcaagctaggatcaaggtcaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagagcccacaacccctcactcggc

SEQ ID NO: 173 - DNA, 66 bp, synthetic construct
  source 1..66: mol_type = other DNA
atgctgctgctggtgacaagcctgctgctgtgcgagctgcctcacccagcctttctgctgatcccc

SEQ ID NO: 174 - protein, 246 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 175 - DNA, 318 bp, synthetic construct
  source 1..318: mol_type = other DNA
gatatcgtgctgacccagtcccccgccatcctgtccgcctctcctggagagaaggtgaccatgacatgtcgggccagctcctctgtgaactacatggactggtatcagaagaagcctggcagctcccccaagccttggatctacgccacctccaatctggcctctggagtgccagcaagattcagcggatccggatctggcacaagctattccctgaccatctccagggtggaggcagaggatgcagcaacatactattgccagcagtggtctttcaacccccctacatttggcggcggcaccaagctggagatcaag

SEQ ID NO: 176 - protein, 106 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 177 - DNA, 54 bp, synthetic construct
  source 1..54: mol_type = other DNA
ggctctaccagcggaggaggaagcggaggaggatccggaggcggcggctctagc

SEQ ID NO: 178 - DNA, 366 bp, synthetic construct
  source 1..366: mol_type = other DNA
gaggtgcagctgcagcagtccggagcagagctggtgaagcctggagcctctgtgaagatgagctgtaaggcctccggctacaccttcacatcttataatatgcactgggtgaagcagacaccaggacagggactggagtggatcggagcaatctaccctggcaacggcgacaccagctataatcagaagtttaagggcaaggccaccctgacagccgataagtcctctagcacagcctacatgcagctgtcctctctgaccagcgaggactccgccgattactattgcgcccggtccaactactatggcagctcctattggttctttgacgtgtggggagcaggaacaaccgtgaccgtgtctagc

SEQ ID NO: 179 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 180 - DNA, 24 bp, synthetic construct
  source 1..24: mol_type = other DNA
gactacaaagacgatgacgacaag

SEQ ID NO: 181 - DNA, 216 bp, synthetic construct
  source 1..216: mol_type = other DNA
gcaaagccaaccaccacacctgctcctagaccacctacacccgctcctaccatcgccagccagcctctgtctctgagacctgaggcctgtagacctgccgctggaggcgctgtgcacaccagaggactggatttcgcctgcgacatctacatctgggctcctctggctggaacatgcggcgtgctgctcctgagcctggtgatcacactgtactgc

SEQ ID NO: 182 - DNA, 144 bp, synthetic construct
  source 1..144: mol_type = other DNA
gcaaagccaaccaccacacctgctcctagaccacctacacccgctcctaccatcgccagccagcctctgtctctgagacctgaggcctgtagacctgccgctggaggcgctgtgcacaccagaggactggatttcgcctgcgac

SEQ ID NO: 183 - DNA, 72 bp, synthetic construct
  source 1..72: mol_type = other DNA
atctacatctgggctcctctggctggaacatgcggcgtgctgctcctgagcctggtgatcacactgtactgc

SEQ ID NO: 184 - DNA, 126 bp, synthetic construct
  source 1..126: mol_type = other DNA
aagagaggcaggaagaagctgctgtatatctttaagcagcccttcatgcgccctgtgcagaccacacaggaggaggacggctgcagctgtcggtttccagaggaggaggagggaggatgcgagctg

SEQ ID NO: 185 - DNA, 336 bp, synthetic construct
  source 1..336: mol_type = other DNA
cgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacctatgatgccctgcacatgcaggccctgccccctcgg

SEQ ID NO: 186 - DNA, 1482 bp, synthetic construct
  source 1..1482: mol_type = other DNA
gatatcgtgctgacccagtcccccgccatcctgtccgcctctcctggagagaaggtgaccatgacatgtcgggccagctcctctgtgaactacatggactggtatcagaagaagcctggcagctcccccaagccttggatctacgccacctccaatctggcctctggagtgccagcaagattcagcggatccggatctggcacaagctattccctgaccatctccagggtggaggcagaggatgcagcaacatactattgccagcagtggtctttcaacccccctacatttggcggcggcaccaagctggagatcaagggctctaccagcggaggaggaagcggaggaggatccggaggcggcggctctagcgaggtgcagctgcagcagtccggagcagagctggtgaagcctggagcctctgtgaagatgagctgtaaggcctccggctacaccttcacatcttataatatgcactgggtgaagcagacaccaggacagggactggagtggatcggagcaatctaccctggcaacggcgacaccagctataatcagaagtttaagggcaaggccaccctgacagccgataagtcctctagcacagcctacatgcagctgtcctctctgaccagcgaggactccgccgattactattgcgcccggtccaactactatggcagctcctattggttctttgacgtgtggggagcaggaacaaccgtgaccgtgtctagcgctgcaggaggcggaggatctggaggcggcggcggagactacaaagacgatgacgacaagttcgaagcaaagccaaccaccacacctgctcctagaccacctacacccgctcctaccatcgccagccagcctctgtctctgagacctgaggcctgtagacctgccgctggaggcgctgtgcacaccagaggactggatttcgcctgcgacatctacatctgggctcctctggctggaacatgcggcgtgctgctcctgagcctggtgatcacactgtactgcaagagaggcaggaagaagctgctgtatatctttaagcagcccttcatgcgccctgtgcagaccacacaggaggaggacggctgcagctgtcggtttccagaggaggaggagggaggatgcgagctgcgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacctatgatgccctgcacatgcaggccctgccccctcgg

SEQ ID NO: 187 - protein, 13 aa, synthetic construct
ESKYGPPCPPCPM

SEQ ID NO: 188 - protein, 13 aa, synthetic construct
ESKYGPPSPPSPA

SEQ ID NO: 189 - protein, 12 aa, synthetic construct
ESKYGPPSPPSP

SEQ ID NO: 190 - protein, 13 aa, synthetic construct
PSPRPAGQFQTLV

SEQ ID NO: 191 - protein, 12 aa, synthetic construct
EPKSCDKTHTCP

SEQ ID NO: 192 - protein, 13 aa, synthetic construct
GAVHTRGLDFACD

SEQ ID NO: 193 - protein, 13 aa, synthetic construct
LCPSPLFPGPSKP

SEQ ID NO: 194 - DNA, 735 bp, synthetic construct; identical to SEQ ID NO: 102
  source 1..735: mol_type = other DNA

SEQ ID NO: 195 - protein, 245 aa, synthetic construct; identical to SEQ ID NO: 93

SEQ ID NO: 196 - DNA, 39 bp, synthetic construct
  source 1..39: mol_type = other DNA
ggcgctgtgcacaccagaggactggatttcgcctgcgac

SEQ ID NO: 197 - protein, 13 aa, synthetic construct
GAVHTRGLDFACD

SEQ ID NO: 198 - DNA, 81 bp, synthetic construct
  source 1..81: mol_type = other DNA
ttctgggtgctggtggtggtgggaggcgtgctggcctgttactccctgctggtgaccgtggcctttatcatcttctgggtg

SEQ ID NO: 199 - protein, 27 aa, synthetic construct
FWVLVVVGGVLACYSLLVTVAFIIFWV

SEQ ID NO: 200 - DNA, 126 bp, synthetic construct; identical to SEQ ID NO: 184
  source 1..126: mol_type = other DNA

SEQ ID NO: 201 - protein, 42 aa, synthetic construct; identical to SEQ ID NO: 84

SEQ ID NO: 202 - DNA, 336 bp, synthetic construct; identical to SEQ ID NO: 83
  source 1..336: mol_type = other DNA

SEQ ID NO: 203 - protein, 112 aa, synthetic construct; identical to SEQ ID NO: 82

SEQ ID NO: 204 - DNA, 1317 bp, synthetic construct
  source 1..1317: mol_type = other DNA
gacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatcaccggatccacatctggaagcggcaagccaggaagcggagagggatccacaaagggagaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagcggcgctgtgcacaccagaggactggatttcgcctgcgacttctgggtgctggtggtggtgggaggcgtgctggcctgttactccctgctggtgaccgtggcctttatcatcttctgggtgaagagaggcaggaagaagctgctgtatatctttaagcagcccttcatgcgccctgtgcagaccacacaggaggaggacggctgcagctgtcggtttccagaggaggaggagggaggatgcgagctgcgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacatatgatgccctgcacatgcaggccctgccacctagg

SEQ ID NO: 205 - protein, 439 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 206 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 207 - protein, 18 aa, synthetic construct
GSTSGSGKPGSGEGSTKG

SEQ ID NO: 208 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 209 - protein, 494 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSAAGGGGSGGGGGDYKDDDDKFEAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 210 - protein, 106 aa, synthetic construct; identical to SEQ ID NO: 176

SEQ ID NO: 211 - protein, 8 aa, synthetic construct
DYKDDDDK

SEQ ID NO: 212 - protein, 72 aa, synthetic construct
AKPTTTPAPRPPTPAPTIASQPLSLRPEACRPAAGGAVHTRGLDFACDIYIWAPLAGTCGVLLLSLVITLYC

SEQ ID NO: 213 - protein, 42 aa, synthetic construct; identical to SEQ ID NO: 84

SEQ ID NO: 214 - protein, 112 aa, synthetic construct; identical to SEQ ID NO: 82

SEQ ID NO: 215 - protein, 275 aa, synthetic construct; identical to SEQ ID NO: 60

SEQ ID NO: 216 - DNA, 825 bp, synthetic construct
  source 1..825: mol_type = other DNA
atgggcgtgaaagtgctgttcgccctgatctgcatcgccgtggctgaggccttttctcagcaaatctacggcgtggtgtacggcaacgtgaccttccatgtgcctagcaacgtgccactcaaggaagtgctgtggaagaaacagaaagacaaggtggccgagctggaaaacagcgagttcagagccttcagcagcttcaagaacagagtgtacctggacaccgtgtccggcagcctgaccatttacaacctgaccagcagcgacgaggacgagtacgagatggaaagccctaatatcacagataccatgaagttcttcctgtacgtgctggaatctctgggcagcggagttatccacgtgaccaaggaggtgaaagaagtggctacactgagctgcggccacaacgtctccgtcgaggaactggcccagaccagaatctattggcaaaaggaaaagaagatggtgctgacaatgatgagcggagatatgaacatctggcctgagtacaagaatcggaccatctttgacatcaccaacaacctgtctatcgtgatcctggctctgcgcccctccgacgaaggaacatacgagtgcgtggtcctcaagtacgagaaggacgccttcaagagagagcacctggccgaggtgaccctgagcgtgaaggccgattttcccacacctgataatctgctgcctagctgggccatcaccctgatcagcgtgaacggcatcttcgtgatttgttgtctgacatactgcttcgcccctagatgcagagagcggcggcggaacgagagactgagacgggaaagcgttagacccgtg

SEQ ID NO: 217 - protein, 286 aa, synthetic construct; identical to SEQ ID NO: 61

SEQ ID NO: 218 - DNA, 858 bp, synthetic construct
  source 1..858: mol_type = other DNA
atggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgaattcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttggctccggagttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 219 - protein, 266 aa, synthetic construct; identical to SEQ ID NO: 62

SEQ ID NO: 220 - DNA, 798 bp, synthetic construct
  source 1..798: mol_type = other DNA
atgggagtgaaggtgcttttcgccctgatctgtatcgccgtggctgaggccgtgattcacgtgaccaaggaagtcaaggaagttgccaccctgtcatgcggccacaacgtgtccgtggaagagctggctcaaacaagaatctattggcagaaggagaagaagatggtgctgaccatgatgagcggcgacatgaacatctggcctgagtacaagaacagaacaatcttcgacattacaaacaacctgagcatcgtgatcctggccctgcggcctagcgatgagggcacctacgagtgcgtggtgctgaaatacgagaaggacgcctttaagcgggaacacctggcagaggtcacactgagcgtgaaggccggctctggattcagccagcaaatctacggcgtggtgtacggcaacgttaccttccacgtgccatctaatgtgcccctgaaagaggtgctgtggaagaaacagaaagataaggtggccgaactcgagaacagcgagttcagagcctttagcagcttcaagaaccgggtgtacctggacaccgtgtccggttctctgacaatctacaacctgacaagcagcgacgaagatgagtatgagatggaaagccccaatatcaccgacaccatgaaattcttcctgtacgtgctggaatccctgccttcttccggacatagcagacacagatacgccctgatccccatccctctggctgtgatcaccacctgcatcgtgctgtacatgaacggcatcctgaagtgcgacagaaagcctgataggaccaacagcaat

SEQ ID NO: 221 - protein, 283 aa, synthetic construct; identical to SEQ ID NO: 63

SEQ ID NO: 222 - DNA, 849 bp, synthetic construct
  source 1..849: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctggctccggattttcccaacaaatatatggtgttgtgtatgggaatgtaactttccatgtaccaagcaatgtgcctttaaaagaggtcctatggaaaaaacaaaaggataaagttgcagaactggaaaattctgaattcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccaagcagcggtcattcaagacacagatatgcacttatacccataccattagcagtaattacaacatgtattgtgctgtatatgaatggtattctgaaatgtgacagaaaaccagacagaaccaactccaat

SEQ ID NO: 223 - protein, 348 aa, synthetic construct; identical to SEQ ID NO: 64

SEQ ID NO: 224 - DNA, 1044 bp, synthetic construct
  source 1..1044: mol_type = other DNA
atgggcgtgaaagtgctgttcgccctgatctgcatcgcagttgctgaagccgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctggctccggattttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtattctgaaatgtgacagaaaaccagacagaaccaactccaat

SEQ ID NO: 225 - protein, 370 aa, synthetic construct; identical to SEQ ID NO: 65

SEQ ID NO: 226 - DNA, 1110 bp, synthetic construct
  source 1..1110: mol_type = other DNA
atgggcgtgaaagtgctgttcgccctgatctgcatcgcagttgctgaagccttttcccaacaaatatatggtgttgtgtatgggaatgtaactttccatgtaccaagcaatgtgcctttaaaagaggtcctatggaaaaaacaaaaggataaagttgcagaactggaaaattctgaattcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttggctccggagttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 227 - protein, 365 aa, synthetic construct; identical to SEQ ID NO: 66

SEQ ID NO: 228 - DNA, 1095 bp, synthetic construct
  source 1..1095: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctggctccggattttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtattctgaaatgtgacagaaaaccagacagaaccaactccaat

SEQ ID NO: 229 - protein, 381 aa, synthetic construct; identical to SEQ ID NO: 67

SEQ ID NO: 230 - DNA, 1143 bp, synthetic construct
  source 1..1143: mol_type = other DNA
atggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttggctccggagttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 231 - protein, 467 aa, synthetic construct; identical to SEQ ID NO: 68

SEQ ID NO: 232 - DNA, 1401 bp, synthetic construct
  source 1..1401: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacggctccggattttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtattctgaaatgtgacagaaaaccagacagaaccaactccaat

SEQ ID NO: 233 - protein, 472 aa, synthetic construct; identical to SEQ ID NO: 32

SEQ ID NO: 234 - DNA, 1416 bp, synthetic construct
  source 1..1416: mol_type = other DNA
atggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagaggctccggagttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 235 - protein, 613 aa, synthetic construct; identical to SEQ ID NO: 69

SEQ ID NO: 236 - DNA, 1839 bp, synthetic construct
  source 1..1839: mol_type = other DNA
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgccggttcttctggaggatcaggaggtggaggatcaggtggaggtggatcaggtggaggaggatcagttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctggctccggattttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtattctgaaatgtgacagaaaaccagacagaaccaactccaat

SEQ ID NO: 237 - protein, 635 aa, synthetic construct; identical to SEQ ID NO: 70

SEQ ID NO: 238 - DNA, 1905 bp, synthetic construct
  source 1..1905: mol_type = other DNA
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgccggttcttctggaggatcaggaggtggaggatcaggtggaggtggatcaggtggaggaggatcattttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttggctccggagttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 239 - protein, 632 aa, synthetic construct; identical to SEQ ID NO: 71

SEQ ID NO: 240 - DNA, 1896 bp, synthetic construct
  source 1..1896: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctggctccggagacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgccggttcttctggaggatcaggaggtggaggatcaggtggaggtggatcaggtggaggaggatcatccggattttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtattctgaaatgtgacagaaaaccagacagaaccaactccaat

SEQ ID NO: 241 - protein, 648 aa, synthetic construct; identical to SEQ ID NO: 33

SEQ ID NO: 242 - DNA, 1944 bp, synthetic construct
  source 1..1944: mol_type = other DNA
atggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttggctccggagacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgccggttcttctggaggatcaggaggtggaggatcaggtggaggtggatcaggtggaggaggatcatccggagttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 243 - protein, 726 aa, synthetic construct; identical to SEQ ID NO: 73

SEQ ID NO: 244 - DNA, 2178 bp, synthetic construct
  source 1..2178: mol_type = other DNA
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgccggttcttctggaggatcaggaggtggaggatcaggtggaggtggatcaggtggaggaggatcattttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagaggctccggagttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 245 - protein, 648 aa, synthetic construct; identical to SEQ ID NO: 33

SEQ ID NO: 246 - DNA, 2217 bp, synthetic construct
  source 1..2217: mol_type = other DNA
atggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagaggctccggagacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgccggttcttctggaggatcaggaggtggaggatcaggtggaggtggatcaggtggaggaggatcatccggagttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 247 - protein, 512 aa, synthetic construct
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 248 - protein, 96 aa, synthetic construct
FSQQIYGVVYGNVTFHVPSNVPLKEVLWKKQKDKVAELENSEFRAFSSFKNRVYLDTVSGSLTIYNLTSSDEDEYEMESPNITDTMKFFLYVLESL

SEQ ID NO: 249 - protein, 244 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSA

SEQ ID NO: 250 - protein, 254 aa, synthetic construct
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 251 - protein, 90 aa, synthetic construct
MEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 252 - protein, 93 aa, synthetic construct
ILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 253 - protein, 106 aa, synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKL

SEQ ID NO: 254 - protein, 819 aa, synthetic construct
MEMWHEGLEEASRLYFGERNVKGMFEVLEPLHAMMERGPQTLKETSFNQAYGRDLMEAQEWCRKYMKSGNVKDLLQAWDLYYHVFRRISKASRRKRGSGEGRGSLLTCGDVEENPGPMPLPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 255 - protein, 1301 aa, synthetic construct
MEMWHEGLEEASRLYFGERNVKGMFEVLEPLHAMMERGPQTLKETSFNQAYGRDLMEAQEWCRKYMKSGNVKDLLQAWDLYYHVFRRISKASRRKRGSGEGRGSLLTCGDVEENPGPMPLPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGATNFSLLKQAGDVEENPGPLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 256 - DNA, 270 bp, synthetic construct
  source 1..270: mol_type = other DNA
atggagatgtggcacgagggactggaggaggcaagcagactgtactttggcgagaggaacgtgaagggcatgttcgaggtgctggagccactgcacgcaatgatggagaggggacctcagacactgaaggagacctccttcaaccaggcctatggcagagacctgatggaggcccaggagtggtgcaggaagtacatgaagtctggcaatgtgaaggacctgctgcaggcctgggatctgtactatcacgtgtttcggagaatcagcaag

SEQ ID NO: 257 - DNA, 279 bp, synthetic construct
  source 1..279: mol_type = other DNA
atcctgtggcacgagatgtggcacgagggcctggaggaggctagcagactgtacttcggcgagagaaacgtgaagggcatgttcgaggtgctggagcccctgcacgccatgatggagagaggccctcagaccctgaaggagacaagcttcaaccaagcctacggcagagacctgatggaggcccaagagtggtgcagaaagtacatgaagagcggcaacgtgaaggacctgctgcaagcctgggacctgtactaccacgtgttcagaagaatcagcaag

SEQ ID NO: 258 - DNA, 279 bp, synthetic construct
  source 1..279: mol_type = other DNA
atggagatgtggcacgagggactggaggaggcaagcagactgtactttggcgagaggaacgtgaagggcatgttcgaggtgctggagccactgcacgcaatgatggagaggggacctcagacactgaaggagacctccttcaaccaggcctatggcagagacctgatggaggcccaggagtggtgcaggaagtacatgaagtctggcaatgtgaaggacctgctgcaggcctgggatctgtactatcacgtgtttcggagaatcagcaagggctccggc

SEQ ID NO: 259 - skipped

SEQ ID NO: 260 - protein, 93 aa, synthetic construct
MEMWHEGLEEASRLYFGERNVKGMFEVLEPLHAMMERGPQTLKETSFNQAYGRDLMEAQEWCRKYMKSGNVKDLLQAWDLYYHVFRRISKGSG

SEQ ID NO: 261 - DNA, 369 bp, synthetic construct
  source 1..369: mol_type = other DNA
ggcagcaatacaagcaaggaaaaccccttcctgttcgccctggaggccgtggtgatcagcgtgggcagcatgggcctgatcatcagcctgctgtgcgtgtacttctggctggagagaaccatgcctagaatccccaccctgaagaacctggaggacctggtgaccgagtaccacggcaacttcagcgcctggagcggcgtgagcaagggcctggccgagagcctgcagcccgactacagcgagcgactgtgcctggtgagcgagattccccctaagggcggggccttgggtgagggacccggggcaagcccgtgcaatcagcacagcccctactgggcccccccctgttacaccctgaagcccgagacc

SEQ ID NO: 262 - DNA, 762 bp, synthetic construct
  source 1..762: mol_type = other DNA
atgccactgggactgctgtggctgggactggccctgctgggcgccctgcacgcccaggcaggagtgcaggtggagaccatctccccaggcgacggacgcacatttccaaagaggggacagacctgcgtggtgcactacacaggcatgctggaggatggcaagaagttcgacagctcccgcgatcggaataagccctttaagttcatgctgggcaagcaggaagtgatcagaggatgggaggagggagtggcacagatgtccgtgggacagagggccaagctgaccatctctcctgactacgcctatggagcaacaggacacccaggaatcatcccacctcacgccaccctggtgtttgatgtggagctgctgaagctgggcgagggctctaacacaagcaaggagaatccttttctgttcgcactggaggcagtggtcatctccgtgggctctatgggcctgatcatcagcctgctgtgcgtgtacttttggctggagagaaccatgccaaggatccccacactgaagaacctggaggacctggtgaccgagtatcacggcaatttcagcgcctggtccggcgtgtctaagggactggcagagtccctgcagccagattactctgagcggctgtgcctggtgtccgagatcccacccaagggaggcgccctgggagagggaccaggagccagcccttgcaaccagcactccccatattgggcccctccatgttacacactgaagcccgagaccggctctggc

SEQ ID NO: 263 - DNA, 753 bp, synthetic construct
  source 1..753: mol_type = other DNA
atgccactgggactgctgtggctgggactggccctgctgggcgccctgcacgcccaggcaggagtgcaggtggagaccatctccccaggcgacggacgcacatttccaaagaggggacagacctgcgtggtgcactacacaggcatgctggaggatggcaagaagttcgacagctcccgcgatcggaataagccctttaagttcatgctgggcaagcaggaagtgatcagaggatgggaggagggagtggcacagatgtccgtgggacagagggccaagctgaccatctctcctgactacgcctatggagcaacaggacacccaggaatcatcccacctcacgccaccctggtgtttgatgtggagctgctgaagctgggcgagggctctaacacaagcaaggagaatccttttctgttcgcactggaggcagtggtcatctccgtgggctctatgggcctgatcatcagcctgctgtgcgtgtacttttggctggagagaaccatgccaaggatccccacactgaagaacctggaggacctggtgaccgagtatcacggcaatttcagcgcctggtccggcgtgtctaagggactggcagagtccctgcagccagattactctgagcggctgtgcctggtgtccgagatcccacccaagggaggcgccctgggagagggaccaggagccagcccttgcaaccagcactccccatattgggcccctccatgttacacactgaagcccgagacc

SEQ ID NO: 264 - protein, 123 aa, synthetic construct
GSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 265 - protein, 251 aa, synthetic construct
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 266 - DNA, 648 bp, synthetic construct
  source 1..648: mol_type = other DNA
atcctgtggcacgagatgtggcacgagggcctggaggaggctagcagactgtacttcggcgagagaaacgtgaagggcatgttcgaggtgctggagcccctgcacgccatgatggagagaggccctcagaccctgaaggagacaagcttcaaccaagcctacggcagagacctgatggaggcccaagagtggtgcagaaagtacatgaagagcggcaacgtgaaggacctgctgcaagcctgggacctgtactaccacgtgttcagaagaatcagcaagggcagcaatacaagcaaggaaaaccccttcctgttcgccctggaggccgtggtgatcagcgtgggcagcatgggcctgatcatcagcctgctgtgcgtgtacttctggctggagagaaccatgcctagaatccccaccctgaagaacctggaggacctggtgaccgagtaccacggcaacttcagcgcctggagcggcgtgagcaagggcctggccgagagcctgcagcccgactacagcgagcgactgtgcctggtgagcgagattccccctaagggcggggccttgggtgagggacccggggcaagcccgtgcaatcagcacagcccctactgggcccccccctgttacaccctgaagcccgagacc

SEQ ID NO: 267 - protein, 216 aa, synthetic construct
ILWHEMWHEGLEEASRLYFGERNVKGMFEVLEPLHAMMERGPQTLKETSFNQAYGRDLMEAQEWCRKYMKSGNVKDLLQAWDLYYHVFRRISKGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 268 - DNA, 318 bp, synthetic construct
  source 1..318: mol_type = other DNA
ggcgttcaagtggagactatctcccccggcgacggcagaaccttccctaagagagggcagacctgcgtggtgcactacaccggcatgctggaggacggcaagaagttcgacagcagcagagacagaaacaagcccttcaagttcatgctgggcaagcaagaggtgatcagaggctgggaggagggtgtggcccaaatgagcgtggggcagagagccaaattgaccatctcacccgactacgcttacggggccaccggccatcccggcatcatccccccccacgccaccctggtgttcgacgtggagctgctgaagctg

SEQ ID NO: 269 - DNA, 318 bp, synthetic construct
  source 1..318: mol_type = other DNA
ggagtgcaggtggagaccatctccccaggcgacggacgcacatttccaaagaggggacagacctgcgtggtgcactacacaggcatgctggaggatggcaagaagttcgacagctcccgcgatcggaataagccctttaagttcatgctgggcaagcaggaagtgatcagaggatgggaggagggagtggcacagatgtccgtgggacagagggccaagctgaccatctctcctgactacgcctatggagcaacaggacacccaggaatcatcccacctcacgccaccctggtgtttgatgtggagctgctgaagctg

SEQ ID NO: 270 - DNA, 945 bp, synthetic construct
  source 1..945: mol_type = other DNA
ggcaaggacaccatcccctggctgggccacctgctggtgggcctgagcggcgccttcggcttcatcatcctggtgtacctgctgatcaactgcagaaacaccggcccctggctgaagaaggtgctgaagtgcaacacccccgaccctagcaagttcttctctcagctgagcagcgagcacggcggcgacgtgcagaagtggctgagcagcccatttcctagcagcagctttagccccggcggcctagctcccgagatcagccccctggaagtactggagagagataaggtgacacagctgctgctgcagcaagacaaggtgcccgagcccgctagcctgagcagcaaccacagcctgacaagctgcttcaccaaccaaggctacttcttcttccacctgcccgacgccctggagatcgaggcctgccaagtgtacttcacctacgatccgtacagcgaggaagaccccgacgagggagtggccggcgcccctaccggcagctcgccacaaccgctgcaacctctgagcggcgaggacgacgcctactgcaccttccctagcagagatgatctgctgctcttttcccctagcttgctggggggcccttcgcctccaagtactgcccccggaggtagcggggccggcgaagaaagaatgcccccaagcctgcaagaaagagtgcctagagactgggaccctcaaccactgggcccacccactccgggcgtgcccgacctggtagattttcagccccctcccgagctggtgctacgcgaggccggcgaagaggtgcccgacgctgggccaagagagggtgtgtcattcccctggagccgaccccccggtcaaggcgagttcagagccctgaacgctagactgcccctgaacaccgacgcctacctgagcctgcaagagctgcaaggccaagaccccacccacctggtg

SEQ ID NO: 271 - DNA, 946 bp, synthetic construct
  source 1..946: mol_type = other DNA
gggcaaggataccatcccctggctgggacacctgctggtgggactgagcggagcctttggcttcatcatcctggtgtacctgctgatcaactgcagaaataccggcccttggctgaagaaggtgctgaagtgtaacacaccagacccctccaagttcttttctcagctgtctagcgagcacggcggcgatgtgcagaagtggctgtcctctccttttccaagctcctctttcagcccaggaggactggcaccagagatctcccccctggaggtgctggagagggacaaggtgacccagctgctgctgcagcaggataaggtgcccgagcctgccagcctgagctccaaccacagcctgacatcctgctttaccaatcagggctatttctttttccacctgccagacgccctggagatcgaggcctgtcaggtgtacttcacctatgatccatactccgaagaggacccagatgagggagtggccggcgcccccacaggctctagcccacagccactgcagcctctgtctggagaggacgatgcctactgtacctttcccagccgcgacgatctgctgctgttcagcccttccctgctgggaggaccatctccacctagcacagcaccaggaggaagcggggcaggggaggagcggatgccaccatctctgcaggagagggtgcctagggactgggatcctcagccactgggacctccaacccctggagtgccagacctggtggattttcagccccctccagagctggtgctgcgggaggcaggagaggaggtgcctgacgcaggaccacgcgagggcgtgagctttccatggtccaggccacctggacagggagagttcagagccctgaacgccaggctgcctctgaatacagacgcctatctgtctctgcaggagctgcagggccaggatccaacccacctggtg

SEQ ID NO: 272 - protein, 315 aa, synthetic construct
GKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 273 - protein, 315 aa, synthetic construct; identical to SEQ ID NO: 272

SEQ ID NO: 274 - DNA, 1269 bp, synthetic construct
  source 1..1269: mol_type = other DNA
ggcgttcaagtggagactatctcccccggcgacggcagaaccttccctaagagagggcagacctgcgtggtgcactacaccggcatgctggaggacggcaagaagttcgacagcagcagagacagaaacaagcccttcaagttcatgctgggcaagcaagaggtgatcagaggctgggaggagggtgtggcccaaatgagcgtggggcagagagccaaattgaccatctcacccgactacgcttacggggccaccggccatcccggcatcatccccccccacgccaccctggtgttcgacgtggagctgctgaagctgggcgagggcaaggacaccatcccctggctgggccacctgctggtgggcctgagcggcgccttcggcttcatcatcctggtgtacctgctgatcaactgcagaaacaccggcccctggctgaagaaggtgctgaagtgcaacacccccgaccctagcaagttcttctctcagctgagcagcgagcacggcggcgacgtgcagaagtggctgagcagcccatttcctagcagcagctttagccccggcggcctagctcccgagatcagccccctggaagtactggagagagataaggtgacacagctgctgctgcagcaagacaaggtgcccgagcccgctagcctgagcagcaaccacagcctgacaagctgcttcaccaaccaaggctacttcttcttccacctgcccgacgccctggagatcgaggcctgccaagtgtacttcacctacgatccgtacagcgaggaagaccccgacgagggagtggccggcgcccctaccggcagctcgccacaaccgctgcaacctctgagcggcgaggacgacgcctactgcaccttccctagcagagatgatctgctgctcttttcccctagcttgctggggggcccttcgcctccaagtactgcccccggaggtagcggggccggcgaagaaagaatgcccccaagcctgcaagaaagagtgcctagagactgggaccctcaaccactgggcccacccactccgggcgtgcccgacctggtagattttcagccccctcccgagctggtgctacgcgaggccggcgaagaggtgcccgacgctgggccaagagagggtgtgtcattcccctggagccgaccccccggtcaaggcgagttcagagccctgaacgctagactgcccctgaacaccgacgcctacctgagcctgcaagagctgcaaggccaagaccccacccacctggtg

SEQ ID NO: 275 - protein, 423 aa, synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 276 - DNA, 1287 bp, synthetic construct
  source 1..1287: mol_type = other DNA
atggcactgcctgtgaccgccctgctgctgccactggccctgctgctgcacgcagcaaggcctatcctgtggcacgaaatgtggcatgaaggcctggaggaggcaagcaggctgtattttggcgagcggaatgtgaaaggaatgtttgaagtcctggaacctctgcatgccatgatggaaaggggaccacagacactgaaggagaccagctttaaccaggcctatggaagggacctgatggaggcacaggagtggtgccggaagtacatgaagtccggaaatgtgaaagacctgctgcaggcctgggatctgtattatcacgtgttcaggcgcatctctaagggcaaggataccatcccctggctgggacacctgctggtgggactgagcggagcctttggcttcatcatcctggtgtacctgctgatcaactgcagaaataccggcccttggctgaagaaggtgctgaagtgtaacacaccagacccctccaagttcttttctcagctgtctagcgagcacggcggcgatgtgcagaagtggctgtcctctccttttccaagctcctctttcagcccaggaggactggcaccagagatctcccccctggaggtgctggagagggacaaggtgacccagctgctgctgcagcaggataaggtgcccgagcctgccagcctgagctccaaccacagcctgacatcctgctttaccaatcagggctatttctttttccacctgccagacgccctggagatcgaggcctgtcaggtgtacttcacctatgatccatactccgaagaggacccagatgagggagtggccggcgcccccacaggctctagcccacagccactgcagcctctgtctggagaggacgatgcctactgtacctttcccagccgcgacgatctgctgctgttcagcccttccctgctgggaggaccatctccacctagcacagcaccaggaggaagcggggcaggggaggagcggatgccaccatctctgcaggagagggtgcctagggactgggatcctcagccactgggacctccaacccctggagtgccagacctggtggattttcagccccctccagagctggtgctgcgggaggcaggagaggaggtgcctgacgcaggaccacgcgagggcgtgagctttccatggtccaggccacctggacagggagagttcagagccctgaacgccaggctgcctctgaatacagacgcctatctgtctctgcaggagctgcagggccaggatccaacccacctggtg

SEQ ID NO: 277 - protein, 429 aa, synthetic construct
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 278 - protein, 18 aa, synthetic construct
GSTSGGGSGGGSGGGGSS

SEQ ID NO: 279 - DNA, 738 bp, synthetic construct
  source 1..738: mol_type = other DNA
gatatcgtgctgacccagtcccccgccatcctgtccgcctctcctggagagaaggtgaccatgacatgtcgggccagctcctctgtgaactacatggactggtatcagaagaagcctggcagctcccccaagccttggatctacgccacctccaatctggcctctggagtgccagcaagattcagcggatccggatctggcacaagctattccctgaccatctccagggtggaggcagaggatgcagcaacatactattgccagcagtggtctttcaacccccctacatttggcggcggcaccaagctggagatcaagggctctaccagcggaggaggaagcggaggaggatccggaggcggcggctctagcgaggtgcagctgcagcagtccggagcagagctggtgaagcctggagcctctgtgaagatgagctgtaaggcctccggctacaccttcacatcttataatatgcactgggtgaagcagacaccaggacagggactggagtggatcggagcaatctaccctggcaacggcgacaccagctataatcagaagtttaagggcaaggccaccctgacagccgataagtcctctagcacagcctacatgcagctgtcctctctgaccagcgaggactccgccgattactattgcgcccggtccaactactatggcagctcctattggttctttgacgtgtggggagcaggaacaaccgtgaccgtgtctagc

SEQ ID NO: 280 - protein, 48 aa, synthetic construct
AKPTTTPAPRPPTPAPTIASQPLSLRPEACRPAAGGAVHTRGLDFACD

SEQ ID NO: 281 - protein, 24 aa, synthetic construct
IYIWAPLAGTCGVLLLSLVITLYC

(19) United States
(12) Patent Application Publication
(10) Pub. No.: US 2026/0308622 A1
(43) Pub. Date: Oct. 8, 2026

(54) LENTIVIRAL DELIVERY OF ANTI-CD20 CHIMERIC ANTIGEN RECEPTORS

(71) Applicant: Umoja Biopharma, Inc., Seattle, WA (US)

(72) Inventors: Christopher Nicolai, Seattle, WA (US); Weiliang Tang, Seattle, WA (US); Byoung Ryu, Seattle, WA (US); Ryan Larson, Seattle, WA (US); Jim Qin, Seattle, WA (US); Rebecca Jane Gottschalk, Seattle, WA (US); Wai-hang Leung, Seattle, WA (US); Lisa Yun Park, Seattle, WA (US); Maura Parker, Seattle, WA (US)

(21) Appl. No.: 19/481,481

(22) PCT Filed: May 1, 2024
(86) PCT No.: PCT/US2024/027211

§ 371 Date: Nov. 4, 2025

Related U.S. Application Data
(60) Provisional application No. 63/466,471, filed on May 15, 2023.
(60) Provisional application No. 63/579,188, filed on Aug. 28, 2023.

(51) Int. Cl. C12N 15/86; C07K 14/705; C07K 16/28; C12N 9/90

(52) U.S. Cl. CPC C12N 15/86; C07K 14/70528; C07K 14/70532; C07K 16/2809; C07K 16/2887; C12N 9/90; C12Y 502/01008; C07K 2317/33; C07K 2317/622; C07K 2319/00; C12N 2740/15043

(57) ABSTRACT

Provided herein are particles comprising polynucleotide constructs for generating cells expressing anti-CD20 chimeric antigen receptor, as well as vectors, such as lentiviral vectors, comprising same, cells comprising same, and methods of using same.

### CROSS-REFERENCE

This application claims the benefit of U.S. Provisional Application No. 63/466,471, filed May 15, 2023, U.S. Provisional Application No. 63/579,188, filed Aug. 28, 2023, and PCT Application No. PCT/US2023/078688, filed Nov. 3, 2023, which applications are incorporated herein by reference in their entireties.

### INCORPORATION BY REFERENCE OF SEQUENCE LISTING

The present application is being filed along with a Sequence Listing in electronic format. The Sequence Listing is provided as a file entitled 061479_506002WO_SeqList.xml, created Mar. 17, 2024, which is 343 kilobytes in size. The information in the electronic format of the Sequence Listing is incorporated by reference in its entirety.

### FIELD OF THE INVENTION

This disclosure relates generally to cell biology, immunology, and medicine—more particularly to lentiviral particles for use as medical treatments.

### BACKGROUND

T cells may be genetically engineered for use as therapeutic agents. For example, some chimeric antigen receptor (CAR) T cells have been approved as treatments for liquid tumors. Improved methods and compositions for enhancing T cells are needed. Genetic engineering of T cells may require delivery of polynucleotides into the T cells selected for engineering, a procedure termed transduction. Transduction of T cells may be achieved using various viral and non-viral delivery vehicles. In one example, a recombinant lentivirus is used for transduction. The lentiviral particle may be engineered to display, on its surface, molecules that enhance transduction. An antibody or antibody fragment against a component of a T cell receptor, such as CD3, may be surface-displayed on the lentivirus to target the virus to T cells. Additionally, engagement of CD3 by a lentivirus comprising a binding domain targeting CD3 may cause the T cells to activate via a primary activation signal. Surface display of one or more ligands for a co-receptor, such as CD28, a molecule expressed by T cells, may cause the T cells to activate via a secondary activation signal. Activation of a T cell, via the primary and optionally secondary activation signals, which may make them more susceptible to transduction. Ligands for CD28 may include, for example, CD80 and CD86.

There remains a need in the art for new compositions and methods that further improve transduction of T cells by delivery vehicles such as recombinant lentiviruses. The present disclosure addresses this need.

### SUMMARY

Disclosed herein, in some embodiments, are lentiviral particles comprising: a polynucleotide encoding a chimeric antigen receptor (CAR) that specifically binds cluster of differentiation-20 (CD20). Disclosed herein, in some embodiments, are lentiviral particles comprising: a polynucleotide encoding a chimeric antigen receptor (CAR) that specifically binds cluster of differentiation-20 (CD20), wherein the CAR comprises a VL or VH sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 176 or 179; and wherein the lentiviral particle comprises any of the following: a heterologous viral glycoprotein (G protein), a surface displayed polypeptide selected from: an anti-CD3 antibody or antigen-binding fragment, a CD28 ligand, or a 4-1BB ligand, or a fusion molecule comprising: a CD58 extracellular domain, or a functional fragment thereof, a CD80 or CD86 extracellular domain, or a functional fragment thereof, and an anti-CD3 antibody or antigen-binding fragment thereof. Some embodiments include a heterologous viral glycoprotein (G protein). In some embodiments, the viral G protein comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 74. Some embodiments include a surface displayed polypeptide selected from: an anti-CD3 antibody or antigen-binding fragment, a CD28 ligand, or a 4-1BB ligand. Some embodiments include a fusion molecule comprising: a CD58 extracellular domain, or a functional fragment thereof, a CD80 or CD86 extracellular domain, or a functional fragment thereof, and an anti-CD3 antibody or antigen-binding fragment thereof. In some embodiments, the CD58 extracellular domain comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 10. In some embodiments, the CD80 extracellular domain comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 12. In some embodiments, the CD86 extracellular domain comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 13. In some embodiments, the anti-CD3 antigen-binding fragment comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 31. In some embodiments, the VL comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 176. In some embodiments, the VL is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 175. In some embodiments, the VH comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 179. In some embodiments, the VH is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 178. In some embodiments, the CAR comprises a CD8 hinge and transmembrane domain sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 212. In some embodiments, the CD8 hinge and transmembrane domain are encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 181. In some embodiments, the CAR comprises a 4-1BB sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 213. In some embodiments, the 4-1BB is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 184. In some embodiments, the CAR comprises a CD3zeta sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 214. In some embodiments, the CD3zeta is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 185. In some embodiments, the lentiviral particle comprises a polynucleotide encoding a free FKBP12-rapamycin binding (FRB) domain polypeptide. In some embodiments, the free FRB polypeptide comprises a sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 251, 252, or 260. In some embodiments, the free FRB polypeptide is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 256, 257, or 258. In some embodiments, the lentiviral particle comprises a polynucleotide encoding a FRB polypeptide. In some embodiments, the FRB polypeptide comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 252. In some embodiments, the FRB polypeptide is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 257. In some embodiments, the lentiviral particle comprises a polynucleotide encoding a cytokine gamma chain polypeptide. In some embodiments, the cytokine gamma chain polypeptide comprises a polypeptide sequence at least 80%, at least 85%, at least 90%, at least 91%, at least 92%, at least 93%, at least 94%, at least 95%, at least 96%, at least 97%, at least 98%, at least 99%, or 100% identical to SEQ ID NO: 264 or 265. In some embodiments, the cytokine gamma chain polypeptide is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 261, 262, or 263. In some embodiments, the FRB polypeptide and the cytokine gamma chain polypeptide are fused together. In some embodiments, the lentiviral particle comprises a polynucleotide encoding a FKBP polypeptide. In some embodiments, the FKBP polypeptide comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 253. In some embodiments, the FKBP polypeptide is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: SEQ ID NO: 268. In some embodiments, the lentiviral particle comprises a polynucleotide encoding a cytokine beta chain polypeptide. In some embodiments, the cytokine beta chain polypeptide comprises a polypeptide sequence at least 80%, at least 85%, at least 90%, at least 91%, at least 92%, at least 93%, at least 94%, at least 95%, at least 96%, at least 97%, at least 98%, at least 99%, or 100% identical to SEQ ID NO: 272 or 273. In some embodiments, the cytokine beta chain polypeptide is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 270 or 271. In some embodiments, the FKBP polypeptide and the cytokine beta chain polypeptide are fused together. Disclosed herein, in some embodiments, are methods that include administering a lentiviral particle to a subject. In some embodiments, the subject has cancer or is in need of cancer treatment. In some embodiments, the administration treats the cancer. In some embodiments, the cancer comprises CD20+ cancer cells. In some embodiments, the cancer is a B-cell malignancy. In some embodiments, the subject has an autoimmune disease or is in need of treatment for an autoimmune disease. In some embodiments the autoimmune disease is caused or exacerbated by B cells.

### BRIEF DESCRIPTION OF THE DRAWINGS

FIG. 1 depicts T-cell activation by a lentiviral particle displaying a single-chain variable fragment specific for CD3, a viral envelope protein (Cocal G), and two costimulatory molecules.

FIG. 2A shows activation of CD8+ T cells as measured by % CD25+ cells with a lentiviral particle displaying CD3scfv or CD3scfv+CD80.

FIG. 2B shows activation of CD8+ T cells as measured by % CD25+ cells with a lentiviral particle displaying CD3scfv only, CD3scfv+CD80 or CD3scfv+CD58.

FIGS. 2C-2D show levels of CAR expression in CD8+ T cells as determined by % CAR expression (FIG. 2C) or total CAR+CD8+ T cells (FIG. 2D) generated using lentiviral particles with CD3scfv only or CD3scfv+CD80.

FIGS. 2E-2F show levels of CAR expression in CD3+ T cells as determined by % CAR expression (FIG. 2E) or total CAR+CD3+ T cells (FIG. 2F) generated using lentiviral particles with CD3scfv only, CD3scfv+CD80 or CD3scfv+CD58.

FIGS. 2G-2H show fold expansion of CAR+CD8+ T cells generated with lentiviral particles with CD3scfv only or CD3scfv+CD80 stimulated with IL-2 (FIG. 2G) or rapamycin (FIG. 2H).

FIG. 3A shows percentages of CD25(+) CD8 T cells after incubation with a lentiviral particle displaying CD3scfv only, CD3scfv+CD80, CD3scfv+CD58, or CD3scfv+CD80+CD58.

FIG. 3B shows the geometric mean fluorescent intensity (gMFI) of CD25(+) CD8 T cells after incubation with a lentiviral particle displaying CD3scfv only, CD3scfv+CD80, CD3scfv+CD58, or CD3scfv+CD80+CD58.

FIGS. 3C-3E show production of cytokines 3 days after incubation with particles displaying CD3scfv only, CD3scfv+CD80, CD3scfv+CD58, or CD3scfv+CD80+CD58. IFN-γ (FIG. 3C), IL-2 (FIG. 3D), and TNF-α (FIG. 3E) levels were measured.

FIGS. 3F-3G show CAR expression in CD3+ T cells generated with lentiviral particles displaying CD3scfv only, CD3scfv+CD80, CD3scfv+CD58, or CD3scfv+CD80+CD58 (mixed particles). Percentage (%) CAR expression (FIG. 3F) and total CAR+ T cells (FIG. 3G) were measured.

FIGS. 311-3I show CAR expression in CD8+ T cells generated with lentiviral particles displaying CD3scfv only, CD3scfv+CD80, CD3scfv+CD58, or CD3scfv+CD80+CD58 (same particle). Percentage (%) CAR expression (FIG. 3H) and total CAR+ T cells (FIG. 3I) were measured.

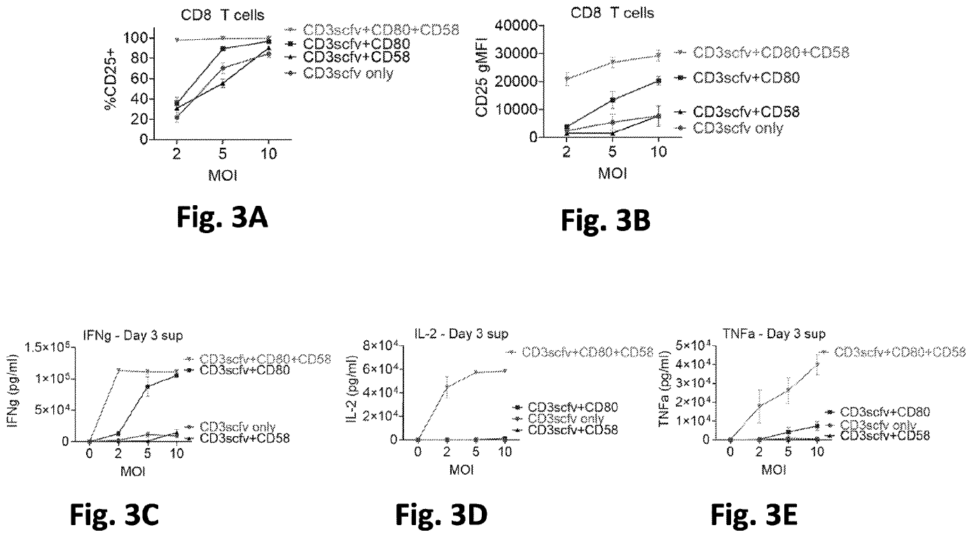

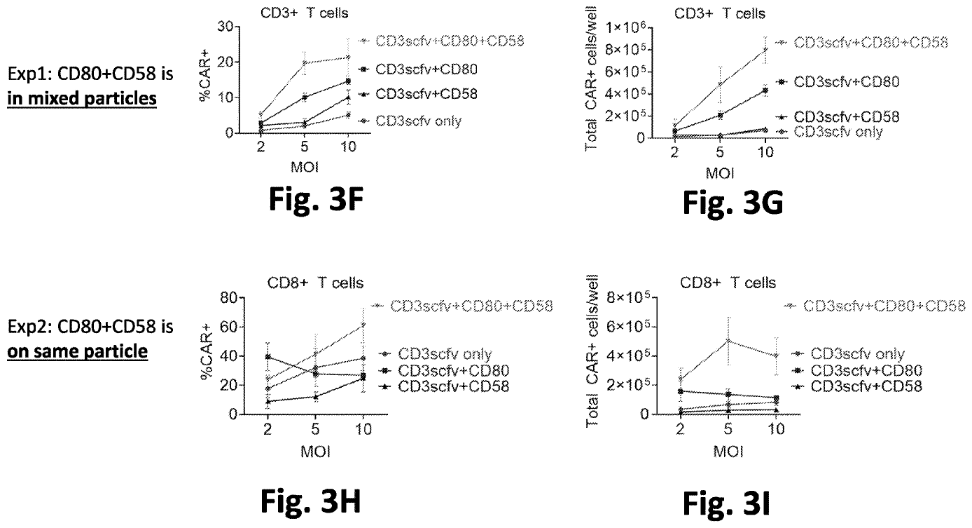

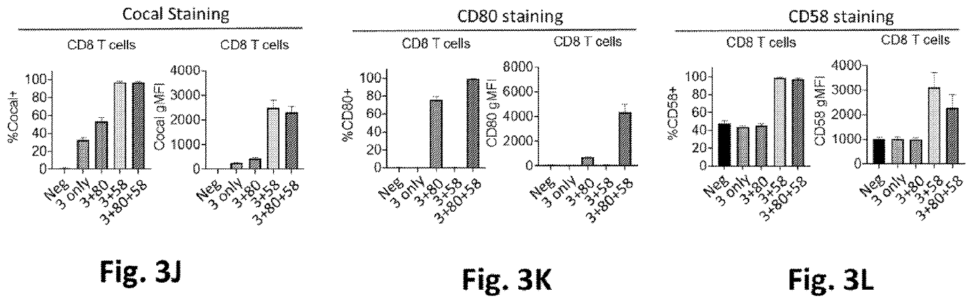

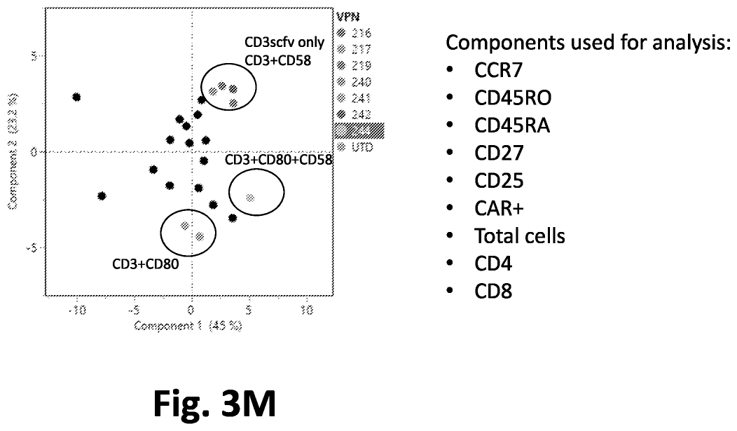

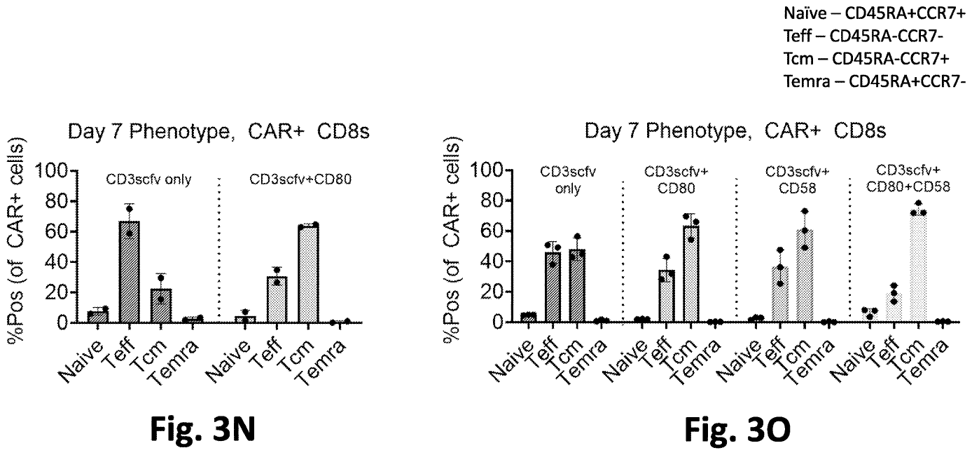

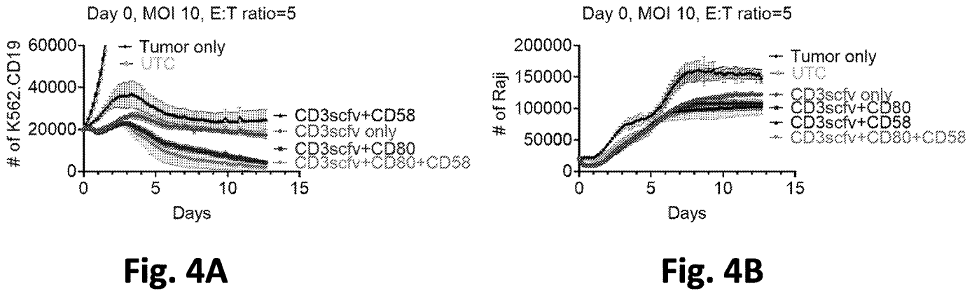

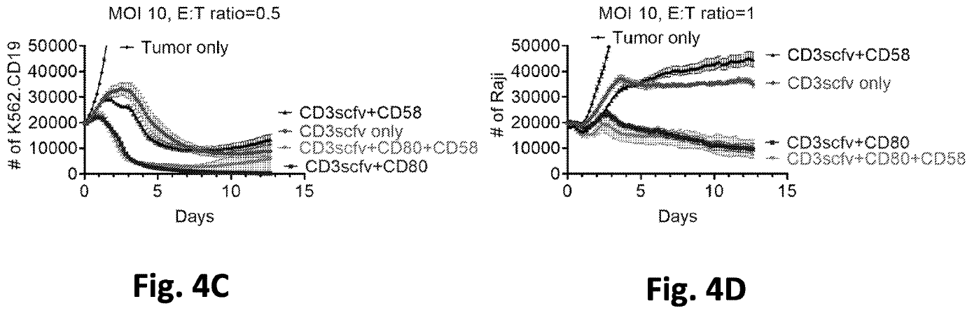

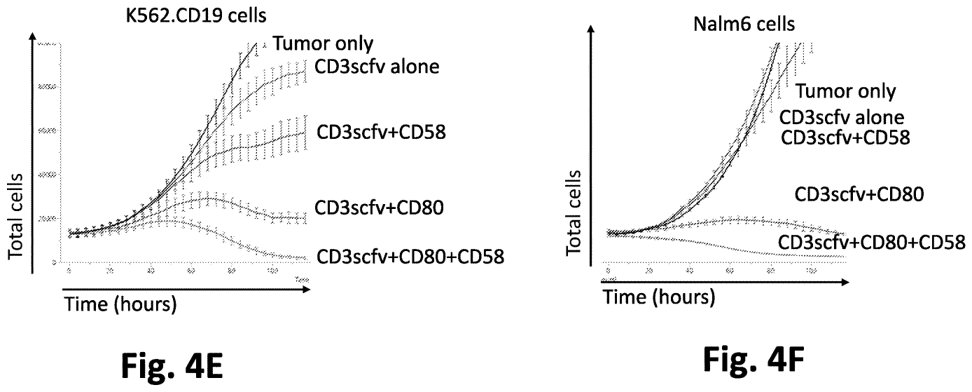

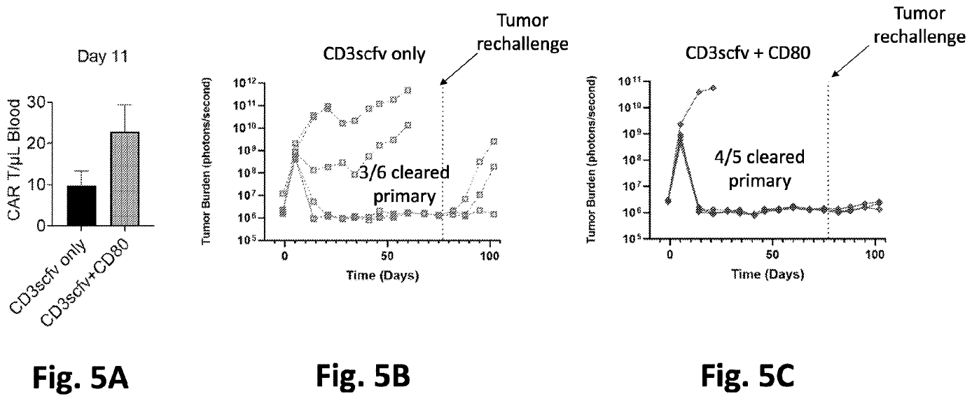

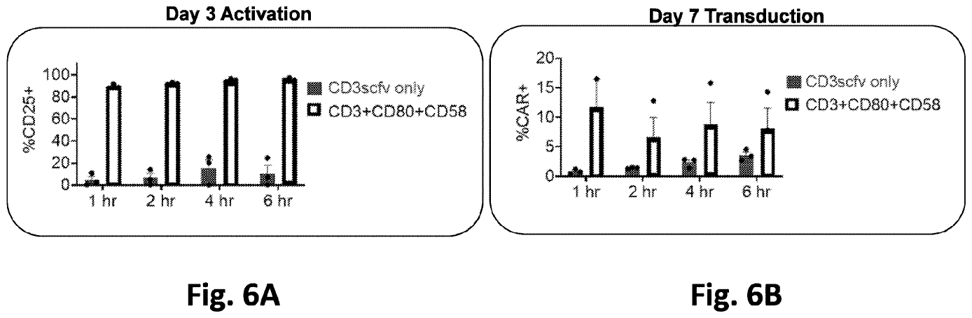

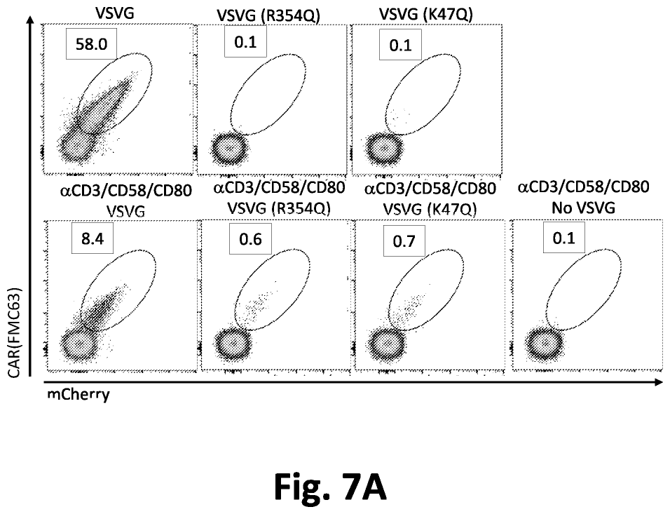

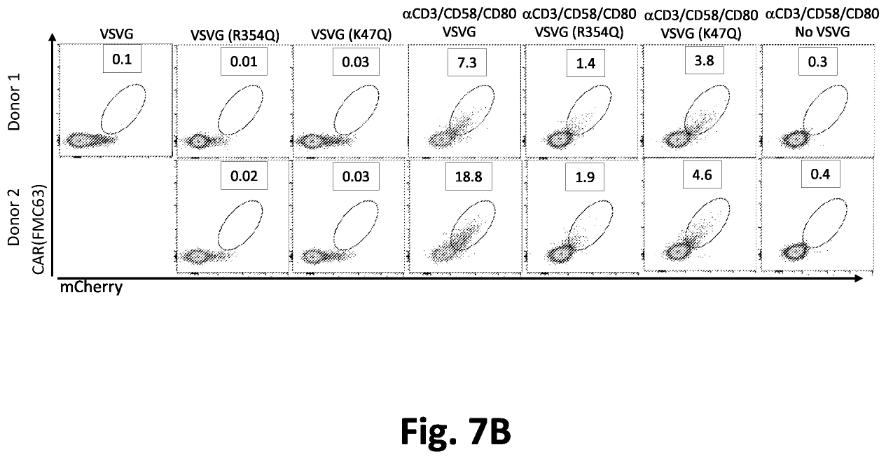

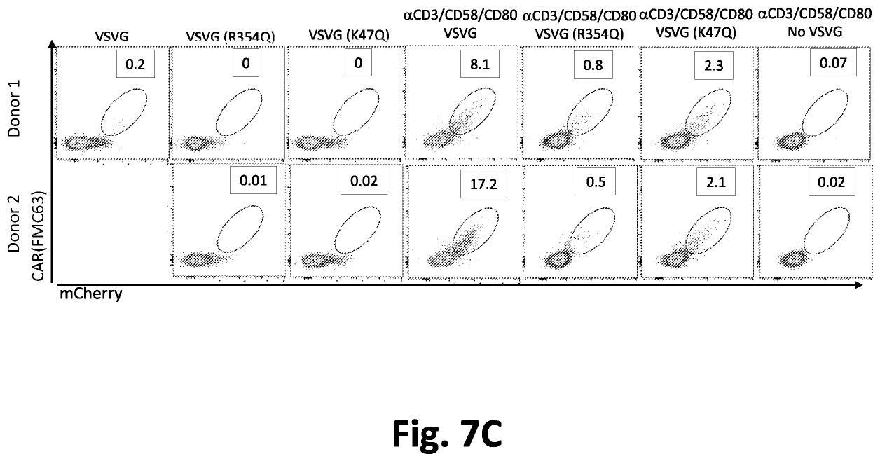

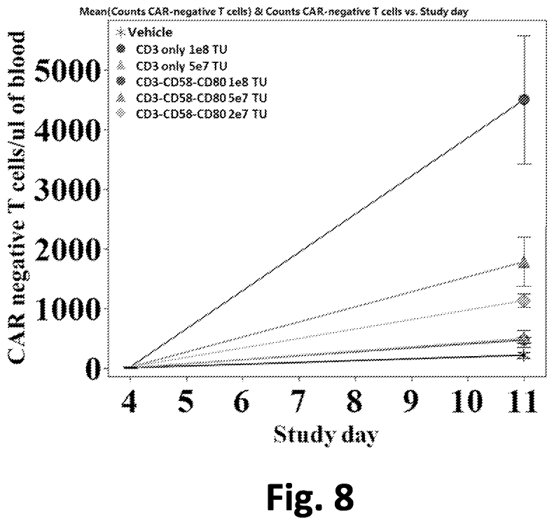

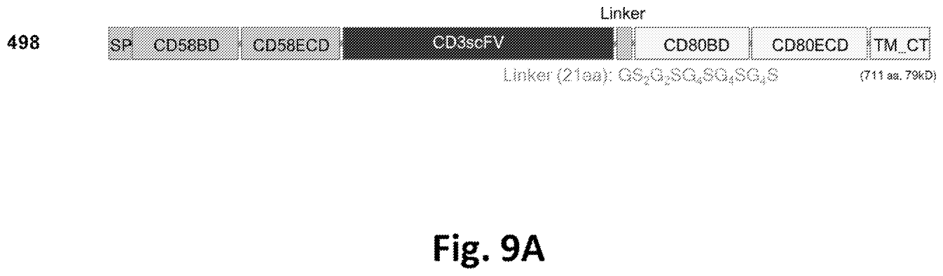

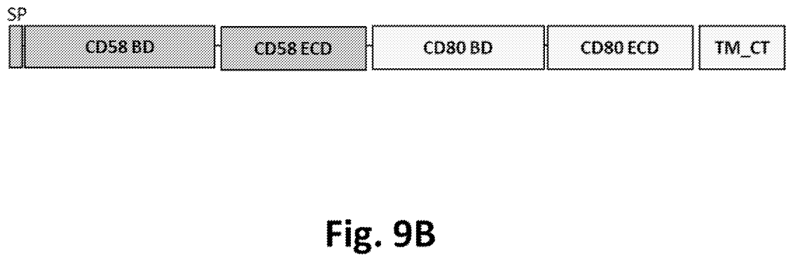

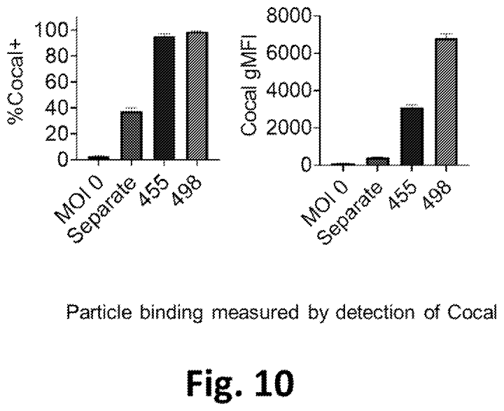

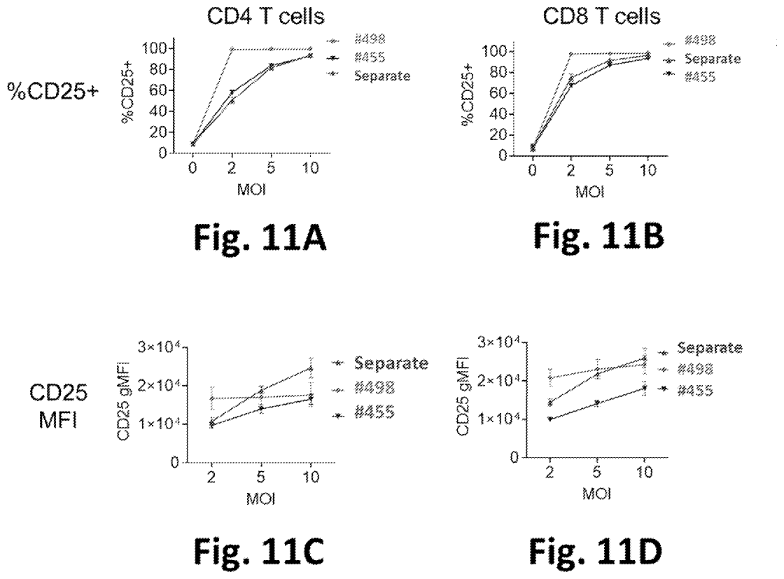

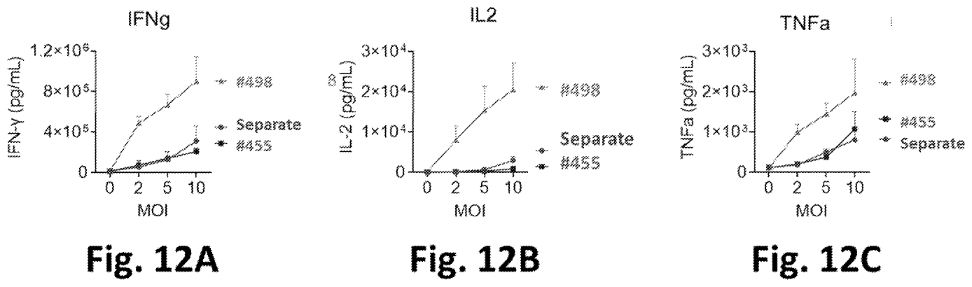

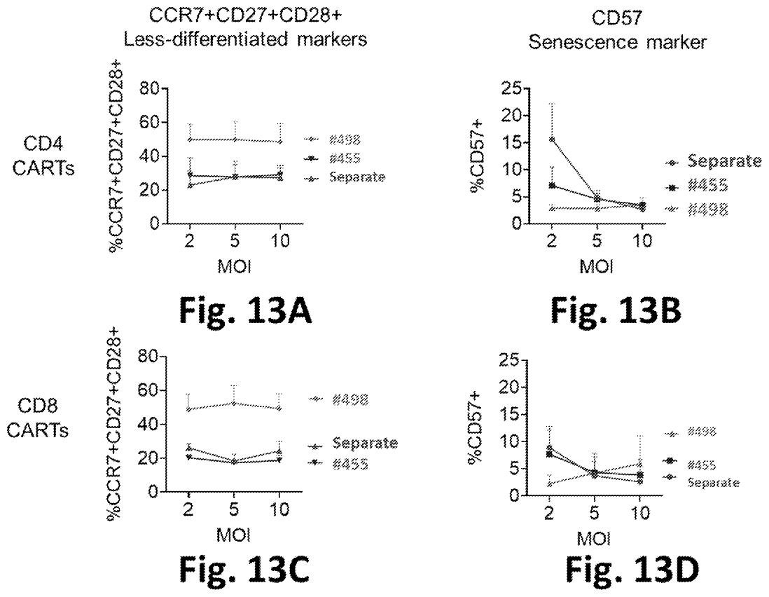

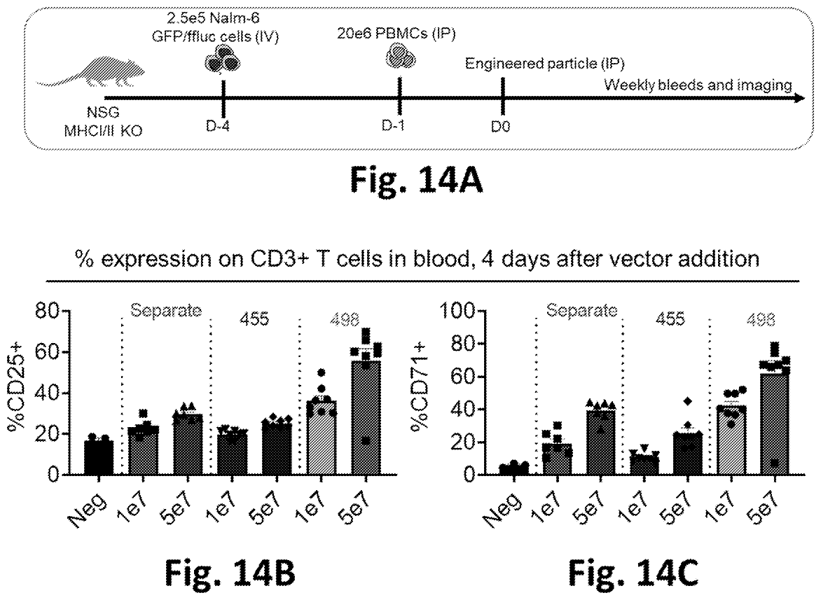

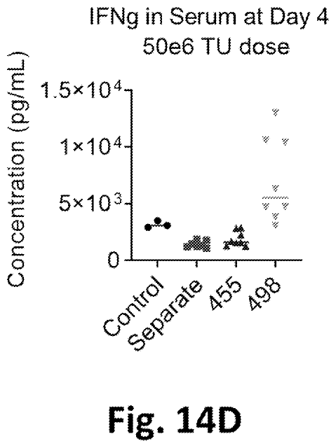

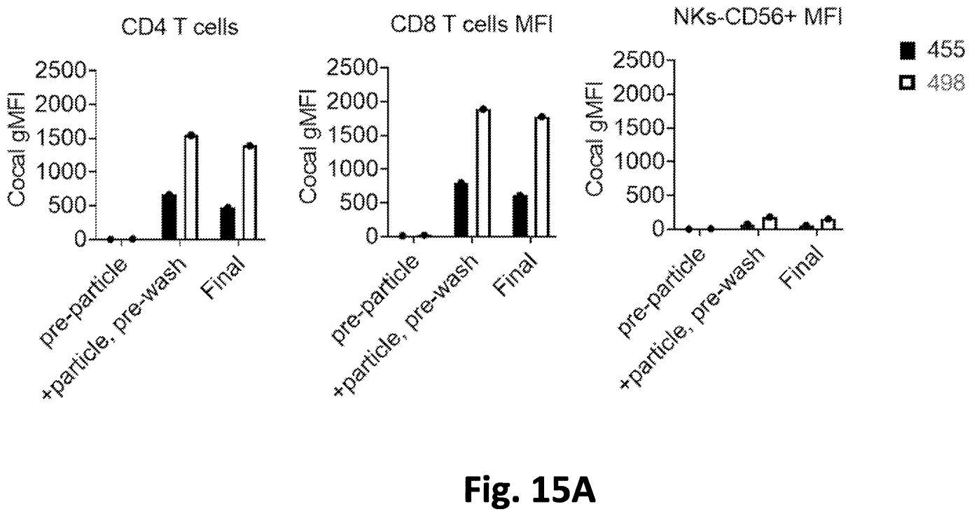

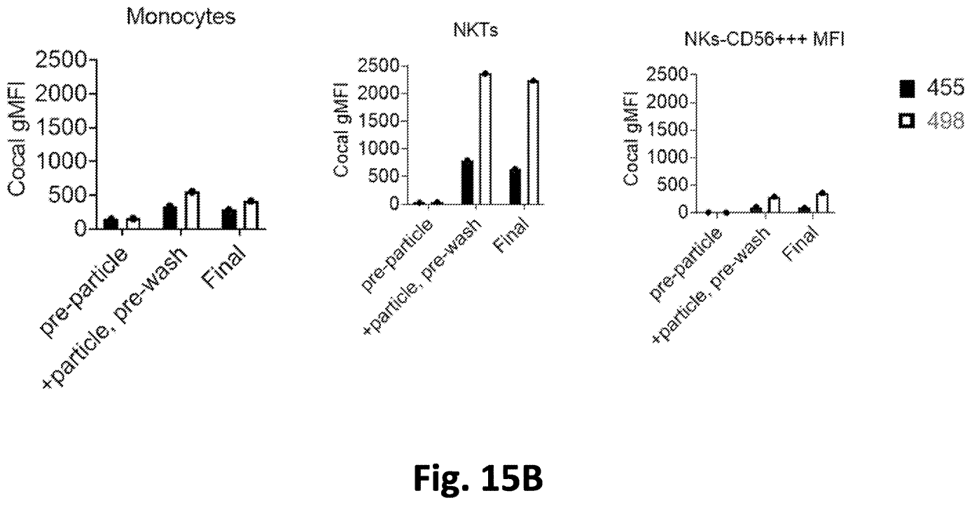

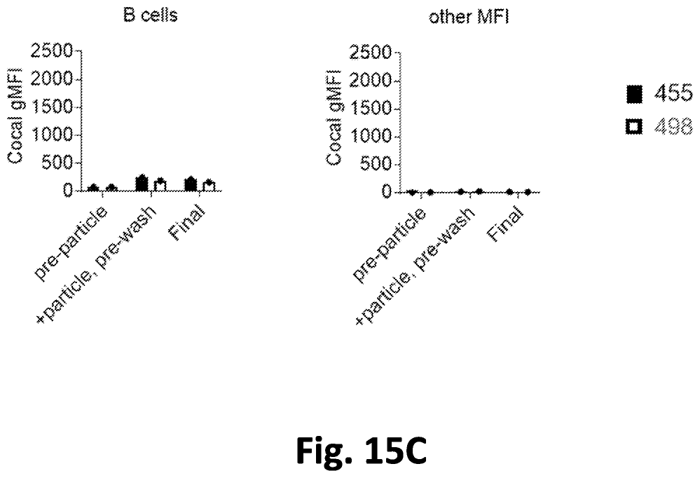

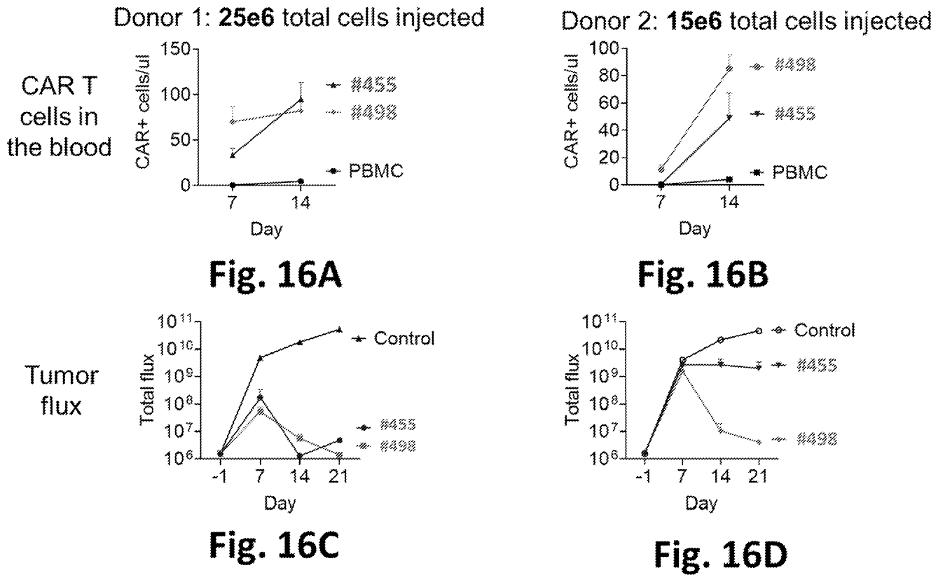

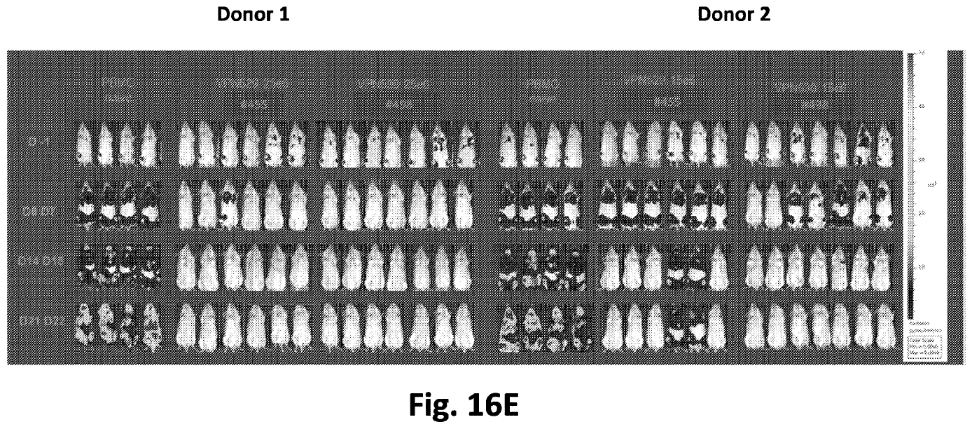

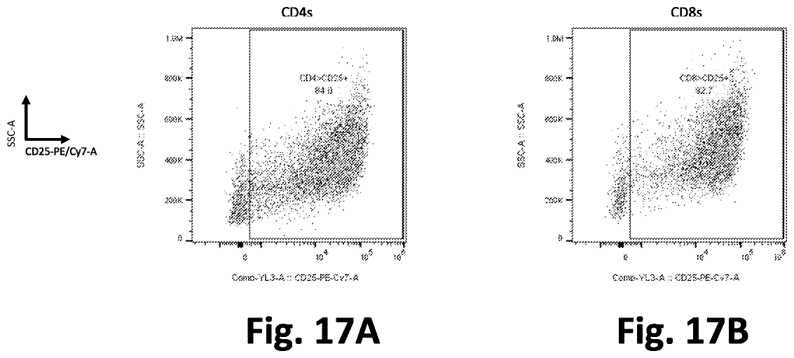

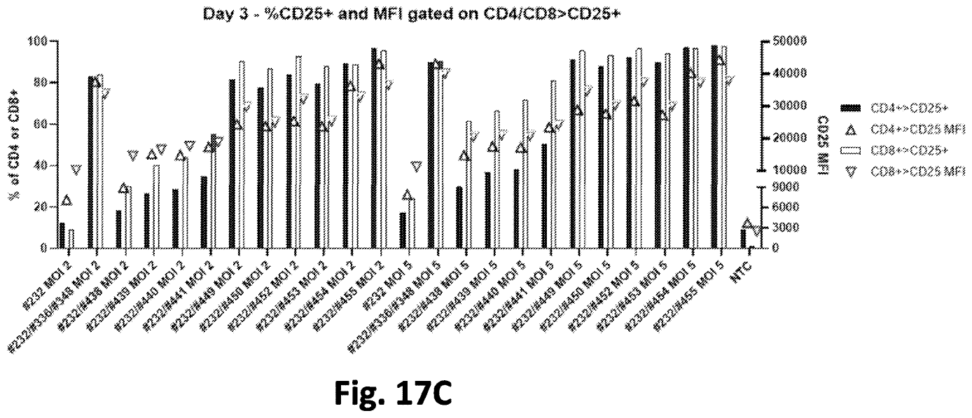

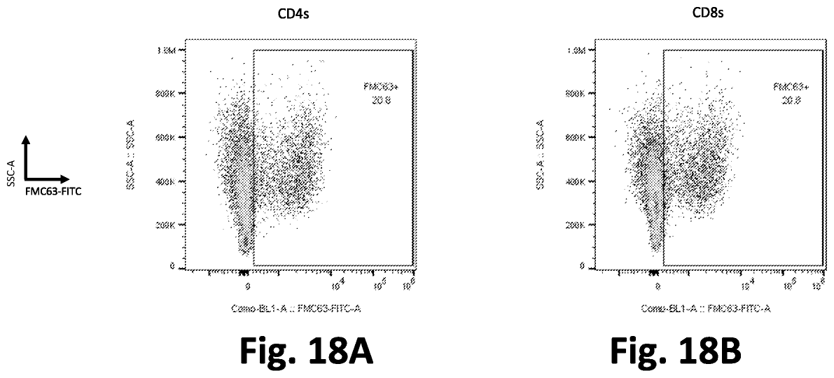

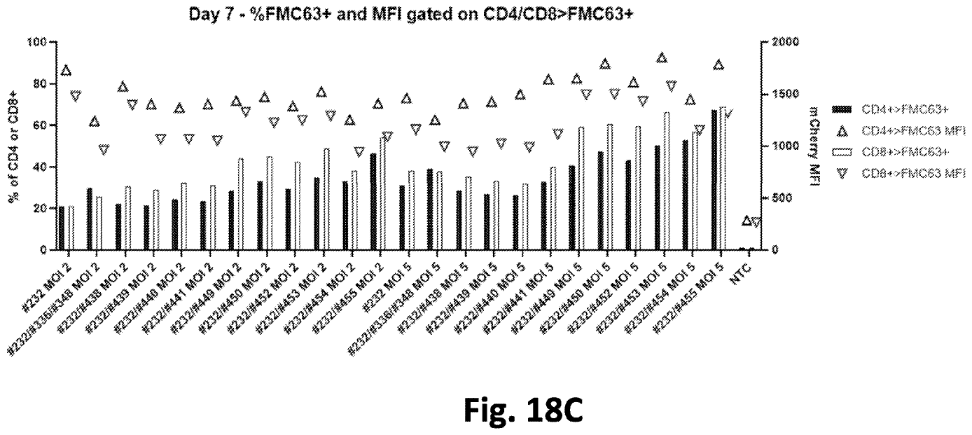

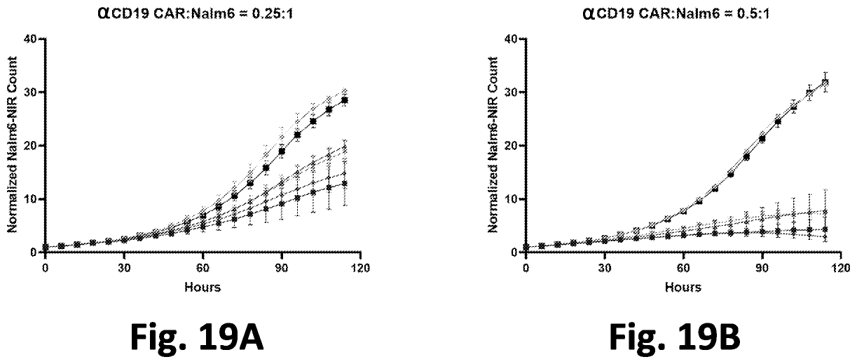

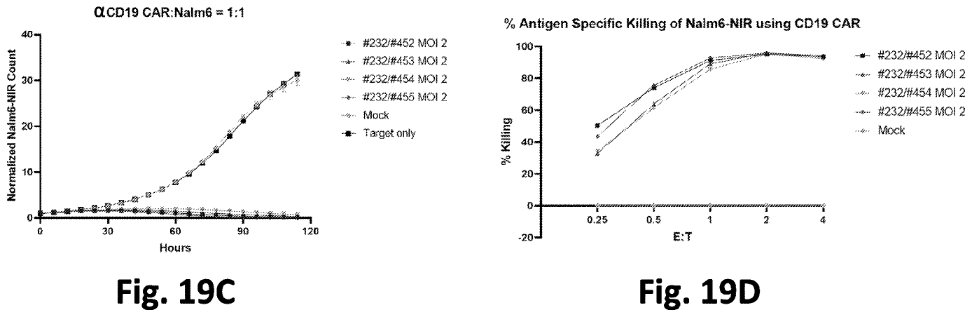

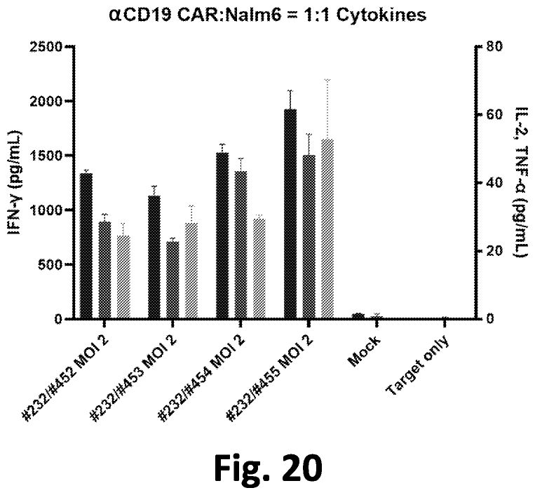

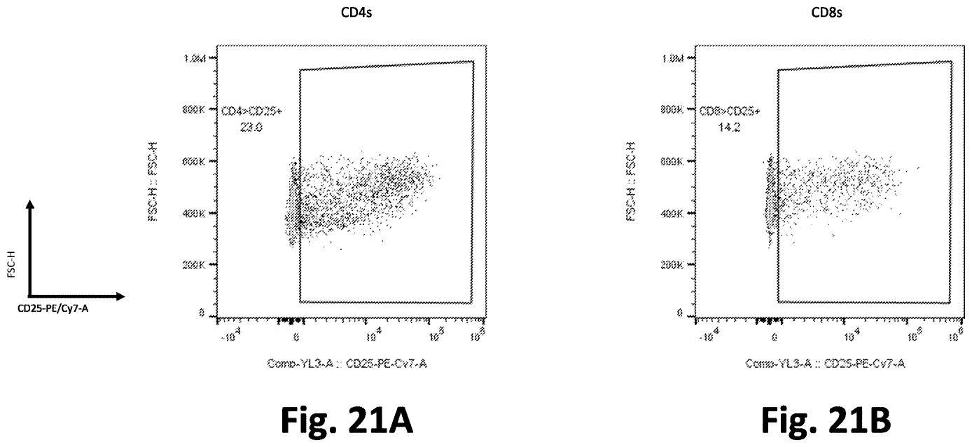

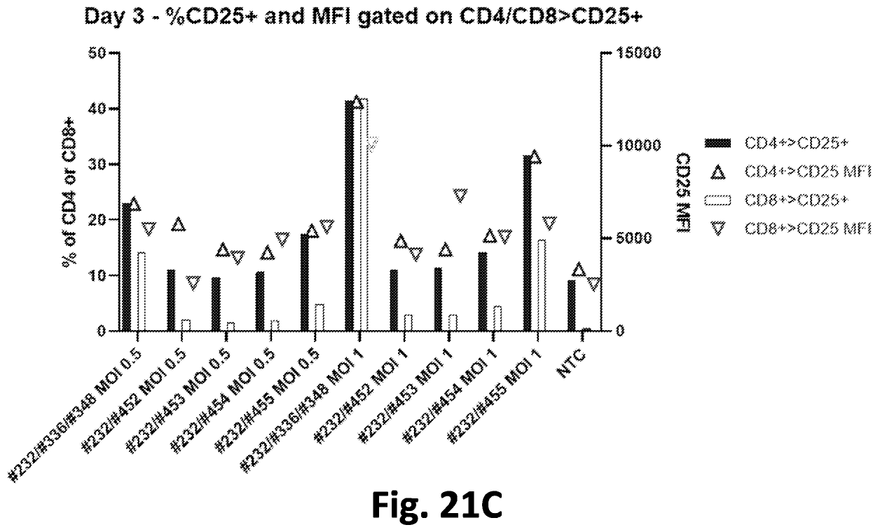

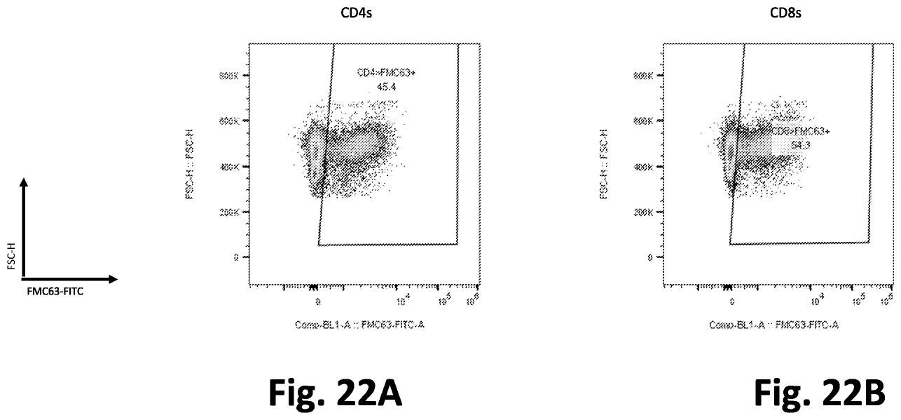

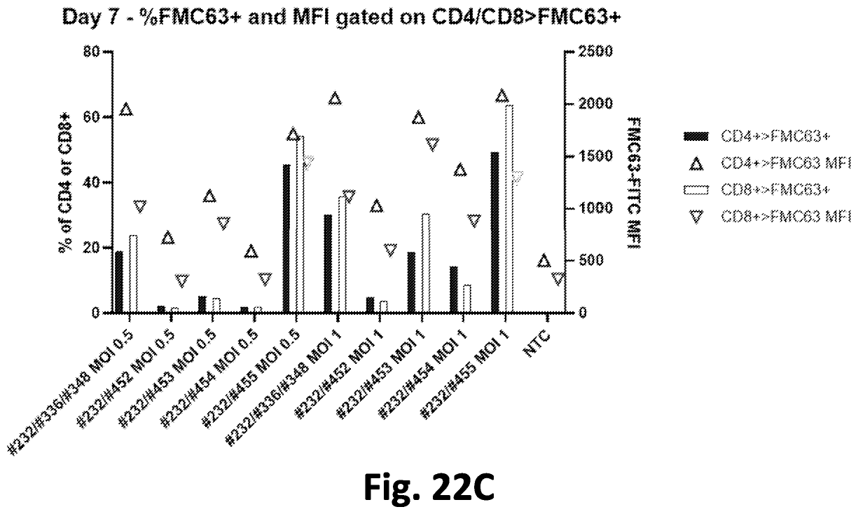

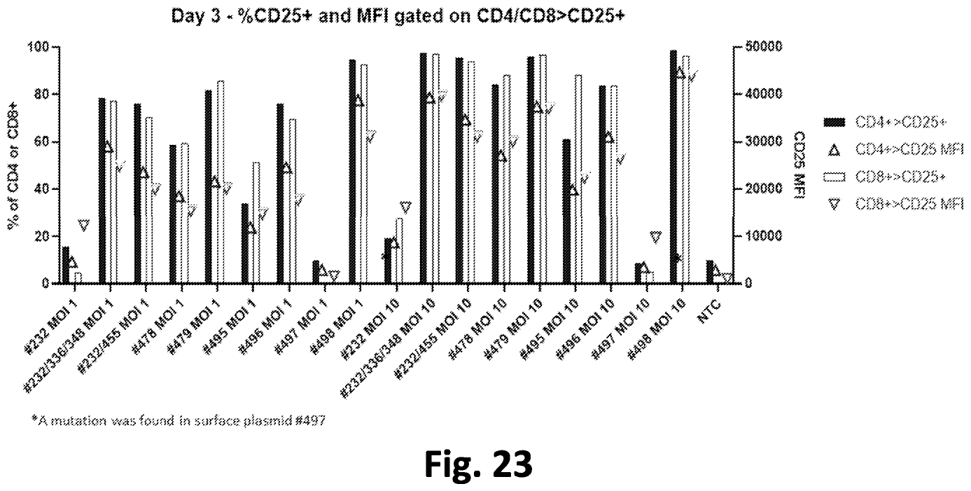

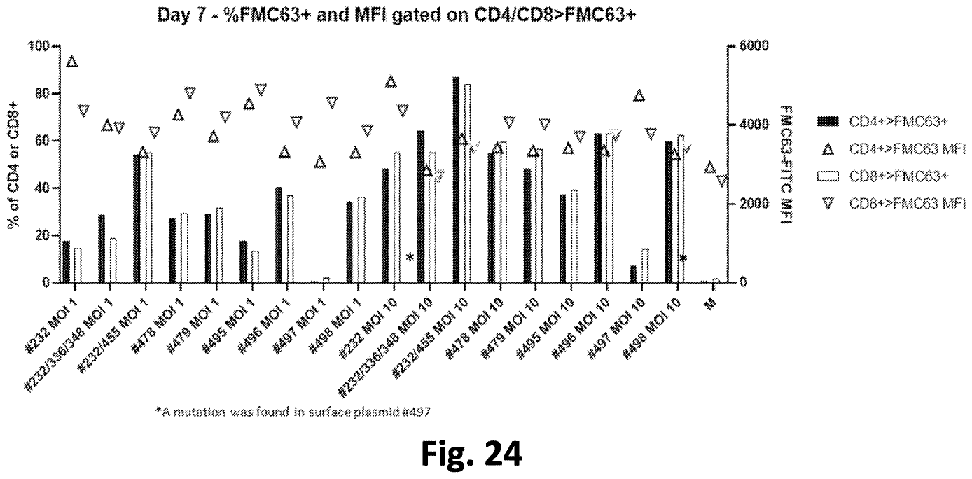

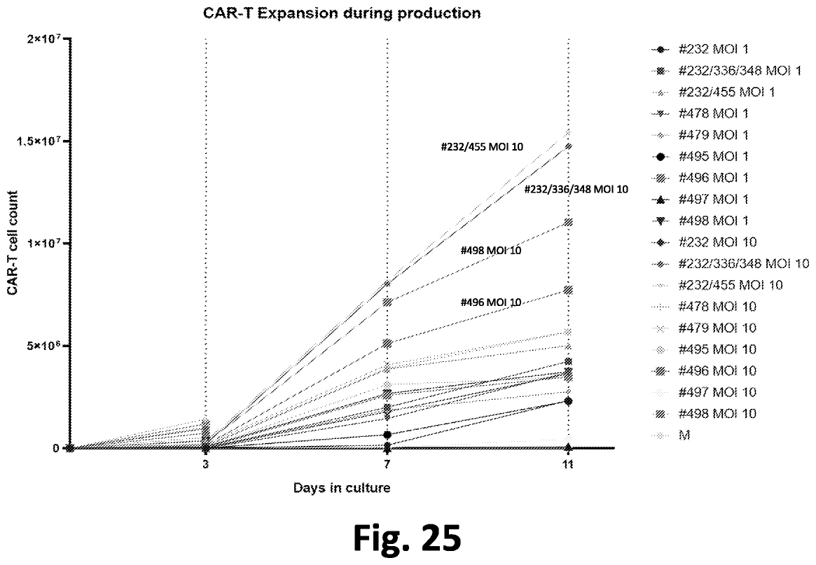

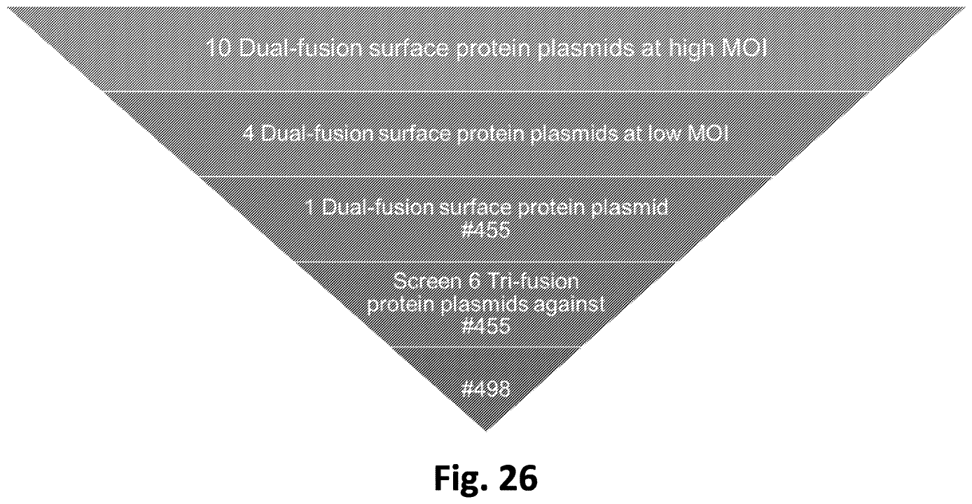

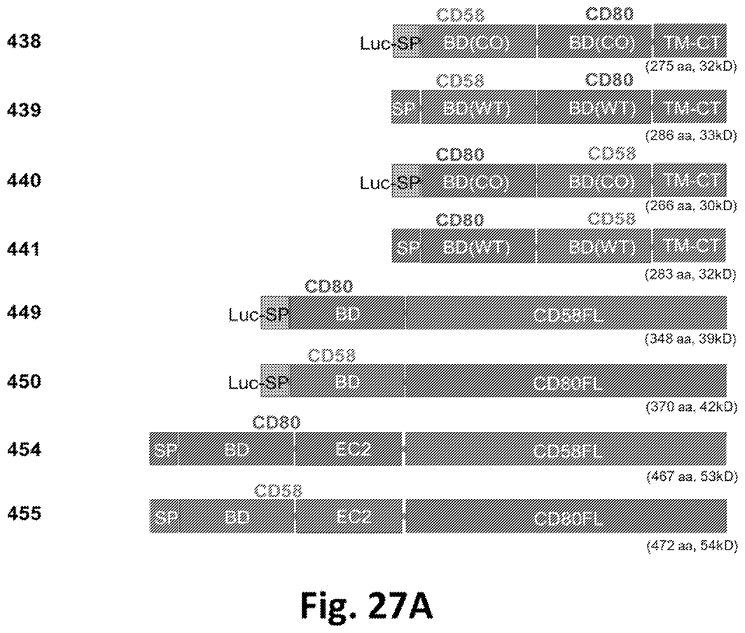

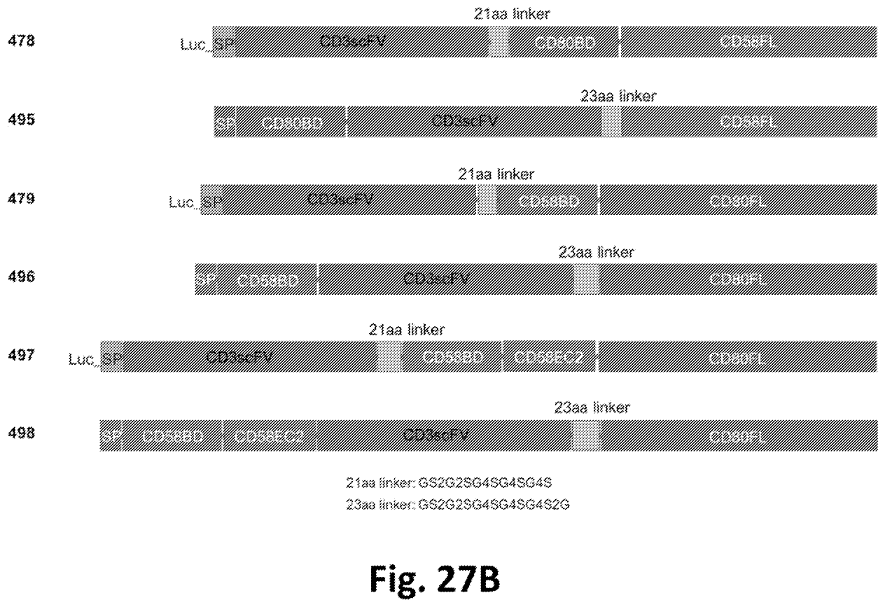

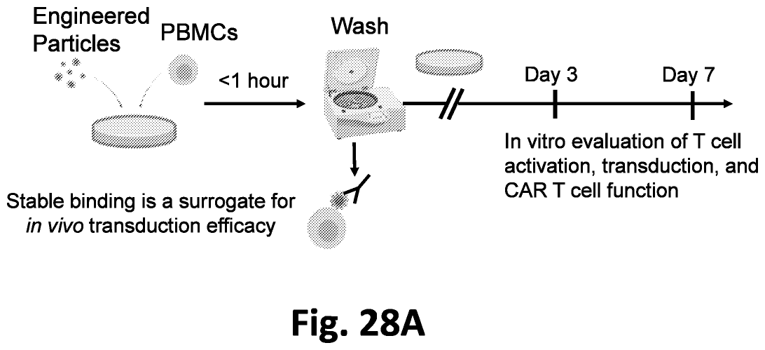

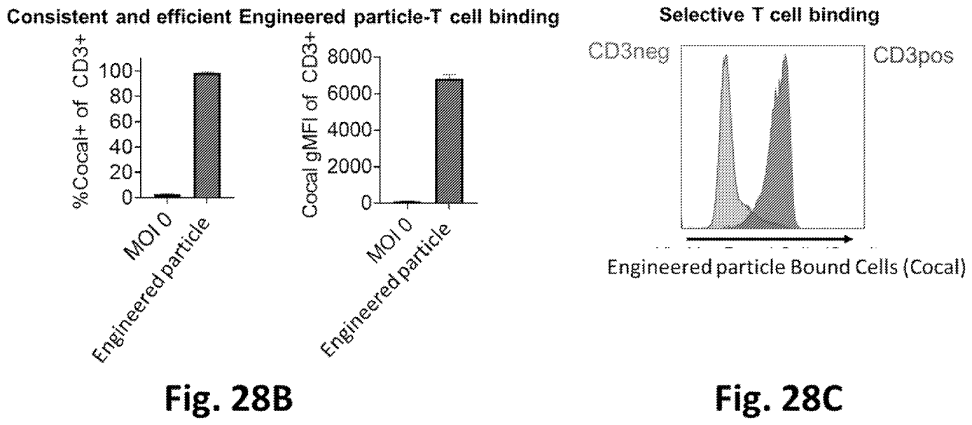

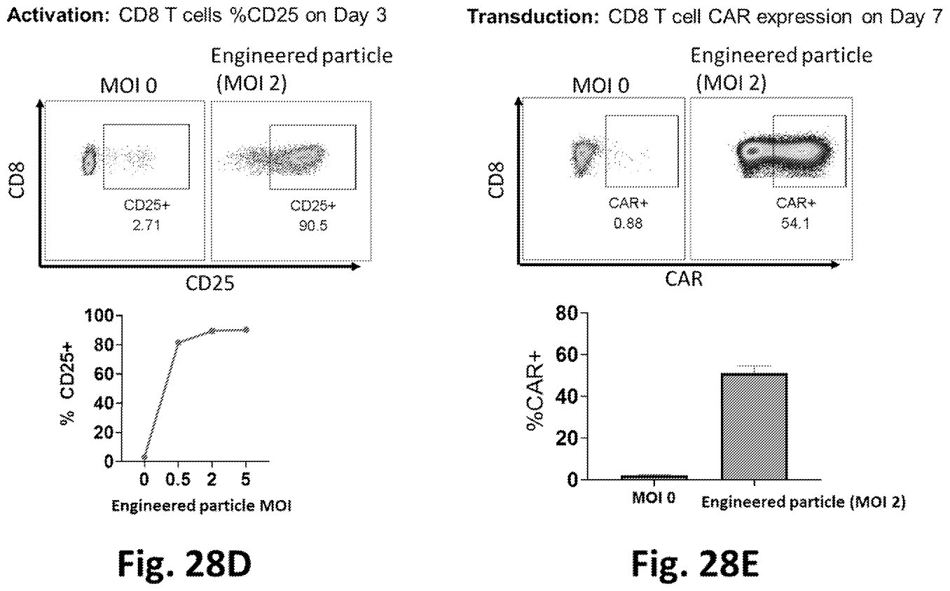

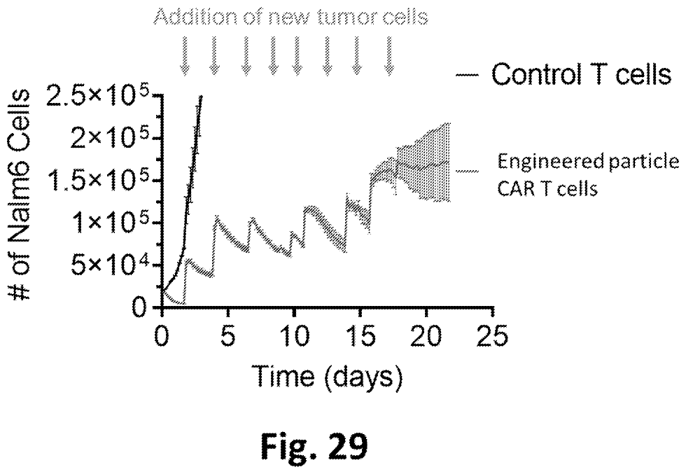

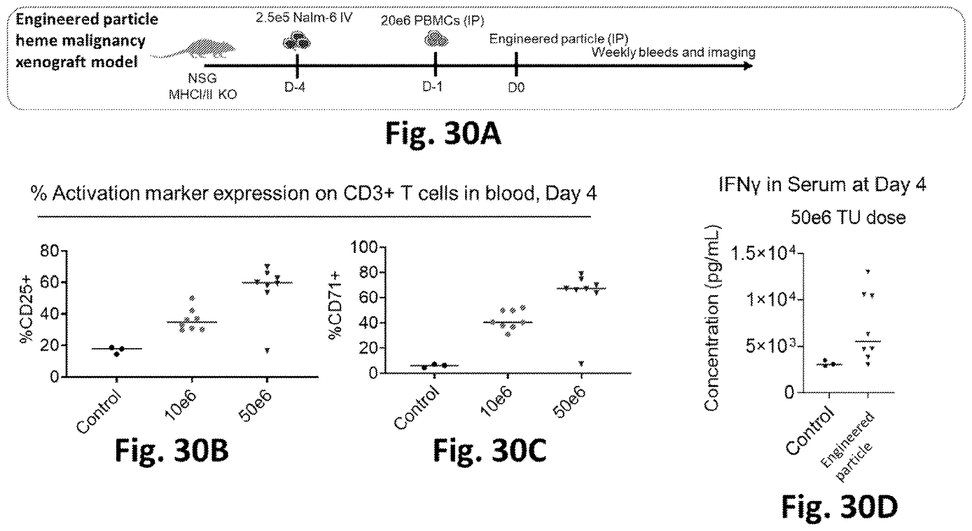

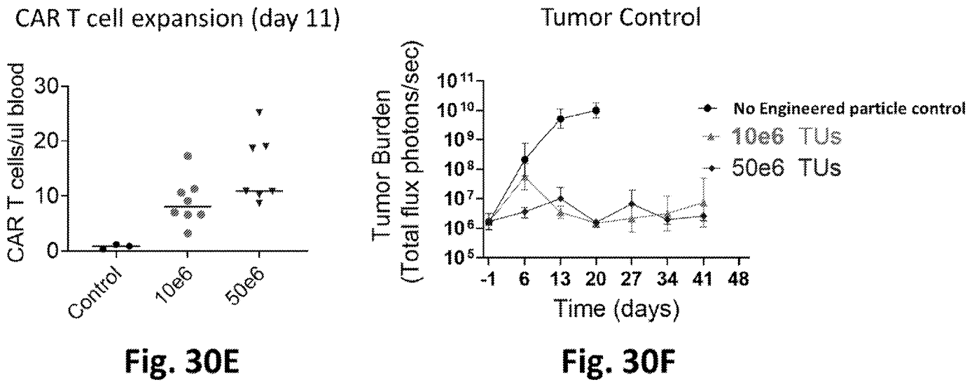

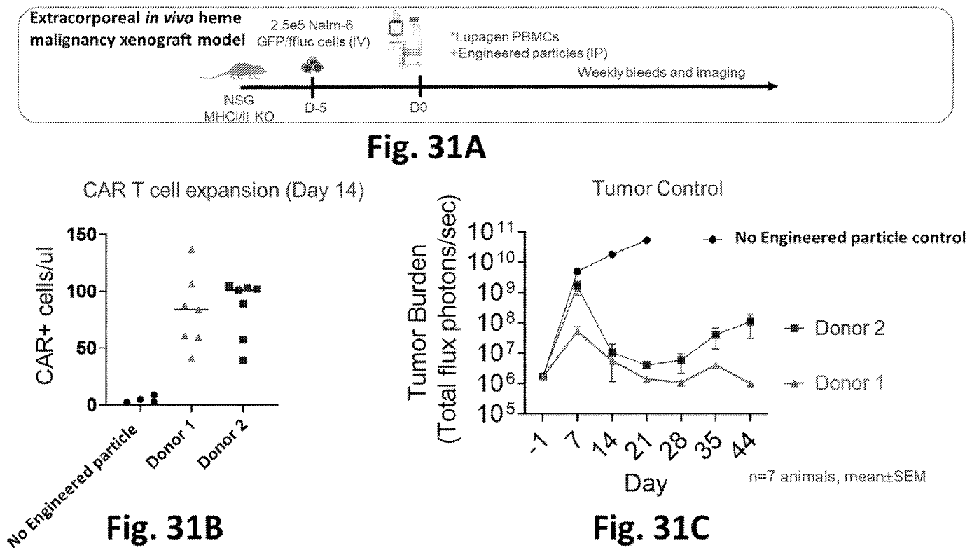

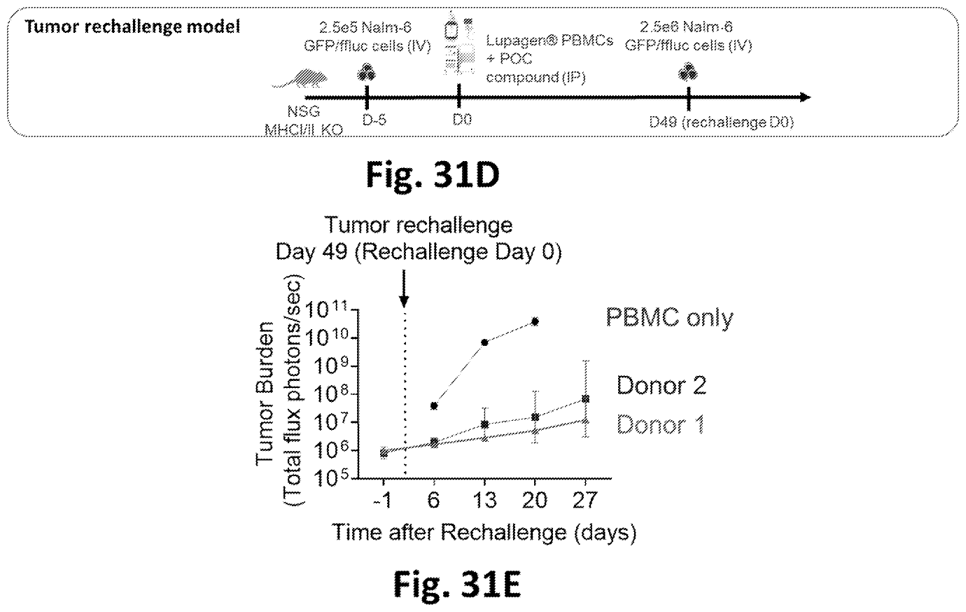

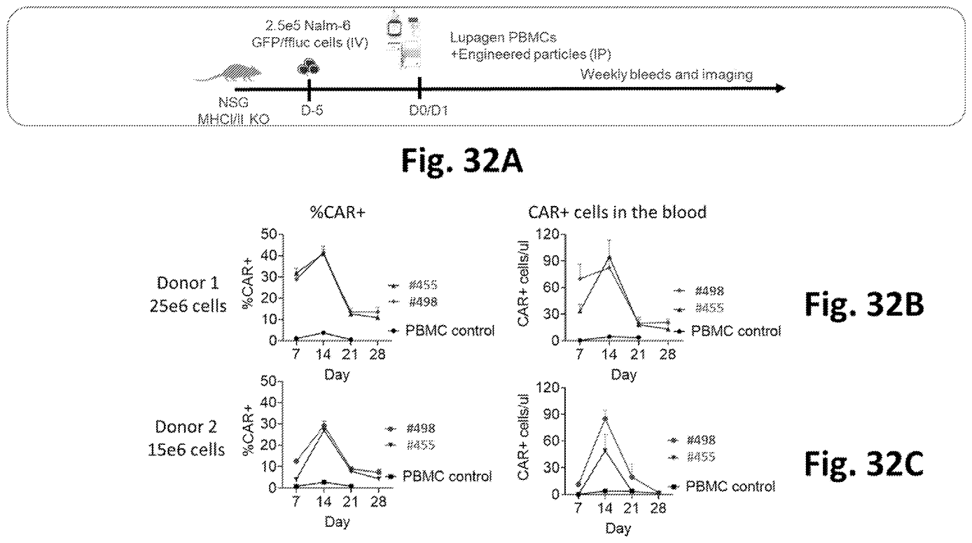

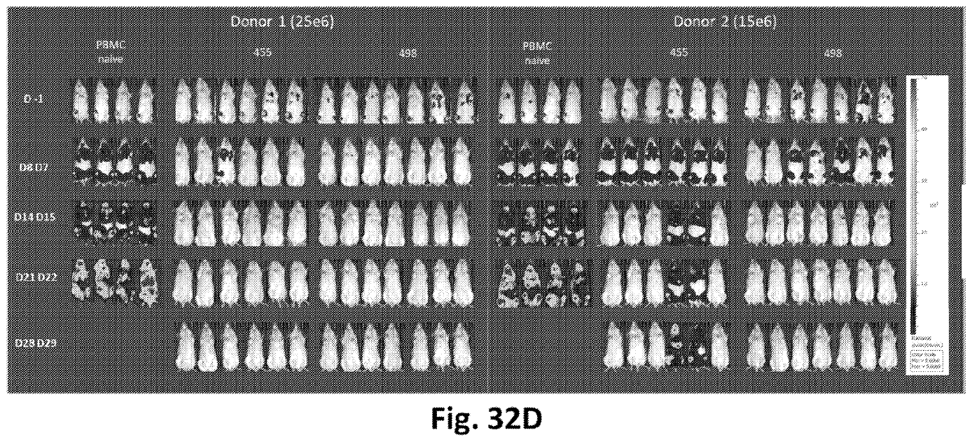

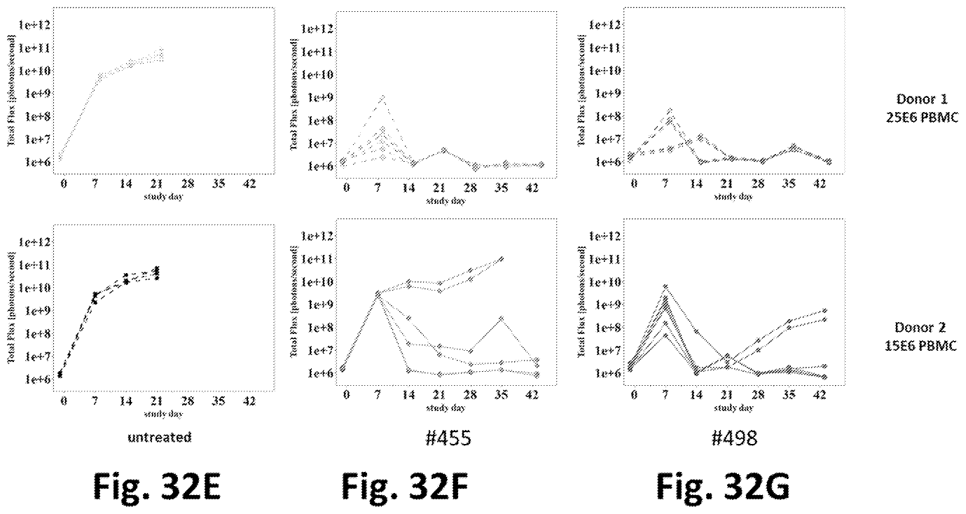

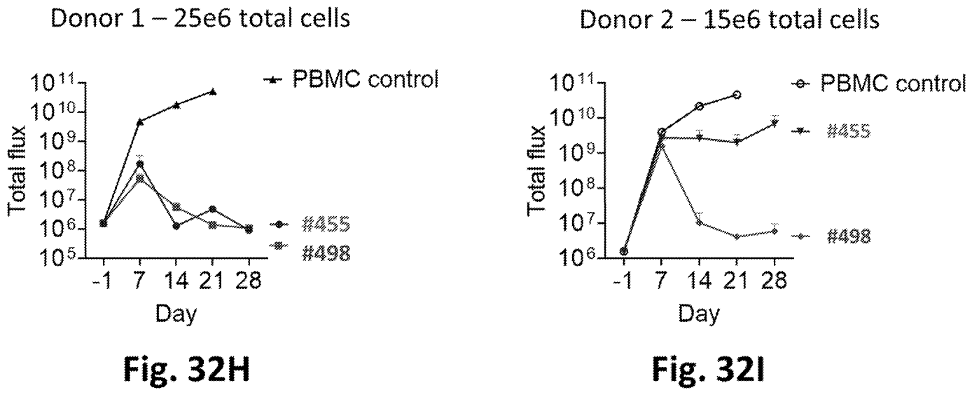

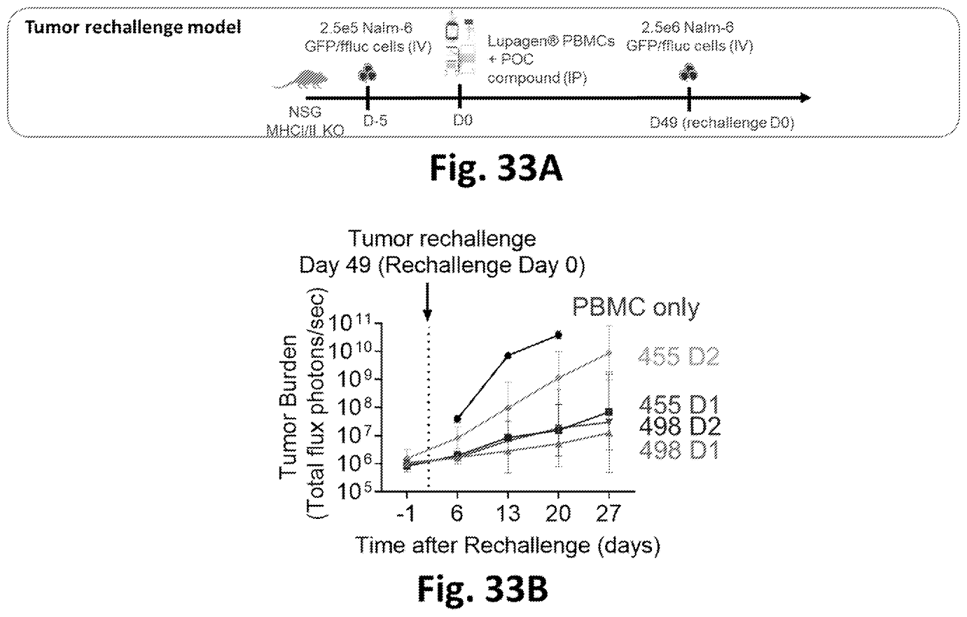

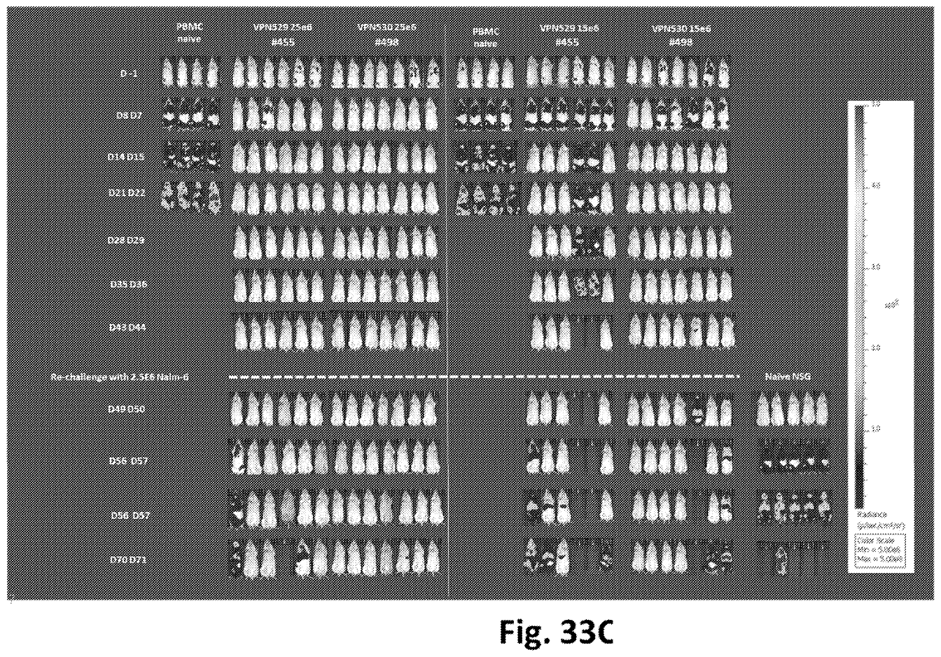

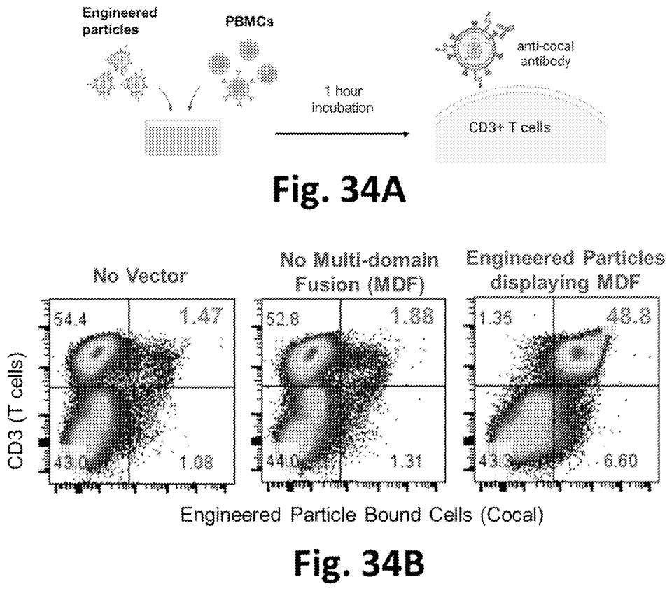

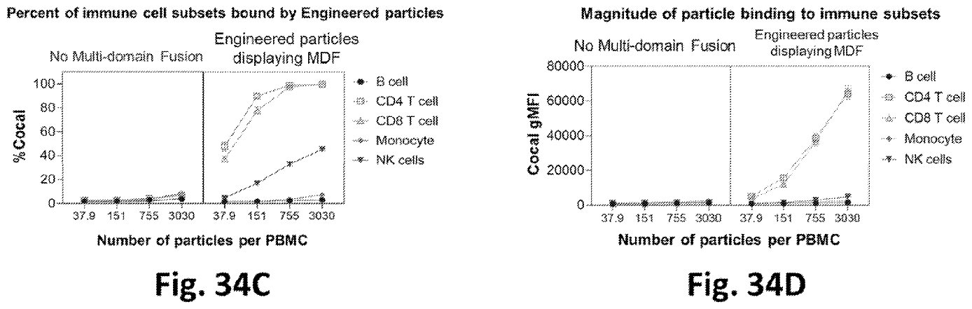

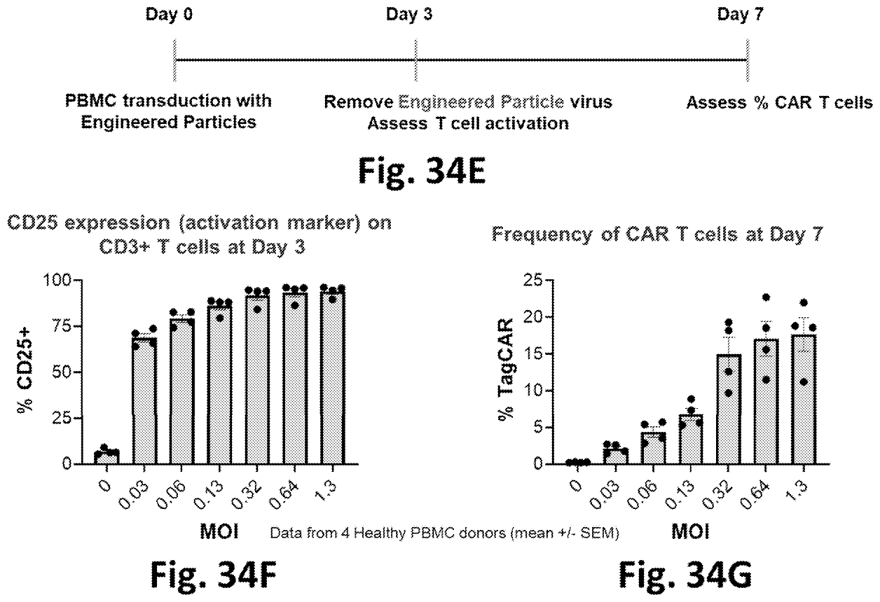

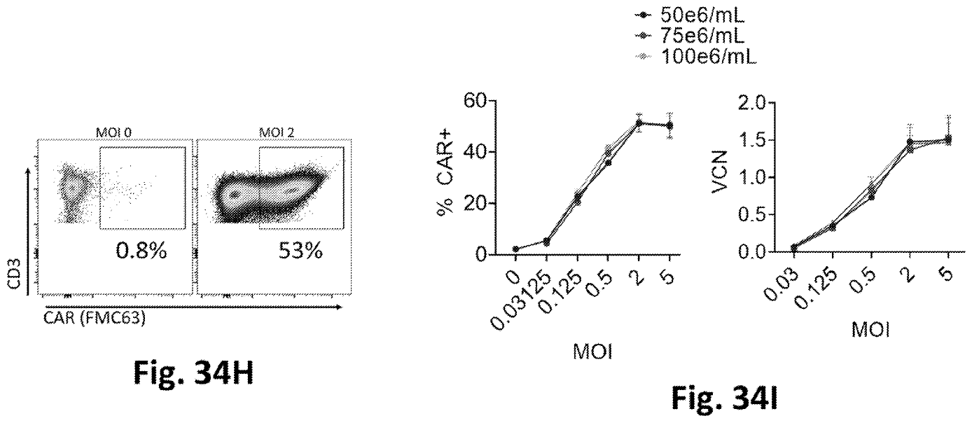

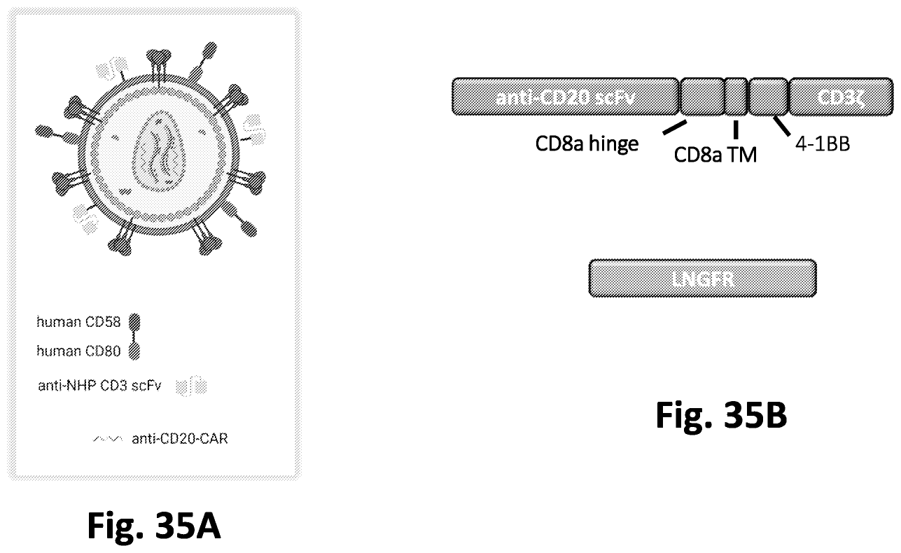

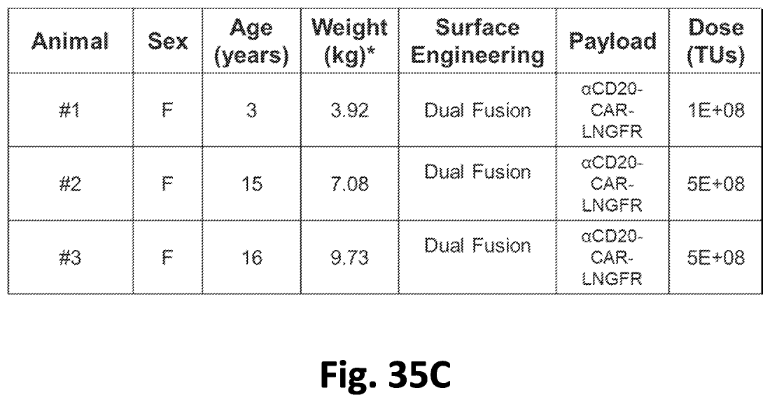

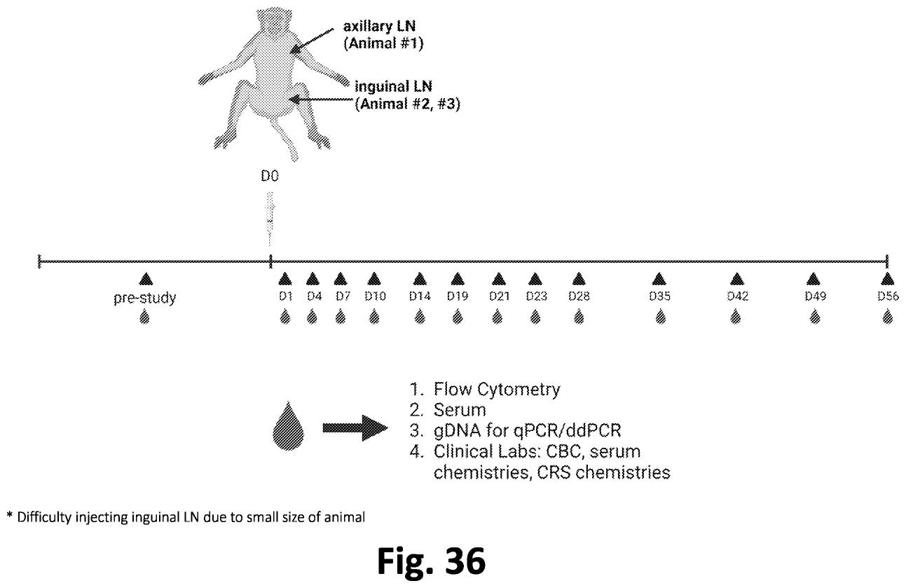

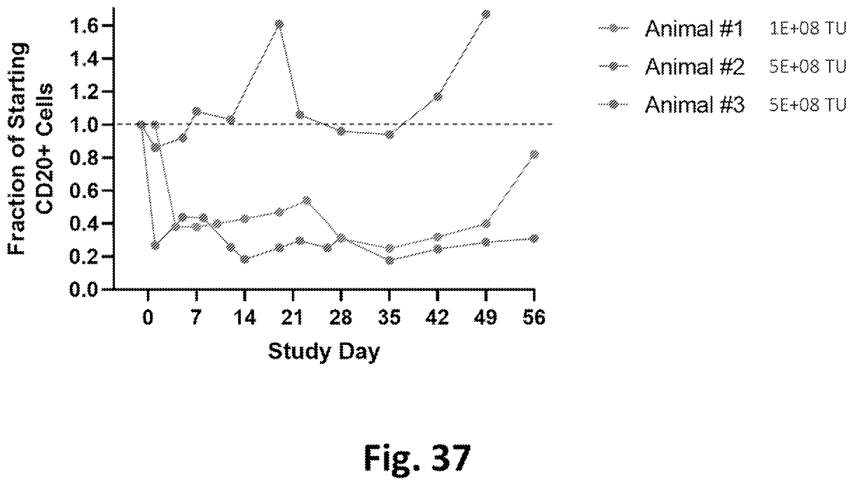

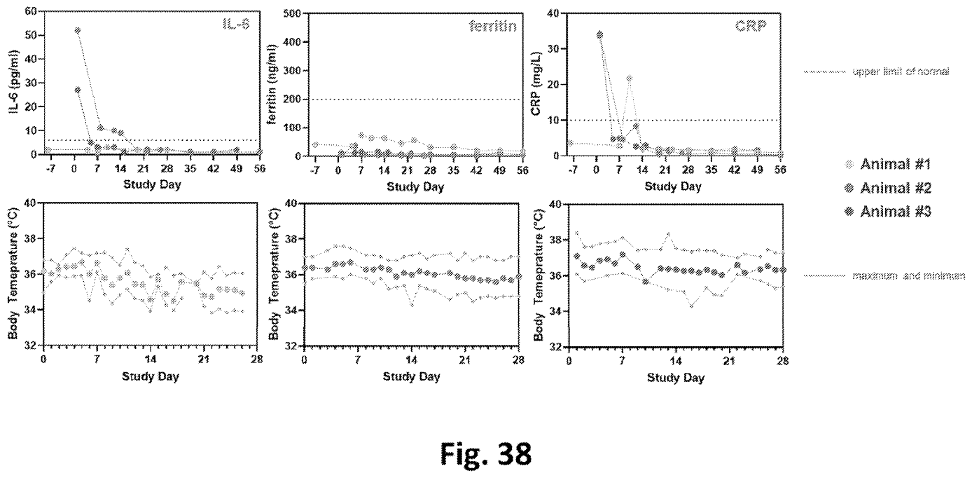

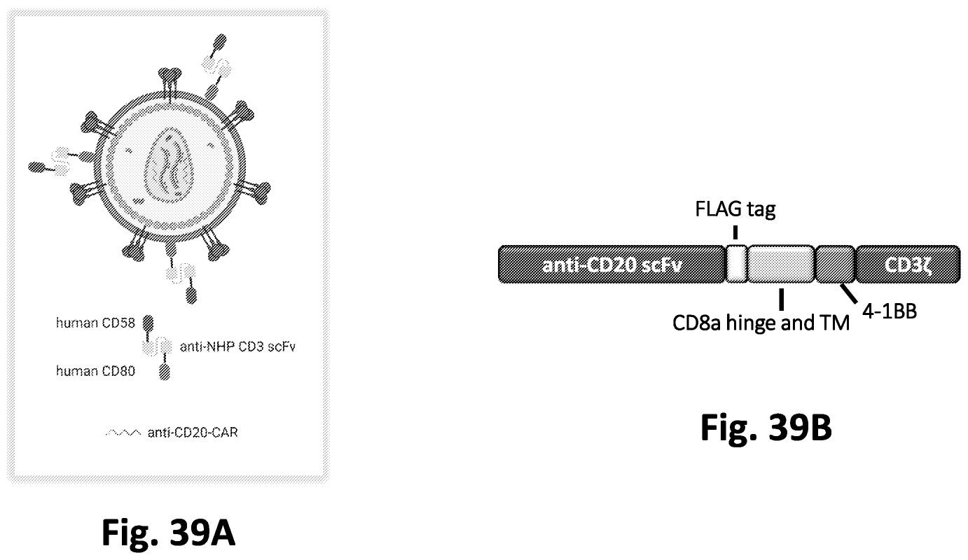

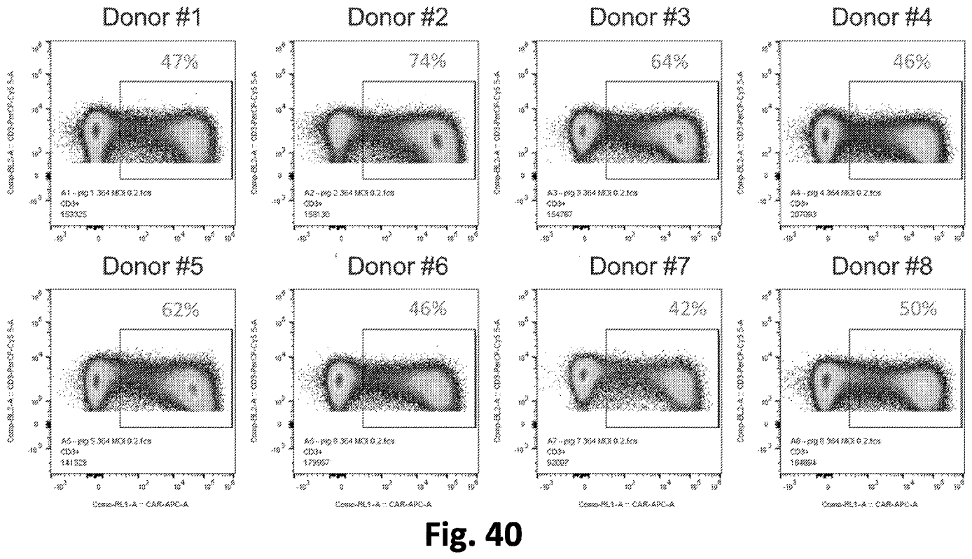

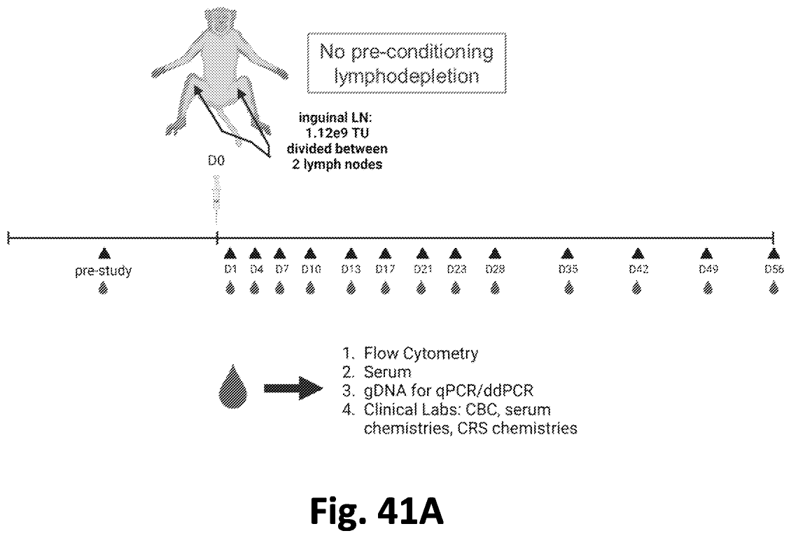

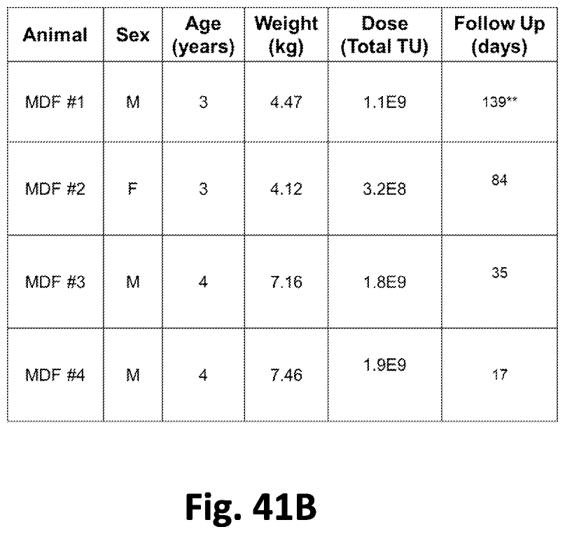

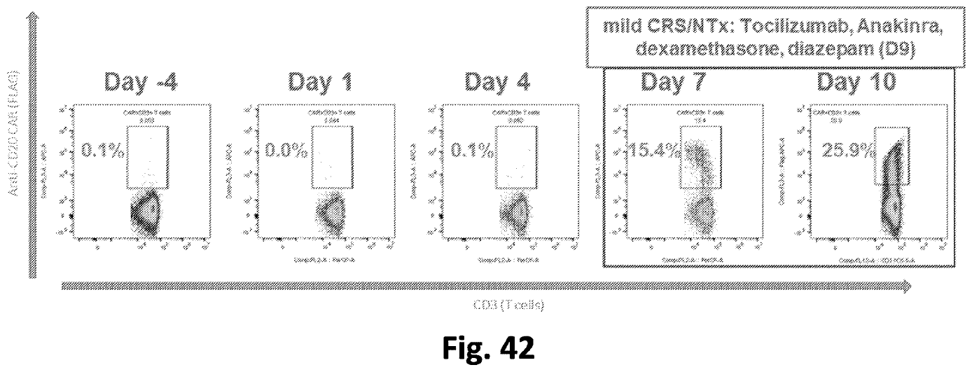

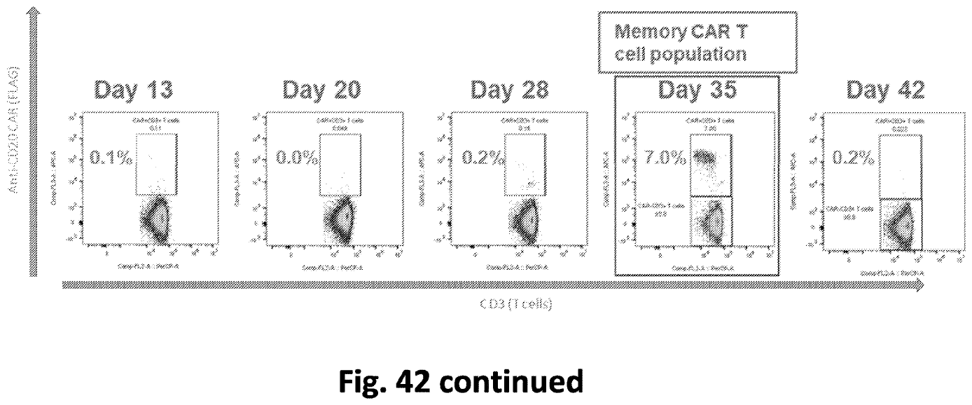

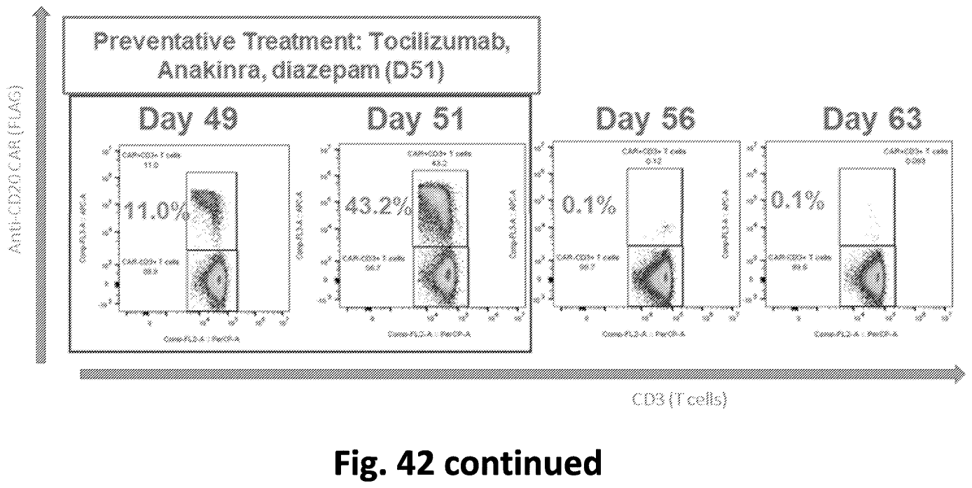

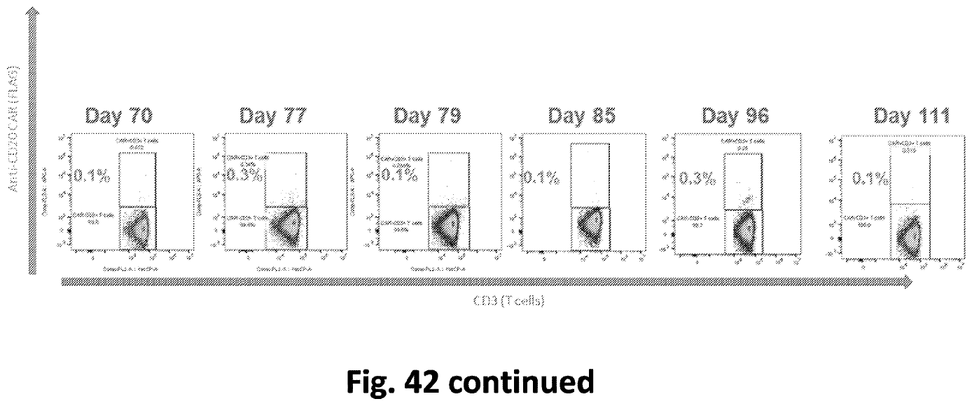

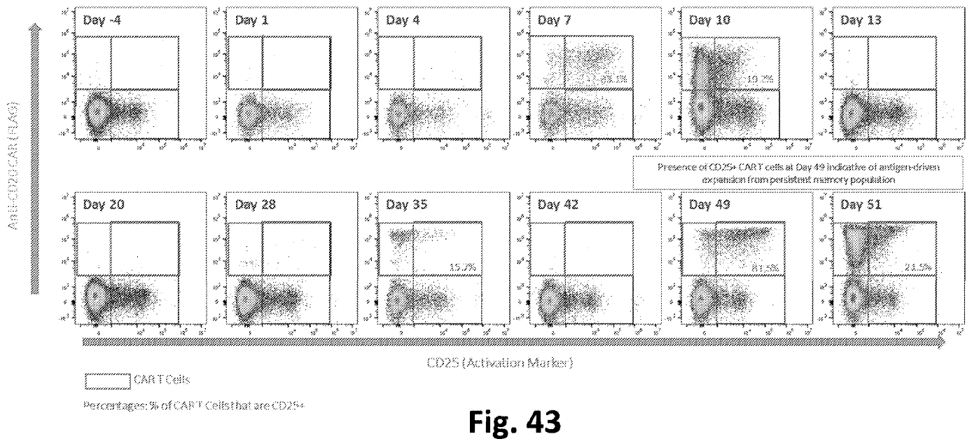

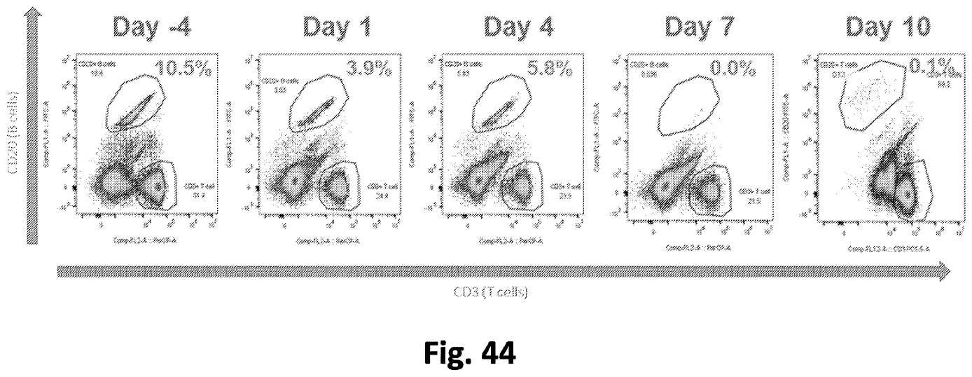

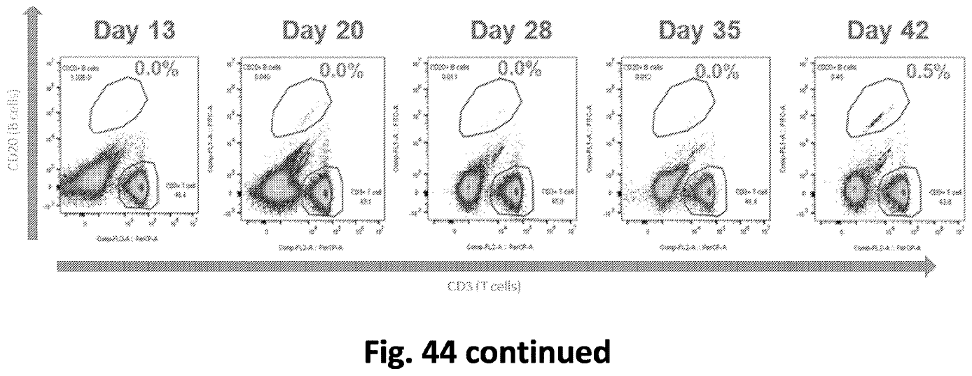

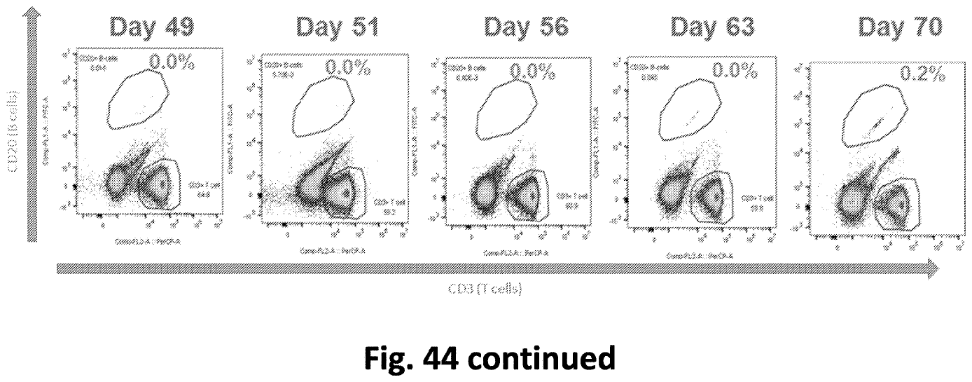

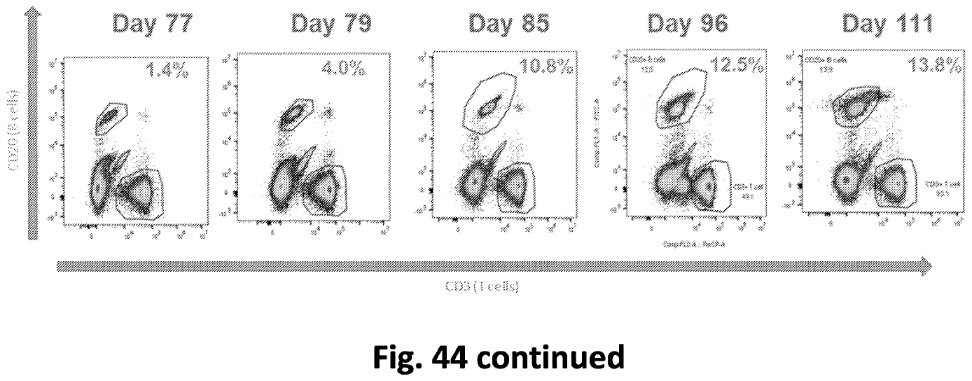

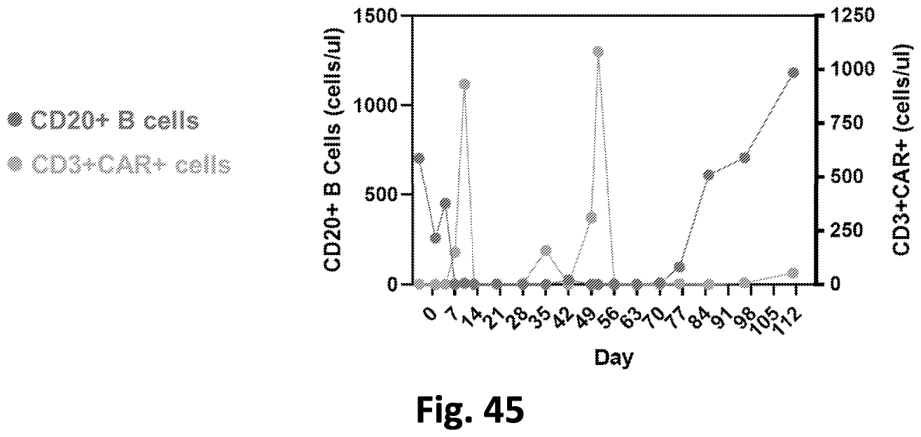

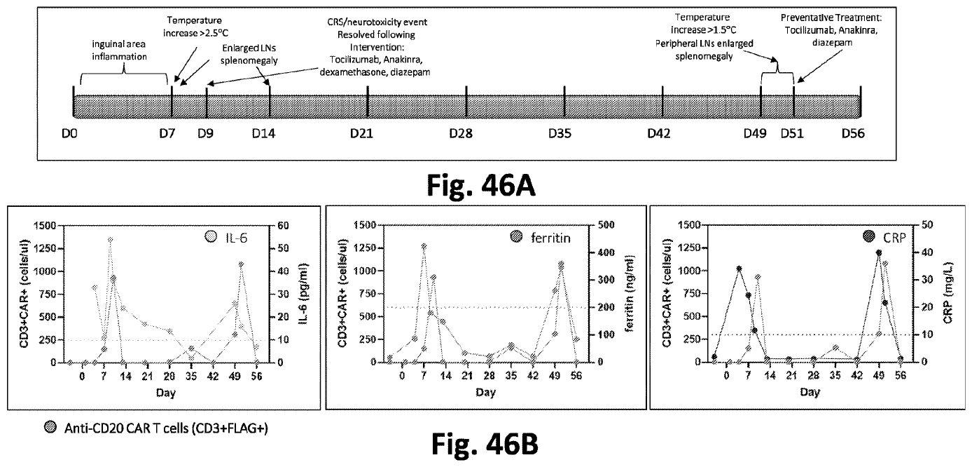

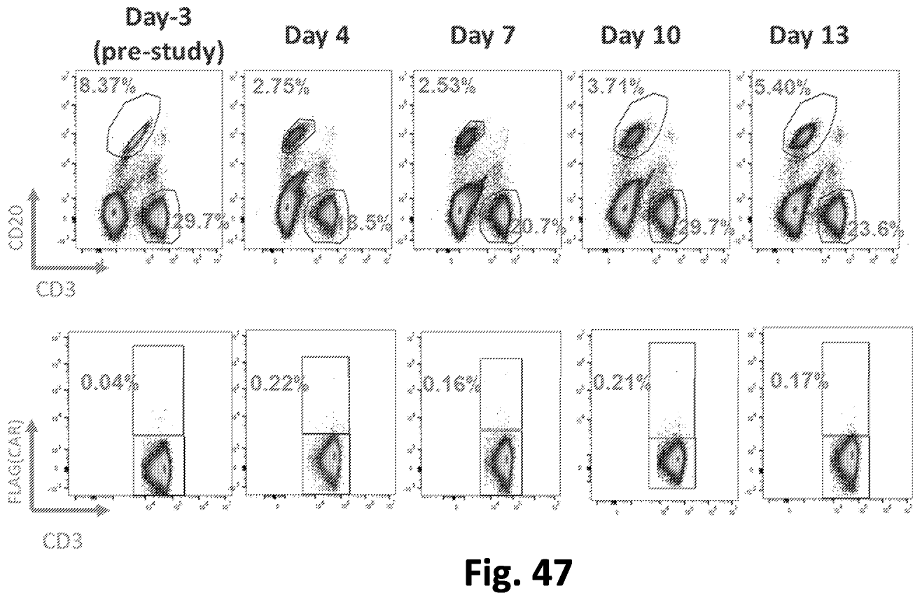

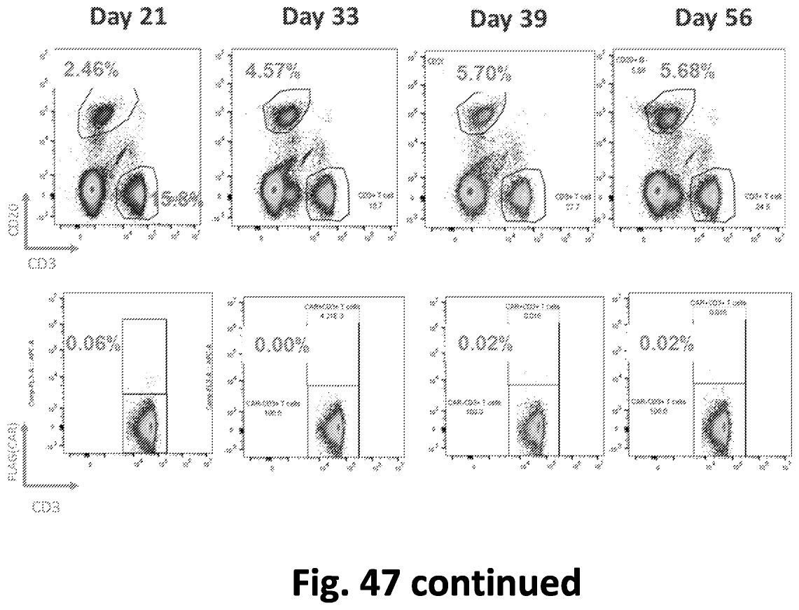

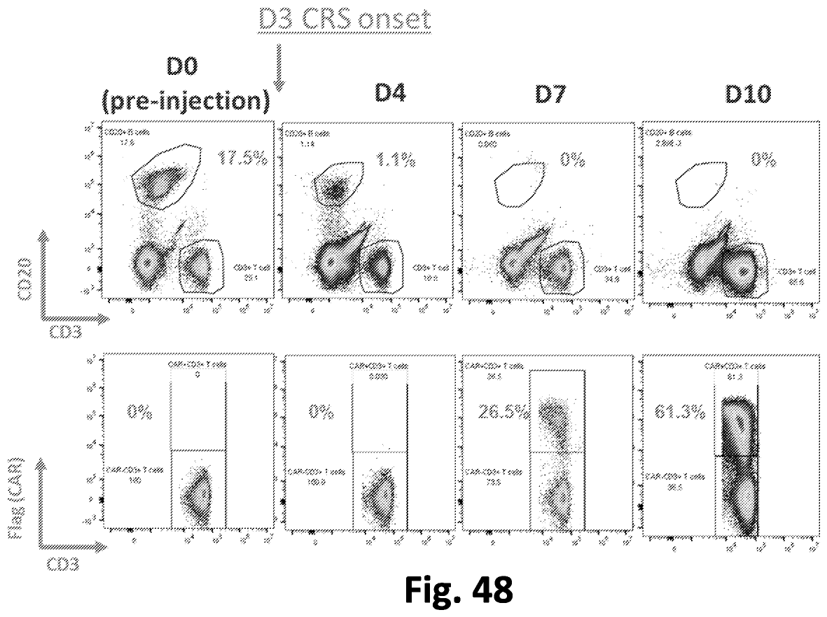

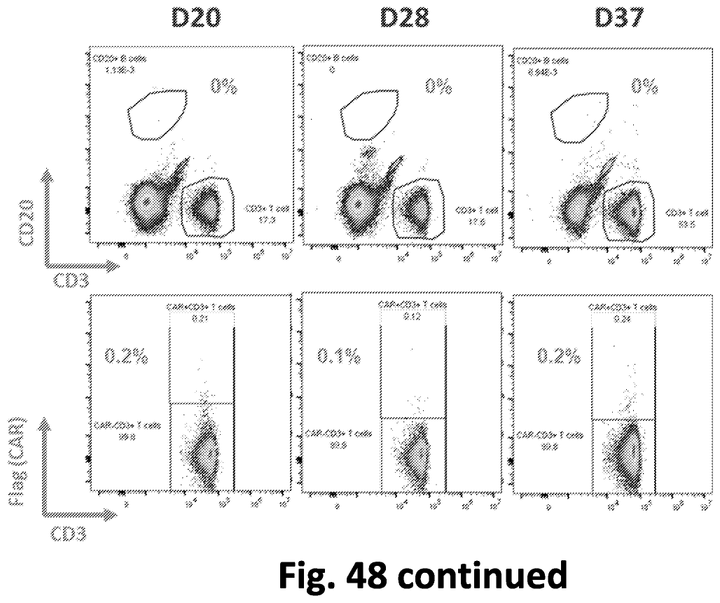

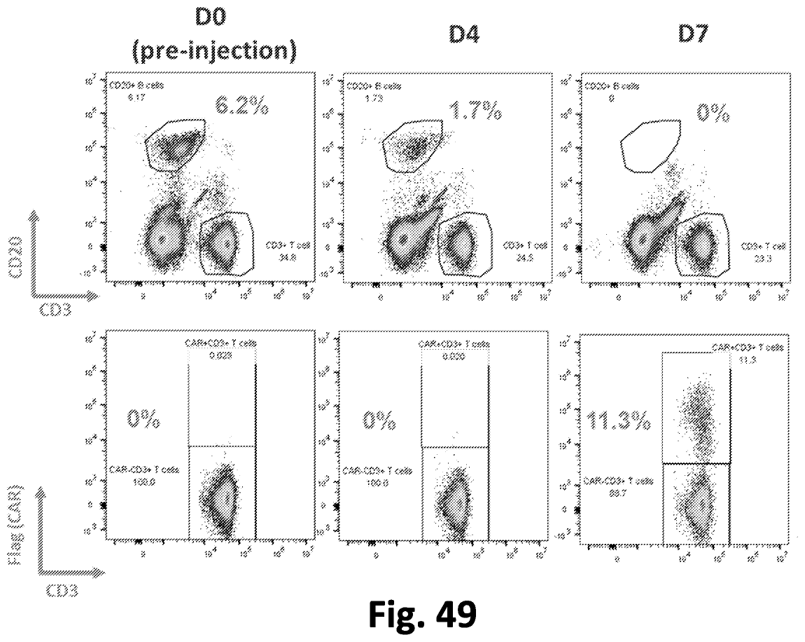

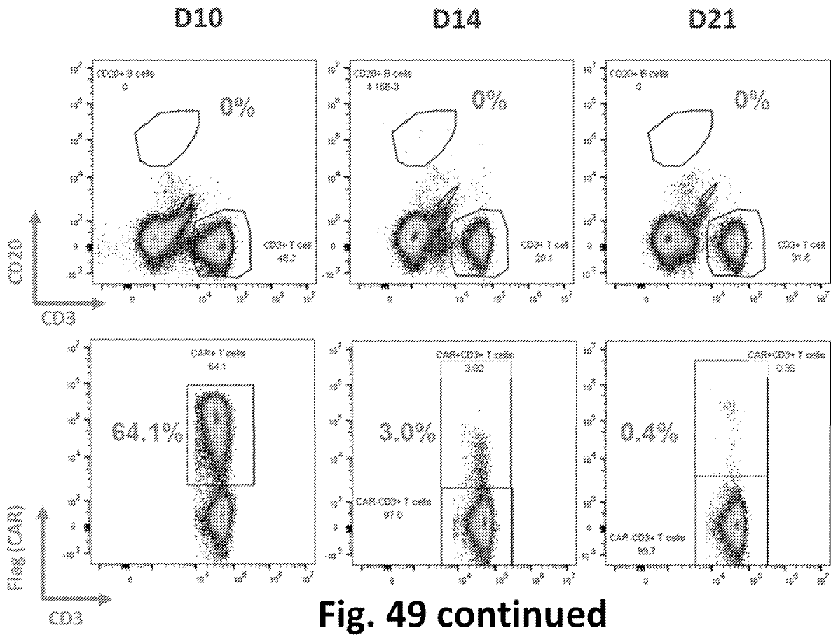

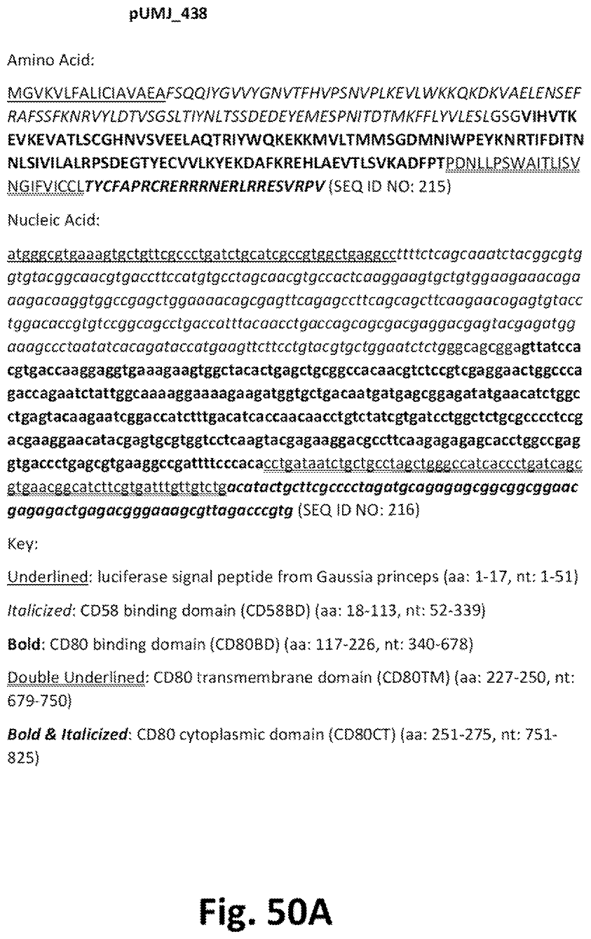

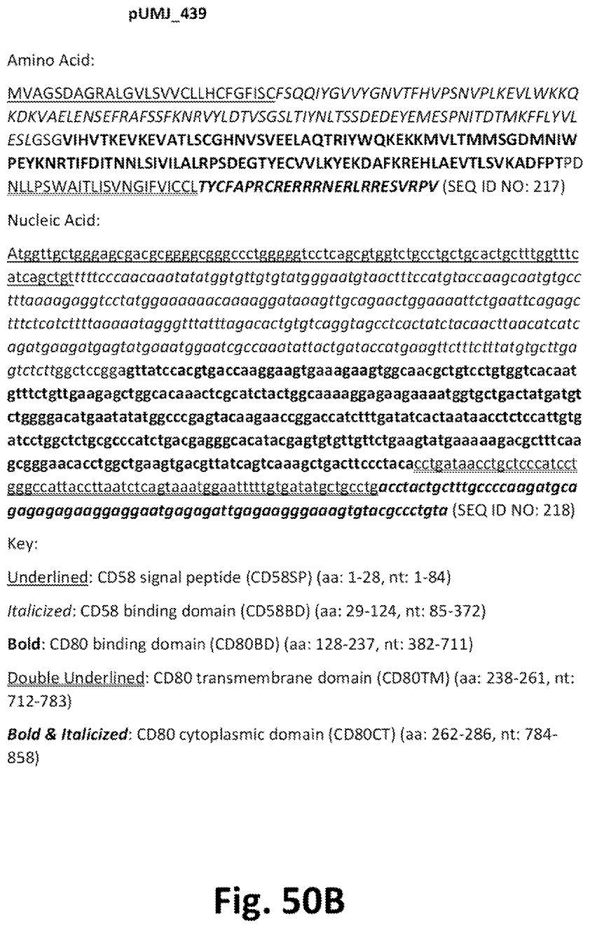

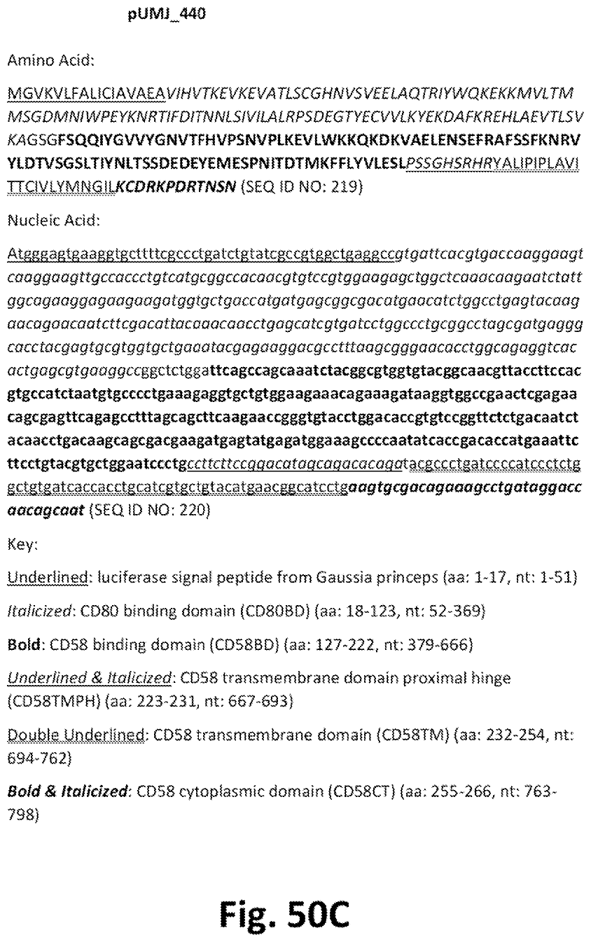

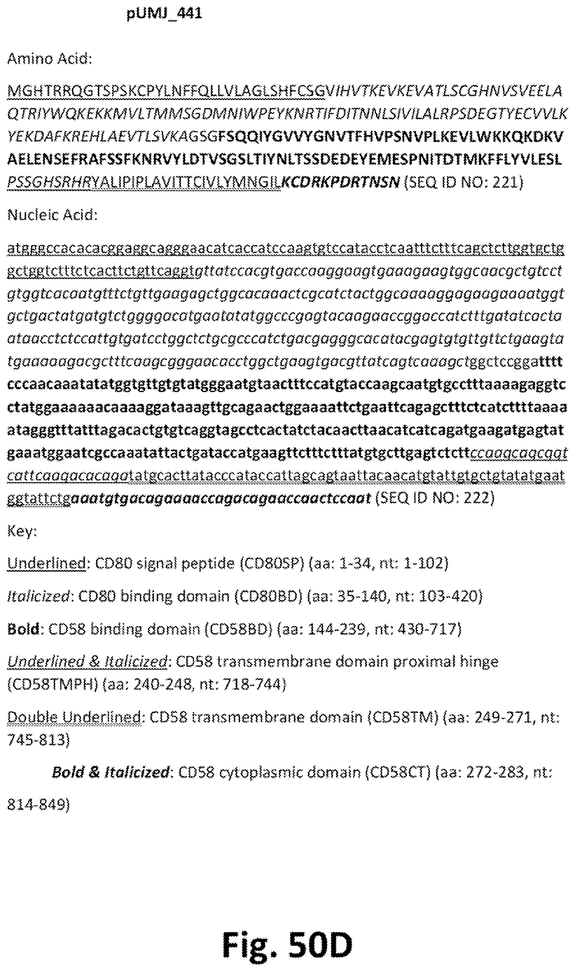

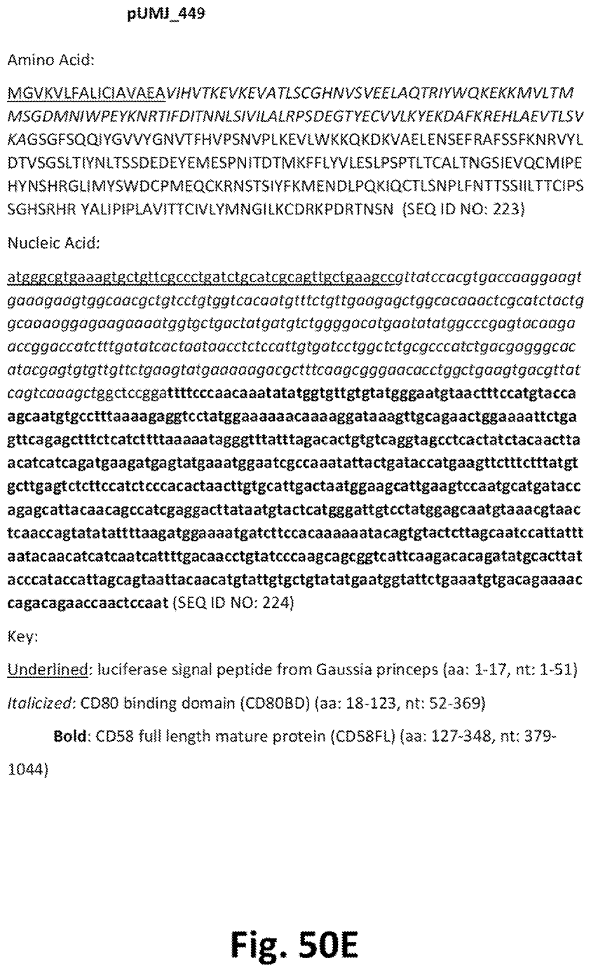

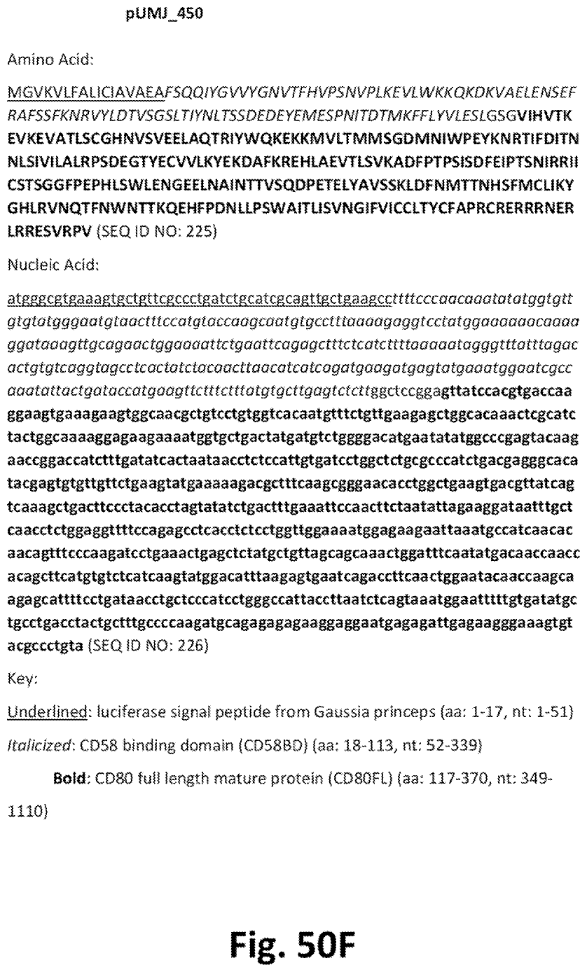

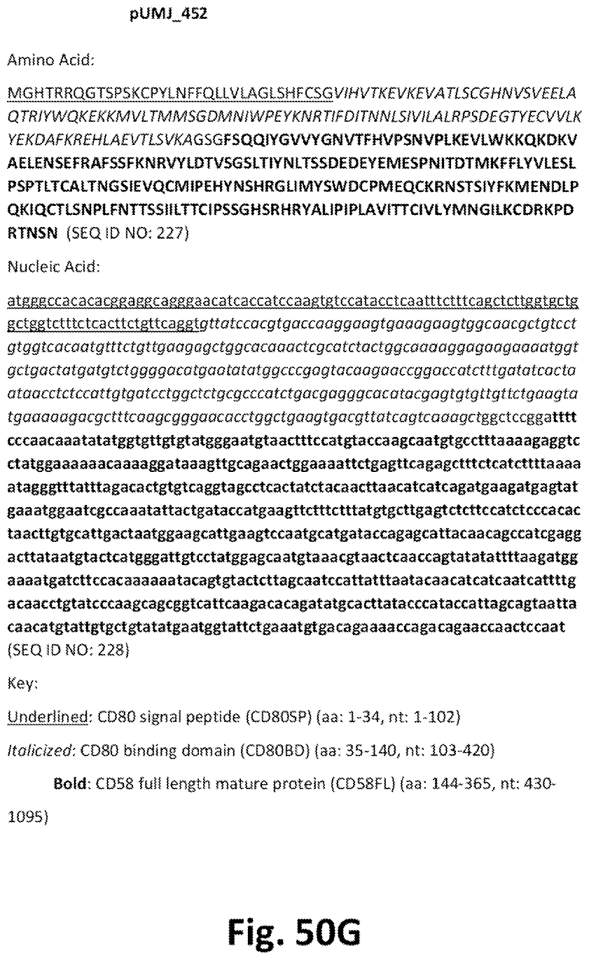

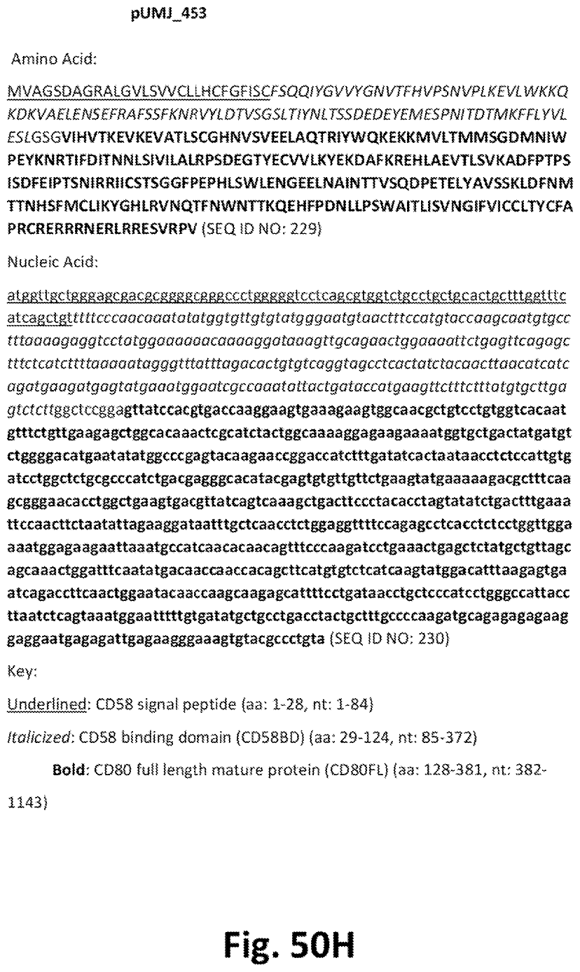

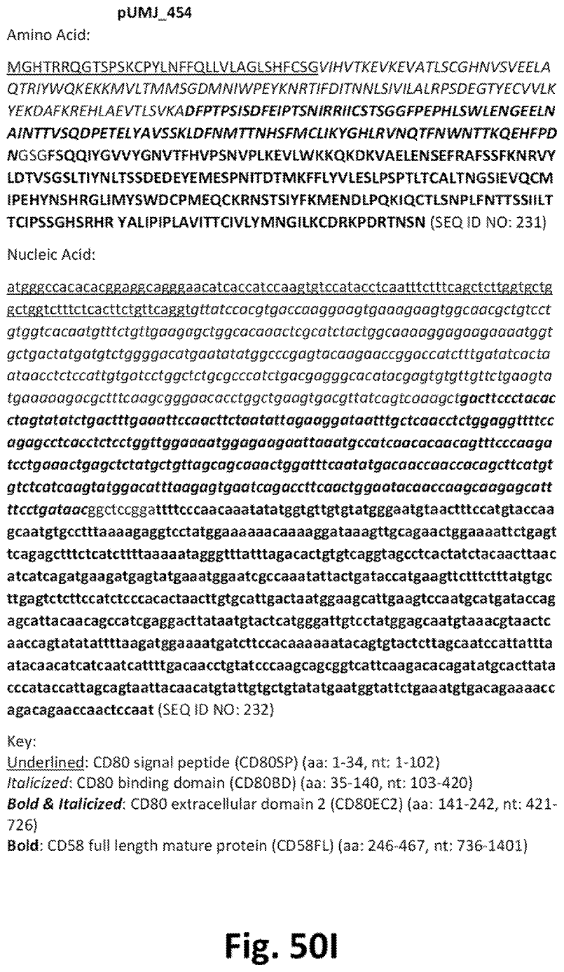

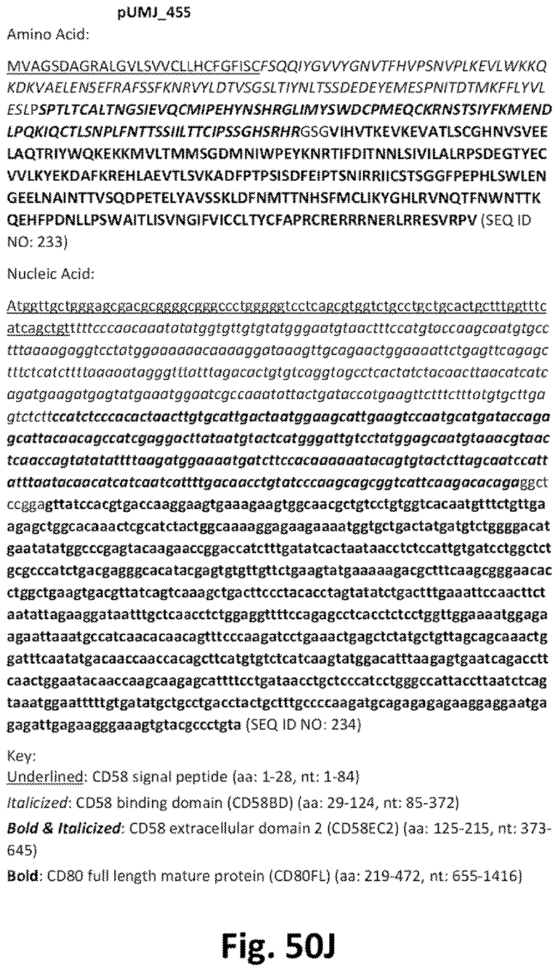

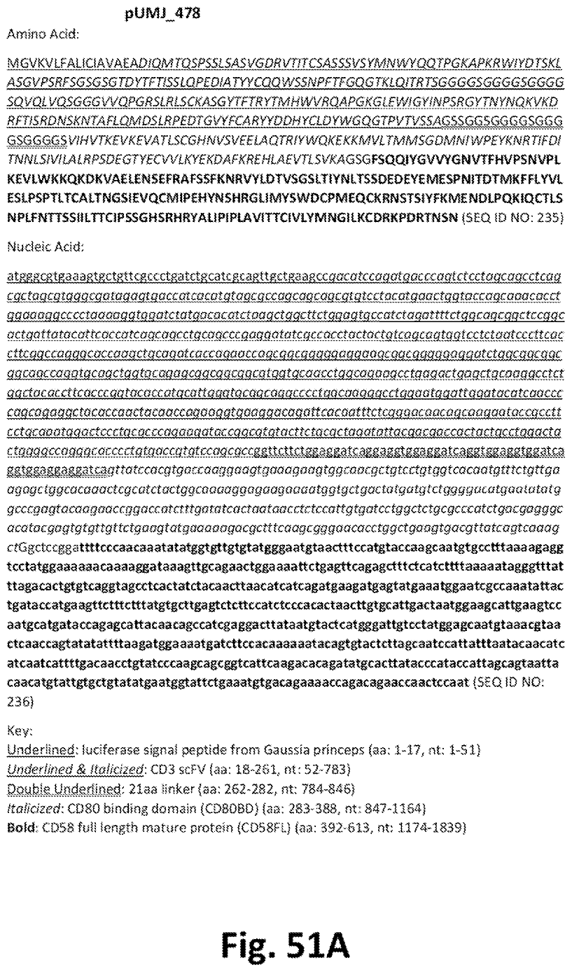

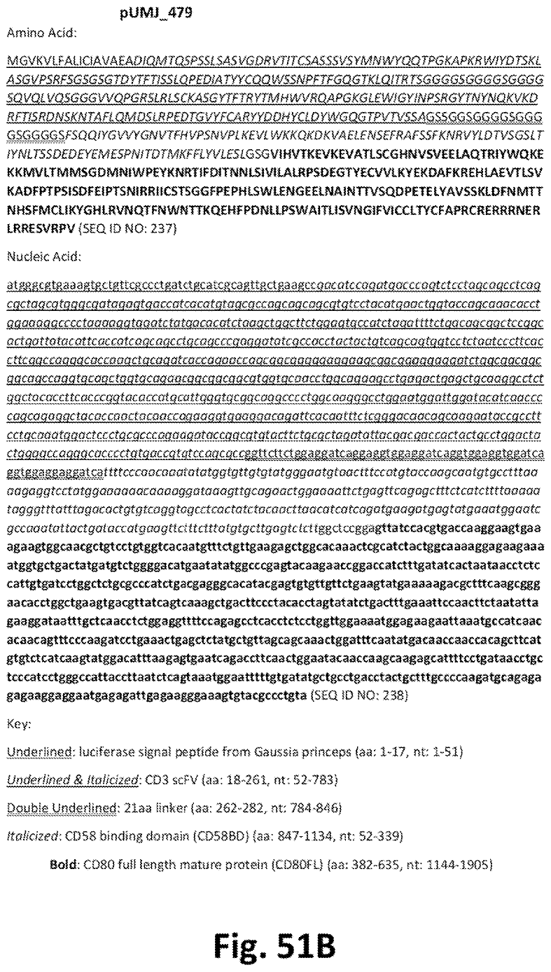

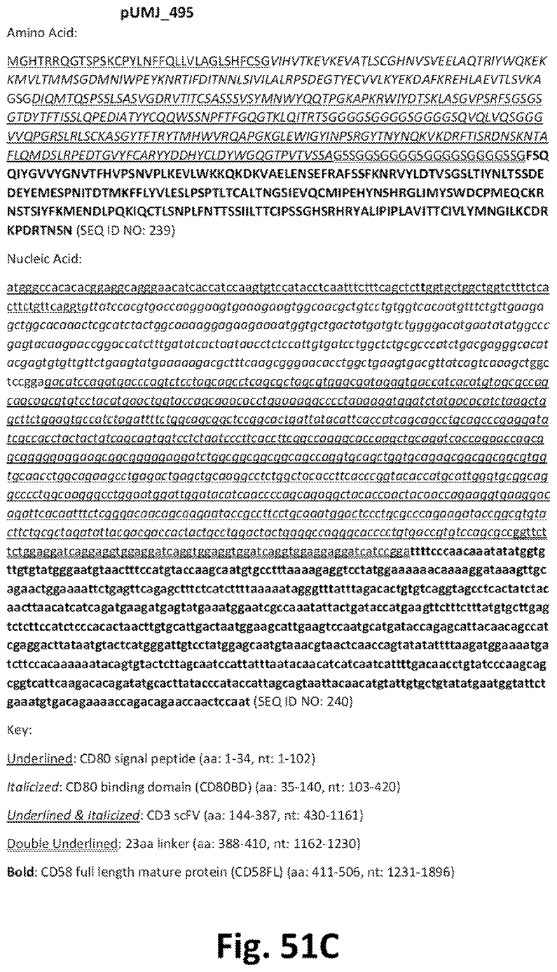

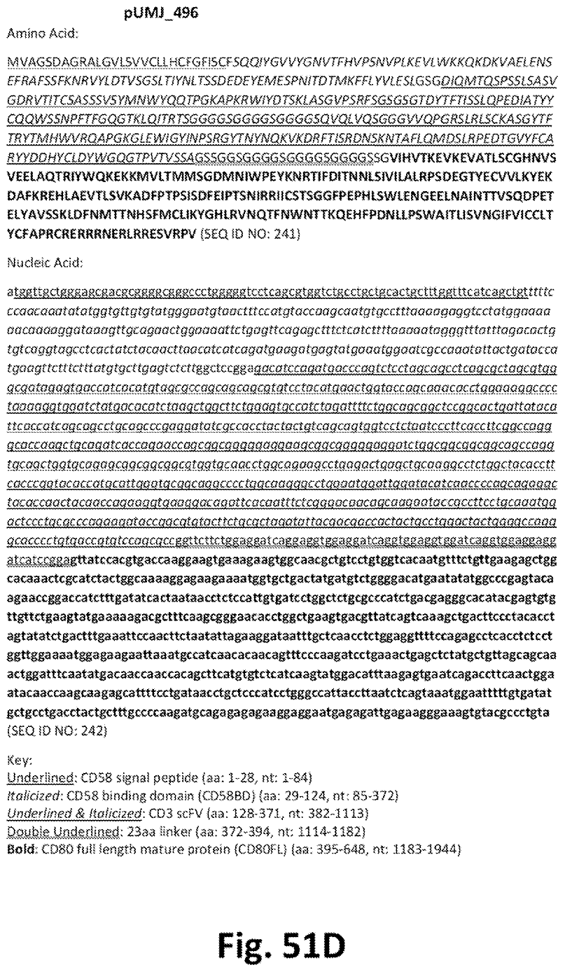

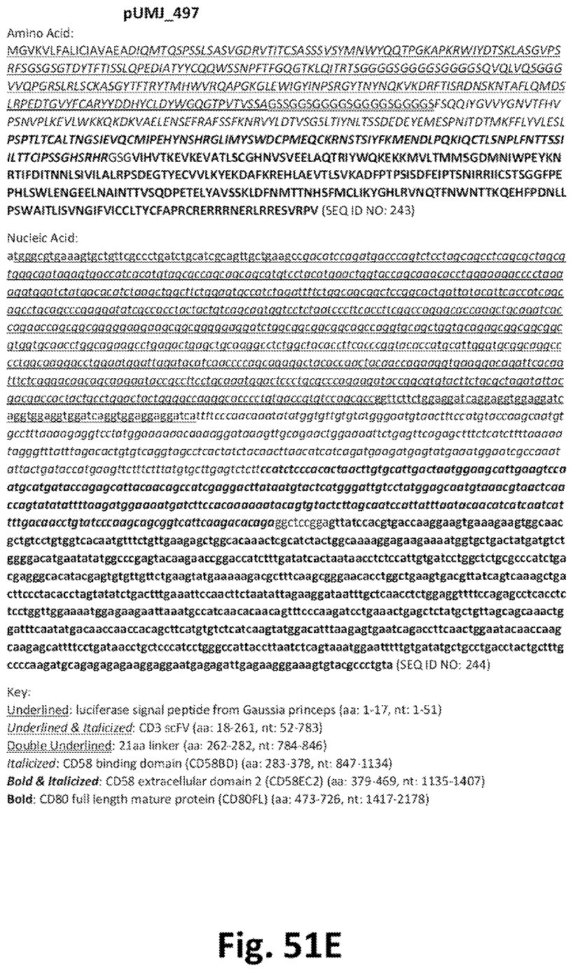

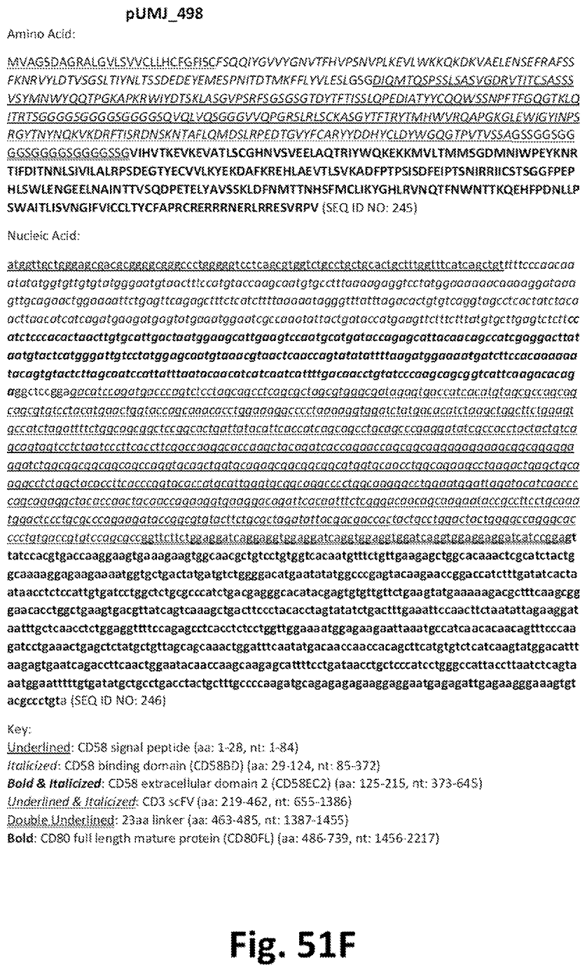

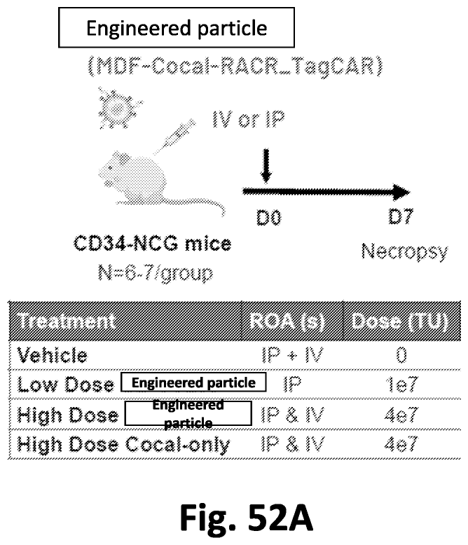

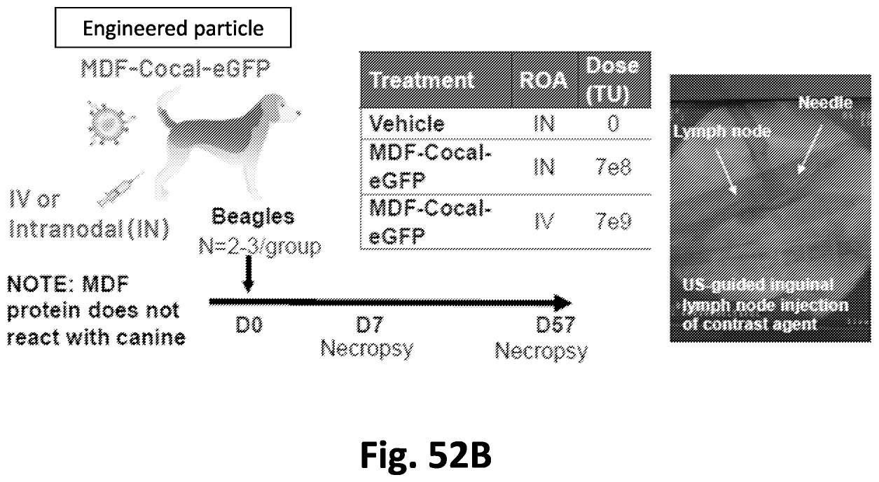

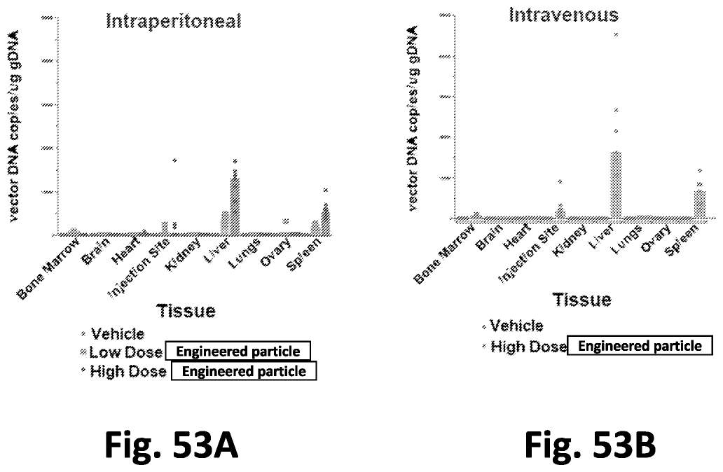

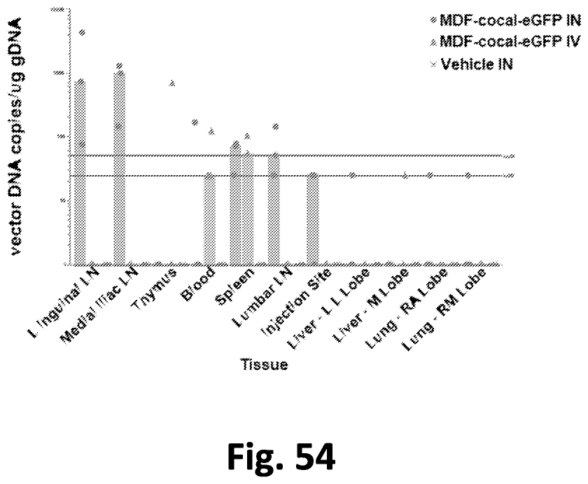

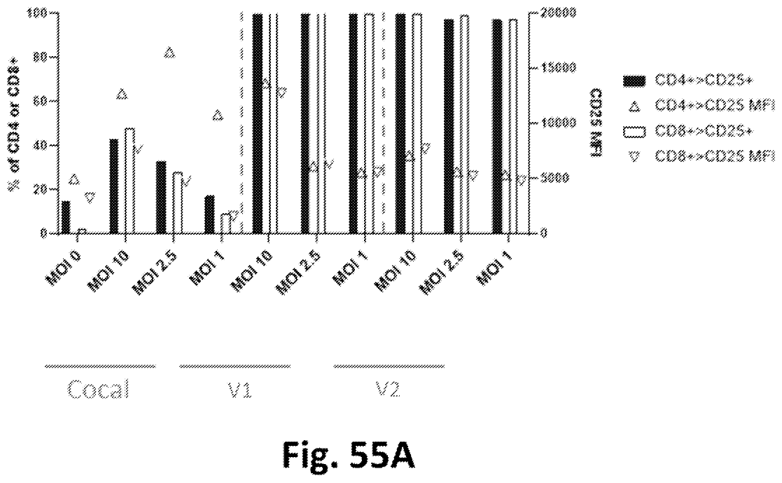

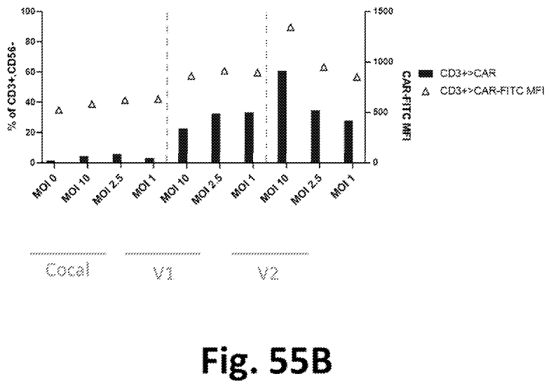

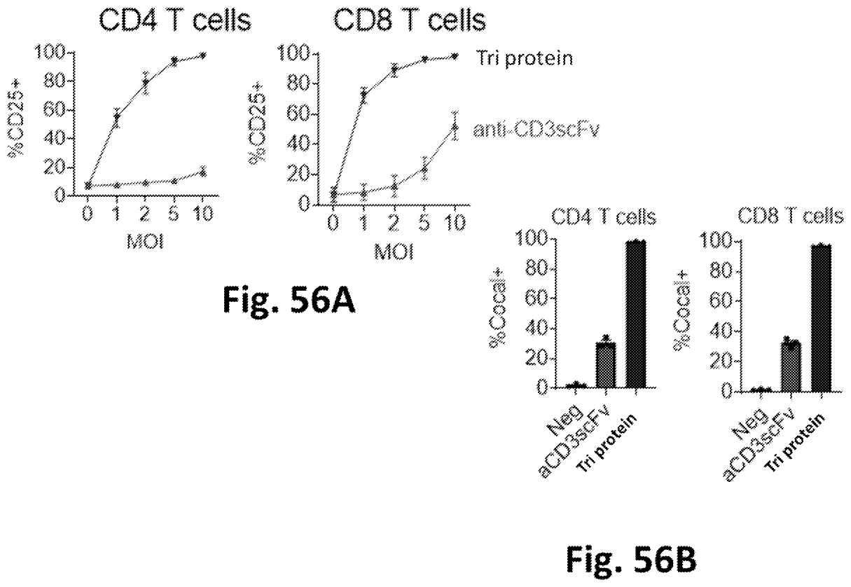

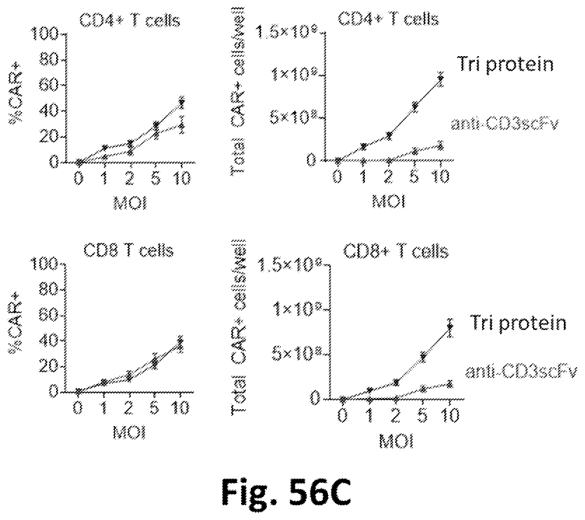

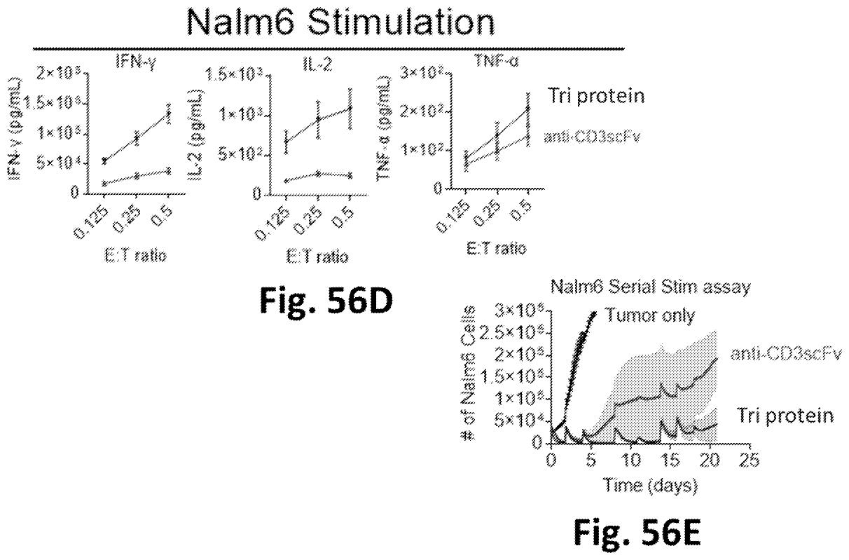

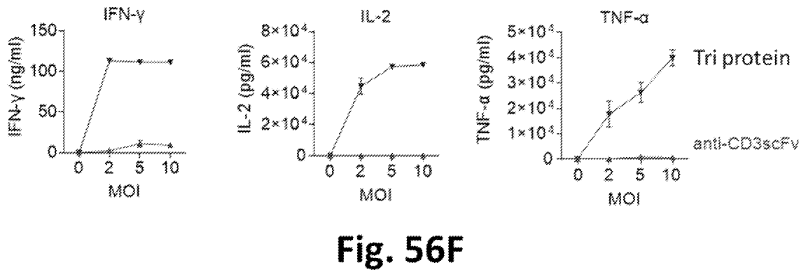

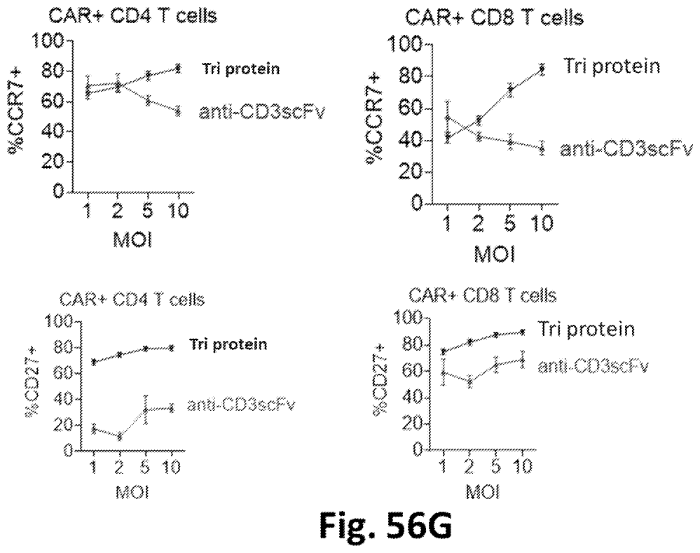

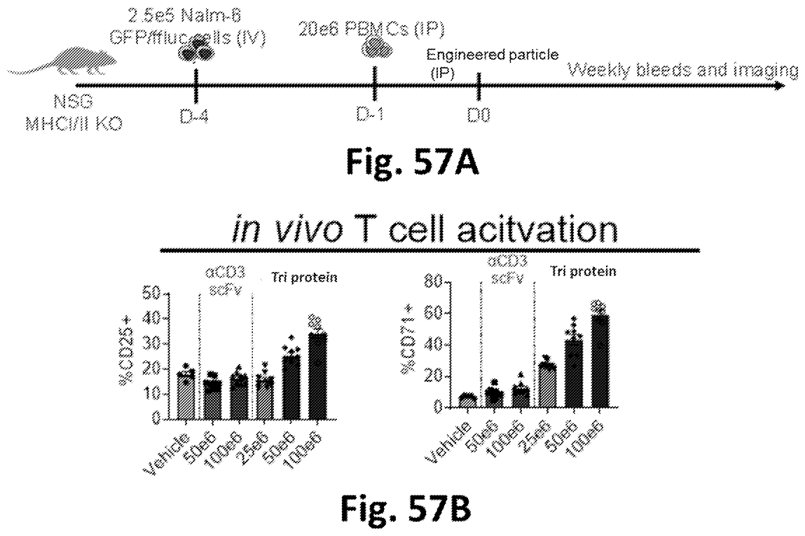

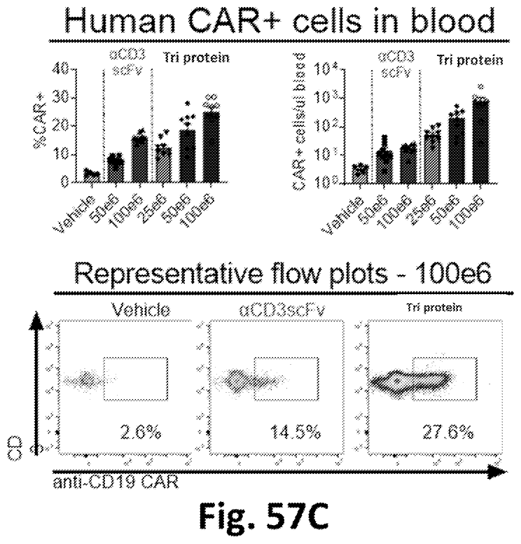

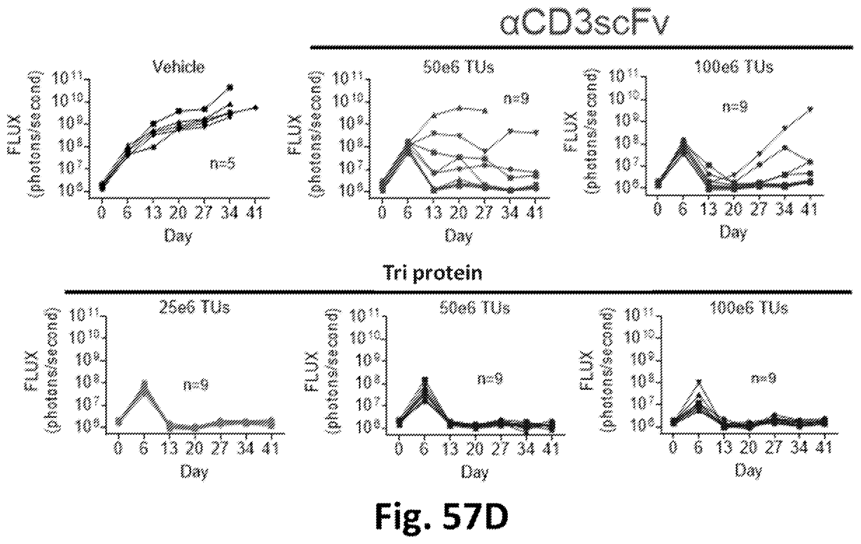

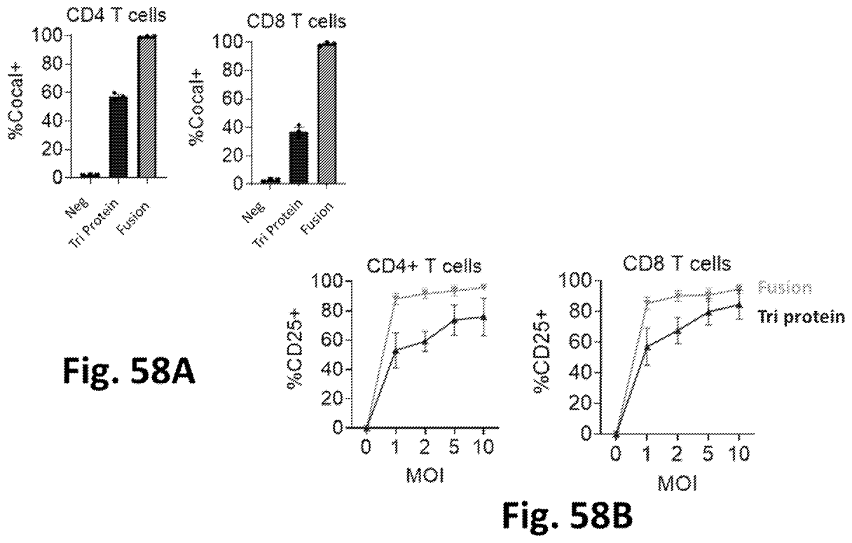

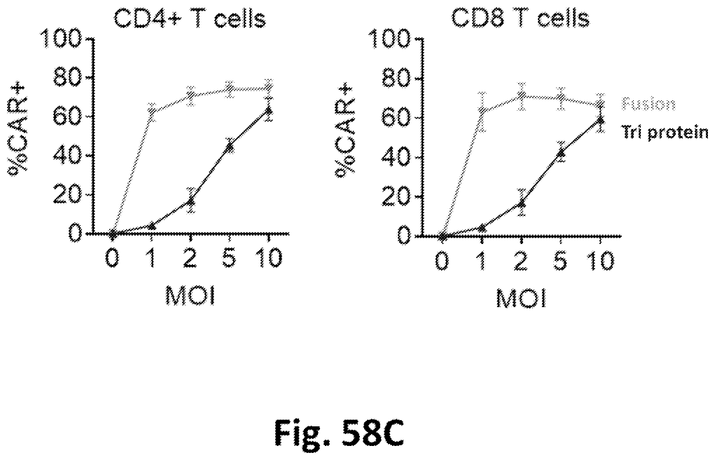

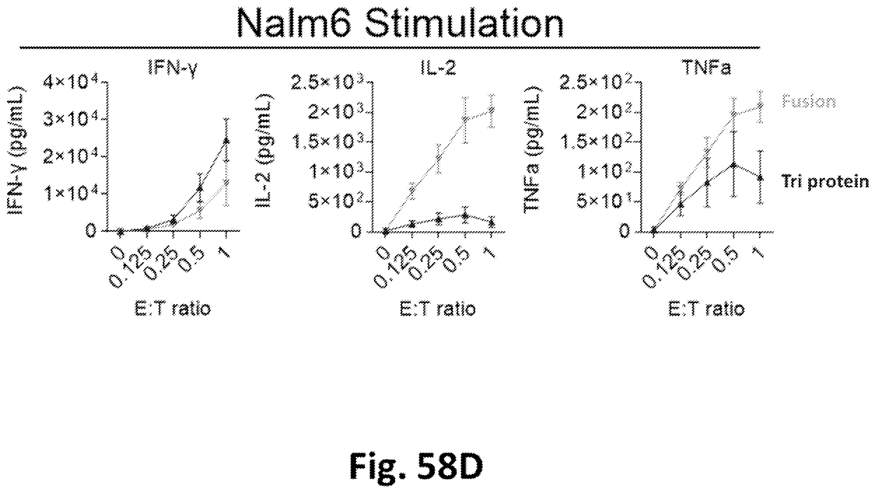

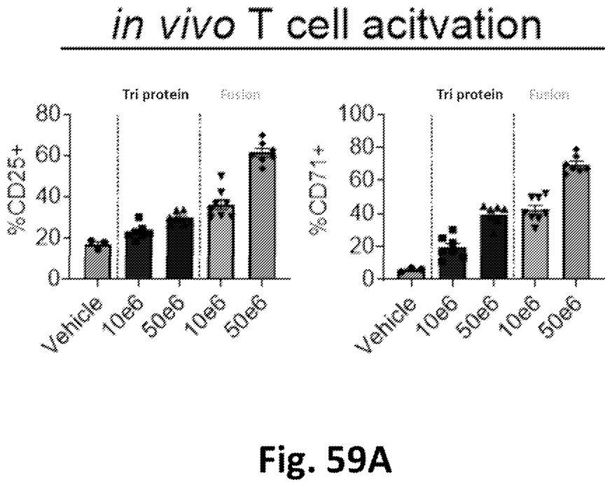

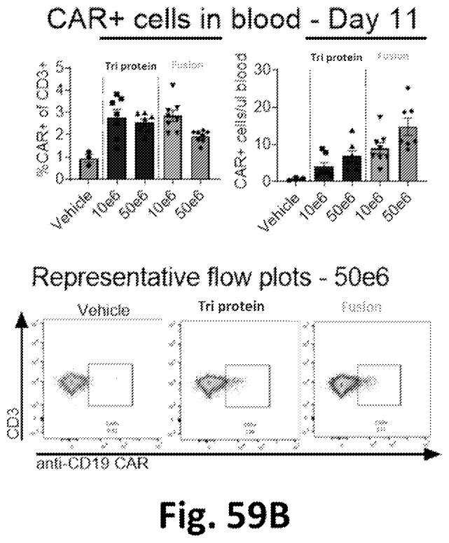

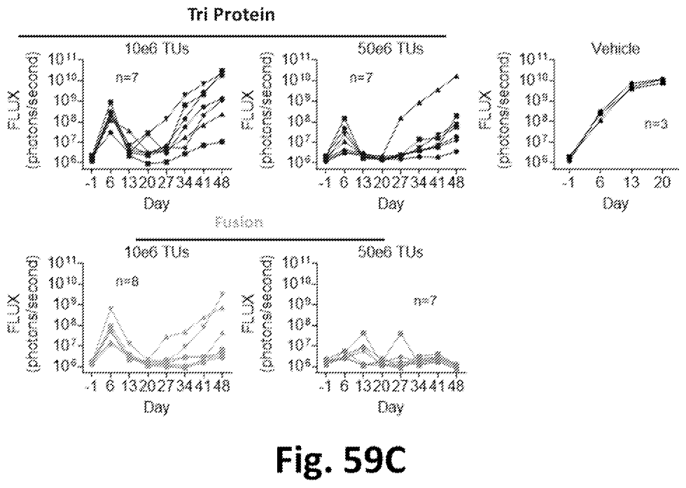

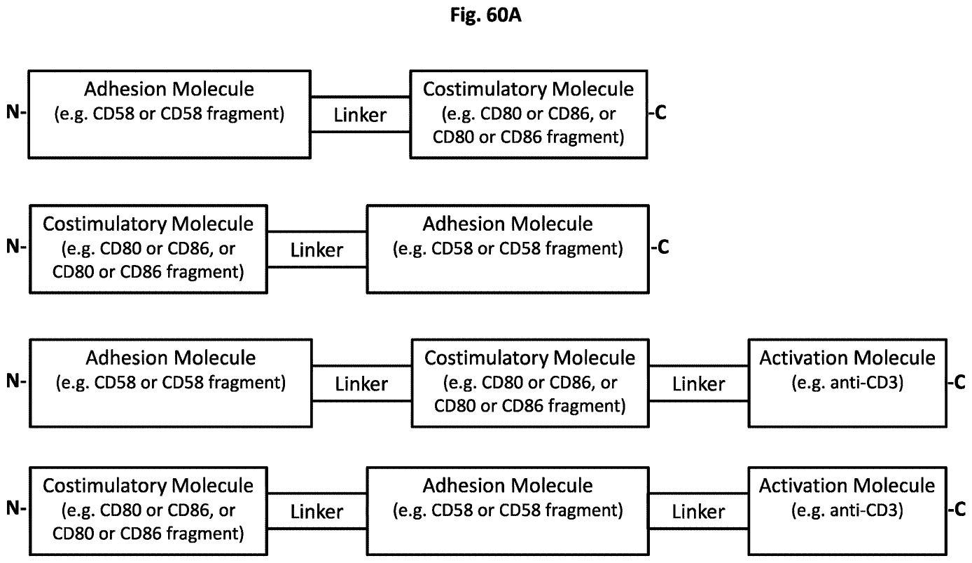

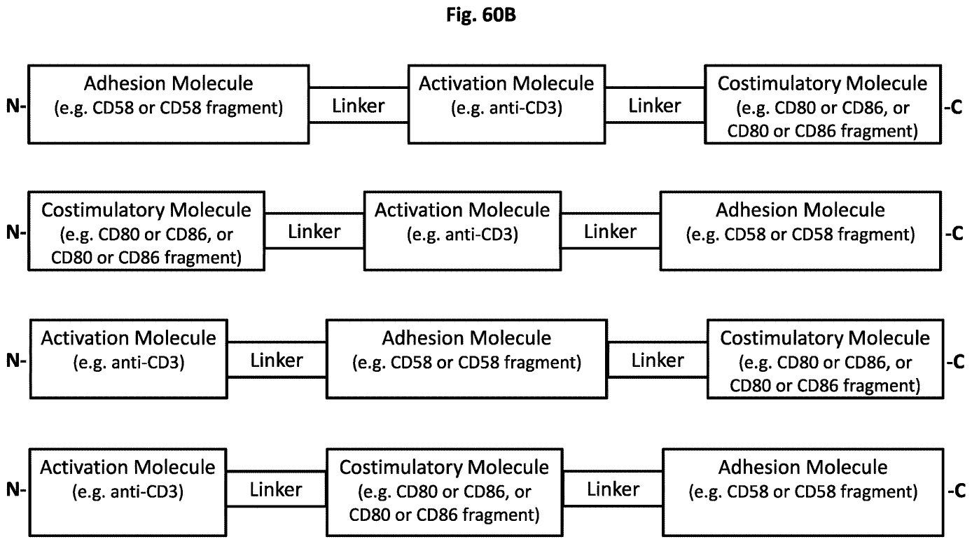

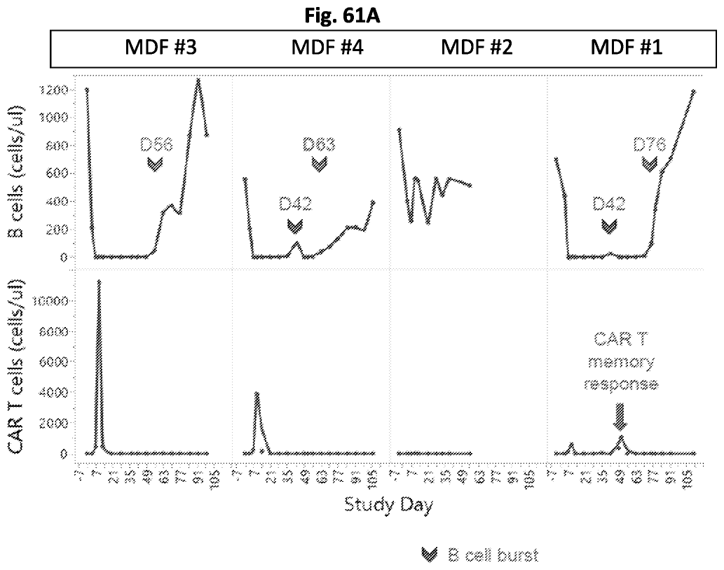

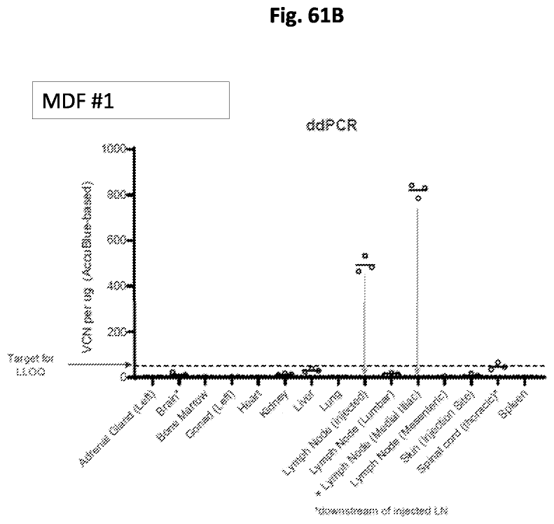

FIGS. 3J-3L show staining of Cocal (FIG. 3J), CD80 (FIG. 3K) or CD58 (FIG. 3L) on CD8+ T cells incubated with lentiviral particles displaying CD3scfv only, CD3scfv+CD80, CD3scfv+CD58, or CD3scfv+CD80+CD58.

FIG. 3M shows a principal components analysis with three main clusters of differentiation based on particle costimulatory-molecule makeup using CCR7, CD45RO, CD45RA, CD27, CD25, CAR+, CD4, and CD8 markers and total cells.

FIG. 3N shows CD3scfv+CD80 particles generate CAR+ T cells with a predominantly central memory (T_{cm}) phenotype compared to CD3scfv only, which produced effector T cells (T_{eff}).

FIG. 3O shows CD3scfv+CD80, CD3scfv+CD58, or CD3scfv+CD80+CD58 particles generate CAR+ T cells with a predominantly central memory (T_{cm}) phenotype compared to CD3scfv only, which produced effector T cells (T_{eff}) central memory T cells (T_{cm}).

FIG. 4A shows the number of K562.CD19 cells over several days after incubation with anti-CD19 CAR+ T cells generated with lentiviral particles encoding an anti-CD19 CAR and displaying CD3scfv only, CD3scfv+CD80, CD3scfv+CD58, or CD3scfv+CD80+CD58 particles. The particles were added to PBMCs at an MOI of 10 along with Tumor cells at PBMC:Tumor ratio of 5:1 and put directly on the Incucyte® live-cell imaging system. CD3scfv+CD80+CD58 CAR T cells were generated using a mixture of individual particles.

FIG. 4B shows the number of Raji cells over several days after incubation with anti-CD19 CAR+ T cells generated with lentiviral particles encoding an anti-CD19 CAR and displaying CD3scfv only, CD3scfv+CD80, CD3scfv+CD58, or CD3scfv+CD80+CD58 particles. The particles were added to PBMCs at an MOI of 10 along with Tumor cells at PBMC:Tumor ratio of 5:1 and put directly on the incucyte. CD3scfv+CD80+CD58 CAR T cells were generated using a mixture of individual particles.

FIG. 4C shows the number of K562.CD19 cells over several days after incubation with anti-CD19 CAR+ T cells generated with lentiviral particles encoding an anti-CD19 CAR and displaying CD3scfv only, CD3scfv+CD80, CD3scfv+CD58, or CD3scfv+CD80+CD58 particles. 7 days after transduction at an MOI of 10, the total CAR+ cells were calculated and incubated with either K562.CD19 at E:T ratios of 0.5 and 1, respectively. CD3scfv+CD80+CD58 CAR T cells were generated using a mixture of individual particles.

FIG. 4D shows the number of Raji cells over several days after incubation with anti-CD19 CAR+ T cells generated with lentiviral particles encoding an anti-CD19 CAR and displaying CD3scfv only, CD3scfv+CD80, CD3scfv+CD58, or CD3scfv+CD80+CD58 particles. 7 days after transduction at an MOI of 10, the total CAR+ cells were calculated and incubated with either Raji cells at E:T ratios of 0.5 and 1, respectively. CD3scfv+CD80+CD58 CAR T cells were generated using a mixture of individual particles.

FIG. 4E shows the number of K562.CD19 cells over several days after incubation with anti-CD19 CAR+ T cells generated with lentiviral particles encoding an anti-CD19 CAR and displaying CD3scfv only, CD3scfv+CD80, CD3scfv+CD58, or CD3scfv+CD80+CD58 particles. 7 days after transduction at an MOI of 10, the total CAR+ cells were calculated and incubated with K562.CD19 cells at E:T ratios of 1:1, respectively. CD3scfv+CD80+CD58 CAR T cells were generated using a single particle with both costimulatory and adhesion molecules.

FIG. 4F shows the number of Nalm6 cells over several days after incubation with anti-CD19 CAR+ T cells generated with lentiviral particles encoding an anti-CD19 CAR and displaying CD3scfv only, CD3scfv+CD80, CD3scfv+CD58, or CD3scfv+CD80+CD58 particles. 7 days after transduction at an MOI of 10, the total CAR+ cells were calculated and incubated with Nalm6 cells at E:T ratios of 1:1, respectively. CD3scfv+CD80+CD58 CAR T cells were generated using a single particle with both costimulatory and adhesion molecules. FIG. 4A-4F are labeled with a key to the right of each plot with labels that correspond (in order) to the right end of each line in the plot.

FIG. 5A shows the number of CAR T cells in blood samples of NSG MHCI/II KO mice 11 days after injection of PMBCs and lentiviral particles displaying CD3scfv only or CD3scfv+CD80 particles.

FIGS. 5B-5C show the tumor burden in NSG MHCI/II KO mice over 100 days after administration with lentiviral particles displaying CD3scfv only (FIG. 5B) or CD3scfv+CD80 (FIG. 5C).

FIGS. 6A-6B show number of cells expressing a CAR 3 days (FIG. 6A) or 7 days (FIG. 6B) after transduction of PBMCs from three healthy donors with lentiviral particles displaying CD3scfv only or CD3scfv+CD80+CD58 particles.

FIGS. 7A-7C show expression of CAR in cells transduced with lentiviral particles pseudotyped with mutant VSV-G envelope proteins. SupT1 cells (FIG. 7A) or PBMCs from two healthy donors (FIGS. 7B-7C) were cultured with lentiviral particles having an anti-CD19 CAR payload and displaying mutant VSV-G envelope proteins with or without CD3scfv+CD80+CD58. CAR expression was assessed in CD4+ T cells (FIG. 7B) and CD8+ T cells (FIG. 7C) after transduction of the PBMCs.

FIG. 8 shows the number of CAR negative T cells in the blood of mice after administration of particles at indicated doses encoding an anti-CD19 CAR and displaying CD3scfv only or CD3scfv+CD80+CD58.

FIG. 9A is a schematic that shows an illustrative fusion protein comprising a CD58 extracellular region and α-CD3 scFv fused to the N-terminus of a CD80 via a linker. The construct is termed “498.”

FIG. 9B is a schematic that shows an illustrative fusion protein comprising a CD58 extracellular region fused to the N-terminus of a CD80 via a linker. The construct is termed “455.” α-CD3 scFv is expressed as a separate polypeptide in the producer cells.

FIG. 10 shows staining of Cocal in CD8+ T cells generated with lentiviral particles displaying α-CD3 scFv, CD80, and CD58 which were expressed by the lentiviral particle producer cells as separate polypeptides (“Separate”); lentiviral particles displaying α-CD3 scFv, CD80, and CD58 which were expressed by the lentiviral particle producer cells as fusion polypeptide comprising CD58 fused to CD80, with α-CD3 scFv expressed as a separate polypeptide (“455”); and lentiviral particles displaying a fusion protein comparing CD58, α-CD3 scFv, and CD80 (“498”), or control without lentiviral particles (“MOI 0”).

FIG. 11A shows the percent CD25(+) CD4+ T cells after incubation with lentiviral particles, labelled as in FIG. 10.

FIG. 11B shows the percent of CD25(+) CD8+ T cells after incubation with lentiviral particles, labelled as in FIG. 10.

FIG. 11C shows the geometric mean fluorescent intensity (gMFI) CD25(+) CD4+ T cells after incubation with lentiviral particles, labelled as in FIG. 10.

FIG. 11D shows the geometric mean fluorescent intensity (gMFI) of CD25(+) CD8+ T cells after incubation with lentiviral particles, labelled as in FIG. 10.

FIGS. 12A-12C show production of cytokines 3 days after incubation with lentiviral particles, labelled as in FIG. 10. IFN-γ (FIG. 12A), IL-2 (FIG. 12B), and TNF-α (FIG. 12C) levels were measured. Particles comprised an anti-CD19-FRB-RACR payload. FRB=FKBP-rapamycin complex binding domain; RACR=rapamycin-activated cell-surface receptor.

FIGS. 13A-13D show “#498” displaying particles generate CAR+ T cells with a larger proportion of memory-like CD4+ (FIG. 13A and FIG. 13B) or memory-like CD8+ (FIG. 13C and FIG. 13D) CAR T cells compared to “#455” or “Separate” displaying particles.

FIG. 14A is a schematic that shows an illustrative experimental timeline.

FIG. 14B shows the percent CD25(+) CD3+ T cells in blood after incubation with lentiviral particles, labelled as in FIG. 10.

FIG. 14C shows the percent CD71(+) CD3+ T cells in blood after incubation with lentiviral particles, labelled as in FIG. 10.

FIG. 14D shows level of IFN-γ cytokine measured 4 days after incubation with lentiviral particles, labelled as in FIG. 10.

FIG. 15A-15C is a panel of graphs showing geometric mean fluorescent intensity (gMFI) of Cocal in cells generated with lentiviral particles via extracorporeal in vivo incubation. Cells were stained prior to incubation with lentiviral particles “pre-particle”, following lentiviral particle incubation with cell but before washing (“particle, pre-wash”), or following lentiviral particle incubation with cell and after washing (“Final”). Lentiviral particles display CD58 and CD80 expressed as a fusion polypeptide are labeled as in FIG. 10. Lentiviral particle incubation with CD4+ T cells, CD8+ T cells, NK T cells, NK cells, CD56+NK cells, monocytes, B cells, and other mean fluorescent intensity (MFI) was evaluated.

FIG. 16A shows CAR+ T cells in the blood of mice injected with PBMCs from Donor 1 either after Lupagen™ wash or after incubation with lentiviral particles labeled as in FIG. 10.

FIG. 16B shows CAR+ T cells in the blood of mice injected with PBMCs from Donor 2 either after Lupagen™ wash or after incubation with lentiviral particles labeled as in FIG. 10.

FIG. 16C shows total tumor burden (Total flux) over the course of 21 days of the study in the blood of mice injected with PBMCs from Donor 1, either after Lupagen™ wash or after incubation labeled as in FIG. 10.

FIG. 16D shows total tumor burden (Total flux) over the course of 21 days of the study in the blood of mice injected with PBMCs from Donor 2 either after Lupagen™ wash or after incubation labeled as in FIG. 10.

FIG. 16E shows Bioluminescence imaging using the IVIS™ spectrum system depicting total tumor burden quantitated in FIG. 16C and FIG. 16D.

FIGS. 17A-17B show expression of CD25 in CD4+ (FIG. 17A) cells transduced with lentiviral particles produced using the indicated surface plasmids encoding variations of CD58 and CD80 fusion polypeptide expression or expression of CD25 in CD8+ cells (FIG. 17B).

FIG. 17C shows a bar graph of the effects of lentivirus particles produced using the indicated surface plasmids. Non-stimulated human PBMCs were cultured labeled as in FIG. 10. T cell early activation measured by CD25 expression level on Day 3 post PBMC culturing was analyzed by flow cytometry. NTC (non-transduced cells) are included for comparison. Lentiviral particles were added at multiplicity of infection (MOI) 2 and 5.

FIGS. 18A-18B show CAR expression in CD4+ (FIG. 18A) cells transduced with lentiviral particles produced using the indicated surface plasmids encoding variations of CD58 and CD80 fusion polypeptide expression or expression of CD25 in CD8+ cells (FIG. 18B).

FIG. 18C shows a bar graph of the effects of lentivirus particles produced using the indicated surface plasmids. Non-stimulated human PBMCs were cultured with lentiviral particles displaying variations of CD58 and CD80 fusion polypeptide. CAR expression as measured by FMC63 expression level on Day 7 post PBMC culturing was analyzed by flow cytometry. NTC (non-transduced cells) are included for comparison. Lentiviral particles were added at multiplicity of infection (MOI) 2 and 5.

FIGS. 19A-19D show the effects of indicated lentiviral surface proteins on FMC63 CAR-T induced cytotoxicity in the presence of NucLight™ Red-labeled Nalm6 target cells expressing hCD19 antigen. The IncuCyte™ kinetic killing curves for each CAR-T variant transduced using lentivirus displaying variations of CD58 and CD80 fusion polypeptides at MOI 2. FIG. 19A shows a CAR-T to target cell ratio of 0.25:1. FIG. 19B shows killing curves when CAR-T to target cell ratio is 0.5:1. FIG. 19C shows killing curves when CAR-T to target cell ratio is 1:1. FIG. 19D shows target-cell lytic capabilities of CAR-T cells by integrating the area under the normalized target cell killing curves (AUC) when CAR-T cells to target cell ratio range from 0.25 to 4. Percent (%) Antigen specific, CAR mediated Killing=(1−(AUC/AUC_{Mock}))*100.

FIG. 20 is a bar graph showing target-dependent IFN-γ, IL-2, and TNFα secretion in FMC63 CAR-T cells, which were transduced lentiviral particles displaying indicated variations of CD58 and CD80 fusion polypeptides at MOI 2. The transduced T cells were co-cultured with Nalm6 target cells at CAR-T cell to target cell ratio of 1:1.

FIGS. 21A-21B show CD25 expression in CD4+ (FIG. 21A) cells transduced with lentiviral particles produced using the indicated surface plasmids encoding variations of CD58 and CD80 fusion polypeptide expression or expression of CD25 in CD8+ cells (FIG. 21B).

FIG. 21C shows a bar graph of the effects of lentivirus particles produced using the indicated surface plasmids. Non-stimulated human PBMCs were cultured with lentiviral particles displaying variations of CD58 and CD80 fusion polypeptide. T cell early activation measured by CD25 expression level on Day 3 post PBMC culturing was analyzed by flow cytometry. NTC (non-transduced cells) are included for comparison. Lentiviral particles were added at multiplicity of infection (MOI) 0.5 and 1.

FIGS. 22A-22B show CAR expression in CD4+ (FIG. 22A) cells transduced with lentiviral particles produced using the indicated surface plasmids encoding variations of CD58 and CD80 fusion polypeptide expression or expression of CD25 in CD8+ cells (FIG. 22B).

FIG. 22C shows a bar graph of the effects of lentivirus particles produced using the indicated surface plasmids. Non-stimulated human PBMCs were cultured with lentiviral particles displaying variations of CD58 and CD80 fusion polypeptide. CAR expression as measured by FMC63 expression level on Day 7 post PBMC culturing was analyzed by flow cytometry. NTC (non-transduced cells) are included for comparison. Lentiviral particles were added at multiplicity of infection (MOI) 0.5 and 1.

FIG. 23 shows a bar graph of the effects of lentivirus particles produced using the indicated surface plasmids. Non-stimulated human PBMCs were cultured with lentiviral particles displaying variations of CD58, CD80, and anti-CD3 scFv fusion polypeptides. T cell early activation measured by CD25 expression level on Day 3 post PBMC culturing was analyzed by flow cytometry. NTC (non-transduced cells) are included for comparison. Lentiviral particles were added at multiplicity of infection (MOI) 1 and 10.

FIG. 24 shows a bar graph of the effects of lentivirus particles produced using the indicated surface plasmids. Non-stimulated human PBMCs were cultured with lentiviral particles displaying variations of CD58, CD80, and anti-CD3 scFv fusion polypeptides. CAR expression as measured by FMC63 expression level on Day 7 post PBMC culturing was analyzed by flow cytometry. NTC (non-transduced cells) are included for comparison. Lentiviral particles were added at multiplicity of infection (MOI) 1 and 10.

FIG. 25 shows a graph of CAR+ T cell expansion over 11 days post transduction. The CAR+ T cells were transduced with lentiviral particles displaying variations of CD58, CD80, and anti-CD3 scFv fusion polypeptides. On Day 3 post transduction, PBMCs are washed to remove lentiviral particles, and seeded in fresh culture media at 0.5E6 cells per well. CAR+ cells were determined by staining for surface expression of anti-FMC63 scFv, and analyzed by flow cytometry.

FIG. 26 is a schematic of the fusion polypeptide screening approach depicted in FIGS. 17-25.

FIG. 27A shows diagrams of illustrative fusion proteins.

FIG. 27B shows diagrams of illustrative fusion proteins. The 21aa linker may have the polypeptide sequence GSSGGSGGGGSGGGGSGGGGS (SEQ ID NO: 34). The 23aa linker may have the polypeptide sequence GSSGGSGGGGSGGGGSGGGGSSG (SEQ ID NO: 35).

FIG. 28A shows a study design and timeline.

FIG. 28B is a graph showing staining of Cocal on CD3+ T cells incubated with engineered particles displaying CD58, CD80, and anti-CD3 scFv tri-fusion polypeptide.

FIG. 28C is a graph showing staining of Cocal on engineered particle bound T cells The left peak shows CD3− T cells and the right peak shows CD3+ T cells. The engineered particles display a CD58, CD80, and anti-CD3 scFv tri-fusion polypeptide.

FIG. 28D shows CD25 expression in CD8+ T cells on day 3 after transduction with lentiviral particles displaying CD58, CD80, and anti-CD3 scFv tri-fusion polypeptide “Engineered particle”.

FIG. 28E shows CAR expression in CD8+ T cells on day 7 after transduction with lentiviral particles displaying CD58, CD80, and anti-CD3 scFv tri-fusion polypeptide “Engineered particle”.

FIG. 29 shows the number of Nalm6 cells after anti-CD19 CAR+ T cells were serial-stimulated with Nalm6 tumor cells every 2-3 days. anti-CD19 CAR+ T cells were generated with lentiviral particles encoding an anti-CD19 CAR transgene and displaying CD3scfv-CD80-CD58 tri-fusion polypeptide particles “Engineered particle”. Arrows denote stimulation with Nalm6 tumor cells. Error bars denote mean±SEM.

FIG. 30A shows the study design and timeline. FIG. 30B shows the number of cells expressing activation marker CD25 in circulation four days after transduction with lentiviral particles displaying CD3scfv-CD80-CD58− tri-fusion polypeptide. FIG. 30C shows the number of cells expressing activation marker CD71 in circulation four days after transduction with lentiviral particles displaying CD3scfv-CD80-CD58 tri-fusion polypeptide. FIG. 30D shows production of IFN-γ 4 days after incubation with particles displaying CD3scfv-CD80-CD58 tri-fusion polypeptide “Engineered particle”. FIG. 30E shows the number of T cells expressing an anti-CD19 CAR in the blood 11 days after transduction with lentiviral particles displaying CD3scfv-CD80-CD58− tri-fusion polypeptide at a lentiviral dose of 10 Million or 50 Million transducing units (TU). FIG. 30F shows the tumor burden in NSG MHCI/II KO mice after administration of lentiviral particles displaying CD3scfv-CD80-CD58 tri-fusion polypeptide at a lentiviral dose of 10 Million or 50 Million transducing units (TU).

FIG. 31A shows the study design and timeline. FIG. 31B shows the number of T cells from Donor 1 and Donor 2 expressing an anti-CD19 CAR in the blood 14 days after extracorporeal incubation with lentiviral particles. FIG. 31C shows the tumor burden in Donor 1 and Donor 2 NSG MHCI/II KO mice after administration of T cells produced by via extracorporeal incubation with lentiviral particles. N=7 animals; error bars denote mean±SEM. FIG. 31D shows the study design and timeline for re-challenge study. FIG. 31E shows the tumor burden in NSG MHCI/II KO mice after administration of T cells produced via extracorporeal incubation of PBMCs from Donor 1 or Donor 2 incubated with lentiviral particles following tumor cell re-challenge at Day 49. Error bars denote mean±SEM.

FIG. 32A shows the study design and timeline. FIG. 32B shows % CAR+ T cells (left panel) and total CAR+ T cells (right panel) in the blood of mice injected with PBMCs from Donor 1 after Lupagen™ incubation with lentiviral particles displaying α-CD3 scFv, CD80, and CD58 which were expressed by the lentiviral particle producer cells as a bi-fusion polypeptide comprising CD58 fused to CD80, with α-CD3 scFv expressed as a separate polypeptide (“#455”); and lentiviral particles displaying a tri-fusion protein comparing CD58, α-CD3 scFv, and CD80 (“#498”), or control PBMCs not incubated with lentiviral particles. FIG. 32C shows % CAR+ T cells (left panel) and total CAR+ T cells (right panel) in the blood of mice injected with PBMCs from Donor 2 after Lupagen™ incubation with lentiviral particles displaying α-CD3 scFv, CD80, and CD58 which were expressed by the lentiviral particle producer cells as a bi-fusion polypeptide comprising CD58 fused to CD80, with α-CD3 scFv expressed as a separate polypeptide (“#455”); and lentiviral particles displaying a tri-fusion protein comparing CD58, α-CD3 scFv, and CD80 (“#498”), or control PBMCs not incubated with lentiviral particles. FIG. 32D shows Bioluminescence imaging using the IVIS™ spectrum system depicting total tumor burden. Images show mice injected with PBMCs from Donor 1 after Lupagen™ incubation with lentiviral particles displaying “#455” a dual fusion or “#498” a triple fusion (left panel), and mice injected with PBMCs from Donor 2 after Lupagen™ incubation with lentiviral particles displaying “#455” a dual fusion or “#498” a triple fusion (right panel). FIGS. 32E-32G show total tumor burden (Total flux) over the course of 45 days of the study in the blood of mice injected with PBMCs from Donor 1 (top row of panels) or Donor 2 (bottom row of panels) after Lupagen™ incubation with untreated PBMCs (FIG. 32E), lentiviral particles displaying “#455” a dual fusion (FIG. 32F), or “#498” a triple fusion (FIG. 32G). FIGS. 32H-32I show total tumor burden (Total flux) over the course of 28 days of the study in the blood of mice injected with PBMCs from Donor 1 (FIG. 32H) or Donor 2 (FIG. 32I) after Lupagen™ incubation with untreated PBMC control, lentiviral particles displaying “#455”, or “#498” a dual fusion or a triple fusion polypeptide, respectively.

FIG. 33A shows the study design and timeline for re-challenge study. FIG. 33B shows the tumor burden in NSG MHCI/II KO mice after administration of T cells produced via extracorporeal incubation of PBMCs from Donor 1 (D1) or Donor 2 (D2) incubated with lentiviral particles displaying “#455” a dual fusion construct, or “#498” a triple fusion construct following tumor cell re-challenge at Day 49. FIG. 33C shows Bioluminescence imaging using the IVIS™ spectrum system depicting total tumor burden. Images show mice injected with PBMCs from Donor 1 after Lupagen™ incubation with lentiviral particles displaying “#455” a dual fusion or “#498” a triple fusion (left panel), and mice injected with PBMCs from Donor 2 after Lupagen™ incubation with lentiviral particles displaying “#455” a dual fusion or “#498” a triple fusion (right panel) following tumor cell rechallenge at Day 49. FIG. 34A shows the study design.

FIG. 34B is a graph showing staining of Cocal on CD3+ T cells incubated with no vector control (left panel), engineered particles displaying CD58, CD80, and anti-CD3 scFv individually expressed polypeptides (middle panel), and engineered particles displaying a CD58, CD80, and anti-CD3 scFv multi-domain fusion (MDF) polypeptide (right panel).

FIG. 34C is a graph showing % Cocal on B cells, CD4+ T cells, CD8+ T cells, monocytes, and NK cells incubated with viral particles displaying individually expressed CD3scfv+CD80+CD58 (left panel) and viral particles displaying CD58, CD80, and anti-CD3 scFv multi-domain fusion (MDF) (right panel).

FIG. 34D is a graph showing geometric mean fluorescence intensity (gMFI) Cocal on B cells, CD4+ T cells, CD8+ T cells, monocytes, and NK cells incubated with viral particles displaying individually expressed CD3scfv+CD80+CD58 (left panel) and viral particles displaying CD58, CD80, and anti-CD3 scFv multi-domain fusion (MDF) (right panel).

FIG. 34E shows the study timeline.

FIG. 34F shows activation of CD3+ T cells as measured by % CD25+ cells 3 days after transductions with a viral particle displaying CD58, CD80, and anti-CD3 scFv multi-domain fusion (MDF).

FIG. 34G shows the level of CD3+ T cells transduction as measured by % CAR expression 7 days after transduction with a viral particle displaying CD58, CD80, and anti-CD3 scFv multi-domain fusion (MDF).

FIG. 34H is a flow cytometry staining showing CAR expression in CD3+ T cells on day 7 after 1 hour of contact with viral particles displaying CD58, CD80, and anti-CD3 scFv multi domain fusion polypeptide.

FIG. 341 is a graph showing the percent of CAR positive T cells (left panel) and viral copy number (VCN) (right panel) on day 7 after 1 hour of contact with viral particles displaying CD58, CD80, and anti-CD3 scFv tri-fusion polypeptide multi domain fusion polypeptide (left panel).

FIG. 35A is a diagram of an illustrative surface engineered viral particle displaying a CD58, CD80, dual-fusion polypeptide and an anti-CD3 scFv that binds NHP CD3 and a payload comprising a human-specific anti-CD20 CAR.

FIG. 35B is a diagram of an illustrative payload comprising a human-specific anti-CD20 CAR. The payload comprises:
a. an MND promoter,
b. an anti-CD20-based CAR comprising:
   i. Leu16 scFv—murine anti-human CD20 scFv (cross-reactive with NHP)
   ii. CD8a hinge and transmembrane domain
   iii. 4-1BB co-stimulatory domain and CD3ζ domain
c. low-affinity nerve growth factor receptor (LNGFR) molecule as a marker of transduction.

FIG. 35C is a summary chart of the animals treated in an illustrative non-human primate study. Three animals were studied. * denotes at start of study.

FIG. 36 is a diagram of an illustrative study design and timeline for an NHP study. The engineered viral particles were injected into auxiliary lymph nodes (LN) of Animal #1 due to difficulty injecting inguinal lymph nodes (LN) due to the small size of the animal. The engineered viral particles were injected into inguinal lymph nodes in Animal #2 and Animal #3.

FIG. 37 is a graph showing the fraction of starting CD20+ cells in Animal #1, Animal #2, and Animal #3 over the course of the study to Day 56.

FIG. 38 is a panel of graphs showing serum levels of IL-6, ferritin, and C-reactive protein (CRP) in Animal #1, Animal #2, and Animal #3 over the course of the study to Day 56 and body temperature to Day 28.

FIG. 39A is a diagram of an illustrative surface engineered viral particle displaying a CD58, CD80, and anti-CD3 scFv tri-fusion polypeptide comprising an anti-CD3 scFv that binds NHP CD3 and a payload comprising a human-specific anti-CD20 CAR.

FIG. 39B is a diagram of an illustrative payload comprising a human-specific anti-CD20 CAR. The payload comprises:
a. an MND promoter,
b. an anti-CD20-based CAR comprising:
   i. Leu16 scFv—murine anti-human CD20 scFv (cross-reactive with NHP)
   ii. FLAG tag
   iii. CD8a hinge and transmembrane domain
   iv. 4-1BB co-stimulatory domain and CD3ζ domain.

FIG. 40 is a panel of flow cytometry staining graphs showing CAR expression in CD3+ T cells after 8 different donor PBMCs were transduced with viral particles displaying Human CD58-NHP-specific anti-CD3 scFv-Human CD80 multi-domain fusion (MDF) polypeptide at MOI=0.2.

FIG. 41A is a diagram of an illustrative study design and timeline for an NHP study.

FIG. 41B is a summary chart of the animals treated in an illustrative non-human primate study. Four animals were studied.

FIG. 42 is a panel of flow cytometry staining graphs showing anti-CD20 CAR expression in CD3+ T cells through to Day 111 of the study in Animal #1.

FIG. 43 is a panel of flow cytometry staining graphs showing anti-CD20 CAR expression and CD25 expression in T cells through to Day 51 of the study in Animal #1.

FIG. 44 is a panel of flow cytometry staining graphs showing CD20+ cells (B cells) and CD3+ T cells through to Day 111 of the study in Animal #1.

FIG. 45 is a graph showing CD20+B cells and CD3+ CAR+ T cells through to Day 111 of the study in Animal #1.

FIG. 46A is a timeline of observed clinical symptoms over the course of the study.

FIG. 46B is a panel of graphs showing serum levels of IL-6, ferritin, and C-reactive protein (CRP) in Animal #1 over the course of the study to Day 56. The line depicting Anti-CD20 CAR T cells (CD3+ FLAG+) is identical in the 3 plots.

FIG. 47 is a panel of flow cytometry staining graphs showing CD20+ cells (B cells—top panels) and CAR+(FLAG+) CD3+ T cells (bottom panels) through to Day 56 of the study in Animal #2.

FIG. 48 is a panel of flow cytometry staining graphs showing CD20+ cells (B cells—top panels) and CAR+(FLAG+) CD3+ T cells (bottom panels) through to Day 37 of the study in Animal #3.

FIG. 49 is a panel of flow cytometry staining graphs showing CD20+ cells (B cells—top panels) and CAR+(FLAG+) CD3+ T cells (bottom panels) through to Day 21 of the study in Animal #4.

FIG. 50A-50J include examples of CD58 and CD80 dual fusion sequences.

FIG. 51A-51F include examples of CD58, CD80 and CD3 scFV triple fusion sequences.

FIG. 52A-52B illustrate details of assessment of engineered particle's in vivo biodistribution.

FIG. 53A-53B include plots showing vector DNA copies/μg genomic DNA (gDNA) in an experiment herein.

FIG. 54 includes a plot showing vector DNA copies/μg genomic DNA (gDNA) in an experiment herein.

FIG. 55A-55B include data comparing the function of particles comprising two variations of fusion proteins (“V1” and “V2”) against control particles comprising a glycoprotein only.

FIG. 56 depicts studies comparing the function of engineered lentiviral particles comprising an anti-CD3 scFv and cocal glycoprotein (“anti-CD3scFv”) with engineered lentiviral particles comprising an anti-CD3scFv, a CD58 protein, and a CD80 protein in addition to a cocal glycoprotein (“Tri protein”). FIG. 56A includes plots illustrating comparative activation data showing the dose-dependent activation of CD4 and CD8 T cells in response to incubation with each particle type. FIG. 56B includes plots illustrating comparative particle-T cell binding data. FIG. 56C includes plots illustrating comparative transduction data showing the dose-dependent transduction efficiency and total number of transduced CD4 and CD8 T cells after incubation with each particle type. FIG. 56D includes plots illustrating comparative cytokine production data showing dose-dependent stimulation of IFN-γ, IL-2, and TNF-α after incubation with each particle type. FIG. 56E includes a plot illustrating comparative serial stimulation data. FIG. 56F includes plots demonstrating that cells incubated with particles comprising an anti-CD3scFv, a CD58 protein, and a CD80 protein (“Tri protein”) produced more inflammatory cytokines than cells incubated with particles comprising an anti-CD3 scFv (“anti-CD3scFv”), but no costimulatory or adhesion molecules. FIG. 56G includes plots demonstrating that particles comprising an anti-CD3scFv, a CD58 protein, and a CD80 protein were able to generate a higher proportion of CCR7+ and CD27+CD4 and CD8 T cells compared with particles comprising an anti-CD3 scFv, but no costimulatory or adhesion molecules.

FIG. 57 describes an in vivo mouse study to evaluate the function of particles comprising an anti-CD3 scFv, but no costimulatory or adhesion molecules and particles comprising an anti-CD3scFv, a CD58 protein, and a CD80 protein. FIG. 57A depicts the study design. FIG. 57B includes plots illustrating in vivo activation data at various dose levels of particles. FIG. 57C includes plots illustrating in vivo transduction of T cells at varying dose levels of particles. FIG. 57D includes plots demonstrating tumor growth and control across the study and in particular showing that the tri protein particles controlled tumor growth to a greater extent than the anti-CD3 scFv particles.

FIG. 58 includes data comparing engineered particles comprising CD58, CD80, and an anti-CD3 scFv separately expressed with engineered particles comprising a fusion protein comprising CD58, anti-CD3 scFv, and CD80 expressed together. FIG. 58A includes plots illustrating particle-T cell binding data. FIG. 58B includes plots illustrating comparative activation data across varying MOIs. FIG. 58C includes plots illustrating transduction data across varying MOIs. FIG. 58D includes plots demonstrating cytokine production by cells following incubation with the two variations of engineered particles.

FIG. 59 describes an in vivo mouse study to evaluate the function of particles comprising CD58, CD80, and an anti-CD3 scFv separately expressed with engineered particles comprising a fusion protein comprising CD58, anti-CD3 scFv, and CD80 expressed together. FIG. 59A includes plots illustrating in vivo activation data at various dose levels of particles. FIG. 59B includes plots illustrating in vivo transduction of T cells at varying dose levels of particles. FIG. 59C includes plots demonstrating tumor growth and control across the study and in particular showing that the fusion protein particles controlled tumor growth to a greater extent than the particles comprising CD58, CD80, and an anti-CD3 scFv separately expressed.

FIG. 60A includes diagrams illustrating example fusion molecules.

FIG. 60B includes diagrams illustrating example fusion molecules.

FIG. 61A shows B cell levels and CAR T cell levels in the four non-human primates tested in an exemplary study. The top panels show B cell concentrations over time and the lower panels show CAR T cell concentrations over time. FIG. 61B includes a plot and table showing some biodistribution details for a composition that was administered to primate subjects.

### DETAILED DESCRIPTION

The disclosure relates generally to a surface-engineered viral particle comprising a vector genome comprising a polynucleotide sequence encoding an anti-CD20 chimeric antigen receptor (CAR), wherein the viral particle transduces immune cells in vivo. The present disclosure includes particles comprising fusion molecules for use in transduction of target cells, such as immune cells, or specifically T cells. In one aspect, the disclosure provides, a particle for in vivo generation of CAR-T cells, comprising, displayed on the surface of the particle, a fusion molecule comprising an adhesion molecule linked to a costimulatory molecule, an activation molecule, or both.

Some embodiments relate to or include a lentiviral particle comprising: a polynucleotide encoding a chimeric antigen receptor (CAR) that specifically binds cluster of differentiation-20 (CD20). Some embodiments relate to or include an anti-CD20 CAR. Some embodiments relate to or include a particle such as a lentiviral particle for transducing an immune cell such as a T cell.

The present disclosure includes particles and fusion molecules for use in transduction of target cells, such as immune cells, or specifically T cells. In one aspect, the disclosure provides, a particle for transduction of target cells, comprising, displayed on the surface of the particle, a fusion molecule comprising an adhesion molecule linked to a costimulatory molecule, an activation molecule, or both.

The term “transduction” is used in its broadest sense to mean delivery of an agent to a cell, such as a therapeutic agent. The agent may be a small molecule, polynucleotide, or polypeptide. A combination of agents may be delivered, such as several polynucleotides or a protein-nucleic acid complex (e.g., a gene-editing nuclease in complex with guide nucleic acid). The term “particle” includes but is not limited to viral particles (i.e., a virion), lipid nanoparticles (LNPs), lipoplexes, liposomes, and nanocarriers.

The fusion molecules of the disclosure combine an adhesion molecule with a costimulatory molecule, an activation molecule, or both. Without being bound by theory, it is believed that the inclusion of two or more of these types of molecule in a fusion molecule may cause such a particle, when it encounters a target cell, to form a macromolecular complex at the interface of the particle and cell that acts as artificial supramolecular activation cluster (SMAC).

T cells that encounter an antigen-presenting cell (APC) form an immune synapse known as a SMAC. In natural SMACs, the APC presents an antigen in complex with a major histocompatibility complex (MHC) molecule to the T cell receptor (TCR) on a T cell; CD80 or CD86 interact with CD28 to provide a costimulatory signal; and CD58 interacts with CD2 to adhere the APC to the T cell. The interactions between CD58 and CD2 may also provide an activatory or costimulatory signal. The adhesion molecule displayed on a particle may be CD58, a ligand for LFA-1 (ICAM-1, ICAM-2, ICAM-3, ICAM-4, ICAM-5, or JAM-A), or a ligand for DNAM-1 (CD155 or CD112), which may bind cognate T cell molecules such as CD2, LFA-1, and DNAM-1. SMACs may further present costimulatory molecules. Costimulatory molecules that may be displayed on a particle include CD80 and CD86, CD40L (also known as CD154), GITRL, OX40L, 41BBL, ICOSL, CD27, CD30L, LIGHT, LTalpha, MICA, and MICB.

As contemplated by the present disclosure, a particle may be engineered to display on its surface any of the foregoing adhesion molecules or costimulatory molecules; extracellular fragments thereof; or functional fragments thereof. Extracellular portions of these molecules may be identified in databases such as UniProt, which is available at www.uniprot.org, or may be predicted using methods, such as a method implemented by the TMHMM 2.0 program available at services.healthtech.dtu.dk. Furthermore, in some cases, functional fragments of each are identified in scientific literature or they may be identified using laboratory methods. For example, one may predict the identify fragments of a protein likely to form well-folded domains. Fragments may be tested in binding assays against a cognate molecule, or used in pull-down assays compared to the full molecule. Functional assays, such as expression of a fluorescence reporter under the control of a promoter activated by T-cell signaling (e.g., the NKkB promoter) when a T cell is contacted with a cell or particle expressing a putative functional fragment. The sequence of the adhesion molecule, costimulatory molecule, or activation molecule may be varied to identify and use variants that retain function. For example, conservative mutations may be made to a molecule or a molecule may be randomly mutated with the function of the variant confirmed experimentally.

In each case, a molecule may be displayed as a full-length form, including its native transmembrane portion. Alternatively, the extracellular portion of the molecule may be displayed with a heterologous transmembrane portion (e.g., a transmembrane portion from another membrane protein) or a membrane anchor (e.g., a glycosylphosphatidylinisotol anchor). Each fusion molecule may be associated with the particle, when the particle comprises a lipid envelope, directly or via the transmembrane portion or anchor connected to one of the other molecules in the fusion molecule. For example, an adhesion molecule, costimulatory molecule, and activation molecule may be linked in any order with only the most N-terminal or C-terminal of the molecules connected to a transmembrane region or anchor. In a variant, the fusion molecule comprises or is associated with another membrane-associated molecule, thereby displaying the fusion molecule on the particle. The term “display” is used, in a broad sense, to me position on the surface of the particle such that the molecule may contact cognate molecules on the target cell. It is further contemplated that for particles lacking an envelope (such as a non-enveloped viral particle) the fusion molecule may be displayed on the particle either by association with a component of the particle (e.g., a capsid protein) or by a direct linkage with the particle (e.g., as a fusion protein comprises a capsid protein).

Disclosed herein, in some embodiments, are lentiviral particles, comprising, displayed on the surface of the particle: a fusion molecule comprising: a) a CD58 extracellular domain, or a functional fragment thereof, b) a CD80 or CD86 extracellular domain, or a functional fragment thereof, c) an antigen-binding fragment of an anti-CD3 antibody; and a viral glycoprotein (G protein), wherein the lentiviral particle comprises a polynucleotide encoding a chimeric antigen receptor that specifically binds CD20 (e.g. an anti-CD20 CAR).

Disclosed herein, in some embodiments are lentiviral particles, comprising, displayed on the surface of the particle: a fusion molecule comprising a) a CD58 extracellular domain, or a functional fragment thereof, b) an antigen-binding fragment of an anti-CD3 antibody, and c) a CD80 extracellular domain, or a functional fragment thereof; and a viral glycoprotein (G protein); wherein the lentiviral particle further comprises a polynucleotide encoding a chimeric antigen receptor that specifically binds CD20 (e.g. an anti-CD20 CAR), a free FRB, a synthetic cytokine gamma chain polypeptide, and a synthetic cytokine beta chain polypeptide, wherein the anti-CD20 CAR comprises a ligand-binding domain comprising an scFv, a hinge domain, a transmembrane domain, a 41BB endodomain, and a CD3ζ endodomain, and wherein the scFv comprises a VL comprising SEQ ID NO: 176 and a VH comprising SEQ ID NO: 179, the hinge domain comprises SEQ ID NO: 280, the transmembrane domain comprises SEQ ID NO: 281, the 41BB endodomain comprises SEQ ID NO: 184, and the CD3ζ endodomain comprises SEQ ID NO: 214. Disclosed herein, in some embodiments are lentiviral particles, comprising, displayed on the surface of the particle: a fusion molecule comprising a) a CD58 extracellular domain, or a functional fragment thereof, b) an antigen-binding fragment of an anti-CD3 antibody, and c) a CD80 extracellular domain, or a functional fragment thereof; and a viral glycoprotein (G protein); wherein the lentiviral particle further comprises a polynucleotide encoding a chimeric antigen receptor that specifically binds CD20 (e.g. anti-CD20 CAR), wherein the anti-CD20 CAR comprises a ligand-binding domain comprising an scFv, a hinge domain, a transmembrane domain, a 41BB endodomain, and a CD3ζ endodomain, and wherein the scFv comprises a VL comprising SEQ ID NO: 176 and a VH comprising SEQ ID NO: 179, the hinge domain comprises SEQ ID NO: 280, the transmembrane domain comprises SEQ ID NO: 281, the 41BB endodomain comprises SEQ ID NO: 184, and the CD3ζ endodomain comprises SEQ ID NO: 214.

Disclosed herein, in some embodiments are engineered cells, comprising a chimeric antigen receptor that specifically binds CD20 (e.g. an anti-CD20 CAR); wherein the anti-CD20 CAR comprises a ligand-binding domain comprising an scFv, a hinge domain, a transmembrane domain, a 41BB endodomain, and a CD3ζ endodomain, and wherein the scFv comprises a VL comprising SEQ ID NO: 176 and a VH comprising SEQ ID NO: 179, the hinge domain comprises SEQ ID NO: 280, the transmembrane domain comprises SEQ ID NO: 281, the 41BB endodomain comprises SEQ ID NO: 184, and the CD3ζ endodomain comprises SEQ ID NO: 214. In some embodiments, the engineered cell further comprises a synthetic cytokine gamma chain polypeptide, a synthetic cytokine beta chain polypeptide, and, optionally, a free FRB. In some embodiments, the engineered cell is a CD3+ T cell.

### Adhesion Molecules

Disclosed herein, in some embodiments, are adhesion molecules. The adhesion molecule may be included as part of a fusion molecule. The adhesion molecule may be included as part of a particle (e.g. on a particle surface).

As used herein, the term “adhesion molecule” refers, in a broad sense, to a molecular component of a SMAC or other immune synapse, other than an activation molecule (e.g. TCR-binding agent) or a costimulatory molecule, which in contributes to adhesion of a particle to target cells. Adhesion molecules from natural sources may be molecules expressed, natively, on antigen-presenting cells and adapted for use here on particles. Both naturally occurring adhesion molecules, and their variants, and artificial adhesion molecules, such as antibodies, or fragments thereof, are contemplated. An adhesion molecule, as the term is used herein, specifically binds a conjugate molecule with affinity sufficient to cause increased adhesion between the particle and the target cell compared to the adhesion of a reference particle lacking the adhesion molecule to the same or similar target cell. The term adhesion molecule includes but is not limited to CD58, a CD58 extracellular portion, and functional fragments of CD58. As described above, the term “functional fragment” is used herein to a fragment of a polypeptide, or other molecule, that retains the desired function of the polypeptide. For example, a functional fragment of CD58 is a fragment of CD58 that specifically binds CD2. The adhesion molecule may be a protein, termed herein an “adhesion protein.”

In some embodiments, the costimulatory and/or adhesion molecule comprises an amino acid sequence 100% identical to a sequence in Table 1A or Table 1B. In some embodiments, the costimulatory and/or adhesion molecule shares at least 80%, at least 85%, at least 90%, at least 91%, at least 92%, at least 93%, at least 94%, at least 95%, at least 96%, at least 97%, at least 98% or at least 99% identity to a sequence in Table 1A or Table 1B. In some embodiments, the costimulatory and/or adhesion molecule shares less than 80%, less than 85%, less than 90%, less than 91%, less than 92%, less than 93%, less than 94%, less than 95%, less than 96%, less than 97%, less than 98%, less than 99%, or less than 100% identity to a sequence in Table 1A or Table 1B.

Polypeptide sequences of illustrative adhesion molecules are provided in Table 1A, with the “start” and “end” positions of the extracellular portion of each. In each case, the adhesion molecule may comprise a polypeptide at least 75%, at least 80%, at least 85%, at least 90%, at least 95%, at least 99%, or 100% sequence identity to any sequence in Table 1A, or functional fragments thereof. Functional fragments may be or include any 10, 20, 30, 40, 50, 75, 100, 200, 300, 400, 500, or 600 (or any range thereof) amino acid portion that retains binding affinity to its cognate molecule, when measured using affinity assays such as biolayer interferometry or other assays that may be known in the art.

TABLE 1A

| | | | Start of | End of |
|---|---|---|---|---|
| | SEQ | | the extra- | the extra- |
| Adhesion | ID | | cellular | cellular |
| Molecule | NO: | Sequence | portion | portion |
| CD58 | 1 | MVAGSDAGRALGVLSVVCLLHCFGFISCFSQQIYGVVY | 29 | 215 |
| | | GNVTFHVPSNVPLKEVLWKKQKDKVAELENSEFRAFS | | |
| | | SFKNRVYLDTVSGSLTIYNLTSSDEDEYEMESPNITDTM | | |
| | | KFFLYVLESLPSPTLTCALTNGSIEVQCMIPEHYNSHRG | | |
| | | LIMYSWDCPMEQCKRNSTSIYFKMENDLPQKIQCTLSN | | |
| | | PLFNTTSSIILTTCIPSSGHSRHRYALIPIPLAVITTCIVLY | | |
| | | MNGILKCDRKPDRTNSN | | |
| ICAM-1 | 2 | MAPSSPRPALPALLVLLGALFPGPGNAQTSVSPSKVILP | 28 | 480 |
| | | RGGSVLVTCSTSCDQPKLLGIETPLPKKELLLPGNNRK | | |
| | | VYELSNVQEDSQPMCYSNCPDGQSTAKTFLTVYWTPE | | |
| | | RVELAPLPSWQPVGKNLTLRCQVEGGAPRANLTVVLL | | |
| | | RGEKELKREPAVGEPAEVTTTVLVRRDHHGANFSCRT | | |
| | | ELDLRPQGLELFENTSAPYQLQTFVLPATPPQLVSPRVL | | |
| | | EVDTQGTVVCSLDGLFPVSEAQVHLALGDQRLNPTVT | | |
| | | YGNDSFSAKASVSVTAEDEGTQRLTCAVILGNQSQETL | | |
| | | QTVTIYSFPAPNVILTKPEVSEGTEVTVKCEAHPRAKVT | | |
| | | LNGVPAQPLGPRAQLLLKATPEDNGRSFSCSATLEVAG | | |
| | | QLIHKNQTRELRVLYGPRLDERDCPGNWTWPENSQQT | | |
| | | PMCQAWGNPLPELKCLKDGTFPLPIGESVTVTRDLEGT | | |
| | | YLCRARSTQGEVTRKVTVNVLSPRYEIVIITVVAAAVI | | |
| | | MGTAGLSTYLYNRQRKIKKYRLQQAQKGTPMKPNTQ | | |
| | | ATPP | | |
| ICAM-2 | 3 | MSSFGYRTLTVALFTLICCPGSDEKVFEVHVRPKKLAV | 25 | 223 |
| | | EPKGSLEVNCSTTCNQPEVGGLETSLDKILLDEQAQWK | | |
| | | HYLVSNISHDTVLQCHFTCSGKQESMNSNVSVYQPPRQ | | |
| | | VILTLQPTLVAVGKSFTIECRVPTVEPLDSLTLFLFRGNE | | |
| | | TLHYETFGKAAPAPQEATATFNSTADREDGHRNFSCLA | | |
| | | VLDLMSRGGNIFHKHSAPKMLEIYEPVSDSQMVIIVTV | | |
| | | VSVLLSLFVTSVLLCFIFGQHLRQQRMGTYGVRAAWR | | |
| | | RLPQAFRP | | |
| ICAM-3 | 4 | MATMVPSVLWPRACWTLLVCCLLTPGVQGQEFLLRV | 30 | 485 |
| | | EPQNPVLSAGGSLFVNCSTDCPSSEKIALETSLSKELVA | | |
| | | SGMGWAAFNLSNVTGNSRILCSVYCNGSQITGSSNITV | | |
| | | YRLPERVELAPLPPWQPVGQNFTLRCQVEDGSPRTSLT | | |
| | | VVLLRWEEELSRQPAVEEPAEVTATVLASRDDHGAPFS | | |
| | | CRTELDMQPQGLGLFVNTSAPRQLRTFVLPVTPPRLVA | | |
| | | PRFLEVETSWPVDCTLDGLFPASEAQVYLALGDQMLN | | |
| | | ATVMNHGDTLTATATATARADQEGAREIVCNVTLGGE | | |
| | | RREARENLTVFSFLGPIVNLSEPTAHEGSTVTVSCMAG | | |
| | | ARVQVTLDGVPAAAPGQPAQLQLNATESDDGRSFFCS | | |
| | | ATLEVDGEFLHRNSSVQLRVLYGPKIDRATCPQHLKW | | |
| | | KDKTRHVLQCQARGNPYPELRCLKEGSSREVPVGIPFF | | |
| | | VNVTHNGTYQCQASSSRGKYTLVVVMDIEAGSSHFVP | | |
| | | VFVAVLLTLGVVTIVLALMYVFREHQRSGSYHVREEST | | |
| | | YLPLTSMQPTEAMGEEPSRAE | | |
| ICAM-4 | 5 | MGSLFPLSLLFFLAAAYPGVGSALGRRTKRAQSPKGSP | 23 | 240 |
| | | LAPSGTSVPFWVRMSPEFVAVQPGKSVQLNCSNSCPQP | | |
| | | QNSSLRTPLRQGKTLRGPGWVSYQLLDVRAWSSLAHC | | |
| | | LVTCAGKTRWATSRITAYKPPHSVILEPPVLKGRKYTL | | |
| | | RCHVTQVFPVGYLVVTLRHGSRVIYSESLERFTGLDLA | | |
| | | NVTLTYEFAAGPRDFWQPVICHARLNLDGLVVRNSSA | | |
| | | PITLMLAWSPAPTALASGSIAALVGILLTVGAAYLCKC | | |
| | | LAMKSQA | | |
| ICAM-5 | 6 | MPGPSPGLRRALLGLWAALGLGLFGLSAVSQEPFWAD | 32 | 835 |
| | | LQPRVAFVERGGSLWLNCSTNCPRPERGGLETSLRRNG | | |
| | | TQRGLRWLARQLVDIREPETQPVCFFRCARRTLQARGL | | |
| | | IRTFQRPDRVELMPLPPWQPVGENFTLSCRVPGAGPRA | | |
| | | SLTLTLLRGAQELIRRSFAGEPPRARGAVLTATVLARRE | | |
| | | DHGANFSCRAELDLRPHGLGLFENSSAPRELRTFSLSPD | | |
| | | APRLAAPRLLEVGSERPVSCTLDGLFPASEARVYLALG | | |
| | | DQNLSPDVTLEGDAFVATATATASAEQEGARQLVCNV | | |
| | | TLGGENRETRENVTIYSFPAPLLTLSEP | | |
| JAM-A | 7 | MGTKAQVERKLLCLFILAILLCSLALGSVTVHSSEPEVR | 28 | 238 |
| | | IPENNPVKLSCAYSGFSSPRVEWKFDQGDTTRLVCYNN | | |
| | | KITASYEDRVTFLPTGITFKSVTREDTGTYTCMVSEEGG | | |
| | | NSYGEVKVKLIVLVPPSKPTVNIPSSATIGNRAVLTCSE | | |
| | | QDGSPPSEYTWFKDGIVMPTNPKSTRAFSNSSYVLNPT | | |
| | | TGELVFDPLSASDTGEYSCEARNGYGTPMTSNAVRME | | |
| | | AVERNVGVIVAAVLVTLILLGILVFGIWFAYSRGHFDR | | |
| | | TKKGTSSKKVIYSQPSARSEGEFKQTSSFLV | | |
| CD155 | 8 | MARAMAAAWPLLLVALLVLSWPPPGTGDVVVQAPTQ | 21 | 343 |
| | | VPGFLGDSVTLPCYLQVPNMEVTHVSQLTWARHGESG | | |
| | | SMAVFHQTQGPSYSESKRLEFVAARLGAELRNASLRM | | |
| | | FGLRVEDEGNYTCLFVTFPQGSRSVDIWLRVLAKPQNT | | |
| | | AEVQKVQLTGEPVPMARCVSTGGRPPAQITWHSDLGG | | |
| | | MPNTSQVPGFLSGTVTVTSLWILVPSSQVDGKNVTCKV | | |
| | | EHESFEKPQLLTVNLTVYYPPEVSISGYDNNWYLGQNE | | |
| | | ATLTCDARSNPEPTGYNWSTTMGPLPPFAVAQGAQLLI | | |
| | | RPVDKPINTTLICNVTNALGARQAELTVQVKEGPPSEH | | |
| | | SGISRNAIIFLVLGILVFLILLGIGIYFYWSKCSREVLWH | | |
| | | CHLCPSSTEHASASANGHVSYSAVSRENSSSQDPQTEG | | |
| | | TR | | |
| CD112 | 9 | MARAAALLPSRSPPTPLLWPLLLLLLLETGAQDVRVQV | 32 | 360 |
| | | LPEVRGQLGGTVELPCHLLPPVPGLYISLVTWQRPDAP | | |
| | | ANHQNVAAFHPKMGPSFPSPKPGSERLSFVSAKQSTGQ | | |
| | | DTEAELQDATLALHGLTVEDEGNYTCEFATFPKGSVR | | |
| | | GMTWLRVIAKPKNQAEAQKVTFSQDPTTVALCISKEG | | |
| | | RPPARISWLSSLDWEAKETQVSGTLAGTVTVTSRFTLV | | |
| | | PSGRADGVTVTCKVEHESFEEPALIPVTLSVRYPPEVSIS | | |
| | | GYDDNWYLGRTDATLSCDVRSNPEPTGYDWSTTSGTF | | |
| | | PTSAVAQGSQLVIHAVDSLFNTTFVCTVTNAVGMGRA | | |
| | | EQVIFVRETPNTAGAGATGGIIGGIIAAIIATAVAATGILI | | |
| | | CRQQRKEQTLQGAEEDEDLEGPPSYKPPTPKAKLEAQE | | |
| | | MPSQLFTLGASEHSPLKTPYFDAGASCTEQEMPRYHEL | | |
| | | PTLEERSGPLHPGATSLGSPIPVPPGPPAVEDVSLDLEDE | | |
| | | EGEEEEEYLDKINPIYDALSYSSPSDSYQGKGFVMSRA | | |
| | | MYV | | |

TABLE 1B

| | | SEQ ID | |
|---|---|---|---|
| Name | Sequence | NO: | Region |
| CD80 | VIHVTKEVKEVATLSCGHNVSVEELAQTRIYWQKEKK | 134 | Val35 - |
| Extracellular | MVLTMMSGDMNIWPEYKNRTIFDITNNLSIVILALRPS | | Asn242 |
| Domain | DEGTYECVVLKYEKDAFKREHLAEVTLSVKADFPTPSI | | |
| | SDFEIPTSNIRRIICSTSGGFPEPHLSWLENGEELNAINTT | | |
| | VSQDPETELYAVSSKLDFNMTTNHSFMCLIKYGHLRV | | |
| | NQTFNWNTTKQEHFPDN | | |
| CD86 | APLKIQAYFNETADLPCQFANSQNQSLSELVVFWQDQ | 135 | Ala24 - |
| Extracellular | ENLVLNEVYLGKEKFDSVHSKYMGRTSFDSDSWTLRL | | Pro247 |
| Domain | HNLQIKDKGLYQCIIHHKKPTGMIRIHQMNSELSVLAN | | |
| | FSQPEIVPISNITENVYINLTCSSIHGYPEPKKMSVLLRT | | |
| | KNSTIEYDGVMQKSQDNVTELYDVSISLSVSFPDVTSN | | |
| | MTIFCILETDKTRLLSSPFSIELEDPQPPPDHIP | | |
| CD58 | FSQQIYGVVYGNVTFHVPSNVPLKEVLWKKQKDKVA | 136 | Phe29 - |
| Extracellular | ELENSEFRAFSSFKNRVYLDTVSGSLTIYNLTSSDEDEY | | Val120 |
| Domain | EMESPNITDTMKFFLYV | | |
| HHLA2 (a/k/a | IFPLAFFIYVPMNEQIVIGRLDEDIILPSSFERGSEVVIHW | 137 | Ile23 - |
| B7-H7) | KYQDSYKVHSYYKGSDHLESQDPRYANRTSLFYNEIQ | | Lys345 |
| Extracellular | NGNASLFFRRVSLLDEGIYTCYVGTAIQVITNKVVLKV | | |
| Domain | GVFLTPVMKYEKRNTNSFLICSVLSVYPRPIITWKMDN | | |
| | TPISENNMEETGSLDSFSINSPLNITGSNSSYECTIENSLL | | |
| | KQTWTGRWTMKDGLHKMQSEHVSLSCQPVNDYFSPN | | |
| | QDFKVTWSRMKSGTFSVLAYYLSSSQNTIINESRFSWN | | |
| | KELINQSDFSMNLMDLNLSDSGEYLCNISSDEYTLLTIH | | |
| | TVHVEPSQETASHNK | | |
| ICAM-1 | QTSVSPSKVILPRGGSVLVTCSTSCDQPKLLGIETPLPK | 138 | Gln28- |
| Extracellular | KELLLPGNNRKVYELSNVQEDSQPMCYSNCPDGQSTA | | Glu480 |
| Domain | KTFLTVYWTPERVELAPLPSWQPVGKNLTLRCQVEGG | | |
| | APRANLTVVLLRGEKELKREPAVGEPAEVTTTVLVRR | | |
| | DHHGANFSCRTELDLRPQGLELFENTSAPYQLQTFVLP | | |
| | ATPPQLVSPRVLEVDTQGTVVCSLDGLFPVSEAQVHLA | | |
| | LGDQRLNPTVTYGNDSFSAKASVSVTAEDEGTQRLTC | | |
| | AVILGNQSQETLQTVTIYSFPAPNVILTKPEVSEGTEVT | | |
| | VKCEAHPRAKVTLNGVPAQPLGPRAQLLLKATPEDNG | | |
| | RSFSCSATLEVAGQLIHKNQTRELRVLYGPRLDERDCP | | |
| | GNWTWPENSQQTPMCQAWGNPLPELKCLKDGTFPLPI | | |
| | GESVTVTRDLEGTYLCRARSTQGEVTRKVTVNVLSPR | | |
| | YE | | |
| OX40-L | QVSHRYPRIQSIKVQFTEYKKEKGFILTSQKEDEIMKVQ | 139 | Gln51 - |
| Extracellular | NNSVIINCDGFYLISLKGYFSQEVNISLHYQKDEEPLFQ | | Leu183 |
| Domain | LKKVRSVNSLMVASLTYKDKVYLNVTTDNTSLDDFH | | |
| | VNGGELILIHQNPGEFCVL | | |
| 4-1BBL | REGPELSPDDPAGLLDLRQGMFAQLVAQNVLLIDGPLS | 140 | Arg71 - |
| Extracellular | WYSDPGLAGVSLTGGLSYKEDTKELVVAKAGVYYVF | | Glu254 |
| Domain | FQLELRRVVAGEGSGSVSLALHLQPLRSAAGAAALAL | | |
| | TVDLPPASSEARNSAFGFQGRLLHLSAGQRLGVHLHTE | | |
| | ARARHAWQLTQGATVLGLFRVTPEIPAGLPSPRSE | | |
| CD40 | EPPTACREKQYLINSQCCSLCQPGQKLVSDCTEFTETEC | 141 | Glu21 - |
| Extracellular | LPCGESEFLDTWNRETHCHQHKYCDPNLGLRVQQKGT | | Arg193 |
| Domain | SETDTICTCEEGWHCTSEACESCVLHRSCSPGFGVKQIA | | |
| | TGVSDTICEPCPVGFFSNVSSAFEKCHPWTSCETKDLV | | |
| | VQQAGTNKTDVVCGPQDRLR | | |
| CD155 | WPPPGTGDVVVQAPTQVPGFLGDSVTLPCYLQVPNME | 142 | Trp21 - |
| Extracellular | VTHVSQLTWARHGESGSMAVFHQTQGPSYSESKRLEF | | Asn343 |
| Domain | VAARLGAELRNASLRMFGLRVEDEGNYTCLFVTFPQG | | |
| | SRSVDIWLRVLAKPQNTAEVQKVQLTGEPVPMARCVS | | |
| | TGGRPPAQITWHSDLGGMPNTSQVPGFLSGTVTVTSL | | |
| | WILVPSSQVDGKNVTCKVEHESFEKPQLLTVNLTVYYP | | |
| | PEVSISGYDNNWYLGQNEATLTCDARSNPEPTGYNWS | | |
| | TTMGPLPPFAVAQGAQLLIRPVDKPINTTLICNVTNALG | | |
| | ARQAELTVQVKEGPPSEHSGISRN | | |
| CD70 | QRFAQAQQQLPLESLGWDVAELQLNHTGPQQDPRLY | 143 | Gln39 - |
| Extracellular | WQGGPALGRSFLHGPELDKGQLRIHRDGIYMVHIQVT | | Pro 193 |
| Domain | LAICSSTTASRHHPTTLAVGICSPASRSISLLRLSFHQGC | | |
| | TIASQRLTPLARGDTLCTNLTGTLLPSRNTDETFFGVQ | | |
| | WVRP | | |
| HVEM | LPSCKEDEYPVGSECCPKCSPGYRVKEACGELTGTVCE | 144 | Leu39 - |
| Extracellular | PCPPGTYIAHLNGLSKCLQCQMCDPAMGLRASRNCSR | | Ser199 |
| Domain | TENAVCGCSPGHFCIVQDGDHCAACRAYATSSPGQRV | | |
| | QKGGTESQDTLCQNCPPGTFSPNGTLEECQHQTKCSW | | |
| | LVTKAGAGTSSS | | |
| GITRL | QLETAKEPCMAKFGPLPSKWQMASSEPPCVNKVSDW | 145 | Gln72 - |
| Extracellular | KLEILQNGLYLIYGQVAPNANYNDVAPFEVRLYKNKD | | Ser199 |
| Domain | MIQTLTNKSKIQNVGGTYELHVGDTIDLIFNSEHQVLK | | |
| | NNTYWGIILLANPQFIS | | |
| CD30L | QRTDSIPNSPDNVPLKGGNCSEDLLCILKRAPFKKSWA | 146 | Gln63 - |
| Extracellular | YLQVAKHLNKTKLSWNKDGILHGVRYQDGNLVIQFPG | | Asp234 |
| Domain | LYFIICQLQFLVQCPNNSVDLKLELLINKHIKKQALVTV | | |
| | CESGMQTKHVYQNLSQFLLDYLQVNTTISVNVDTFQYI | | |
| | DTSTFPLENVLSIFLYSNSD | | |
| SLAMF2 | QGHLVHMTVVSGSNVTLNISESLPENYKQLTWFYTFD | 147 | Gln27 - |
| Extracellular | QKIVEWDSRKSKYFESKFKGRVRLDPQSGALYISKVQK | | Arg219 |
| Domain | EDNSTYIMRVLKKTGNEQEWKIKLQVLDPVPKPVIKIE | | |
| | KIEDMDDNCYLKLSCVIPGESVNYTWYGDKRPFPKEL | | |
| | QNSVLETTLMPHNYSRCYTCQVSNSVSSKNGTVCLSPP | | |
| | CTLAR | | |
| Ly-9 | KDSAPTVVSGILGGSVTLPLNISVDTEIENVIWIGPKNA | 148 | Lys48- |
| Extracellular | LAFARPKENVTIMVKSYLGRLDITKWSYSLCISNLTLN | | Lys454 |
| Domain | DAGSYKAQINQRNFEVTTEEEFTLFVYEQLQEPQVTM | | |
| | KSVKVSENFSCNITLMCSVKGAEKSVLYSWTPREPHAS | | |
| | ESNGGSILTVSRTPCDPDLPYICTAQNPVSQRSSLPVHV | | |
| | GQFCTDPGASRGGTTGETVVGVLGEPVTLPLALPACR | | |
| | DTEKVVWLFNTSIISKEREEAATADPLIKSRDPYKNRV | | |
| | WVSSQDCSLKISQLKIEDAGPYHAYVCSEASSVTSMTH | | |
| | VTLLIYRRLRKPKITWSLRHSEDGICRISLTCSVEDGGN | | |
| | TVMYTWTPLQKEAVVSQGESHLNVSWRSSENHPNLTC | | |
| | TASNPVSRSSHQFLSENICSGPERNTK | | |
| CD84 | KDSEIFTVNGILGESVTFPVNIQEPRQVKIIAWTSKTSVA | 149 | Lys22- |
| Extracellular | YVTPGDSETAPVVTVTHRNYYERIHALGPNYNLVISDL | | Gly225 |
| Domain | RMEDAGDYKADINTQADPYTTTKRYNLQIYRRLGKPK | | |
| | ITQSLMASVNSTCNVTLTCSVEKEEKNVTYNWSPLGEE | | |
| | GNVLQIFQTPEDQELTYTCTAQNPVSNNSDSISARQLC | | |
| | ADIAMGFRTHHTG | | |
| Ly108 (a/k/a | QSSLTPLMVNGILGESVTLPLEFPAGEKVNFITWLFNET | 150 | Gln22 - |
| SLAMF6) | SLAFIVPHETKSPEIHVTNPKQGKRLNFTQSYSLQLSNL | | Lys225 |
| Extracellular | KMEDTGSYRAQISTKTSAKLSSYTLRILRQLRNIQVTN | | |
| Domain | HSQLFQNMTCELHLTCSVEDADDNVSFRWEALGNTLS | | |
| | SQPNLTVSWDPRISSEQDYTCIAENAVSNLSFSVSAQKL | | |
| | CEDVKIQYTDTK | | |
| MICA | EPHSLRYNLTVLSWDGSVQSGFLTEVHLDGQPFLRCD | 151 | Glu24 - |
| Extracellular | RQKCRAKPQGQWAEDVLGNKTWDRETRDLTGNGKD | | His306 |
| Domain | LRMTLAHIKDQKEGLHSLQEIRVCEIHEDNSTRSSQHF | | |
| | YYDGELFLSQNLETKEWTMPQSSRAQTLAMNVRNFLK | | |
| | EDAMKTKTHYHAMHADCLQELRRYLKSGVVLRRTVP | | |
| | PMVNVTRSEASEGNITVTCRASGFYPWNITLSWRQDG | | |
| | VSLSHDTQQWGDVLPDGNGTYQTWVATRICQGEEQR | | |
| | FTCYMEHSGNHSTHPVPSGKVLVLQSH | | |
| MICB | EPHSLRYNLMVLSQDGSVQSGFLAEGHLDGQPFLRYD | 152 | Glu24 - |
| Extracellular | RQKRRAKPQGQWAEDVLGAETWDTETEDLTENGQDL | | Asp266 |
| Domain | RRTLTHIKDQKGVPQSSRAQTLAMNVTNFWKEDAMK | | |
| | TKTHYRAMQADCLQKLQRYLKSGVAIRRTVPPMVNV | | |
| | TCSEVSEGNITVTCRASSFYPRNITLTWRQDGVSLSHNT | | |
| | QQWGDVLPDGNGTYQTWVATRIRQGEEQRFTCYMEH | | |
| | SGNHGTHPVPSGKALVLQSQRTD | | |
| ULBP1 | MAAAASPAFLLCLPLLHLLSGWSRAGWVDTHCLCYDF | 153 | |
| | IITPKSRPEPQWCEVQGLVDERPFLHYDCVNHKAKAFA | | |
| | SLGKKVNVTKTWEEQTETLRDVVDFLKGQLLDIQVEN | | |
| | LIPIEPLTLQARMSCEHEAHGHGRGSWQFLFNGQKFLL | | |
| | FDSNNRKWTALHPGAKKMTEKWEKNRDVTMFFQKIS | | |
| | LGDCKMWLEEFLMYWEQMLDPTKPPSLAPGTTQPKA | | |
| | MATTLSPWSLLIIFLCFILAGR | | |
| ULBP2 | MAAAAATKILLCLPLLLLLSGWSRAGRADPHSLCYDIT | 154 | |
| | VIPKFRPGPRWCAVQGQVDEKTFLHYDCGNKTVTPVS | | |
| | PLGKKLNVTTAWKAQNPVLREVVDILTEQLRDIQLEN | | |
| | YTPKEPLTLQARMSCEQKAEGHSSGSWQFSFDGQIFLL | | |
| | FDSEKRMWTTVHPGARKMKEKWENDKVVAMSFHYF | | |
| | SMGDCIGWLEDFLMGMDSTLEPSAGAPLAMSSGTTQL | | |
| | RATATTLILCCLLIILPCFILPGI | | |
| ULBP3 | MAAAASPAILPRLAILPYLLFDWSGTGRADAHSLWYN | 155 | |
| | FTIIHLPRHGQQWCEVQSQVDQKNFLSYDCGSDKVLS | | |
| | MGHLEEQLYATDAWGKQLEMLREVGQRLRLELADTE | | |
| | LEDFTPSGPLTLQVRMSCECEADGYIRGSWQFSFDGRK | | |
| | FLLFDSNNRKWTVVHAGARRMKEKWEKDSGLTTFFK | | |
| | MVSMRDCKSWLRDFLMHRKKRLEPTAPPTMAPGLAQ | | |
| | PKAIATTLSPWSFLIILCFILPGI | | |
| ULBP4 | HSLCFNFTIKSLSRPGQPWCEAQVFLNKNLFLQYNSDN | 156 | His31- |
| Extracellular | NMVKPLGLLGKKVYATSTWGELTQTLGEVGRDLRML | | Asp225 |
| Domain | LCDIKPQIKTSDPSTLQVEMFCQREAERCTGASWQFAT | | |
| | NGEKSLLFDAMNMTWTVINHEASKIKETWKKDRGLE | | |
| | KYFRKLSKGDCDHWLREFLGHWEAMPEPTVSPVNAS | | |
| | DIHWSSSSLPD | | |
| ULBP5 | GLADPHSLCYDITVIPKFRPGPRWCAVQGQVDEKTFLH | 157 | Gly26- |
| Extracellular | YDCGSKTVTPVSPLGKKLNVTTAWKAQNPVLREVVDI | | Arg223 |
| Domain | LTEQLLDIQLENYIPKEPLTLQARMSCEQKAEGHGSGS | | |
| | WQLSFDGQIFLLFDSENRMWTTVHPGARKMKEKWEN | | |
| | DKDMTMSFHYISMGDCTGWLEDFLMGMDSTLEPSAG | | |
| | APPTMSSGTAQPR | | |
| ULBP6 | MAAAAIPALLLCLPLLFLLFGWSRARRDDPHSLCYDIT | 158 | |
| | VIPKFRPGPRWCAVQGQVDEKTFLHYDCGNKTVTPVS | | |
| | PLGKKLNVTMAWKAQNPVLREVVDILTEQLLDIQLEN | | |
| | YTPKEPLTLQARMSCEQKAEGHSSGSWQFSIDGQTFLL | | |
| | FDSEKRMWTTVHPGARKMKEKWENDKDVAMSFHYIS | | |
| | MGDCIGWLEDFLMGMDSTLEPSAGAPLAMSSGTTQLR | | |
| | ATATTLILCCLLIILPCFILPGI | | |
| B7-H2 (a/k/a | DTQEKEVRAMVGSDVELSCACPEGSRFDLNDVYVYW | 159 | Asp19 - |
| ICOSL) | QTSESKTVVTYHIPQNSSLENVDSRYRNRALMSPAGM | | Thr256 |
| Extracellular | LRGDFSLRLFNVTPQDEQKFHCLVLSQSLGFQEVLSVE | | |
| Domain | VTLHVAANFSVPVVSAPHSPSQDELTFTCTSINGYPRPN | | |
| | VYWINKTDNSLLDQALQNDTVFLNMRGLYDVVSVLRI | | |
| | ARTPSVNIGCCIENVLLQQNLTVGSQTGNDIGERDKITE | | |
| | NPVSTGEKNAAT | | |
| B7-H6 | DLKVEMMAGGTQITPLNDNVTIFCNIFYSQPLNITSMGI | 160 | Asp25 - |
| Extracellular | TWFWKSLTFDKEVKVFEFFGDHQEAFRPGAIVSPWRL | | Ser262 |
| Domain | KSGDASLRLPGIQLEEAGEYRCEVVVTPLKAQGTVQLE | | |
| | VVASPASRLLLDQVGMKENEDKYMCESSGFYPEAINIT | | |
| | WEKQTQKFPHPIEISEDVITGPTIKNMDGTFNVTSCLKL | | |
| | NSSQEDPGTVYQCVVRHASLHTPLRSNFTLTAARHSLS | | |
| | ETEKTDNFS | | |
| B7-H5 | FKVATPYSLYVCPEGQNVTLTCRLLGPVDKGHDVTFY | 161 | Phe33 - |
| Extracellular | KTWYRSSRGEVQTCSERRPIRNLTFQDLHLHHGGHQA | | Ala194 |
| Domain | ANTSHDLAQRHGLESASDHHGNFSITMRNLTLLDSGL | | |
| | YCCLVVEIRHHHSEHRVHGAMELQVQTGKDAPSNCV | | |
| | VYPSSSQDSENITAA | | |
| B7-H3 | LEVQVPEDPVVALVGTDATLCCSFSPEPGFSLAQLNLI | 162 | Leu29 - |
| Extracellular | WQLTDTKQLVHSFAEGQDQGSAYANRTALFPDLLAQ | | Ala248 |
| Domain | GNASLRLQRVRVADEGSFTCFVSIRDFGSAAVSLQVAA | | |
| | PYSKPSMTLEPNKDLRPGDTVTITCSSYRGYPEAEVFW | | |
| | QDGQGVPLTGNVTTSQMANEQGLFDVHSVLRVVLGA | | |
| | NGTYSCLVRNPVLQQDAHGSVTITGQPMTFPPEA | | |
| B7x (a/k/a B7- | LIIGFGISGRHSITVTTVASAGNIGEDGILSCTFEPDIKLS | 163 | Leu25 - |
| H4) | DIVIQWLKEGVLGLVHEFKEGKDELSEQDEMFRGRTA | | Ser259 |
| Extracellular | VFADQVIVGNASLRLKNVQLTDAGTYKCYIITSKGKG | | |
| Domain | NANLEYKTGAFSMPEVNVDYNASSETLRCEAPRWFPQ | | |
| | PTVVWASQVDQGANFSEVSNTSFELNSENVTMKVVSV | | |
| | LYNVTINNTYSCMIENDIAKATGDIKVTESEIKRRSHLQ | | |
| | LLNSKASTTENLYFQG | | |
| TMIGD2 | LSVQQGPNLLQVRQGSQATLVCQVDQATAWERLRVK | 164 | Leu23 - |
| Extracellular | WTKDGAILCQPYITNGSLSLGVCGPQGRLSWQAPSHLT | | Gly150 |
| Domain | LQLDPVSLNHSGAYVCWAAVEIPELEEAEGNITRLFVD | | |
| | PDDPTQNRNRIASFPG | | |
| IL-2 | APTSSSTKKTQLQLEHLLLDLQMILNGINNYKNPKLTR | 165 | |
| | MLTFKFYMPKKATELKHLQCLEEELKPLEEVLNLAQS | | |
| | KNFHLRPRDLISNINVIVLELKGSETTFMCEYADETATI | | |
| | VEFLNRWITFCQSIISTLT | | |
| IL-7 | DCDIEGKDGKQYESVLMVSIDQLLDSMKEIGSNCLNNE | 166 | |
| | FNFFKRHICDANKEGMFLFRAARKLRQFLKMNSTGDF | | |
| | DLHLLKVSEGTTILLNCTGQVKGRKPAALGEAQPTKSL | | |
| | EENKSLKEQKKLNDLCFLKRLLQEIKTCWNKILMGTK | | |
| | EH | | |
| IL-12 subunit | RNLPVATPDPGMFPCLHHSQNLLRAVSNMLQKARQTL | 167 | |
| alpha | EFYPCTSEEIDHEDITKDKTSTVEACLPLELTKNESCLN | | |
| | SRETSFITNGSCLASRKTSFMMALCLSSIYEDLKMYQV | | |
| | EFKTMNAKLLMDPKRQIFLDQNMLAVIDELMQALNFN | | |
| | SETVPQKSSLEEPDFYKTKIKLCILLHAFRIRAVTIDRV | | |
| | MSYLNAS | | |
| IL-12 subunit | IWELKKDVYVVELDWYPDAPGEMVVLTCDTPEEDGIT | 168 | |
| beta | WTLDQSSEVLGSGKTLTIQVKEFGDAGQYTCHKGGEV | | |
| | LSHSLLLLHKKEDGIWSTDILKDQKEPKNKTFLRCEAK | | |
| | NYSGRFTCWWLTTISTDLTFSVKSSRGSSDPQGVTCGA | | |
| | ATLSAERVRGDNKEYEYSVECQEDSACPAAEESLPIEV | | |
| | MVDAVHKLKYENYTSSFFIRDIIKPDPPKNLQLKPLKNS | | |
| | RQVEVSWEYPDTWSTPHSYFSLTFCVQVQGKSKREKK | | |
| | DRVFTDKTSATVICRKNASISVRAQDRYYSSSWSEWAS | | |
| | VPCS | | |
| IL-15 | NWVNVISDLKKIEDLIQSMHIDATLYTESDVHPSCKVT | 169 | |
| | AMKCFLLELQVISLESGDASIHDTVENLIILANNSLSSN | | |
| | GNVTESGCKECEELEEKNIKEFLQSFVHIVQMFINTS | | |
| IL-18 | YFGKLESKLSVIRNLNDQVLFIDQGNRPLFEDMTDSDC | 170 | |
| | RDNAPRTIFIISMYKDSQPRGMAVTISVKCEKISTLSCE | | |
| | NKIISFKEMNPPDNIKDTKSDIIFFQRSVPGHDNKMQFE | | |
| | SSSYEGYFLACEKERDLFKLILKKEDELGDRSIMFTVQ | | |
| | NED | | |
| IL-21 | HKSSSQGQDRHMIRMRQLIDIVDQLKNYVNDLVPEFLP | 171 | |
| | APEDVETNCEWSAFSCFQKAQLKSANTGNNERIINVSI | | |
| | KKLKRKPPSTNAGRRQKHRLTCPSCDSYEKKPPKEFLE | | |
| | RFKSLLQKMIHQHLSSRTHGSEDS | | |

In some embodiments, the costimulatory and/or adhesion molecule is linked to a transmembrane domain. In some embodiments, the transmembrane domain may be the transmembrane domain of CD8, an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1 BB (CD137), 4-1 BBL, GITR, CD40, BAFFR, HVEM (LIGHTR), SLAMF7, NKp80 (KLRFI), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAMI, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain may be the transmembrane domain of CD28. In some embodiments, the transmembrane domain of may be the transmembrane domain of CD8, for example, CD8a.

Without wishing to be bound by theory, reducing foreign junctions (i.e., between an adhesion molecule and a transmembrane domain) in the foreign nucleic acid incorporated into a lentiviral particle may reduce the immunogenicity of a subject to a lentiviral particle. In some embodiments, an engineered lentiviral particle displaying a multi-domain fusion polypeptide will comprise a transmembrane domain from the polypeptide domain which is membrane proximal.

In some embodiments, the adhesion molecule is CD58. CD58 is also known as lymphocyte function-associated antigen 3 (LFA-3). CD58 binds to CD2 (LFA-2) on T cells. The extracellular portion of CD58 is residues 29-215 of SEQ ID NO: 1 (SEQ ID NO: 10):

(SEQ ID NO: 10)
FSQQIYGVVYGNVTFHVPSNVPLKEVLWKKQKDKVAELENSEFRA
FSSFKNRVYLDTVSGSLTIYNLTSSDEDEYEMESPNITDTMKFFL
YVLESLPSPTLTCALTNGSIEVQCMIPEHYNSHRGLIMYSWDCPM
EQCKRNSTSIYFKMENDLPQKIQCTLSNPLFNTTSSIILTTCIPS
SGHSRHR

In some embodiments, the polypeptide sequence of CD58 shares at least 50%, at least 75%, at least 80%, at least 85%, at least 90%, at least 91%, at least 92%, at least 93%, at least 94%, at least 95%, at least 96%, at least 97%, at least 98% or at least 99% identity to SEQ ID NO: 248:

(SEQ ID NO: 248)
FSQQIYGVVYGNVTFHVPSNVPLKEVLWKKQKDKVAELENSEFRA
FSSFKNRVYLDTVSGSLTIYNLTSSDEDEYEMESPNITDTMKFFL
YVLESL

A crystal structure of CD58 is described in Ikemizu et al. PNAS USA 96(8):4289-94 (1999). The extracellular portion of CD58 has a ligand-binding domain and a second extracellular domain. In embodiments, the ligand-binding domain may be used as the functional fragment of CD58—i.e., without the second extracellular domain.

In some embodiments, the adhesion molecule (or the fusion protein) comprises the polypeptide sequence of SEQ ID NO: 1 or 10, or a sequence having at least 75%, at least 80%, at least 85%, at least 90%, at least 91%, at least 92%, at least 93%, at least 94%, at least 95%, at least 96%, at least 97%, at least 98% or at least 99% identity to SEQ ID NO: 1 or 10. In some embodiments, the adhesion molecule (or the fusion protein) comprises a sequence having less than 75%, less than 80%, less than 85%, less than 90%, less than 91%, less than 92%, less than 93%, less than 94%, less than 95%, less than 96%, less than 97%, less than 98%, less than 99%, or less than 100% identity to SEQ ID NO: 1 or 10. The adhesion molecule may encoded by a polynucleotide (e.g. a DNA or RNA polynucleotide).

The adhesion molecule may encoded by the polynucleotide sequence of CD58, SEQ ID NO: 11, or by a subsequence encoding the extracellular portion or a functional fragment. SEQ ID NO: 11 (5′ to 3′):

ATGGTTGCTGGGAGCGACGCGGGGGGGGCCCTGGGGGTCCTCAGC
GTGGTCTGCCTGCTGCACTGCTTTGGTTTCATCAGCTGTTTTTCC
CAACAAATATATGGTGTTGTGTATGGGAATGTAACTTTCCATGTA
CCAAGCAATGTGCCTTTAAAAGAGGTCCTATGGAAAAAACAAAAG
GATAAAGTTGCAGAACTGGAAAATTCTGAGTTCAGAGCTTTCTCA
TCTTTTAAAAATAGGGTTTATTTAGACACTGTGTCAGGTAGCCTC
ACTATCTACAACTTAACATCATCAGATGAAGATGAGTATGAAATG
GAATCGCCAAATATTACTGATACCATGAAGTTCTTTCTTTATGTG
CTTGAGTCTCTTCCATCTCCCACACTAACTTGTGCATTGACTAAT
GGAAGCATTGAAGTCCAATGCATGATACCAGAGCATTACAACAGC
CATCGAGGACTTATAATGTACTCATGGGATTGTCCTATGGAGCAA
TGTAAACGTAACTCAACCAGTATATATTTTAAGATGGAAAATGAT
CTTCCACAAAAAATACAGTGTACTCTTAGCAATCCATTATTTAAT
ACAACATCATCAATCATTTTGACAACCTGTATCCCAAGCAGCGGT
CATTCAAGACACAGATATGCACTTATACCCATACCATTAGCAGTA
ATTACAACATGTATTGTGCTGTATATGAATGGTATTCTGAAATGT
GACAGAAAACCAGACAGAACCAACTCCAAT.

The polynucleotide sequence may be varied by codon-optimization or other methods to generate polynucleotide sequences having at least 50%, at least 75%, at least 80%, at least 85%, at least 90%, at least 91%, at least 92%, at least 93%, at least 94%, at least 95%, at least 96%, at least 97%, at least 98% or at least 99% identity to SEQ ID NO: 11, or a suitable subsequence, which may be used to express the adhesion molecule.

It will be appreciated that further variants of CD58 may be used. For example, homologs of CD58 from other species (mice, ape, horse, etc.) may be identified and tested for use in transducing human, or non-human, target cells. It is expected that at least some non-human homologs will retain adhesion molecule function when used with human target cells.

Further adhesion molecules useful in the practice of the present invention may include any molecule that specifically binds CD2, LFA-1, or DNAM-1. For example, the adhesion molecule may be a molecule that comprises an antibody, or antigen-binding fragment thereof, specific to CD2, LFA-1, or DNAM-1.

In some embodiments, the adhesion molecule binds to CD2. CD2 is also known as T11, LFA-2, and the erythrocyte rosette receptor. In its native state, CD2 is a surface protein expressed on T lymphocytes and NK cells. CD2 is a natural ligand for CD58. In addition to performing adhesion functions, engagement of CD2 by CD58 provides a costimulatory signal that may enhance activation and effector functions. In some embodiments, the particle comprises an adhesion molecule that binds to CD2, which may be CD58 or a fragment thereof. In some embodiments, the lentiviral particle comprises an antibody, single domain antibody, antibody fragment, and/or nanobody specific for CD2.

The foregoing description of CD58 and its derivatives as the adhesion molecule and CD2 as the cognate molecule may be extrapolated to the other adhesion molecules described herein. The adhesion molecule (or the fusion protein) may comprise any polypeptide sequence of in Table 1, to an extracellular portion thereof, or to a functional fragment thereof, or a sequence having at least 75%, at least 80%, at least 85%, at least 90%, at least 95%, at least 96%, at least 97%, at least 98% or at least 99% identity to a sequence in Table 1, to an extracellular portion thereof, or to a functional fragment thereof.

### Costimulatory Molecules

Disclosed herein, in some embodiments, are costimulatory molecules. The costimulatory molecule may be included as part of a fusion molecule. The costimulatory molecule may be included as part of a particle (e.g. displayed on a particle surface).

The fusion molecule displayed on the particle may include a costimulatory molecule. However, in some embodiments, the fusion molecule does not include a costimulatory molecule. The particle may display a costimulatory molecule as a separate molecule on the surface of the particle, or the particle may lack any costimulatory molecule. The costimulatory molecule may be a protein, termed herein a “costimulatory protein.”

As used herein, the term “costimulatory molecule” refers to a molecule capable of providing a costimulatory signal to target cells. In T cell biology, the binding of the T cell receptor by an antigen can provide the primary stimulatory signal to the cell. So-called costimulatory signals are provided by accessory molecules. An example costimulatory signal is the signal provided by binding of CD28 on T cells by a ligand. Some examples of ligands of CD28 include CD80 and CD86.

Illustrative costimulatory molecules include, but are not limited to, CD80, CD86, CD40L (also known as CD154), GITRL, OX40L, 41BBL, ICOSL, CD27, CD30L, LIGHT, LTalpha, MICA, and MICB. Each of the foregoing may be employed as a costimulatory molecules as a full-length protein, an extracellular domain, or functional fragment.

Polypeptide sequences of illustrative costimulatory molecules are provided in Table 2, with the “start” and “end” positions of the extracellular portion of each. In each case, the costimulatory molecule may comprise a polypeptide at least 75%, at least 80%, at least 85%, at least 90%, at least 91%, at least 92%, at least 93%, at least 94%, at least 95%, at least 96%, at least 97%, at least 98%, at least 99%, or 100% sequence identity to any sequence in Table 2, or functional fragments thereof. In some embodiments, the costimulatory molecule comprises a polypeptide having less than 75%, less than 80%, less than 85%, less than 90%, less than 91%, less than 92%, less than 93%, less than 94%, less than 95%, less than 96%, less than 97%, less than 98%, less than 99%, or 100% sequence identity to any sequence in Table 2, or a functional fragment thereof. Functional fragments may be or include any 10, 20, 30, 40, 50, 75, 100, 200, 300, 400, 500, or 600 amino acid portion that retains binding affinity to its cognate molecule, when measured using affinity assays such as biolayer interferometry or other assays known in the art.

TABLE 2

| Costimulatory | SEQ | | | |
|---|---|---|---|---|
| Molecule | ID NO: | Sequence | start | end |
| CD80 | 12 | MGHTRRQGTSPSKCPYLNFFQLLVLAGLSHFCSGVIHVT | 35 | 242 |
| | | KEVKEVATLSCGHNVSVEELAQTRIYWQKEKKMVLTM | | |
| | | MSGDMNIWPEYKNRTIFDITNNLSIVILALRPSDEGTYEC | | |
| | | VVLKYEKDAFKREHLAEVTLSVKADFPTPSISDFEIPTSNI | | |
| | | RRIICSTSGGFPEPHLSWLENGEELNAINTTVSQDPETELY | | |
| | | AVSSKLDFNMTTNHSFMCLIKYGHLRVNQTFNWNTTKQE | | |
| | | HFPDNLLPSWAITLISVNGIFVICCLTYCFAPRCRERRRN | | |
| | | ERLRRESVRPV | | |
| CD80 | 250 | VIHVTKEVKEVATLSCGHNVSVEELAQTRIYWQKEKKM | | |
| | | VLTMMSGDMNIWPEYKNRTIFDITNNLSIVILALRPSDEG | | |
| | | TYECVVLKYEKDAFKREHLAEVTLSVKADFPTPSISDFEI | | |
| | | PTSNIRRIICSTSGGFPEPHLSWLENGEELNAINTTVSQD | | |
| | | PETELYAVSSKLDFNMTTNHSFMCLIKYGHLRVNQTFNWN | | |
| | | TTKQEHFPDNLLPSWAITLISVNGIFVICCLTYCFAPRCR | | |
| | | ERRRNERLRRESVRPV | | |
| CD86 | 13 | MDPQCTMGLSNILFVMAFLLSGAAPLKIQAYFNETADLP | 24 | 247 |
| | | CQFANSQNQSLSELVVFWQDQENLVLNEVYLGKEKFDS | | |
| | | VHSKYMGRTSFDSDSWTLRLHNLQIKDKGLYQCIIHHKK | | |
| | | PTGMIRIHQMNSELSVLANFSQPEIVPISNITENVYINLT | | |
| | | CSSIHGYPEPKKMSVLLRTKNSTIEYDGVMQKSQDNVTELY | | |
| | | DVSISLSVSFPDVTSNMTIFCILETDKTRLLSSPFSIELED | | |
| | | PQPPPDHIPWITAVLPTVIICVMVFCLILWKWKKKKRPRNS | | |
| | | YKCGTNTMEREESEQTKKREKIHIPERSDEAQRVFKSSKTS | | |
| | | SCDKSDTCF | | |
| CD40 | 14 | MVRLPLQCVLWGCLLTAVHPEPPTACREKQYLINSQCCS | 21 | 193 |
| | | LCQPGQKLVSDCTEFTETECLPCGESEFLDTWNRETHCH | | |
| | | QHKYCDPNLGLRVQQKGTSETDTICTCEEGWHCTSEACE | | |
| | | SCVLHRSCSPGFGVKQIATGVSDTICEPCPVGFFSNVSSAF | | |
| | | EKCHPWTSCETKDLVVQQAGTNKTDVVCGPQDRLRALV | | |
| | | VIPIIFGILFAILLVLVFIKKVAKKPTNKAPHPKQEPQEI | | |
| | | NFPDDLPGSNTAAPVQETLHGCQPVTQEDGKESRISVQERQ | | |
| GITRL | 15 | MCLSHLENMPLSHSRTQGAQRSSWKLWLFCSIVMLLFLC | 49 | 177 |
| | | SFSWLIFIFLQLETAKEPCMAKFGPLPSKWQMASSEPPCV | | |
| | | NKVSDWKLEILQNGLYLIYGQVAPNANYNDVAPFEVRL | | |
| | | YKNKDMIQTLTNKSKIQNVGGTYELHVGDTIDLIFNSEH | | |
| | | QVLKNNTYWGIILLANPQFIS | | |
| OX40L | 16 | MERVQPLEENVGNAARPRFERNKLLLVASVIQGLGLLLC | 51 | 183 |
| | | FTYICLHFSALQVSHRYPRIQSIKVQFTEYKKEKGFILTSQ | | |
| | | KEDEIMKVQNNSVIINCDGFYLISLKGYFSQEVNISLHYQ | | |
| | | KDEEPLFQLKKVRSVNSLMVASLTYKDKVYLNVTTDNT | | |
| | | SLDDFHVNGGELILIHQNPGEFCVL | | |
| 41BBL | 17 | MEYASDASLDPEAPWPPAPRARACRVLPWALVAGLLLL | 50 | 254 |
| | | LLLAAACAVFLACPWAVSGARASPGSAASPRLREGPELS | | |
| | | PDDPAGLLDLRQGMFAQLVAQNVLLIDGPLSWYSDPGL | | |
| | | AGVSLTGGLSYKEDTKELVVAKAGVYYVFFQLELRRVV | | |
| | | AGEGSGSVSLALHLQPLRSAAGAAALALTVDLPPASSEA | | |
| | | RNSAFGFQGRLLHLSAGQRLGVHLHTEARARHAWQLTQ | | |
| | | GATVLGLFRVTPEIPAGLPSPRSE | | |
| ICOSL | 18 | MRLGSPGLLFLLFSSLRADTQEKEVRAMVGSDVELSCAC | 19 | 256 |
| | | PEGSRFDLNDVYVYWQTSESKTVVTYHIPQNSSLENVDS | | |
| | | RYRNRALMSPAGMLRGDFSLRLFNVTPQDEQKFHCLVL | | |
| | | SQSLGFQEVLSVEVTLHVAANFSVPVVSAPHSPSQDELTF | | |
| | | TCTSINGYPRPNVYWINKTDNSLLDQALQNDTVFLNMRG | | |
| | | LYDVVSVLRIARTPSVNIGCCIENVLLQQNLTVGSQTGND | | |
| | | IGERDKITENPVSTGEKNAATWSILAVLCLLVVVAVAIG | | |
| | | WVCRDRCLQHSYAGAWAVSPETELTGHV | | |
| CD27 | 19 | MARPHPWWLCVLGTLVGLSATPAPKSCPERHYWAQGK | 20 | 191 |
| | | LCCQMCEPGTFLVKDCDQHRKAAQCDPCIPGVSFSPDHH | | |
| | | TRPHCESCRHCNSGLLVRNCTITANAECACRNGWQCRD | | |
| | | KECTECDPLPNPSLTARSSQALSPHPQPTHLPYVSEMLEA | | |
| | | RTAGHMQTLADFRQLPARTLSTHWPPQRSLCSSDFIRILV | | |
| | | IFSGMFLVFTLAGALFLHQRRKYRSNKGESPVEPAEPCHY | | |
| | | SCPREEEGSTIPIQEDYRKPEPACSP | | |
| CD30L | 20 | MDPGLQQALNGMAPPGDTAMHVPAGSVASHLGTTSRSY | 63 | 234 |
| | | FYLTTATLALCLVFTVATIMVLVVQRTDSIPNSPDNVPLK | | |
| | | GGNCSEDLLCILKRAPFKKSWAYLQVAKHLNKTKLSWN | | |
| | | KDGILHGVRYQDGNLVIQFPGLYFIICQLQFLVQCPNNSV | | |
| | | DLKLELLINKHIKKQALVTVCESGMQTKHVYQNLSQFLL | | |
| | | DYLQVNTTISVNVDTFQYIDTSTFPLENVLSIFLYSNSD | | |
| LIGHT | 21 | MEESVVRPSVFVVDGQTDIPFTRLGRSHRRQSCSVARVG | 59 | 240 |
| | | LGLLLLLMGAGLAVQGWFLLQLHWRLGEMVTRLPDGP | | |
| | | AGSWEQLIQERRSHEVNPAAHLTGANSSLTGSGGPLLWE | | |
| | | TQLGLAFLRGLSYHDGALVVTKAGYYYIYSKVQLGGVG | | |
| | | CPLGLASTITHGLYKRTPRYPEELELLVSQQSPCGRATSSS | | |
| | | RVWWDSSFLGGVVHLEAGEKVVVRVLDERLVRLRDGT | | |
| | | RSYFGAFMV | | |
| LTalpha | 22 | MTPPERLFLPRVCGTTLHLLLLGLLLVLLPGAQGLPGVGL | | |
| | | TPSAAQTARQHPKMHLAHSTLKPAAHLIGDPSKQNSLLW | | |
| | | RANTDRAFLQDGFSLSNNSLLVPTSGIYFVYSQVVFSGKA | | |
| | | YSPKATSSPLYLAHEVQLFSSQYPFHVPLLSSQKMVYPGL | | |
| | | QEPWLHSMYHGAAFQLTQGDQLSTHTDGIPHLVLSPSTV | | |
| | | FFGAFAL | | |
| MICA | 23 | MGLGPVFLLLAGIFPFAPPGAAAEPHSLRYNLTVLSWDG | 24 | 307 |
| | | SVQSGFLTEVHLDGQPFLRCDRQKCRAKPQGQWAEDVL | | |
| | | GNKTWDRETRDLTGNGKDLRMTLAHIKDQKEGLHSLQE | | |
| | | IRVCEIHEDNSTRSSQHFYYDGELFLSQNLETKEWTMPQS | | |
| | | SRAQTLAMNVRNFLKEDAMKTKTHYHAMHADCLQELR | | |
| | | RYLKSGVVLRRTVPPMVNVTRSEASEGNITVTCRASGFY | | |
| | | PWNITLSWRQDGVSLSHDTQQWGDVLPDGNGTYQTWV | | |
| | | ATRICQGEEQRFTCYMEHSGNHSTHPVPSGKVLVLQSHW | | |
| | | QTFHVSAVAAAAIFVIIIFYVRCCKKKTSAAEGPELVSLQ | | |
| | | VLDQHPVGTSDHRDATQLGFQPLMSDLGSTGSTEGA | | |
| MICB | 24 | MGLGRVLLFLAVAFPFAPPAAAAEPHSLRYNLMVLSQD | 23 | 309 |
| | | GSVQSGFLAEGHLDGQPFLRYDRQKRRAKPQGQWAENV | | |
| | | LGAKTWDTETEDLTENGQDLRRTLTHIKDQKGGLHSLQ | | |
| | | EIRVCEIHEDSSTRGSRHFYYDGELFLSQNLETQESTVPQS | | |
| | | SRAQTLAMNVTNFWKEDAMKTKTHYRAMQADCLQKL | | |
| | | QRYLKSGVAIRRTVPPMVNVTCSEVSEGNITVTCRASSFY | | |
| | | PRNITLTWRQDGVSLSHNTQQWGDVLPDGNGTYQTWV | | |
| | | ATRIRQGEEQRFTCYMEHSGNHGTHPVPSGKALVLQSQR | | |
| | | TDFPYVSAAMPCFVIIIILCVPCCKKKTSAAEGPELVSLQV | | |
| | | LDQHPVGTGDHRDAAQLGFQPLMSATGSTGSTEGT | | |

In some embodiments, the costimulatory molecule is or includes CD80. In some embodiments, the costimulatory molecule is or includes a molecule that binds CD28. CD80 binds to CD28. The extracellular portion of CD80 includes residues 35-230 of SEQ ID NO: 12, which includes an Ig-like V-type domain (SEQ ID NO: 25) and an Ig-like C2-type domain (SEQ ID NO: 26), either or both of which may be included to form the costimulatory molecule.

(SEQ ID NO: 25)
VIHVTKEVKEVATLSCGHNVSVEELAQTRIYWQKEKKMVLTMMSGD
MNIWPEYKNRTIFDITNNLSIVILALRPSDEGTYECVVLKYEKDAF
KREHLAEVT
(SEQ ID NO: 26)
PSISDFEIPTSNIRRIICSTSGGFPEPHLSWLENGEELNAINTTVS
QDPETELYAVSSKLDFNMTTNHSFMCLIKYGHLRVNQTFN

The crystal structure of CD80 (also known as B7-1) is described in Ikemizu et al. Immunity 12:51-60 (2000). The extracellular portion of CD80 has two domains, described above. In embodiments, one or both of the domains may be used as the functional fragment of CD80.

In some embodiments, the costimulatory molecule is or includes CD86. CD86 binds to CD28. The extracellular portion of CD86 includes residues 33-225 of SEQ ID NO: 13, which includes an Ig-like V-type domain (SEQ ID NO: 27) and an Ig-like C2-type domain (SEQ ID NO: 28), either or both of which may be included to form the costimulatory molecule.

(SEQ ID NO: 27)
NETADLPCQFANSQNQSLSELVVFWQDQENLVLNEVYLGKEKFDSVH
SKYMGRTSFDSDSWTLRLHNLQIKDKGLYQCIIHHKKPTGMIRIHQM
NSELS
(SEQ ID NO: 28)
NVYINLTCSSIHGYPEPKKMSVLLRTKNSTIEYDGVMQKSQDNVTE
LYDVSISLSVSFPDVTSNMTIFCILETDKT

The crystal structure of CD86 (also known as B7-1) is described in Schwartz et al. Nature 410: 604-608 (2001). The extracellular portion of CD86 has two domains, described above. In embodiments, one or both of the domains may be used as the functional fragment of CD86.

It will be appreciated that further variants of CD80 or CD86 may be used. For example, homologs of CD80 or CD86 from other species (mice, ape, horse, etc.) may be identified and tested for use in transducing human, or non-human, target cells. It is expected that at least some non-human homologs will retain costimulatory molecule function when used with human target cells.

In some embodiments, the costimulatory molecule (or the fusion protein) comprises the polypeptide sequence of one or more of SEQ ID NO: 12-13 and 25-28, or a sequence having at least 75%, at least 80%, at least 85%, at least 90%, at least 91%, at least 92%, at least 93%, at least 94%, at least 95%, at least 96%, at least 97%, at least 98% or at least 99% identity to one or more of SEQ ID NO: 12-13 and 25-28.

In some embodiments, the costimulatory molecule CD80 comprises the polypeptide sequence of SEQ ID NO: 250, or a sequence having at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 250.

In some embodiments, the costimulatory molecule (or the fusion protein) comprises a polypeptide sequence having less than 75%, less than 80%, less than 85%, less than 90%, less than 91%, less than 92%, less than 93%, less than 94%, less than 95%, less than 96%, less than 97%, less than 98%, less than 99%, or less than 100% identity to one or more of SEQ ID NO: 12-13 and 25-28. The costimulatory molecule may encoded by a polynucleotide (e.g. a DNA or RNA polynucleotide). The costimulatory molecule may encoded by the polynucleotide sequence of CD80 (SEQ ID NO: 29) or CD86 (SEQ ID NO: 30), or by a subsequence encoding the extracellular portion or a functional fragment.

(SEQ ID NO: 29)
ATGGGCCACACACGGAGGCAGGGAACATCACCATCCAAGTGTCCA
TACCTCAATTTCTTTCAGCTCTTGGTGCTGGCTGGTCTTTCTCAC
TTCTGTTCAGGTGTTATCCACGTGACCAAGGAAGTGAAAGAAGTG
GCAACGCTGTCCTGTGGTCACAATGTTTCTGTTGAAGAGCTGGCA
CAAACTCGCATCTACTGGCAAAAGGAGAAGAAAATGGTGCTGACT
ATGATGTCTGGGGACATGAATATATGGCCCGAGTACAAGAACCGG
ACCATCTTTGATATCACTAATAACCTCTCCATTGTGATCCTGGCT
CTGCGCCCATCTGACGAGGGCACATACGAGTGTGTTGTTCTGAAG
TATGAAAAAGACGCTTTCAAGCGGGAACACCTGGCTGAAGTGACG
TTATCAGTCAAAGCTGACTTCCCTACACCTAGTATATCTGACTTT
GAAATTCCAACTTCTAATATTAGAAGGATAATTTGCTCAACCTCT
GGAGGTTTTCCAGAGCCTCACCTCTCCTGGTTGGAAAATGGAGAA
GAATTAAATGCCATCAACACAACAGTTTCCCAAGATCCTGAAACT
GAGCTCTATGCTGTTAGCAGCAAACTGGATTTCAATATGACAACC
AACCACAGCTTCATGTGTCTCATCAAGTATGGACATTTAAGAGTG
AATCAGACCTTCAACTGGAATACAACCAAGCAAGAGCATTTTCCT
GATAACCTGCTCCCATCCTGGGCCATTACCTTAATCTCAGTAAAT
GGAATTTTTGTGATATGCTGCCTGACCTACTGCTTTGCCCCAAGA
TGCAGAGAGAGAAGGAGGAATGAGAGATTGAGAAGGGAAAGTGTA
CGCCCTGTA
(SEQ ID NO: 30)
ATGGATCCCCAGTGCACTATGGGACTGAGTAACATTCTCTTTGTG
ATGGCCTTCCTGCTCTCTGGTGCTGCTCCTCTGAAGATTCAAGCT
TATTTCAATGAGACTGCAGACCTGCCATGCCAATTTGCAAACTCT
CAAAACCAAAGCCTGAGTGAGCTAGTAGTATTTTGGCAGGACCAG
GAAAACTTGGTTCTGAATGAGGTATACTTAGGCAAAGAGAAATTT
GACAGTGTTCATTCCAAGTATATGGGCCGCACAAGTTTTGATTCG
GACAGTTGGACCCTGAGACTTCACAATCTTCAGATCAAGGACAAG
GGCTTGTATCAATGTATCATCCATCACAAAAAGCCCACAGGAATG
ATTCGCATCCACCAGATGAACTCTGAACTGTCAGTGCTTGCTAAC
TTCAGTCAACCTGAAATAGTACCAATTTCTAATATAACAGAAAAT
GTGTACATAAATTTGACCTGCTCATCTATACACGGTTACCCAGAA
CCTAAGAAGATGAGTGTTTTGCTAAGAACCAAGAACTCAACTATC
GAGTATGATGGTGTTATGCAGAAATCTCAAGATAATGTCACAGAA
CTGTACGACGTTTCCATCAGCTTGTCTGTTTCATTCCCTGATGTT
ACGAGCAATATGACCATCTTCTGTATTCTGGAAACTGACAAGACG
CGGCTTTTATCTTCACCTTTCTCTATAGAGCTTGAGGACCCTCAG
CCTCCCCCAGACCACATTCCTTGGATTACAGCTGTACTTCCAACA
GTTATTATATGTGTGATGGTTTTCTGTCTAATTCTATGGAAATGG
AAGAAGAAGAAGCGGCCTCGCAACTCTTATAAATGTGGAACCAAC
ACAATGGAGAGGGAAGAGAGTGAACAGACCAAGAAAAGAGAAAAA
ATCCATATACCTGAAAGGTCTGATGAAGCCCAGCGTGTTTTTAAA
AGTTCGAAGACATCTTCATGCGACAAAAGTGATACATGTTTT

The polynucleotide sequence may be varied by codon-optimization or other methods to generate polynucleotide sequences having at least 50%, at least 75%, at least 80%, at least 85%, at least 90%, at least 91%, at least 92%, at least 93%, at least 94%, at least 95%, at least 96%, at least 97%, at least 98% or at least 99% identity to SEQ ID NO: 29 or 30, or a suitable subsequence, which may be used to express the costimulatory molecule.

Further costimulatory molecules useful in the practice of the present invention may include any molecule that specifically binds CD28. For example, the costimulatory molecule may be a molecule that comprises an antibody, or antigen-binding fragment thereof, specific to CD28.

CD28 is a receptor expressed on T cells that provide costimulatory signal. T cell costimulation through CD28, resulting in, for example, the production of various interleukins (in particular IL-6). In some embodiments, the costimulatory molecule is an antibody, or fragment thereof, that specifically binds to CD28. Examples of such antibodies include 15E8, TGN1412, CD28.2, and 10F3, as well as humanized variants thereof.

15E8 is a mouse monoclonal antibody to human CD28. Its complementarity determining regions (CDRs) are as follows:

CDRH1:
(SEQ ID NO: 36)
GFSLTSY
CDRH2:
(SEQ ID NO. 37)
WAGGS
CDRH3:
(SEQ ID NO. 38)
DKRAPGKLYYGYPDY
CDRL1:
(SEQ ID NO. 39)
RASESVEYYVTSLMQ
CDRL2:
(SEQ ID NO. 40)
AASNYES
CDRL3:
(SEQ ID NO. 41)
QQTRKVPST

TGN1412 (also known as CD28-SuperMAB) is a humanized monoclonal antibody that not only binds to, but also is a strong agonist for, the CD28 receptor. Its CDRs are as follows.

CDRH1:
(SEQ ID NO. 42)
GYTFSY
CDRH2:
(SEQ ID NO. 43)
YPGNVN
CDRH3:
(SEQ ID NO. 44)
SHYGLDWNFDV
CDRL1:
(SEQ ID NO. 45)
HASQNIYVLN
CDRL2:
(SEQ ID NO. 46)
KASNLHT
CDRL3:
(SEQ ID NO. 47)
QQGQTYPYT

The foregoing description of CD80, CD86, and their derivatives as the costimulatory molecule and CD28 as the cognate molecule may be extrapolated to the other costimulatory molecules described herein, including but not limited to those listed in Table 2. The costimulatory molecule (or the fusion protein) may comprise any polypeptide sequence of in Table 2, to an extracellular portion thereof, or to a functional fragment thereof, or a sequence having at least 75%, at least 80%, at least 85%, at least 90%, at least 95%, at least 96%, at least 97%, at least 98% or at least 99% identity to a sequence in Table 2, to an extracellular portion thereof, or to a functional fragment thereof.

### Activation Molecules

Disclosed herein, in some embodiments, are activation molecules. The activation molecule may be included as part of a fusion molecule. The activation molecule may be included as part of a particle (e.g. displayed on a particle surface). An example of an activation molecule may include a TCR-binding molecule.

The fusion molecule displayed on the particle may include an activation molecule (e.g. a TCR-binding molecule) or other subunit that provides an activation signal to a target cell. However, in some embodiments, the fusion molecule does not include a TCR-binding molecule or other activation domain. The particle may display a TCR-binding molecule as a separate molecule on the surface of the particle, or the particle may lack any TCR-binding molecule. The TCR-binding molecule may be a protein, termed herein a “TCR-binding protein.” The activation molecule may be or include an activation protein.

As used herein, the term “TCR-binding molecule” refers to a molecule capable of directly binding the extracellular portion of the T cell receptor (TCR) by contacting one or more components of the TCR or otherwise providing a primary or “signal 1” activation signal to a target cell (e.g. a T cell or NK cell). The structure of the TCR, its components, and function is described in Susac et al. Cell 185(17):3201-3213.e19 (2022). Some examples of TCR-binding molecules may include an antibody, or antigen binding fragment, that specifically binds CD3 (an anti-CD3 monoclonal antibody, or antigen binding fragment thereof). In some embodiments, the activation molecule comprises an antibody, single domain antibody, antibody fragment, nanobody, or other binding protein specific for CD3. Illustrative antibodies include OKT3 (also known as Muromonab-CD3), otelixizumab, teplizumab and visilizumab. The complementarity determining regions of OKT3 are as follows:

CDRH1:
(SEQ ID NO. 48)
GYTFTRY
CDRH2:
(SEQ ID NO. 49)
NPSRGY
CDRH3:
(SEQ ID NO. 50)
YYDDHYCLDY
CDRL1:
(SEQ ID NO. 51)
SASSSVSYMN
CDRL2:
(SEQ ID NO. 52)
DTSKLAS
CDRL3:
(SEQ ID NO. 53)
QQWSSNPFT

The activation molecule (e.g. TCR-binding molecule) may be a single chain variable fragment (scFv) displayed on the particle as linked to a transmembrane region or an anchor. OKT3 in scFv format may be used.

In some embodiments, the activation molecule (e.g. TCR-binding molecule) is or includes an scFv comprising a polypeptide sequence at least 75%, at least 80%, at least 85%, at least 90%, at least 91%, at least 92%, at least 93%, at least 94%, at least 95%, at least 99%, or 100% identical to the anti-CD3 scFv of SEQ ID NO: 31, which includes a variable light (VL) and variable heavy (VH) domain with a 3×GGGS linker:

(SEQ ID NO: 31)
DIQMTQSPSSLSASVGDRVTITCSASSSVSYMNWYQQTPGKAPKRW
IYDTSKLASGVPSRFSGSGSGTDYTFTISSLQPEDIATYYCQQWS
SNPFTFGQGTKLQITRTSGGGGSGGGGSGGGGSQVQLVQSGGGVV
QPGRSLRLSCKASGYTFTRYTMHWVRQAPGKGLEWIGYINPSRGY
TNYNQKVKDRFTISRDNSKNTAFLQMDSLRPEDTGVYFCARYYDD
HYCLDYWGQGTPVTVSSAAAKP

In some embodiments, the activation molecule (e.g. TCR-binding molecule) is or includes an scFv comprising a polypeptide sequence at least 75%, at least 80%, at least 85%, at least 90%, at least 91%, at least 92%, at least 93%, at least 94%, at least 95%, at least 99%, or 100% identical to the anti-CD3 scFv of SEQ ID NO: 249, which includes a variable light (VL) and variable heavy (VH) domain with a 3×GGGS linker:

(SEQ ID NO: 249)
DIQMTQSPSSLSASVGDRVTITCSASSSVSYMNWYQQTPGKAPKRW
IYDTSKLASGVPSRFSGSGSGTDYTFTISSLQPEDIATYYCQQWS
SNPFTFGQGTKLQITRTSGGGGSGGGGSGGGGSQVQLVQSGGGVV
QPGRSLRLSCKASGYTFTRYTMHWVRQAPGKGLEWIGYINPSRGY
TNYNQKVKDRFTISRDNSKNTAFLQMDSLRPEDTGVYFCARYYDD
HYCLDYWGQGTPVTVSSA

The complementarity determining regions of this scFv are as follows:

CDRH1:
(SEQ ID NO: 54)
RYTMH
CDRH2:
(SEQ ID NO: 55)
YINPSRGYTNYNQKVKD
CDRH3:
(SEQ ID NO: 56)
YYDDHYCLDY
CDRL1:
(SEQ ID NO: 57)
SASSSVSYMN
CDRL2:
(SEQ ID NO: 58)
DTSKLASG
CDRL3:
(SEQ ID NO: 59)
QQWSSNPFT

Other activation molecules and/or domains may comprise the binding regions of other proteins commonly found in the supramolecular activation complex (SMAC) between T lymphocytes and antigen presenting cells. For example, CD3, CD2, CD4, CD8, CD28, LFA-1, CD45, CD43, CD40, ICAM-1, CTLA-4, CD80, CD86, MHC, LFA-3, AND CD40L are proteins that may be present within the SMAC. The fusion proteins disclosed herein may comprise portions of these proteins or domains that bind to these proteins. For example, without wishing to be bound by theory, T cells may express one or both of CD4 and/or CD8 and fusion molecules disclosed herein may comprise domains that engage with either or both of CD4 and/or CD8.

When cells other than T cells are the intended target of the particles comprising a fusion molecule as disclosed herein, other binding domains may be more appropriate. For example, particles targeting NK cells may comprise domains that engage with proteins found on NK cells. In some embodiments, these proteins include CD2, CD16, NKp46, NKp30, and NKG2D. In some such embodiments, fusion proteins intended to target and/or activate NK cells may comprise domains that bind to CD2, CD16, NKp46, NKG2D, etc. Domains that bind to NKG2D may be derived from NKG2D ligands including, but not limited to: MICA, MICB, ULBP1, ULBP2, ULBP3, ULBP4, ULBP5, and ULBP6. In some embodiments, the fusion proteins described herein comprise a CD58 domain, a domain that binds NKG2D, and optionally a third domain which enhances activation of the target NK cell.

The activation molecule may be encoded by a polynucleotide (e.g. a DNA or RNA polynucleotide).

### Fusion Molecule

Disclosed herein, in some embodiments, are fusion molecules. The fusion molecule may include an adhesion molecule, a costimulatory molecule, or an activation molecule. The fusion molecule may include an adhesion molecule. The fusion molecule may include a costimulatory molecule. The fusion molecule may include an activation molecule. The fusion molecule may include an adhesion molecule, a costimulatory molecule, and an activation molecule. The fusion molecule may include an adhesion molecule and an activation molecule. The fusion molecule may include a costimulatory molecule and an activation molecule. The fusion molecule may be or include a fusion protein. The fusion molecule may be included as part of a particle. The fusion molecule may be used in a method described herein.

In some embodiments, the disclosure provides a fusion molecule comprising a combination of an adhesion molecule, a costimulatory molecule, and an activation molecule (e.g. a TCR-binding molecule), thereof each component linked directly or indirectly to the other components. In some embodiments, the fusion molecule comprises adhesion molecule, a costimulatory molecule, and an activation molecule (e.g. a TCR-binding molecule). In some embodiments, the fusion molecule comprises adhesion molecule and a costimulatory molecule, but not a TCR-binding molecule. In some embodiments, the fusion molecule comprises adhesion molecule and an activation molecule (e.g. a TCR-binding molecule), but not a costimulatory molecule. The fusion molecule may further comprise one or more additional adhesion molecules, costimulatory molecules, or activation molecules (e.g. TCR-binding molecules).

As used herein, the term “fusion molecule” refers to any molecule having multiple components link together, directly or indirectly, covalently or non-covalently. The fusion molecule may be made up of several proteins. When those proteins are linked together into a single molecule by peptide bonds, the fusion molecule is termed a “fusion protein.”

The fusion molecule may be made using various linkers, including chemical (covalent) bonds (e.g., by click chemistry) or by peptide bounds. When the fusion molecule is a fusion protein, the linker between each component of the fusion protein may be a single peptide bound (i.e., a direct C- to N-peptide bound in a polypeptide chain) or via a polypeptide linker. Illustrative polypeptide linkers may include, but are not limited to, the glycine-serine linkers, such as GGSGGS, GSSGSS, or others.

In some embodiments, the fusion molecule is or includes a fusion protein. The fusion protein may comprise an adhesion protein, a polypeptide linker, and a costimulatory portion. In some embodiments, the fusion protein comprises an adhesion molecule, a costimulatory molecule, and an activation molecule.

In some embodiments of the fusion protein, the adhesion molecule is N-terminal to the costimulatory molecule. In some embodiments, the adhesion molecule is N-terminal to the activation molecule. In some embodiments, the adhesion molecule is C-terminal to the costimulatory molecule. In some embodiments, the adhesion molecule is C-terminal to the activation molecule.

In some embodiments of the fusion protein, the activation molecule is N-terminal to the costimulatory molecule. In some embodiments, the activation molecule is N-terminal to the adhesion molecule. In some embodiments, the activation molecule is C-terminal to the costimulatory molecule. In some embodiments, the activation molecule is C-terminal to the adhesion molecule.

In some embodiments of the fusion protein, the costimulatory molecule is N-terminal to the activation molecule. In some embodiments, the costimulatory molecule is N-terminal to the adhesion molecule. In some embodiments, the costimulatory molecule is C-terminal to the activation molecule. In some embodiments, the costimulatory molecule is C-terminal to the adhesion molecule.

Some embodiments of the fusion protein includes a linker. Some embodiments include multiple linkers. In some embodiments, a linker directly connects the costimulatory molecule with the adhesion molecule. In some embodiments, a linker directly connects the costimulatory molecule with the activation molecule. In some embodiments, a linker directly connects the adhesion molecule with the activation molecule.

In some embodiments of the fusion protein, an N terminal end of the costimulatory molecule is juxtaposed (directly or via a linker) with an end of the adhesion molecule. In some embodiments of the fusion protein, a C terminal end of the costimulatory molecule is juxtaposed (directly or via a linker) with an end of the adhesion molecule. In some embodiments of the fusion protein, an N terminal end of the costimulatory molecule is juxtaposed (directly or via a linker) with an end of the activation molecule. In some embodiments of the fusion protein, a C terminal end of the costimulatory molecule is juxtaposed (directly or via a linker) with an end of the activation molecule.

In some embodiments of the fusion protein, an N terminal end of the activation molecule is juxtaposed (directly or via a linker) with an end of the adhesion molecule. In some embodiments of the fusion protein, a C terminal end of the activation molecule is juxtaposed (directly or via a linker) with an end of the adhesion molecule. In some embodiments of the fusion protein, an N terminal end of the activation molecule is juxtaposed (directly or via a linker) with an end of the costimulatory molecule. In some embodiments of the fusion protein, a C terminal end of the activation molecule is juxtaposed (directly or via a linker) with an end of the costimulatory molecule.

In some embodiments of the fusion protein, an N terminal end of the adhesion molecule is juxtaposed (directly or via a linker) with an end of the costimulatory molecule. In some embodiments of the fusion protein, a C terminal end of the adhesion molecule is juxtaposed (directly or via a linker) with an end of the costimulatory molecule. In some embodiments of the fusion protein, an N terminal end of the adhesion molecule is juxtaposed (directly or via a linker) with an end of the activation molecule. In some embodiments of the fusion protein, a C terminal end of the adhesion molecule is juxtaposed (directly or via a linker) with an end of the activation molecule.

Some non-limiting examples of fusion proteins are included in FIG. 60A-60B. For example, a fusion protein shown in FIG. 60A-60B may further comprise a viral protein such as a viral glycoprotein. Any of such fusion proteins may be included as part of a particle (e.g. a viral particle such as a lentiviral particle), or may be displayed on a particle surface.

The fusion protein may comprise, in any order, a CD80, a CD80 extracellular portion, or a functional fragment of CD80; a CD58, a CD58 extracellular portion; or a functional fragment of CD58; an activation molecule (e.g. a TCR-binding molecule); and polypeptide linkers.

The fusion protein may comprise, in N- to C-terminal order, CD80, a CD80 extracellular portion, or a functional fragment of CD80; a polypeptide linker; and CD58, a CD58 extracellular portion; or a functional fragment of CD58.

The fusion protein may comprise, in N- to C-terminal order, CD58, a CD58 extracellular portion; or a functional fragment of CD58; a polypeptide linker; and CD80, a CD80 extracellular portion, or a functional fragment of CD80.

The fusion protein may comprise, in N- to C-terminal order, an activation molecule (e.g. a TCR-binding protein); a polypeptide linker; CD80, a CD80 extracellular portion, or a functional fragment of CD80; a polypeptide linker; and CD58, a CD58 extracellular portion; or a functional fragment of CD58.

The fusion protein may comprise, in N- to C-terminal order, CD80, a CD80 extracellular portion, or a functional fragment of CD80; a polypeptide linker; CD58, a CD58 extracellular portion; or a functional fragment of CD58; a polypeptide linker; and an activation molecule (e.g. a TCR-binding protein).

The fusion protein may comprise, in N- to C-terminal order, an activation molecule (e.g. a TCR-binding protein); a polypeptide linker; CD58, a CD58 extracellular portion; or a functional fragment of CD58; a polypeptide linker; and CD80, a CD80 extracellular portion, or a functional fragment of CD80.

The fusion protein may comprise, in N- to C-terminal order, CD58, a CD58 extracellular portion; or a functional fragment of CD58; a polypeptide linker; CD80, a CD80 extracellular portion, or a functional fragment of CD80; a polypeptide linker; and an activation molecule (e.g. a TCR-binding protein).

An illustrative fusion protein comprises a CD58 extracellular region and α-CD3 scFv fused to the N-terminus of a CD80 via a linker; this construct is termed a tri-fusion polypeptide and/or termed “498.”

An illustrative fusion protein comprises a CD58 extracellular region fused to the N-terminus of a CD80 via a linker; this construct is termed a bi-fusion polypeptide and/or termed “455.” In this construct, an αCD3 scFv is expressed as a separate polypeptide in the producer cells.

In each case, the polypeptide linker may be optional. It may be omitted by directly linking protein molecule to the next via a peptide bound. Although one my generate fusion proteins through chemical synthesis, fusion protein are more made by expressing the fusion protein from a single polynucleotide comprising a polynucleotide sequence encoding the entire fusion protein. Methods for designing and cloning polynucleotides are known in the art.

The fusion molecule may encoded by a polynucleotide (e.g. a DNA or RNA polynucleotide). In some embodiments, the disclosure provides polynucleotides encoding such fusion proteins. The polynucleotide may be an isolated polynucleotide, or it may be part of a vector (e.g., a plasmid) or it may be introduced into and propagated in a host cell.

Polypeptide sequences of illustrative dual CD58+CD80 fusion proteins are provided in Table 3. In each case, the fusion protein may comprise a polypeptide at least 75%, at least 80%, at least 85%, at least 90%, at least 91%, at least 92%, at least 93%, at least 94%, at least 95%, at least 99%, or 100% sequence identity to any sequence in Table 3. In some embodiments, the fusion protein may comprise a polypeptide having less than 75%, less than 80%, less than 85%, less than 90%, less than 91%, less than 92%, less than 93%, less than 94%, less than 95%, less than 99%, or less than 100% sequence identity to any sequence in Table 3. In each case, an optional signal peptide is shown in parentheses. The signal peptide is cleaved during expression of the sequence. Sequence identity to a reference sequence is determined without the optional residues. Diagrams of each fusion are provided in FIGS. 9A and 9B.

TABLE 3

| Fusion | SEQ ID | |
|---|---|---|
| Protein | NO: | Sequence |
| #438 | 60 | (MGVKVLFALICIAVAEA)FSQQIYGVVYGNVTFHVPSNVPLKEVL |
| | | WKKQKDKVAELENSEFRAFSSFKNRVYLDTVSGSLTIYNLTSSDE |
| | | DEYEMESPNITDTMKFFLYVLESLGSGVIHVTKEVKEVATLSCGH |
| | | NVSVEELAQTRIYWQKEKKMVLTMMSGDMNIWPEYKNRTIFDIT |
| | | NNLSIVILALRPSDEGTYECVVLKYEKDAFKREHLAEVTLSVKADF |
| | | PTPDNLLPSWAITLISVNGIFVICCLTYCFAPRCRERRRNERLRRESV |
| | | RPV |
| #439 | 61 | (MVAGSDAGRALGVLSVVCLLHCFGFISC)FSQQIYGVVYGNVTFH |
| | | VPSNVPLKEVLWKKQKDKVAELENSEFRAFSSFKNRVYLDTVSGS |
| | | LTIYNLTSSDEDEYEMESPNITDTMKFFLYVLESLGSGVIHVTKEV |
| | | KEVATLSCGHNVSVEELAQTRIYWQKEKKMVLTMMSGDMNIWP |
| | | EYKNRTIFDITNNLSIVILALRPSDEGTYECVVLKYEKDAFKREHLA |
| | | EVTLSVKADFPTPDNLLPSWAITLISVNGIFVICCLTYCFAPRCRER |
| | | RRNERLRRESVRPV |
| #440 | 62 | (MGVKVLFALICIAVAEA)VIHVTKEVKEVATLSCGHNVSVEELAQ |
| | | TRIYWQKEKKMVLTMMSGDMNIWPEYKNRTIFDITNNLSIVILAL |
| | | RPSDEGTYECVVLKYEKDAFKREHLAEVTLSVKAGSGFSQQIYGV |
| | | VYGNVTFHVPSNVPLKEVLWKKQKDKVAELENSEFRAFSSFKNR |
| | | VYLDTVSGSLTIYNLTSSDEDEYEMESPNITDTMKFFLYVLESLPSS |
| | | GHSRHRYALIPIPLAVITTCIVLYMNGILKCDRKPDRTNSN |
| #441 | 63 | (MGHTRRQGTSPSKCPYLNFFQLLVLAGLSHFCSG)VIHVTKEVKE |
| | | VATLSCGHNVSVEELAQTRIYWQKEKKMVLTMMSGDMNIWPEY |
| | | KNRTIFDITNNLSIVILALRPSDEGTYECVVLKYEKDAFKREHLAEV |
| | | TLSVKAGSGFSQQIYGVVYGNVTFHVPSNVPLKEVLWKKQKDKV |
| | | AELENSEFRAFSSFKNRVYLDTVSGSLTIYNLTSSDEDEYEMESPNI |
| | | TDTMKFFLYVLESLPSSGHSRHRYALIPIPLAVITTCIVLYMNGILK |
| | | CDRKPDRTNSN |
| #449 | 64 | (MGVKVLFALICIAVAEA)VIHVTKEVKEVATLSCGHNVSVEELAQ |
| | | TRIYWQKEKKMVLTMMSGDMNIWPEYKNRTIFDITNNLSIVILAL |
| | | RPSDEGTYECVVLKYEKDAFKREHLAEVTLSVKAGSGFSQQIYGV |
| | | VYGNVTFHVPSNVPLKEVLWKKQKDKVAELENSEFRAFSSFKNR |
| | | VYLDTVSGSLTIYNLTSSDEDEYEMESPNITDTMKFFLYVLESLPSP |
| | | TLTCALTNGSIEVQCMIPEHYNSHRGLIMYSWDCPMEQCKRNSTSI |
| | | YFKMENDLPQKIQCTLSNPLFNTTSSIILTTCIPSSGHSRHRYALIPIP |
| | | LAVITTCIVLYMNGILKCDRKPDRTNSN |
| #450 | 65 | (MGVKVLFALICIAVAEA)FSQQIYGVVYGNVTFHVPSNVPLKEVL |
| | | WKKQKDKVAELENSEFRAFSSFKNRVYLDTVSGSLTIYNLTSSDE |
| | | DEYEMESPNITDTMKFFLYVLESLGSGVIHVTKEVKEVATLSCGH |
| | | NVSVEELAQTRIYWQKEKKMVLTMMSGDMNIWPEYKNRTIFDIT |
| | | NNLSIVILALRPSDEGTYECVVLKYEKDAFKREHLAEVTLSVKADF |
| | | PTPSISDFEIPTSNIRRIICSTSGGFPEPHLSWLENGEELNAINTTVSQ |
| | | DPETELYAVSSKLDFNMTTNHSFMCLIKYGHLRVNQTFNWNTTK |
| | | QEHFPDNLLPSWAITLISVNGIFVICCLTYCFAPRCRERRRNERLRR |
| | | ESVRPV |
| #452 | 66 | (MGHTRRQGTSPSKCPYLNFFQLLVLAGLSHFCSG)VIHVTKEVKE |
| | | VATLSCGHNVSVEELAQTRIYWQKEKKMVLTMMSGDMNIWPEY |
| | | KNRTIFDITNNLSIVILALRPSDEGTYECVVLKYEKDAFKREHLAEV |
| | | TLSVKAGSGFSQQIYGVVYGNVTFHVPSNVPLKEVLWKKQKDKV |
| | | AELENSEFRAFSSFKNRVYLDTVSGSLTIYNLTSSDEDEYEMESPNI |
| | | TDTMKFFLYVLESLPSPTLTCALTNGSIEVQCMIPEHYNSHRGLIM |
| | | YSWDCPMEQCKRNSTSIYFKMENDLPQKIQCTLSNPLFNTTSSIILT |
| | | TCIPSSGHSRHRYALIPIPLAVITTCIVLYMNGILKCDRKPDRTNSN |
| #453 | 67 | (MVAGSDAGRALGVLSVVCLLHCFGFISC)FSQQIYGVVYGNVTFH |
| | | VPSNVPLKEVLWKKQKDKVAELENSEFRAFSSFKNRVYLDTVSGS |
| | | LTIYNLTSSDEDEYEMESPNITDTMKFFLYVLESLGSGVIHVTKEV |
| | | KEVATLSCGHNVSVEELAQTRIYWQKEKKMVLTMMSGDMNIWP |
| | | EYKNRTIFDITNNLSIVILALRPSDEGTYECVVLKYEKDAFKREHLA |
| | | EVTLSVKADFPTPSISDFEIPTSNIRRIICSTSGGFPEPHLSWLENGEE |
| | | LNAINTTVSQDPETELYAVSSKLDFNMTTNHSFMCLIKYGHLRVN |
| | | QTFNWNTTKQEHFPDNLLPSWAITLISVNGIFVICCLTYCFAPRCRE |
| | | RRRNERLRRESVRPV |
| #454 | 68 | (MGHTRRQGTSPSKCPYLNFFQLLVLAGLSHFCSG)VIHVTKEVKE |
| | | VATLSCGHNVSVEELAQTRIYWQKEKKMVLTMMSGDMNIWPEY |
| | | KNRTIFDITNNLSIVILALRPSDEGTYECVVLKYEKDAFKREHLAEV |
| | | TLSVKADFPTPSISDFEIPTSNIRRIICSTSGGFPEPHLSWLENGEELN |
| | | AINTTVSQDPETELYAVSSKLDFNMTTNHSFMCLIKYGHLRVNQT |
| | | FNWNTTKQEHFPDNGSGFSQQIYGVVYGNVTFHVPSNVPLKEVL |
| | | WKKQKDKVAELENSEFRAFSSFKNRVYLDTVSGSLTIYNLTSSDE |
| | | DEYEMESPNITDTMKFFLYVLESLPSPTLTCALTNGSIEVQCMIPEH |
| | | YNSHRGLIMYSWDCPMEQCKRNSTSIYFKMENDLPQKIQCTLSNP |
| | | LFNTTSSIILTTCIPSSGHSRHRYALIPIPLAVITTCIVLYMNGILKCD |
| | | RKPDRTNSN |
| #455 | 32 | (MVAGSDAGRALGVLSVVCLLHCFGFISC)FSQQIYGVVYGNVTFH |
| | | VPSNVPLKEVLWKKQKDKVAELENSEFRAFSSFKNRVYLDTVSGS |
| | | LTIYNLTSSDEDEYEMESPNITDTMKFFLYVLESLPSPTLTCALTNG |
| | | SIEVQCMIPEHYNSHRGLIMYSWDCPMEQCKRNSTSIYFKMENDL |
| | | PQKIQCTLSNPLFNTTSSIILTTCIPSSGHSRHRGSGVIHVTKEVKEV |
| | | ATLSCGHNVSVEELAQTRIYWQKEKKMVLTMMSGDMNIWPEYK |
| | | NRTIFDITNNLSIVILALRPSDEGTYECVVLKYEKDAFKREHLAEVT |
| | | LSVKADFPTPSISDFEIPTSNIRRIICSTSGGFPEPHLSWLENGEELNA |
| | | INTTVSQDPETELYAVSSKLDFNMTTNHSFMCLIKYGHLRVNQTF |
| | | NWNTTKQEHFPDNLLPSWAITLISVNGIFVICCLTYCFAPRCRERRR |
| | | NERLRRESVRPV |

Polypeptide sequences of illustrative triple CD58+CD80+αCD3scFv fusion proteins are provided in Table 4A. In each case, the fusion protein may comprise a polypeptide at least 75%, at least 80%, at least 85%, at least 90%, at least 91%, at least 92%, at least 93%, at least 94%, at least 95%, at least 99%, or 100% sequence identity to any sequence in Table 4A. In some embodiments, the fusion protein may comprise a polypeptide less than 75%, less than 80%, less than 85%, less than 90%, less than 91%, less than 92%, less than 93%, less than 94%, less than 95%, less than 99%, or less than 100% sequence identity to any sequence in Table 4A. In each case, an optional signal peptide is shown in parentheses. The signal peptide is cleaved during expression of the sequence. Sequence identity to a reference sequence is determined without the optional residues. Diagrams of each fusion are provided in FIG. 27B.

TABLE 4A

| Fusion | | |
|---|---|---|
| Protein | SEQ ID NO: | Sequence |
| #478 | 69 | (MGVKVLFALICIAVAEA)DIQMTQSPSSLSASVGDRVTITCSASSSV |
| | | SYMNWYQQTPGKAPKRWIYDTSKLASGVPSRFSGSGSGTDYTFTI |
| | | SSLQPEDIATYYCQQWSSNPFTFGQGTKLQITRTSGGGGSGGGGSG |
| | | GGGSQVQLVQSGGGVVQPGRSLRLSCKASGYTFTRYTMHWVRQ |
| | | APGKGLEWIGYINPSRGYTNYNQKVKDRFTISRDNSKNTAFLQMD |
| | | SLRPEDTGVYFCARYYDDHYCLDYWGQGTPVTVSSAGSSGGSGG |
| | | GGSGGGGSGGGGSVIHVTKEVKEVATLSCGHNVSVEELAQTRIY |
| | | WQKEKKMVLTMMSGDMNIWPEYKNRTIFDITNNLSIVILALRPSD |
| | | EGTYECVVLKYEKDAFKREHLAEVTLSVKAGSGFSQQIYGVVYG |
| | | NVTFHVPSNVPLKEVLWKKQKDKVAELENSEFRAFSSFKNRVYLD |
| | | TVSGSLTIYNLTSSDEDEYEMESPNITDTMKFFLYVLESLPSPTLTC |
| | | ALTNGSIEVQCMIPEHYNSHRGLIMYSWDCPMEQCKRNSTSIYFK |
| | | MENDLPQKIQCTLSNPLFNTTSSIILTTCIPSSGHSRHRYALIPIPLAV |
| | | ITTCIVLYMNGILKCDRKPDRTNSN |
| #479 | 70 | (MGVKVLFALICIAVAEA)DIQMTQSPSSLSASVGDRVTITCSASSSV |
| | | SYMNWYQQTPGKAPKRWIYDTSKLASGVPSRFSGSGSGTDYTFTI |
| | | SSLQPEDIATYYCQQWSSNPFTFGQGTKLQITRTSGGGGSGGGGSG |
| | | GGGSQVQLVQSGGGVVQPGRSLRLSCKASGYTFTRYTMHWVRQ |
| | | APGKGLEWIGYINPSRGYTNYNQKVKDRFTISRDNSKNTAFLQMD |
| | | SLRPEDTGVYFCARYYDDHYCLDYWGQGTPVTVSSAGSSGGSGG |
| | | GGSGGGGSGGGGSFSQQIYGVVYGNVTFHVPSNVPLKEVLWKKQ |
| | | KDKVAELENSEFRAFSSFKNRVYLDTVSGSLTIYNLTSSDEDEYEM |
| | | ESPNITDTMKFFLYVLESLGSGVIHVTKEVKEVATLSCGHNVSVEE |
| | | LAQTRIYWQKEKKMVLTMMSGDMNIWPEYKNRTIFDITNNLSIVI |
| | | LALRPSDEGTYECVVLKYEKDAFKREHLAEVTLSVKADFPTPSISD |
| | | FEIPTSNIRRIICSTSGGFPEPHLSWLENGEELNAINTTVSQDPETELY |
| | | AVSSKLDFNMTTNHSFMCLIKYGHLRVNQTFNWNTTKQEHFPDN |
| | | LLPSWAITLISVNGIFVICCLTYCFAPRCRERRRNERLRRESVRPV |
| #495 | 71 | (MGHTRRQGTSPSKCPYLNFFQLLVLAGLSHFCSG)VIHVTKEVKE |
| | | VATLSCGHNVSVEELAQTRIYWQKEKKMVLTMMSGDMNIWPEY |
| | | KNRTIFDITNNLSIVILALRPSDEGTYECVVLKYEKDAFKREHLAEV |
| | | TLSVKAGSGDIQMTQSPSSLSASVGDRVTITCSASSSVSYMNWYQ |
| | | QTPGKAPKRWIYDTSKLASGVPSRFSGSGSGTDYTFTISSLQPEDIA |
| | | TYYCQQWSSNPFTFGQGTKLQITRTSGGGGSGGGGSGGGGSQVQL |
| | | VQSGGGVVQPGRSLRLSCKASGYTFTRYTMHWVRQAPGKGLEWI |
| | | GYINPSRGYTNYNQKVKDRFTISRDNSKNTAFLQMDSLRPEDTGV |
| | | YFCARYYDDHYCLDYWGQGTPVTVSSAGSSGGSGGGGSGGGGS |
| | | GGGGSSGFSQQIYGVVYGNVTFHVPSNVPLKEVLWKKQKDKVAE |
| | | LENSEFRAFSSFKNRVYLDTVSGSLTIYNLTSSDEDEYEMESPNITD |
| | | TMKFFLYVLESLPSPTLTCALTNGSIEVQCMIPEHYNSHRGLIMYS |
| | | WDCPMEQCKRNSTSIYFKMENDLPQKIQCTLSNPLFNTTSSIILTTC |
| | | IPSSGHSRHRYALIPIPLAVITTCIVLYMNGILKCDRKPDRTNSN |
| #496 | 72 | (MVAGSDAGRALGVLSVVCLLHCFGFISC)FSQQIYGVVYGNVTFH |
| | | VPSNVPLKEVLWKKQKDKVAELENSEFRAFSSFKNRVYLDTVSGS |
| | | LTIYNLTSSDEDEYEMESPNITDTMKFFLYVLESLGSGDIQMTQSPS |
| | | SLSASVGDRVTITCSASSSVSYMNWYQQTPGKAPKRWIYDTSKLA |
| | | SGVPSRFSGSGSGTDYTFTISSLQPEDIATYYCQQWSSNPFTFGQGT |
| | | KLQITRTSGGGGSGGGGSGGGGSQVQLVQSGGGVVQPGRSLRLSC |
| | | KASGYTFTRYTMHWVRQAPGKGLEWIGYINPSRGYTNYNQKVKD |
| | | RFTISRDNSKNTAFLQMDSLRPEDTGVYFCARYYDDHYCLDYWG |
| | | QGTPVTVSSAGSSGGSGGGGSGGGGSGGGGSSGVIHVTKEVKEVA |
| | | TLSCGHNVSVEELAQTRIYWQKEKKMVLTMMSGDMNIWPEYKN |
| | | RTIFDITNNLSIVILALRPSDEGTYECVVLKYEKDAFKREHLAEVTL |
| | | SVKADFPTPSISDFEIPTSNIRRIICSTSGGFPEPHLSWLENGEELNAI |
| | | NTTVSQDPETELYAVSSKLDFNMTTNHSFMCLIKYGHLRVNQTFN |
| | | WNTTKQEHFPDNLLPSWAITLISVNGIFVICCLTYCFAPRCRERRRN |
| | | ERLRRESVRPV |
| #497 | 73 | (MGVKVLFALICIAVAEA)DIQMTQSPSSLSASVGDRVTITCSASSSV |
| | | SYMNWYQQTPGKAPKRWIYDTSKLASGVPSRFSGSGSGTDYTFTI |
| | | SSLQPEDIATYYCQQWSSNPFTFGQGTKLQITRTSGGGGSGGGGSG |
| | | GGGSQVQLVQSGGGVVQPGRSLRLSCKASGYTFTRYTMHWVRQ |
| | | APGKGLEWIGYINPSRGYTNYNQKVKDRFTISRDNSKNTAFLQMD |
| | | SLRPEDTGVYFCARYYDDHYCLDYWGQGTPVTVSSAGSSGGSGG |
| | | GGSGGGGSGGGGSFSQQIYGVVYGNVTFHVPSNVPLKEVLWKKQ |
| | | KDKVAELENSEFRAFSSFKNRVYLDTVSGSLTIYNLTSSDEDEYEM |
| | | ESPNITDTMKFFLYVLESLPSPTLTCALTNGSIEVQCMIPEHYNSHR |
| | | GLIMYSWDCPMEQCKRNSTSIYFKMENDLPQKIQCTLSNPLFNTTS |
| | | SIILTTCIPSSGHSRHRGSGVIHVTKEVKEVATLSCGHNVSVEELAQ |
| | | TRIYWQKEKKMVLTMMSGDMNIWPEYKNRTIFDITNNLSIVILAL |
| | | RPSDEGTYECVVLKYEKDAFKREHLAEVTLSVKADFPTPSISDFEIP |
| | | TSNIRRIICSTSGGFPEPHLSWLENGEELNAINTTVSQDPETELYAVS |
| | | SKLDFNMTTNHSFMCLIKYGHLRVNQTFNWNTTKQEHFPDNLLPS |
| | | WAITLISVNGIFVICCLTYCFAPRCRERRRNERLRRESVRPV |
| #498 | 33 | (MVAGSDAGRALGVLSVVCLLHCFGFISC)FSQQIYGVVYGNVTFH |
| | | VPSNVPLKEVLWKKQKDKVAELENSEFRAFSSFKNRVYLDTVSGS |
| | | LTIYNLTSSDEDEYEMESPNITDTMKFFLYVLESLGSGDIQMTQSPS |
| | | SLSASVGDRVTITCSASSSVSYMNWYQQTPGKAPKRWIYDTSKLA |
| | | SGVPSRFSGSGSGTDYTFTISSLQPEDIATYYCQQWSSNPFTFGQGT |
| | | KLQITRTSGGGGSGGGGSGGGGSQVQLVQSGGGVVQPGRSLRLSC |
| | | KASGYTFTRYTMHWVRQAPGKGLEWIGYINPSRGYTNYNQKVKD |
| | | RFTISRDNSKNTAFLQMDSLRPEDTGVYFCARYYDDHYCLDYWG |
| | | QGTPVTVSSAGSSGGSGGGGSGGGGSGGGGSSGVIHVTKEVKEVA |
| | | TLSCGHNVSVEELAQTRIYWQKEKKMVLTMMSGDMNIWPEYKN |
| | | RTIFDITNNLSIVILALRPSDEGTYECVVLKYEKDAFKREHLAEVTL |
| | | SVKADFPTPSISDFEIPTSNIRRIICSTSGGFPEPHLSWLENGEELNAI |
| | | NTTVSQDPETELYAVSSKLDFNMTTNHSFMCLIKYGHLRVNQTFN |
| | | WNTTKQEHFPDNLLPSWAITLISVNGIFVICCLTYCFAPRCRERRRN |
| | | ERLRRESVRPV |

FIG. 50A-50J include examples of CD58 and CD80 dual fusion sequences. Some embodiments include a nucleic acid sequence having at least at least 75%, at least 80%, at least 85%, at least 90%, at least 91%, at least 92%, at least 93%, at least 94%, at least 95%, at least 99%, or 100% sequence identity to a nucleic acid sequence in any of FIG. 50A-50J or any of SEQ ID NOs: 215-234, or to a fragment or portion thereof such as may be identified in the figure keys. Some embodiments include a nucleic acid sequence having less than less than 75%, less than 80%, less than 85%, less than 90%, less than 91%, less than 92%, less than 93%, less than 94%, less than 95%, less than 99%, or less than 100% sequence identity to a nucleic acid sequence in any of FIG. 50A-50J or any of SEQ ID NOs: 215-234, or to a fragment or portion thereof such as may be identified in the figure keys. Some embodiments include an amino acid sequence having at least at least 75%, at least 80%, at least 85%, at least 90%, at least 91%, at least 92%, at least 93%, at least 94%, at least 95%, at least 99%, or 100% sequence identity to an amino acid sequence in any of FIG. 50A-50J or any of SEQ ID NOs: 215-234, or to a fragment or portion thereof such as may be identified in the figure keys. Some embodiments include an amino acid sequence having less than less than 75%, less than 80%, less than 85%, less than 90%, less than 91%, less than 92%, less than 93%, less than 94%, less than 95%, less than 99%, or less than 100% sequence identity to an amino acid sequence in any of FIG. 50A-50J or any of SEQ ID NOs: 215-234, or to a fragment or portion thereof such as may be identified in the figure keys.

FIG. 51A-51F include examples of CD58, CD80 and CD3 scFV triple fusion sequences. Some embodiments include a nucleic acid sequence having at least at least 75%, at least 80%, at least 85%, at least 90%, at least 91%, at least 92%, at least 93%, at least 94%, at least 95%, at least 99%, or 100% sequence identity to a nucleic acid sequence in any of FIG. 51A-51F or any of SEQ ID NOs: 235-246, or to a fragment or portion thereof such as may be identified in the figure keys. Some embodiments include a nucleic acid sequence having less than less than 75%, less than 80%, less than 85%, less than 90%, less than 91%, less than 92%, less than 93%, less than 94%, less than 95%, less than 99%, or less than 100% sequence identity to a nucleic acid sequence in any of FIG. 51A-51F or any of SEQ ID NOs: 235-246, or to a fragment or portion thereof such as may be identified in the figure keys. Some embodiments include an amino acid sequence having at least at least 75%, at least 80%, at least 85%, at least 90%, at least 91%, at least 92%, at least 93%, at least 94%, at least 95%, at least 99%, or 100% sequence identity to an amino acid sequence in any of FIG. 51A-51F or any of SEQ ID NOs: 235-246, or to a fragment or portion thereof such as may be identified in the figure keys. Some embodiments include an amino acid sequence having less than less than 75%, less than 80%, less than 85%, less than 90%, less than 91%, less than 92%, less than 93%, less than 94%, less than 95%, less than 99%, or less than 100% sequence identity to an amino acid sequence in any of FIG. 51A-51F or any of SEQ ID NOs: 235-246, or to a fragment or portion thereof such as may be identified in the figure keys.

### Chimeric Antigen Receptors (CARs)

In some embodiments, a particle such as a lentiviral particle described herein is used to transduce a nucleic acid sequence (polynucleotide) encoding one or more chimeric antigen receptor (CARs) into a cell (e.g., a T lymphocyte). In some embodiments, the transduction of the lentiviral particle results in expression of one or more CARs in the transduced cells.

CARs are artificial membrane-bound proteins that direct a T lymphocyte to an antigen and stimulate the T lymphocyte to kill cells displaying the antigen. See, e.g., Eshhar, U.S. Pat. No. 7,741,465. Generally, CARs are genetically engineered receptors comprising an extracellular domain that binds to an antigen, e.g., an antigen on a cell, an optional linker, a transmembrane domain, and an intracellular (cytoplasmic) domain comprising a costimulatory domain and/or a signaling domain that transmits an activation signal to an immune cell. With a CAR, a single receptor can be programmed to both recognize a specific antigen and, when bound to that antigen, activate the immune cell to attack and destroy the cell bearing that antigen. When these antigens exist on tumor cells, an immune cell that expresses the CAR can target and kill the tumor cell. All other conditions being satisfied, when a CAR is expressed on the surface of, e.g., a T lymphocyte, and the extracellular domain of the CAR binds to an antigen, the intracellular signaling domain transmits a signal to the T lymphocyte to activate and/or proliferate, and, if the antigen is present on a cell surface, to kill the cell expressing the antigen. Because T lymphocytes may require two signals, a primary activation signal and a costimulatory signal, in order to maximally activate, CARs can comprise a stimulatory and a costimulatory domain such that binding of the antigen to the extracellular domain results in transmission of both a primary activation signal and a costimulatory signal. Illustrative CARs may be designed in a modular fashion, e.g. as described in (see, e.g., Guedan S, Calderon H, Posey A D, Maus M V, Molecular Therapy—Methods & Clinical Development. 2019; 12: 145-156), incorporated by reference.

In some embodiments, a lentiviral particle disclosed herein comprises a polynucleotide that encodes a CAR comprising an extracellular domain, optionally a hinge domain, a transmembrane domain, and an intracellular signaling domain. In some embodiments, the intracellular signaling domain comprises a costimulatory domain and an activation domain. In some embodiments, the costimulatory and activation domains are a single domain, for example a single intracellular domain that provides both costimulation and activation signals to a cell. In other embodiments, the intracellular signaling domain comprises either a costimulatory domain or an activation domain. In some embodiments, the CAR comprises an extracellular domain, a CD8a hinge, a CD8a transmembrane domain, a 4-1BB costimulatory domain, and a CD3zeta signaling domain. In some embodiments, a lentiviral particle disclosed herein encodes an extracellular domain, a CD28 hinge domain, a CD28 transmembrane domain, a CD28 co-stimulatory domain, and a CD3zeta signaling domain. In some embodiments, a lentiviral particle disclosed herein encodes an extracellular domain, an IgG4 hinge domain, a CD28 transmembrane domain, a 4-1BB co-stimulatory domain, and a CD3zeta signaling domain. In some embodiments, a lentiviral particle disclosed herein encodes a CAR comprising an extracellular domain, a CD8a hinge, a CD28 transmembrane domain, a 4-1BB costimulatory domain, and a CD3zeta signaling domain.

Disclosed herein, in some embodiments, are lentiviral particles, comprising, displayed on the surface of the particle: a fusion molecule comprising: a) a CD58 extracellular domain, or a functional fragment thereof, b) a CD80 or CD86 extracellular domain, or a functional fragment thereof, c) an antigen-binding fragment of an anti-CD3 antibody; and a viral glycoprotein (G protein), wherein the lentiviral particle comprises a polynucleotide encoding a chimeric antigen receptor that specifically binds CD20 (e.g. an anti-CD20 CAR). In some embodiments, the lentiviral particle comprises a polynucleotide encoding a synthetic cytokine gamma chain polypeptide and a synthetic cytokine beta chain polypeptide. In some embodiments, the anti-CD20 CAR comprises a ligand-binding domain comprising an scFv domain, wherein the scFv further comprises a VL comprising the polypeptide sequence of SEQ ID NO: 176 and a VH comprising the polypeptide sequence of SEQ ID NO: 179; or wherein the VL comprises a sequences at least 80%, 85%, 90%, 91%, 92%, 93%, 94%, 95%, 96%, 97%, 98%, or 99% identical to SEQ ID NO: 176 and the VH comprises a sequences at least 80%, 85%, 90%, 91%, 92%, 93%, 94%, 95%, 96%, 97%, 98%, or 99% identical to SEQ ID NO: 179. In some embodiments, the scFv of the anti-CD20 CAR comprises a spacer comprising a polypeptide sequence at least 80%, 85%, 90%, 91%, 92%, 93%, 94%, 95%, 96%, 97%, 98%, or 99% identical to the polypeptide sequence of SEQ ID NO: 278. In some embodiments, the scFv spacer comprises the polypeptide sequence of SEQ ID NO: 278. In some embodiments, the scFv of the anti-CD20 CAR comprises a polypeptide sequence at least 80%, 85%, 90%, 91%, 92%, 93%, 94%, 95%, 96%, 97%, 98%, or 99% identical to the polypeptide sequence of SEQ ID NO: 174. In some embodiments, the scFv comprises the polypeptide sequence of SEQ ID NO: 174. In some embodiments, the scFv is encoded by a polynucleotide sequence at least 80%, 85%, 90%, 91%, 92%, 93%, 94%, 95%, 96%, 97%, 98%, or 99% identical to the polynucleotide sequence of SEQ ID NO: 279. In some embodiments, the scFv is encoded by the polynucleotide sequence of SEQ ID NO: 279. In some embodiments, the anti-CD20 CAR comprises a CD8 hinge domain. In some embodiments, the CD8 hinge domain is encoded by a polynucleotide sequence at least 80%, 85%, 90%, 91%, 92%, 93%, 94%, 95%, 96%, 97%, 98%, or 99% identical to the polynucleotide sequence of SEQ ID NO: 182. In some embodiments, the CD8 hinge domain is encoded by the polynucleotide sequence of SEQ ID NO: 182. In some embodiments, the CD8 hinge domain comprises a polypeptide sequence at least 80%, 85%, 90%, 91%, 92%, 93%, 94%, 95%, 96%, 97%, 98%, or 99% identical to the polypeptide sequence of SEQ ID NO: 280. In some embodiments, the CD8 hinge domain comprises the polypeptide sequence of SEQ ID NO: 280. In some embodiments, the anti-CD20 CAR comprises a CD8 transmembrane domain. In some embodiments, the CD8 transmembrane domain is encoded by a polynucleotide sequence at least 80%, 85%, 90%, 91%, 92%, 93%, 94%, 95%, 96%, 97%, 98%, or 99% identical to the polynucleotide sequence of SEQ ID NO: 183. In some embodiments, the CD8 transmembrane domain is encoded by the polynucleotide sequence of SEQ ID NO: 183. In some embodiments, the CD8 transmembrane domain comprises a polypeptide sequence at least 80%, 85%, 90%, 91%, 92%, 93%, 94%, 95%, 96%, 97%, 98%, or 99% identical to the polypeptide sequence of SEQ ID NO: 281. In some embodiments, the CD8 transmembrane domain comprises the polypeptide sequence of SEQ ID NO: 281. In some embodiments, the anti-CD20 CAR comprises a 4-1BB endodomain. In some embodiments, the 4-1BB endodomain is encoded by a polynucleotide sequence at least 80%, 85%, 90%, 91%, 92%, 93%, 94%, 95%, 96%, 97%, 98%, or 99% identical to the polynucleotide sequence of SEQ ID NO: 200. In some embodiments, the 4-1BB endodomain is encoded by the polynucleotide sequence of SEQ ID NO: 200. In some embodiments, the 4-1BB endodomain comprises a polypeptide sequence at least 80%, 85%, 90%, 91%, 92%, 93%, 94%, 95%, 96%, 97%, 98%, or 99% identical to the polypeptide sequence of SEQ ID NO: 201. In some embodiments, the 4-1BB endodomain comprises the polypeptide sequence of SEQ ID NO: 201. In some embodiments, the anti-CD20 CAR comprises a CD3ζ endodomain. In some embodiments, the CD3ζ endodomain is encoded by a polynucleotide sequence at least 80%, 85%, 90%, 91%, 92%, 93%, 94%, 95%, 96%, 97%, 98%, or 99% identical to the polynucleotide sequence of SEQ ID NO: 202. In some embodiments, the CD3ζ endodomain is encoded by the polynucleotide sequence of SEQ ID NO: 202. In some embodiments, the CD3ζ endodomain comprises a polypeptide sequence at least 80%, 85%, 90%, 91%, 92%, 93%, 94%, 95%, 96%, 97%, 98%, or 99% identical to the polypeptide sequence of SEQ ID NO: 203. In some embodiments, the CD3ζ endodomain comprises the polypeptide sequence of SEQ ID NO: 203. In some embodiments, the polynucleotide encoding the anti-CD20 CAR comprises a polynucleotide sequence at least 80%, 85%, 90%, 91%, 92%, 93%, 94%, 95%, 96%, 97%, 98%, or 99% identical to the polynucleotide sequence of SEQ ID NO: 186. In some embodiments, the polynucleotide encoding the anti-CD20 CAR comprises the polynucleotide sequence of SEQ ID NO: 186. In some embodiments, the anti-CD20 CAR comprises a polypeptide sequence at least 80%, 85%, 90%, 91%, 92%, 93%, 94%, 95%, 96%, 97%, 98%, or 99% identical to the polypeptide sequence of SEQ ID NO: 209. In some embodiments, the anti-CD20 CAR comprises a polypeptide sequence at least 80%, 85%, 90%, 91%, 92%, 93%, 94%, 95%, 96%, 97%, 98%, or 99% identical to a polypeptide sequence of SEQ ID NO: 209 that excludes a FLAG-tag sequence. In some embodiments, the anti-CD20 CAR comprises the polypeptide sequence of SEQ ID NO: 209. In some embodiments, the anti-CD20 CAR comprises a polypeptide sequence of SEQ ID NO: 209 that excludes a FLAG-tag sequence.

### CAR Intracellular Domain

In some embodiments, the intracellular domain of the CAR is or comprises an intracellular domain or motif of a protein that is expressed on the surface of T lymphocytes and triggers activation and/or proliferation of said T lymphocytes. In some embodiments, such a domain or motif is able to transmit a signal for activation of a T lymphocyte in response to antigen binding to the extracellular portion of the CAR. In some embodiments, this domain or motif comprises, or is, an ITAM (immunoreceptor tyrosine-based activation motif). ITAM-containing polypeptides suitable for CARs include, for example, the zeta CD3 chain (CD3) or ITAM-containing portions thereof. In some embodiments, the intracellular domain is a CD3ζ intracellular signaling domain. In some embodiments, the intracellular domain is from a lymphocyte receptor chain, a TCR/CD3 complex protein, an Fc receptor subunit or an IL-2 receptor subunit. In some embodiments, the intracellular signaling domain of CAR may be the signaling domains of for example CD3, CD3c, CD22, CD79a, CD66d or CD39. “Intracellular signaling domain” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain.

In some embodiments, the intracellular domain of the CAR is the zeta CD3 chain (CD3zeta).

In some embodiments, the lentiviral particle comprises a polypeptide comprising a CAR whose intracellular domain comprises a CD3zeta domain that shares at least 75%, at least 80%, at least 85%, at least 90%, at least 95%, at least 99%, or 100% identity to SEQ ID NO: 82.

(SEQ ID NO: 82)
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR

In some embodiments, the lentiviral particle comprises a nucleic acid encoding the intracellular domain of a CAR comprising a CD3zeta domain that shares at least 75%, at least 80%, at least 85%, at least 90%, at least 95%, at least 99%, or 100% identity to SEQ ID NO: 83.

(SEQ ID NO: 83)
CGCGTGAAGTTCAGCCGGTCCGCCGATGCCCCTGCCTACCAGCAG
GGCCAGAACCAGCTGTATAACGAGCTGAATCTGGGCCGGAGAGAG
GAGTACGACGTGCTGGATAAGAGGAGGGGAAGGGACCCAGAGATG
GGAGGCAAGCCTCGGAGAAAGAACCCACAGGAGGGCCTGTACAAT
GAGCTGCAGAAGGACAAGATGGCCGAGGCCTATTCTGAGATCGGC
ATGAAGGGAGAGAGGCGCCGGGGCAAGGGACACGATGGCCTGTAC
CAGGGCCTGAGCACCGCCACAAAGGACACATATGATGCCCTGCAC
ATGCAGGCCCTGCCACCTAGG

In some embodiments, the CAR additionally comprises one or more co-stimulatory domains or motifs, e.g., as part of the intracellular domain of the polypeptide. Co-stimulatory molecules may include cell surface molecules other than antigen receptors or Fc receptors that provide a second signal useful for efficient activation and function of T lymphocytes upon binding to antigen. The one or more co-stimulatory domains or motifs can, for example, be, or comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell costimulatory (ICOS) polypeptide sequence, or other costimulatory domain or motif, or any combination thereof. In some embodiments, the one or more co-stimulatory domains are selected from the group consisting of intracellular domains of 4-1BB, CD2, CD7, CD27, CD28, CD30, CD40, CD54 (ICAM), CD83, CD134 (OX40), CD150 (SLAMF1), CD152 (CTLA4), CD223 (LAG3), CD270 (HVEM), CD278 (ICOS), DAP10, LAT, NKD2C SLP76, TRIM, and ZAP70.

In some embodiments, the co-stimulatory domain is an intracellular domain of 4-1BB, CD28, or OX40. Illustrative CAR constructs comprising a CD28 signaling domain are disclosed in U.S. Pat. No. 7,446,190, incorporated by reference. Illustrative CAR constructs comprising a 4-1BB signaling domain are disclosed in U.S. Pat. Nos. 9,856,322 and 8,399,964, each incorporated by reference.

In some embodiments, the lentiviral particle encodes a CAR comprising an IgG4 linker operatively linked to a CD28 transmembrane domain operatively linked to a 4-1BB co-stimulatory domain operatively linked to a CD3zeta signaling domain.

In some embodiments, the lentiviral particle encodes a CAR comprising an IgG4 linker operatively linked to a CD8a transmembrane domain operatively linked to a 4-1BB co-stimulatory domain operatively linked to a CD3zeta signaling domain.

In some embodiments, the lentiviral particle encodes a CAR comprising an IgG4 linker operatively linked to a CD8a transmembrane domain operatively linked to a CD28 co-stimulatory domain operatively linked to a CD3zeta signaling domain.

In some embodiments, the lentiviral particle encodes a CAR comprising a CD8a linker operatively linked to a CD8a transmembrane domain operatively linked to a 4-1BB co-stimulatory domain operatively linked to a CD3zeta signaling domain.

In some embodiments, the lentiviral particle encodes a CAR comprising a CD8a linker operatively linked to a CD28 transmembrane domain operatively linked to a 4-1BB co-stimulatory domain operatively linked to a CD3zeta signaling domain.

In some embodiments, the lentiviral particle encodes a CAR comprising a CD28 linker operatively linked to a CD28 transmembrane domain operatively linked to a CD28 co-stimulatory domain operatively linked to a CD3zeta signaling domain.

In some embodiments, the lentiviral particle comprises a polypeptide comprising a CAR whose intracellular domain comprises a co-stimulatory 4-1BB polypeptide sequence that shares at least 75%, at least 80%, at least 85%, at least 90%, at least 95%, at least 99%, or 100% identity to SEQ ID NO: 84.

(SEQ ID NO: 84)
KRGRKKLLYIFKQPFMRPVQTTQEEDGCSCRFPEEEEGGCEL

In some embodiments, the lentiviral particle comprises a nucleic acid encoding the intracellular domain of a CAR comprising a co-stimulatory 4-1BB sequence that shares at least 75%, at least 80%, at least 85%, at least 90%, at least 95%, at least 99%, or 100% identity to SEQ ID NO: 85.

(SEQ ID NO: 85)
CGCGTGAAGTTCAGCCGGTCCGCCGATGCCCCTGCCTACCAGCAG
GGCCAGAACCAGCTGTATAACGAGCTGAATCTGGGCCGGAGAGAG
GAGTACGACGTGCTGGATAAGAGGAGGGGAAGGGACCCAGAGATG
GGAGGCAAGCCTCGGAGAAAGAACCCACAGGAGGGCCTGTACAAT
GAGCTGCAGAAGGACAAGATGGCCGAGGCCTATTCTGAGATCGGC
ATGAAGGGAGAGAGGCGCCGGGGCAAGGGACACGATGGCCTGTAC
CAGGGCCTGAGCACCGCCACAAAGGACACATATGATGCCCTGCAC
ATGCAGGCCCTGCCACCTAGG

In some embodiments, the lentiviral particle comprises a polypeptide comprising a CAR whose intracellular domain comprises an IgG4 linker operatively linked to a CD28 transmembrane domain operatively linked to a co-stimulatory 4-1BB polypeptide operatively linked to a CD3zeta domain that shares at least 75%, at least 80%, at least 85%, at least 90%, at least 95%, at least 99%, or 100% identity to SEQ ID NO: 86.

(SEQ ID NO: 86)
ESKYGPPCPPCPMFWVLVVVGGVLACYSLLVTVAFIIFWVKRGRK
KLLYIFKQPFMRPVQTTQEEDGCSCRFPEEEEGGCELRVKFSRSA
DAPAYQQGQNQLYNELNLGRREEYDVLDKRRGRDPEMGGKPRRKN
PQEGLYNELQKDKMAEAYSEIGMKGERRRGKGHDGLYQGLSTATK
DTYDALHMQALPPR

In some embodiments, the lentiviral particle comprises a nucleic acid encoding the intracellular domain of a CAR comprising an IgG4 linker operatively linked to a CD28 transmembrane domain operatively linked to a co-stimulatory 4-1BB polypeptide operatively linked to a CD3zeta domain that shares at least 75%, at least 80%, at least 85%, at least 90%, at least 95%, at least 99%, or 100% identity to SEQ ID NO: 87.

(SEQ ID NO: 87)
GAGTCTAAGTATGGCCCTCCATGCCCCCCTTGTCCTATGTTCTGG
GTGCTGGTGGTGGTGGGAGGCGTGCTGGCCTGTTACTCCCTGCTG
GTGACCGTGGCCTTTATCATCTTCTGGGTGAAGCGCGGCCGGAAG
AAGCTGCTGTATATCTTTAAGCAGCCCTTCATGAGACCTGTGCAG
ACCACACAGGAGGAGGACGGCTGCAGCTGTAGGTTTCCAGAGGAG
GAGGAGGGAGGATGCGAGCTGCGCGTGAAGTTCTCTCGGAGCGCC
GATGCCCCTGCCTACCAGCAGGGACAGAACCAGCTGTATAACGAG
CTGAATCTGGGCCGGAGAGAGGAGTACGACGTGCTGGATAAGAGG
AGGGGAAGAGACCCAGAGATGGGAGGCAAGCCTCGGAGAAAGAAC
CCACAGGAGGGCCTGTACAATGAGCTGCAGAAGGACAAGATGGCC
GAGGCCTATTCCGAGATCGGCATGAAGGGAGAGAGGCGCCGGGGC
AAGGGACACGATGGCCTGTACCAGGGCCTGAGCACCGCCACAAAG
GACACCTATGATGCCCTGCACATGCAGGCCCTGCCACCCAGG

In some embodiments, the lentiviral particle comprises a polypeptide comprising a CAR whose intracellular domain comprises an IgG4 linker operatively linked to a CD28 transmembrane domain operatively linked to a co-stimulatory 4-1BB polypeptide operatively linked to a CD3zeta domain that shares at least 75%, at least 80%, at least 85%, at least 90%, at least 95%, at least 99%, or 100% identity to SEQ ID NO: 88.

(SEQ ID NO: 88)
ESKYGPPCPPCPMFWVLVVVGGVLACYSLLVTVAFIIFWVKRGRK
KLLYIFKQPFMRPVQTTQEEDGCSCRFPEEEEGGCELRVKFSRSA
DAPAYQQGQNQLYNELNLGRREEYDVLDKRRGRDPEMGGKPRRKN
PQEGLYNELQKDKMAEAYSEIGMKGERRRGKGHDGLYQGLSTATK
DTYDALHMQALPPRGSGATNFSLLKQAGDVEENPGP

In some embodiments, the lentiviral particle comprises a nucleic acid encoding the intracellular domain of a CAR comprising an IgG4 linker operatively linked to a CD28 transmembrane domain operatively linked to a co-stimulatory 4-1BB polypeptide operatively linked to a CD3zeta domain that shares at least 75%, at least 80%, at least 85%, at least 90%, at least 95%, at least 99%, or 100% identity to SEQ ID NO: 89.

(SEQ ID NO: 89)
GAGTCCAAGTACGGCCCACCCTGCCCTCCATGTCCCATGTTTTGG
GTGCTGGTGGTGGTGGGAGGCGTGCTGGCCTGTTATTCCCTGCTG
GTGACCGTGGCCTTCATCATCTTTTGGGTGAAGCGCGGCCGGAAG
AAGCTGCTGTACATCTTCAAGCAGCCCTTCATGAGACCCGTGCAG
ACCACACAGGAGGAGGACGGCTGCAGCTGTAGGTTCCCAGAGGAG
GAGGAGGGAGGATGCGAGCTGAGGGTGAAGTTTTCCCGGTCTGCC
GATGCCCCTGCCTATCAGCAGGGCCAGAATCAGCTGTACAACGAG
CTGAATCTGGGCAGGCGCGAGGAGTACGACGTGCTGGATAAGAGG
AGAGGAAGGGACCCTGAGATGGGAGGCAAGCCAAGGCGCAAGAAC
CCTCAGGAGGGCCTGTATAATGAGCTGCAGAAGGACAAGATGGCC
GAGGCCTACTCCGAGATCGGCATGAAGGGAGAGCGGAGAAGGGGC
AAGGGACACGATGGCCTGTATCAGGGCCTGAGCACCGCCACAAAG
GACACCTACGATGCACTGCACATGCAGGCCCTGCCACCTAGAGGA
TCTGGAGCCACAAACTTCAGCCTGCTGAAGCAGGCCGGCGATGTG
GAGGAGAATCCTGGACCA

In some embodiments, the intracellular domain can be further modified to encode a detectable, for example, a fluorescent, protein (e.g., green fluorescent protein) or any variants thereof.

### CAR Transmembrane Region

The transmembrane region can be any transmembrane region that can be incorporated into a functional CAR, e.g., a transmembrane region from a CD28, CD4, or a CD8 molecule.

In some embodiments, the transmembrane domain of CAR may be the transmembrane domain of CD8, an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1 BB (CD137), 4-1 BBL, GITR, CD40, BAFFR, HVEM (LIGHTR), SLAMF7, NKp80 (KLRFI), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAMI, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, NKG2D “in reverse orientation”, and/or NKG2C. In some embodiments, the transmembrane domain of the CAR may be the transmembrane domain of CD28. In some embodiments, the transmembrane domain of a CAR may be the transmembrane domain of CD8, for example, CD8α.

### CAR Linker Region

The optional linker or hinge of CAR positioned between the extracellular domain and the transmembrane domain may be a polypeptide of about 2 to over 100 amino acids in length. The linker can include or be composed of flexible residues such as glycine and serine so that the adjacent protein domains are free to move relative to one another. Longer linkers may be used, e.g., when it is desirable to ensure that two adjacent domains do not sterically interfere with one another. Longer linkers may also be advantageous when the target antigen is closer to the cell surface.

In some embodiments, the linker is from a hinge region or portion of the hinge region of any immunoglobulin or other transmembrane protein. For example, the hinge region may be from IgG1, IgG2, IgG3, IgG4, PD1, CD8, or CD28, or a portion thereof. In some embodiments, the linker is from a portion of an immunoglobulin, for example IgG4. In some embodiments, the linker is a portion of an immunoglobulin, for example IgG1. In some embodiments, the linker is a portion of the extracellular domain of CD28. In other embodiments, the linker is a portion of the extracellular domain of CD8. In other embodiments, the linker is a portion of the extracellular domain of PD1.

In some embodiments, the linker is an IgG4 linker operably linked to a CD28 transmembrane domain that shares at least 75%, at least 80%, at least 85%, at least 90%, at least 95%, at least 99%, or 100% identity to SEQ ID NO: 90.

(SEQ ID NO: 90)
ESKYGPPCPPCPMFWVLVVVGGVLACYSLLVTVAFIIFWV

In some embodiments, the linker is an IgG4 linker operably linked to a CD28 transmembrane domain that shares at least 75%, at least 80%, at least 85%, at least 90%, at least 95%, at least 99%, or 100% identity to SEQ ID NO: 91.

(SEQ ID NO: 91)
GAGTCTAAGTATGGCCCACCCTGCCCTCCATGTCCAATGTTCTGG
GTGCTGGTGGTGGTGGGAGGCGTGCTGGCCTGTTACTCCCTGCTG
GTGACCGTGGCCTTTATCATCTTCTGGGTG

### CAR Extracellular Domain

In some embodiments, the nucleic acid transduced into cells using the methods described herein comprises a sequence that encodes a polypeptide, wherein the extracellular domain of the polypeptide binds to an antigen of interest. In some embodiments, the extracellular domain comprises a receptor, or a portion of a receptor, that binds to said antigen. In some embodiments, the extracellular domain comprises, or is, an antibody or an antigen-binding portion thereof. In some embodiments, the extracellular domain comprises, or is, a single-chain Fv domain. The single-chain Fv domain can comprise, for example, a VL linked to VH by a flexible linker, wherein said VL and VH are from an antibody that binds said antigen.

In some embodiments, the extracellular domain of CAR may contain any polypeptide that binds the desired antigen (e.g. prostate neoantigen or antigen expressed on a tumor of interest). The extracellular domain may comprise a scFv, a portion of an antibody or an alternative scaffold. CARs may also be engineered to bind two or more desired antigens that may be arranged in tandem and separated by linker sequences. For example, one or more domain antibodies, scFvs, llama VHH antibodies or other VH only antibody fragments may be organized in tandem via a linker to provide bispecificity or multispecificity to the CAR.

The antigen to which the extracellular domain of the polypeptide binds can be any antigen of interest, e.g., can be an antigen on a tumor cell. The tumor cell may be, e.g., a cell in a solid tumor, or a cell of a blood cancer. The antigen can be any antigen that is expressed on a cell of any tumor or cancer type, e.g., cells of a lymphoma, a lung cancer, a breast cancer, a prostate cancer, an adrenocortical carcinoma, a thyroid carcinoma, a nasopharyngeal carcinoma, a melanoma, e.g., a malignant melanoma, a skin carcinoma, a colorectal carcinoma, a desmoid tumor, a desmoplastic small round cell tumor, an endocrine tumor, an Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipoma, or the like. In some embodiments, said lymphoma can be chronic lymphocytic leukemia (small lymphocytic lymphoma), B-cell prolymphocytic leukemia, lymphoplasmacytic lymphoma, Waldenstrom macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, or a non-Hodgkin lymphoma. In some embodiments, in which the cancer is chronic lymphocytic leukemia (CLL), the B cells of the CLL have a normal karyotype. In some embodiments, in which the cancer is chronic lymphocytic leukemia (CLL), the B cells of the CLL carry a 17p deletion, an 11 q deletion, a 12q trisomy, a 13q deletion or a p53 deletion.

In some embodiments, the antigen is expressed on a B-cell malignancy cell, relapsed/refractory CD19-expressing malignancy cell, diffuse large B-cell lymphoma (DLBCL) cell, Burkitt's type large B-cell lymphoma (B-LBL) cell, follicular lymphoma (FL) cell, chronic lymphocytic leukemia (CLL) cell, acute lymphocytic leukemia (ALL) cell, mantle cell lymphoma (MCL) cell, hematological malignancy cell, colon cancer cell, lung cancer cell, liver cancer cell, breast cancer cell, renal cancer cell, prostate cancer cell, ovarian cancer cell, skin cancer cell, melanoma cell, bone cancer cell, brain cancer cell, squamous cell carcinoma cell, leukemia cell, myeloma cell, B cell lymphoma cell, kidney cancer cell, uterine cancer cell, adenocarcinoma cell, pancreatic cancer cell, chronic myelogenous leukemia cell, glioblastoma cell, neuroblastoma cell, medulloblastoma cell, or a sarcoma cell.

In some embodiments, the antigen is a tumor-associated antigen (TAA) or a tumor-specific antigen (TSA). In some embodiments, without limitation, the tumor-associated antigen or tumor-specific antigen is CD20. In some embodiments, the CAR comprises binding domains that target two or more antigens as disclosed herein, in any combination. For example: CD19 and CD20 or CD20 and CD22. In some embodiments, the CAR comprises binding domains that target two or more antigens on the same target protein, for example two epitopes in CD20.

In some embodiments, the lentiviral particle comprises a polypeptide comprising a CAR whose extracellular domain comprises a signal peptide that shares at least 75%, at least 80%, at least 85%, at least 90%, at least 95%, at least 99%, or 100% identity to SEQ ID NO: 92.

(SEQ ID NO: 92)
MLLLVTSLLLCELPHPAFLLIP

In some embodiments, the viral particle comprises a nucleic acid encoding the extracellular domain of a CAR comprising a αCD20 scFv that shares at least 75%, at least 80%, at least 85%, at least 90%, at least 95%, at least 99%, or 100% identity to SEQ ID NO: 174. In some embodiments, the viral particle comprises a nucleic acid encoding the extracellular domain of a CAR comprising a αCD20 scFv and comprises an amino acid sequence that shares at least 75%, at least 80%, at least 85%, at least 90%, at least 95%, at least 99%, or 100% identity to SEQ ID NO: 210. In some embodiments, the αCD20 scFv VL comprises an amino acid sequence at least 80%, 85%, 90%, 95%, 96%, 97%, 98%, 99% or 100% identical to the amino acid sequence of SEQ ID NO: 176. In some embodiments, the αCD20 scFv VL comprises a nucleic acid sequence at least 80%, 85%, 90%, 95%, 96%, 97%, 98%, 99% or 100% identical to the amino acid sequence of SEQ ID NO: 175. In some embodiments, the αCD20 scFv linker comprises a nucleic acid sequence at least 80%, 85%, 90%, 95%, 96%, 97%, 98%, 99% or 100% identical to the amino acid sequence of SEQ ID NO: 177. In some embodiments, the αCD20 scFv VH comprises an amino acid sequence at least 80%, 85%, 90%, 95%, 96%, 97%, 98%, 99% or 100% identical to the amino acid sequence of SEQ ID NO: 179. In some embodiments, the αCD20 scFv VH comprises a nucleic acid sequence at least 80%, 85%, 90%, 95%, 96%, 97%, 98%, 99% or 100% identical to the amino acid sequence of SEQ ID NO: 178.

In some embodiments, the viral particle comprises a nucleic acid encoding the extracellular domain of a CAR comprising a FLAG-tag that shares at least 75%, at least 80%, at least 85%, at least 90%, at least 95%, at least 99%, or 100% identity to SEQ ID NO: 180. Some embodiments exclude a nucleic acid sequence encoding a FLAG-tag, or exclude a nucleic acid sequence encoding a FLAG-tag that shares at least 75%, at least 80%, at least 85%, at least 90%, at least 95%, at least 99%, or 100% identity to SEQ ID NO: 21 LIn some embodiments, the viral particle comprises a nucleic acid encoding the extracellular domain of a CAR comprising a FLAG-tag and the encoded amino acid sequence that shares at least 75%, at least 80%, at least 85%, at least 90%, at least 95%, at least 99%, or 100% identity to SEQ ID NO: 211. Some embodiments exclude a FLAG-tag, or exclude a FLAG-tag that shares at least 75%, at least 80%, at least 85%, at least 90%, at least 95%, at least 99%, or 100% identity to SEQ ID NO: 211.

In some embodiments, the viral particle disclosed herein comprises a nucleic acid encoding a CAR comprising a CD8a hinge and transmembrane domain that shares at least 75%, at least 80%, at least 85%, at least 90%, at least 95%, at least 99%, or 100% identity to SEQ ID NO: 181. In some embodiments, the viral particle disclosed herein comprises a nucleic acid encoding a CAR comprising a CD8a hinge and transmembrane domain and the encoded amino acid sequence shares at least 75%, at least 80%, at least 85%, at least 90%, at least 95%, at least 99%, or 100% identity to SEQ ID NO: 212. In some embodiments, the viral particle disclosed herein comprises a nucleic acid encoding a CAR comprising a CD8a hinge domain that shares at least 75%, at least 80%, at least 85%, at least 90%, at least 95%, at least 99%, or 100% identity to SEQ ID NO: 182. In some embodiments, the viral particle disclosed herein comprises a nucleic acid encoding a CAR comprising a CD8a transmembrane domain that shares at least 75%, at least 80%, at least 85%, at least 90%, at least 95%, at least 99%, or 100% identity to SEQ ID NO: 183.

In some embodiments, the viral particle disclosed herein comprises a nucleic acid encoding a CAR comprising a 4-1BB co-stimulatory domain that shares at least 75%, at least 80%, at least 85%, at least 90%, at least 95%, at least 99%, or 100% identity to SEQ ID NO: 184. In some embodiments, the viral particle disclosed herein comprises a nucleic acid encoding a CAR comprising a 4-1BB co-stimulatory domain and the encoded amino acid sequence shares at least 75%, at least 80%, at least 85%, at least 90%, at least 95%, at least 99%, or 100% identity to SEQ ID NO: 213. In some embodiments, the viral particle disclosed herein comprises a nucleic acid encoding a CAR comprising a CD3zeta signaling domain that shares at least 75%, at least 80%, at least 85%, at least 90%, at least 95%, at least 99%, or 100% identity to SEQ ID NO: 185. In some embodiments, the viral particle disclosed herein comprises a nucleic acid encoding a CAR comprising a CD3zeta signaling domain and the encoded amino acid sequence shares at least 75%, at least 80%, at least 85%, at least 90%, at least 95%, at least 99%, or 100% identity to SEQ ID NO: 214.

In some embodiments, the viral particle disclosed herein comprises a nucleic acid encoding an anti-CD20 CAR comprising a FLAG-tag that shares at least 75%, at least 80%, at least 85%, at least 90%, at least 95%, at least 99%, or 100% identity to SEQ ID NO: 186. In some embodiments, the viral particle disclosed herein comprises a nucleic acid encoding an anti-CD20 CAR comprising a FLAG-tag and the encoded amino acid sequence shares at least 75%, at least 80%, at least 85%, at least 90%, at least 95%, at least 99%, or 100% identity to SEQ ID NO: 209.

In some embodiments, the lentiviral particle disclosed herein comprises a nucleic acid encoding a CAR comprising a modified IgG4 hinge domain that shares at least 75%, at least 80%, at least 85%, at least 90%, at least 95%, at least 99%, or 100% identity to SEQ ID NO: 187, 188, or 189. In some embodiments, the lentiviral particle disclosed herein comprises a nucleic acid encoding a CAR comprising a PD1 hinge domain comprising an amino acid sequence that shares at least 75%, at least 80%, at least 85%, at least 90%, at least 95%, at least 99%, or 100% identity to SEQ ID NO: 190. In some embodiments, the lentiviral particle disclosed herein comprises a nucleic acid encoding a CAR comprising an IgG1 hinge domain that shares at least 75%, at least 80%, at least 85%, at least 90%, at least 95%, at least 99%, or 100% identity to SEQ ID NO: 191. In some embodiments, the lentiviral particle disclosed herein comprises a nucleic acid encoding a CAR comprising a CD8 hinge domain comprising an amino acid sequence that shares at least 75%, at least 80%, at least 85%, at least 90%, at least 95%, at least 99%, or 100% identity to SEQ ID NO: 192. In some embodiments, the lentiviral particle disclosed herein comprises a nucleic acid encoding a CAR comprising a CD28 hinge domain comprising an amino acid sequence that shares at least 75%, at least 80%, at least 85%, at least 90%, at least 95%, at least 99%, or 100% identity to SEQ ID NO: 193.

In some embodiments, the lentiviral particle disclosed herein comprises a nucleic acid encoding a CAR comprising a CD8 hinge domain comprising a nucleic acid sequence that shares at least 75%, at least 80%, at least 85%, at least 90%, at least 95%, at least 99%, or 100% identity to SEQ ID NO: 196. In some embodiments, the lentiviral particle disclosed herein comprises a nucleic acid encoding a CAR comprising a CD8 hinge domain comprising an amino acid sequence that shares at least 75%, at least 80%, at least 85%, at least 90%, at least 95%, at least 99%, or 100% identity to SEQ ID NO: 197.

In some embodiments, the lentiviral particle disclosed herein comprises a nucleic acid encoding a CAR comprising a CD28 transmembrane domain comprising a nucleic acid sequence that shares at least 75%, at least 80%, at least 85%, at least 90%, at least 95%, at least 99%, or 100% identity to SEQ ID NO: 198. In some embodiments, the lentiviral particle disclosed herein comprises a nucleic acid encoding a CAR comprising a CD28 transmembrane domain comprising an amino acid sequence that shares at least 75%, at least 80%, at least 85%, at least 90%, at least 95%, at least 99%, or 100% identity to SEQ ID NO: 199.

In some embodiments, the lentiviral particle disclosed herein encodes a CAR comprising a 4-1BB co-stimulatory domain comprising a nucleic acid sequence that shares at least 75%, at least 80%, at least 85%, at least 90%, at least 95%, at least 99%, or 100% identity to SEQ ID NO: 200. In some embodiments, the lentiviral particle disclosed herein encodes a CAR comprising a 4-1BB co-stimulatory domain comprising an amino acid sequence that shares at least 75%, at least 80%, at least 85%, at least 90%, at least 95%, at least 99%, or 100% identity to SEQ ID NO: 201. In some embodiments, the lentiviral particle disclosed herein encodes a CAR comprising a CD3zeta signaling domain comprising a nucleic acid sequence that shares at least 75%, at least 80%, at least 85%, at least 90%, at least 95%, at least 99%, or 100% identity to SEQ ID NO: 202. In some embodiments, the lentiviral particle disclosed herein encodes a CAR comprising a CD3zeta signaling domain comprising an amino acid sequence that shares at least 75%, at least 80%, at least 85%, at least 90%, at least 95%, at least 99%, or 100% identity to SEQ ID NO: 203.

In some embodiments, the lentiviral particle comprises a nucleic acid encoding an anti-CD20 CAR comprising a nucleic acid sequence that shares at least 75%, at least 80%, at least 85%, at least 90%, at least 95%, at least 99%, or 100% identity to SEQ ID NO: 186.

TABLE 4B

| SEQ ID | | |
|---|---|---|
| NO: | SEQUENCE | Description |
| 174 | DIVLTQSPAILSASPGEKVTMTCRASSSVNYMDWYQKKPGSSPKPWIY | αCD20 CAR |
| | ATSNLASGVPARFSGSGSGTSYSLTISRVEAEDAATYYCQQWSFNPPTF | scFv |
| | GGGTKLEIKGSTSGGGSGGGSGGGGSSEVQLQQSGAELVKPGASVKM | polypeptide |
| | SCKASGYTFTSYNMHWVKQTPGQGLEWIGAIYPGNGDTSYNQKFKGK | |
| | ATLTADKSSSTAYMQLSSLTSEDSADYYCARSNYYGSSYWFFDVWGA | |
| | GTTVTVSS | |
| 279 | GATATCGTGCTGACCCAGTCCCCCGCCATCCTGTCCGCCTCTCCTGG | αCD20 CAR |
| | AGAGAAGGTGACCATGACATGTCGGGCCAGCTCCTCTGTGAACTAC | scFv |
| | ATGGACTGGTATCAGAAGAAGCCTGGCAGCTCCCCCAAGCCTTGGA | polynucleotide |
| | TCTACGCCACCTCCAATCTGGCCTCTGGAGTGCCAGCAAGATTCAG | |
| | CGGATCCGGATCTGGCACAAGCTATTCCCTGACCATCTCCAGGGTG | |
| | GAGGCAGAGGATGCAGCAACATACTATTGCCAGCAGTGGTCTTTCA | |
| | ACCCCCCTACATTTGGCGGCGGCACCAAGCTGGAGATCAAGGGCTC | |
| | TACCAGCGGAGGAGGAAGCGGAGGAGGATCCGGAGGCGGCGGCTC | |
| | TAGCGAGGTGCAGCTGCAGCAGTCCGGAGCAGAGCTGGTGAAGCC | |
| | TGGAGCCTCTGTGAAGATGAGCTGTAAGGCCTCCGGCTACACCTTC | |
| | ACATCTTATAATATGCACTGGGTGAAGCAGACACCAGGACAGGGA | |
| | CTGGAGTGGATCGGAGCAATCTACCCTGGCAACGGCGACACCAGCT | |
| | ATAATCAGAAGTTTAAGGGCAAGGCCACCCTGACAGCCGATAAGTC | |
| | CTCTAGCACAGCCTACATGCAGCTGTCCTCTCTGACCAGCGAGGAC | |
| | TCCGCCGATTACTATTGCGCCCGGTCCAACTACTATGGCAGCTCCTA | |
| | TTGGTTCTTTGACGTGTGGGGAGCAGGAACAACCGTGACCGTGTCT | |
| | AGC | |
| 175 | GATATCGTGCTGACCCAGTCCCCCGCCATCCTGTCCGCCTCTCCTGG | αCD20 CAR |
| | AGAGAAGGTGACCATGACATGTCGGGCCAGCTCCTCTGTGAACTAC | scFv VL |
| | ATGGACTGGTATCAGAAGAAGCCTGGCAGCTCCCCCAAGCCTTGGA | polynucleotide |
| | TCTACGCCACCTCCAATCTGGCCTCTGGAGTGCCAGCAAGATTCAG | |
| | CGGATCCGGATCTGGCACAAGCTATTCCCTGACCATCTCCAGGGTG | |
| | GAGGCAGAGGATGCAGCAACATACTATTGCCAGCAGTGGTCTTTCA | |
| | ACCCCCCTACATTTGGCGGCGGCACCAAGCTGGAGATCAAG | |
| 176 | DIVLTQSPAILSASPGEKVTMTCRASSSVNYMDWYQKKPGSSPKPWIY | αCD20 CAR |
| | ATSNLASGVPARFSGSGSGTSYSLTISRVEAEDAATYYCQQWSFNPPTF | scFv VL |
| | GGGTKLEIK | polypeptide |
| 177 | GGCTCTACCAGCGGAGGAGGAAGCGGAGGAGGATCCGGAGGCGGC | αCD20 CAR |
| | GGCTCTAGC | scFv linker |
| | | polynucleotide |
| 278 | GSTSGGGSGGGSGGGGSS | αCD20 CAR |
| | | scFv linker |
| | | polynucleotide |
| 178 | GAGGTGCAGCTGCAGCAGTCCGGAGCAGAGCTGGTGAAGCCTGGA | αCD20 CAR |
| | GCCTCTGTGAAGATGAGCTGTAAGGCCTCCGGCTACACCTTCACAT | scFv VH |
| | CTTATAATATGCACTGGGTGAAGCAGACACCAGGACAGGGACTGG | polynucleotide |
| | AGTGGATCGGAGCAATCTACCCTGGCAACGGCGACACCAGCTATAA | |
| | TCAGAAGTTTAAGGGCAAGGCCACCCTGACAGCCGATAAGTCCTCT | |
| | AGCACAGCCTACATGCAGCTGTCCTCTCTGACCAGCGAGGACTCCG | |
| | CCGATTACTATTGCGCCCGGTCCAACTACTATGGCAGCTCCTATTGG | |
| | TTCTTTGACGTGTGGGGAGCAGGAACAACCGTGACCGTGTCTAGC | |
| 179 | EVQLQQSGAELVKPGASVKMSCKASGYTFTSYNMHWVKQTPGQGLE | αCD20 CAR |
| | WIGAIYPGNGDTSYNQKFKGKATLTADKSSSTAYMQLSSLTSEDSADY | scFv VH |
| | YCARSNYYGSSYWFFDVWGAGTTVTVSS | polypeptide |
| 180 | GACTACAAAGACGATGACGACAAG | αCD20 CAR |
| | | FLAG tag |
| 211 | DYKDDDDK | αCD20 CAR |
| | | FLAG tag |
| | | polypeptide |
| 181 | GCAAAGCCAACCACCACACCTGCTCCTAGACCACCTACACCCGCTC | αCD20 CAR |
| | CTACCATCGCCAGCCAGCCTCTGTCTCTGAGACCTGAGGCCTGTAG | CD8 hinge and |
| | ACCTGCCGCTGGAGGCGCTGTGCACACCAGAGGACTGGATTTCGCC | transmembrane |
| | TGCGACATCTACATCTGGGCTCCTCTGGCTGGAACATGCGGCGTGC | domain |
| | TGCTCCTGAGCCTGGTGATCACACTGTACTGC | |
| 212 | AKPTTTPAPRPPTPAPTIASQPLSLRPEACRPAAGGAVHTRGLDFACDIY | αCD20 CAR |
| | IWAPLAGTCGVLLLSLVITLYC | CD8 Hinge and |
| | | transmembrane |
| | | domain |
| | | polypeptide |
| 182 | GCAAAGCCAACCACCACACCTGCTCCTAGACCACCTACACCCGCTC | αCD20 CAR |
| | CTACCATCGCCAGCCAGCCTCTGTCTCTGAGACCTGAGGCCTGTAG | CD8 hinge |
| | ACCTGCCGCTGGAGGCGCTGTGCACACCAGAGGACTGGATTTCGCC | polynucleotide |
| | TGCGAC | |
| 280 | AKPTTTPAPRPPTPAPTIASQPLSLRPEACRPAAGGAVHTRGLDFACD | αCD20 CAR |
| | | CD8 hinge |
| | | polypeptide |
| 183 | ATCTACATCTGGGCTCCTCTGGCTGGAACATGCGGCGTGCTGCTCCT | αCD20 CAR |
| | GAGCCTGGTGATCACACTGTACTGC | CD8 |
| | | transmembrane |
| | | domain |
| | | polynucleotide |
| 281 | IYIWAPLAGTCGVLLLSLVITLYC | αCD20 CAR |
| | | CD8 |
| | | transmembrane |
| | | domain |
| | | polypeptide |
| 184 | AAGAGAGGCAGGAAGAAGCTGCTGTATATCTTTAAGCAGCCCTTCA | αCD20 CAR |
| | TGCGCCCTGTGCAGACCACACAGGAGGAGGACGGCTGCAGCTGTC | 41BB |
| | GGTTTCCAGAGGAGGAGGAGGGAGGATGCGAGCTG | |
| 213 | KRGRKKLLYIFKQPFMRPVQTTQEEDGCSCRFPEEEEGGCEL | αCD20 CAR |
| | | 41BB |
| | | polypeptide |
| 185 | CGCGTGAAGTTCAGCCGGTCCGCCGATGCCCCTGCCTACCAGCAGG | αCD20 CAR |
| | GCCAGAACCAGCTGTATAACGAGCTGAATCTGGGCCGGAGAGAGG | CD3z |
| | AGTACGACGTGCTGGATAAGAGGAGGGGAAGGGACCCAGAGATGG | |
| | GAGGCAAGCCTCGGAGAAAGAACCCACAGGAGGGCCTGTACAATG | |
| | AGCTGCAGAAGGACAAGATGGCCGAGGCCTATTCTGAGATCGGCA | |
| | TGAAGGGAGAGAGGCGCCGGGGCAAGGGACACGATGGCCTGTACC | |
| | AGGGCCTGAGCACCGCCACAAAGGACACCTATGATGCCCTGCACAT | |
| | GCAGGCCCTGCCCCCTCGG | |
| 214 | RVKFSRSADAPAYQQGQNQLYNELNLGRREEYDVLDKRRGRDPEMG | αCD20 CAR |
| | GKPRRKNPQEGLYNELQKDKMAEAYSEIGMKGERRRGKGHDGLYQG | CD3z |
| | LSTATKDTYDALHMQALPPR | polypeptide |
| 186 | GATATCGTGCTGACCCAGTCCCCCGCCATCCTGTCCGCCTCTCCTGG | Full length |
| | AGAGAAGGTGACCATGACATGTCGGGCCAGCTCCTCTGTGAACTAC | αCD20 CAR w/ |
| | ATGGACTGGTATCAGAAGAAGCCTGGCAGCTCCCCCAAGCCTTGGA | FLAG tag |
| | TCTACGCCACCTCCAATCTGGCCTCTGGAGTGCCAGCAAGATTCAG | |
| | CGGATCCGGATCTGGCACAAGCTATTCCCTGACCATCTCCAGGGTG | |
| | GAGGCAGAGGATGCAGCAACATACTATTGCCAGCAGTGGTCTTTCA | |
| | ACCCCCCTACATTTGGCGGCGGCACCAAGCTGGAGATCAAGGGCTC | |
| | TACCAGCGGAGGAGGAAGCGGAGGAGGATCCGGAGGCGGCGGCTC | |
| | TAGCGAGGTGCAGCTGCAGCAGTCCGGAGCAGAGCTGGTGAAGCC | |
| | TGGAGCCTCTGTGAAGATGAGCTGTAAGGCCTCCGGCTACACCTTC | |
| | ACATCTTATAATATGCACTGGGTGAAGCAGACACCAGGACAGGGA | |
| | CTGGAGTGGATCGGAGCAATCTACCCTGGCAACGGCGACACCAGCT | |
| | ATAATCAGAAGTTTAAGGGCAAGGCCACCCTGACAGCCGATAAGTC | |
| | CTCTAGCACAGCCTACATGCAGCTGTCCTCTCTGACCAGCGAGGAC | |
| | TCCGCCGATTACTATTGCGCCCGGTCCAACTACTATGGCAGCTCCTA | |
| | TTGGTTCTTTGACGTGTGGGGAGCAGGAACAACCGTGACCGTGTCT | |
| | AGCGCTGCAGGAGGCGGAGGATCTGGAGGCGGCGGCGGAGACTAC | |
| | AAAGACGATGACGACAAGTTCGAAGCAAAGCCAACCACCACACCT | |
| | GCTCCTAGACCACCTACACCCGCTCCTACCATCGCCAGCCAGCCTCT | |
| | GTCTCTGAGACCTGAGGCCTGTAGACCTGCCGCTGGAGGCGCTGTG | |
| | CACACCAGAGGACTGGATTTCGCCTGCGACATCTACATCTGGGCTC | |
| | CTCTGGCTGGAACATGCGGCGTGCTGCTCCTGAGCCTGGTGATCAC | |
| | ACTGTACTGCAAGAGAGGCAGGAAGAAGCTGCTGTATATCTTTAAG | |
| | CAGCCCTTCATGCGCCCTGTGCAGACCACACAGGAGGAGGACGGCT | |
| | GCAGCTGTCGGTTTCCAGAGGAGGAGGAGGGAGGATGCGAGCTGC | |
| | GCGTGAAGTTCAGCCGGTCCGCCGATGCCCCTGCCTACCAGCAGGG | |
| | CCAGAACCAGCTGTATAACGAGCTGAATCTGGGCCGGAGAGAGGA | |
| | GTACGACGTGCTGGATAAGAGGAGGGGAAGGGACCCAGAGATGGG | |
| | AGGCAAGCCTCGGAGAAAGAACCCACAGGAGGGCCTGTACAATGA | |
| | GCTGCAGAAGGACAAGATGGCCGAGGCCTATTCTGAGATCGGCAT | |
| | GAAGGGAGAGAGGCGCCGGGGCAAGGGACACGATGGCCTGTACCA | |
| | GGGCCTGAGCACCGCCACAAAGGACACCTATGATGCCCTGCACATG | |
| | CAGGCCCTGCCCCCTCGG | |
| 209 | DIVLTQSPAILSASPGEKVTMTCRASSSVNYMDWYQKKPGSSPKPWIY | Full length |
| | ATSNLASGVPARFSGSGSGTSYSLTISRVEAEDAATYYCQQWSFNPPTF | αCD20 CAR w/ |
| | GGGTKLEIKGSTSGGGSGGGSGGGGSSEVQLQQSGAELVKPGASVKM | FLAG tag |
| | SCKASGYTFTSYNMHWVKQTPGQGLEWIGAIYPGNGDTSYNQKFKGK | polypeptide |
| | ATLTADKSSSTAYMQLSSLTSEDSADYYCARSNYYGSSYWFFDVWGA | |
| | GTTVTVSSAAGGGGSGGGGGDYKDDDDKFEAKPTTTPAPRPPTPAPTI | |
| | ASQPLSLRPEACRPAAGGAVHTRGLDFACDIYIWAPLAGTCGVLLLSL | |
| | VITLYCKRGRKKLLYIFKQPFMRPVQTTQEEDGCSCRFPEEEEGGCELR | |
| | VKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGL | |
| | STATKDTYDALHMQALPPR | |
| 187 | ESKYGPPCPPCPM | modified IgG4 |
| | | hinge |
| 188 | ESKYGPPSPPSPA | modified IgG4 |
| | | hinge |
| 189 | ESKYGPPSPPSP | modified IgG4 |
| | | hinge |
| 190 | PSPRPAGQFQTLV | PD1 hinge |
| 191 | EPKSCDKTHTCP | IgG1 hinge |
| 192 | GAVHTRGLDFACD | CD8 hinge |
| 193 | LCPSPLFPGPSKP | CD28 hinge |

In some embodiments, the antigen is CD20. In some embodiments, a CAR comprises an extracellular domain comprising a scFv binding domain for CD20 binding. In some embodiments, the CAR is a second-generation CAR comprised of an anti-CD20 scFv linked to the 4-1BB costimulatory domain and the CD3zeta intracellular signaling domain. In some embodiments, a CAR comprises a binding domain for CD20, a CD8a hinge, a CD8a transmembrane domain, a 4-1BB costimulatory domain, and a CD3zeta signaling domain. In some embodiments, a CAR comprises a binding domain for CD20, an IgG4 hinge, a CD28 transmembrane domain, a 4-1BB costimulatory domain, and a CD3zeta signaling domain. In some embodiments, a CAR comprises a binding domain for CD20, a CD28 hinge, a CD28 transmembrane domain, a CD28 costimulatory domain, and CD3zeta signaling domain. In some embodiments, a CAR comprises an extracellular domain comprising a Leu16 scFv binding domain for CD20 binding, a CD8a hinge, a CD8a transmembrane domain, a 4-1BB costimulatory domain, and a CD3zeta signaling domain. In some embodiments, a CAR comprises an extracellular domain comprising a Leu16 scFv binding domain for CD20 binding, an IgG4 hinge, a CD28 transmembrane domain, a 4-1BB costimulatory domain, and a CD3zeta signaling domain. In some embodiments, a CAR comprises an extracellular domain comprising a Leu16 scFv binding domain for CD20 binding, a CD28 hinge, a CD28 transmembrane domain, a CD28 costimulatory domain, and CD3zeta signaling domain. In some embodiments, a CAR comprises an extracellular domain comprising a 2B8 scFv binding domain for CD20 binding, a CD8a hinge, a CD8a transmembrane domain, a 4-1BB costimulatory domain, and a CD3zeta signaling domain. In some embodiments, a CAR comprises an extracellular domain comprising a 2B8 scFv binding domain for CD20 binding, an IgG4 hinge, a CD28 transmembrane domain, a 4-1BB costimulatory domain, and a CD3zeta signaling domain. In some embodiments, a CAR comprises an extracellular domain comprising a 2B8 scFv binding domain for CD20 binding, a CD28 hinge, a CD28 transmembrane domain, a CD28 costimulatory domain, and CD3zeta signaling domain.

In some embodiments of the polypeptides described herein, the extracellular domain is joined to said transmembrane domain directly or by a linker, spacer or hinge polypeptide sequence, e.g., a sequence from CD28 or a sequence from CTLA4.

In some embodiments, the extracellular domain that binds the desired antigen may be from antibodies or their antigen binding fragments generated using the technologies described herein.

Antibody engineering (and likewise CAR engineering) may include precise identification of residues that have an impact on interaction or affinity of an antibody to a target antigen. Six CDRs in an antibody variable domain (e.g., three from the light chain and three from the heavy chain) may fold up together in 3-dimensional space to form an antibody binding site which docks onto the antigen. In some embodiments, the position and length of the CDRs can be defined by Kabat, E. et al., Sequences of Proteins of Immunological Interest, U.S. Department of Health and Human Services, 1983, 1987. In some embodiments, the position and length of the CDRs can be defined using the Chothia numbering scheme, Martin numbering scheme, Gelfnad number scheme, IMGT numbering scheme or Honnegar's numbering scheme, e.g. as described in Dondelinger, M et al. Understanding the Significance and Implications of Antibody Numbering and Antigen-Binding Surface/Residue Definition, Front Immunol., 2018. A part of a variable region not contained in the CDRs may be referred to as a framework region, which may form a three-dimensional environment for the CDRs. A CDR sequence or framework region provided herein may be used in an embodiment that uses a CAR or binding fragment.

Some embodiments relate to or include a CAR that specifically binds an antigen (e.g. CD20) and comprise CDRs that bind to the antigen. The CDRs may be included in a sequence herein such as a VL, VH, or scFv sequence. Some embodiments include a CDR of an anti-CD22 CAR sequence provided herein. Some embodiments include a CDR of the VL sequence of SEQ ID NO: 176, or a sequence thereof having 1 or 2 substitutions, additions, or deletions. Some embodiments include a CDR of the VL sequence of SEQ ID NO: 176. Some embodiments include a CDR of the VH sequence of SEQ ID NO: 179, or a sequence thereof having 1 or 2 substitutions, additions, or deletions. Some embodiments include a CDR of the VH sequence of SEQ ID NO: 179. Some embodiments include a CDR of the VL sequence encoded by a nucleic acid sequence of SEQ ID NO: 175, or by a nucleic acid at least 90%, 91%, 92%, 93%, 94%, 95%, 96%, 97%, 98%, or 99% identical to SEQ ID NO: 175. Some embodiments include a CDR of the VL sequence encoded by a nucleic acid sequence of SEQ ID NO: 175. Some embodiments include a CDR of the VH sequence encoded by a nucleic acid sequence of SEQ ID NO: 178, or by a nucleic acid at least 90%, 91%, 92%, 93%, 94%, 95%, 96%, 97%, 98%, or 99% identical to SEQ ID NO: 178. Some embodiments include a CDR of the VH sequence encoded by a nucleic acid sequence of SEQ ID NO: 178. Some embodiments include 1, 2, 3, 4, 5, or 6 CDRs of the aforesaid sequences. For example, CDR1, CDR2 and CDR3 of a VL sequence may be included, and CDR1, CDR2, and CDR3 of a VH sequence may be included.

### Particles

In some embodiments, the disclosure provides particles of various types, including but not limited to lentiviral particles (i.e., a virion), lipid nanoparticles (LNPs), lipoplexes, liposomes, and nanocarriers. The particle may include an adhesion molecule, a costimulatory molecule, an activation molecule, a combination thereof. Any of the adhesion molecule, costimulatory molecule, or activation may be included in a fusion molecule. The adhesion molecule, costimulatory molecule, activation molecule, or combination thereof may be included at a surface of the particle. The fusion molecule may be included at a surface of the particle.

The particle may be a lipid nanoparticle (LNP) or a poly(beta-amino) esters (PBAE) nanocarriers, both of which have been shown to transduce T cells when administered to a subject in vivo or contacted with T cells ex vivo. Using the compositions and methods described herein, transduction of T cells with LNPs and PBAE-based nanocarriers may be increased.

In some embodiments, the particle is a viral particle. Methods for generating viral vectors from various virus types are known in the art. Exemplary types of viral particles that may be recombinantly engineered as delivery vehicles include retroviruses, lentivirus (e.g. HIV and its derivatives and SIV), adeno-associated virus, adenovirus, MMLV retrovirus, MSCV retrovirus, baculovirus, vesicular stomatitis virus, herpes simplex virus, and vaccinia virus. Examples include adeno-associated virus (AAV) particles used for gene therapy. In a preferred embodiment, the particle is a retroviral particle. In a particularly preferred embodiment, the particle is a lentiviral particle.

Lentiviral particles may be made using packaging cell lines as described in WO 2016/139463 or by using a polycistronic vector as described in Int'l Pat. Pub. No. WO 2020/106992 A1. Each of the foregoing specifically describes methods for making lentiviral particles. Their disclosures are incorporated by reference herein. Numerous other methods for making viral particles, including lentiviral particles, may be useful.

Retroviruses, a group that includes lentiviruses, are enveloped viruses. The fusion molecules described herein may be displayed on such enveloped viruses by expressing the fusion molecule, or its various components, in a host cell under the control of a suitable promotor (or promoters). Each component may include a signal sequence for secretion. At least one component should include a transmembrane region or anchor sequence (such as the C-terminal signal sequence that directs the attachment of a GPI anchor). The other components may associate with the first component either during the secretion process or after secretion. In the case of single fusion proteins, only one transmembrane region or anchor sequence may be required, although those of skill in the art may envision and employ multiple transmembrane regions or anchor sequences. In some embodiments, the fusion molecule is a fusion protein comprising a C-terminal transmembrane region, expressed from a polynucleotide that encodes an N-terminal signal peptide. The signal peptide may be cleaved during expression of the protein at the cell surface of the producer cell, leaving a membrane-tethered fusion protein without the signal sequence. The lentiviral particle may then be made when a virion buds from the surface of the producer cell, incorporating as its envelope portions of the cell membrane that include one or more copies of the fusion protein.

Lentiviral particles generally package a vector genome and, incidentally or intentionally, may package other molecules present in the producer cell. The vector genome may be an artificial vector genome engineered to encode a heterologous protein or polynucleotide.

Lentiviral particles may contain structural and/or functional genetic elements that are primarily from a virus. Lentiviral particles are characterized by the predominant source of genetic or structural material in the lentiviral particle. Thus, the term “retroviral particle” refers to a viral particle containing structural proteins and vector genome elements, primarily from a retrovirus. Likewise, the term “lentiviral particle” refers to a viral particle containing structural proteins and vector genome elements, primarily from a lentivirus. To package its vector genome, a lentiviral particle may generally require at least one copy of the long-terminal repeats (LTRs) that flank a native lentiviral vector genome, or functional variants thereof.

In some embodiments, a viral particle comprises a viral glycoprotein. In some embodiments, the viral particle comprises a viral glycoprotein different from the native viral glycoprotein. When the viral glycoprotein is heterologous to the vector genome, the viral particle is termed a “pseudotyped” viral particle. For example, in some embodiments, the viral particle is derived from HIV, which typically includes the glycoprotein gp120. However, such HIV-based particles may be “pseudotyped” and, instead of expressing their native glycoprotein, express a glycoprotein from a different virus. For example, the viral glycoprotein may be a portion of RD 114 or one of its variants, VSV-G, Gibbon-ape leukemia virus (GALV), the Amphotropic envelope glycoprotein, Measles envelope glycoprotein, or baboon retroviral envelope glycoprotein. In some embodiments, the viral envelope glycoprotein is a G protein from the Cocal strain (Cocal G), or a functional variant thereof. Illustrative viral glycoproteins include the VSV G protein, the Cocal G protein, and variants thereof. Illustrative viral glycoproteins may be expressed as a single protein or in multiple subunits or parts. The viral glycoprotein may serve as ligand for cell-surface receptors on a target cell, and thereby promote transduction or the target cell. The viral glycoprotein may be engineered to lack LDLR binding affinity—for example, by mutation at positions 47 (e.g., K47Q) and/or 354 (e.g., R354A). This may be termed a “blinded” viral glycoprotein. Illustrative envelope variants are provided in, e.g., US 2020/0216502 A1, which is incorporated herein by reference in its entirety. Surprisingly, in some embodiments, the fusion molecules as described herein may permit use of a viral glycoprotein that does not, by itself, cause transduction of target cell. Without being bound by theory, it is believed that the fusion protein may serve as a ligand for cell-surface receptor while the viral glycoprotein retains a structural function, but not a function as a ligand for a cell-surface receptor.

In some embodiments, the viral glycoprotein is a VSV-G glycoprotein that comprises a mutation at position 47. In some embodiments, the viral glycoprotein is a VSV-G glycoprotein that comprises a mutation at position 354. In some embodiments, the viral glycoprotein is a VSV-G glycoprotein that comprises a K47Q mutation. In some embodiments, the viral glycoprotein is a VSV-G glycoprotein that comprises a R354A mutation. In some embodiments, the viral glycoprotein is a VSV-G glycoprotein that comprises a K47Q and a R354A mutation. In some embodiments, the viral glycoprotein is a cocal glycoprotein that comprises a mutation at position 47. In some embodiments, the viral glycoprotein is a cocal glycoprotein that comprises a mutation at position 354. In some embodiments, the viral glycoprotein is a cocal glycoprotein that comprises a K47Q mutation. In some embodiments, the viral glycoprotein is a cocal glycoprotein that comprises a R354A mutation. In some embodiments, the viral glycoprotein is a cocal glycoprotein that comprises a K47Q and a R354A mutation.

In some embodiments, the viral glycoprotein comprises a mutation at position 47. In some embodiments, the viral glycoprotein comprises a mutation at position 354. In some embodiments, the viral glycoprotein comprises a K47Q mutation. In some embodiments, the viral glycoprotein comprises a R354A mutation. In some embodiments, the viral glycoprotein comprises a K47Q and a R354A mutation.

The Cocal G protein may have a polypeptide sequence having at least 75%, at least 80%, at least 85%, at least 90%, at least 95%, at least 99%, or 100% sequence identity to the following sequence:

(SEQ ID NO: 74)
NFLLLTFIVLPLCSHAKFSIVFPQSQKGNWKNVPSSYHYCPSSSD
QNWHNDLLGITMKVKMPKTHKAIQADGWMCHAAKWITTCDFRWYG
PKYITHSIHSIQPTSEQCKESIKQTKQGTWMSPGFPPQNCGYATV
TDSVAVVVQATPHHVLVDEYTGEWIDSQFPNGKCETEECETVHNS
TVWYSDYKVTGLCDATLVDTEITFFSEDGKKESIGKPNTGYRSNY
FAYEKGDKVCKMNYCKHAGVRLPSGVWFEFVDQDVYAAAKLPECP
VGATISAPTQTSVDVSLILDVERILDYSLCQETWSKIRSKQPVSP
VDLSYLAPKNPGTGPAFTIINGTLKYFETRYIRIDIDNPIISKMV
GKISGSQTERELWTEWFPYEGVEIGPNGILKTPTGYKFPLFMIGH
GMLDSDLHKTSQAEVFEHPHLAEAPKQLPEEETLFFGDTGISKNP
VELIEGWFSSWKSTVVTFFFAIGVFILLYVVARIVIAVRYRYQGS
NNKRIYNDIEMSRFRK.

The Cocal G protein may have a polypeptide sequence having at least 75%, at least 80%, at least 85%, at least 90%, at least 95%, at least 99%, or 100% sequence identity to the following sequence:

(SEQ ID NO: 247)
MNFLLLTFIVLPLCSHAKFSIVFPQSQKGNWKNVPSSYHYCPSSS
DQNWHNDLLGITMKVKMPKTHKAIQADGWMCHAAKWITTCDFRWY
GPKYITHSIHSIQPTSEQCKESIKQTKQGTWMSPGFPPQNCGYAT
VTDSVAVVVQATPHHVLVDEYTGEWIDSQFPNGKCETEECETVHN
STVWYSDYKVTGLCDATLVDTEITFFSEDGKKESIGKPNTGYRSN
YFAYEKGDKVCKMNYCKHAGVRLPSGVWFEFVDQDVYAAAKLPEC
PVGATISAPTQTSVDVSLILDVERILDYSLCQETWSKIRSKQPVS
PVDLSYLAPKNPGTGPAFTIINGTLKYFETRYIRIDIDNPIISKM
VGKISGSQTERELWTEWFPYEGVEIGPNGILKTPTGYKFPLFMIG
HGMLDSDLHKTSQAEVFEHPHLAEAPKQLPEEETLFFGDTGISKN
PVELIEGWFSSWKSTVVTFFFAIGVFILLYVVARIVIAVRYRYQG
SNNKRIYNDIEMSRFRK

Illustrative lentiviruses include but are not limited to: HIV (human immunodeficiency virus; including HIV type 1, and HIV type 2; visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, the backbones are HIV-based vector backbones (i.e., HIV cis-acting sequence elements). Retroviral particles have been generated by multiply attenuating the HIV virulence genes, for example, the genes env, vif, vpr, vpu and nef are deleted, making the vector biologically safe.

Illustrative lentiviral particles and methods for making them are described in Naldini et al. Science 272:263-7 (1996); Zufferey et al. J. Virol. 72:9873-9880 (1998); Dull et al. J. Virol. 72:8463-8471 (1998); Miyoshi et al. J. Virol. 72:8150-57 (1998); U.S. Pat. Nos. 6,013,516; and 5,994,136.

Protocols for producing replication-defective recombinant viruses are provided in WO95/14785, WO96/22378, U.S. Pat. Nos. 5,882,877, 6,013,516, 4,861,719, 5,278,056, and WO94/19478.

Viral particles may be assessed in various ways, including, for example, measuring the vector copy number (VCN) or vector genomes (vg) in a sample of viral particle by quantitative polymerase chain reaction (qPCR) or digital droplet PCR (ddPCR), or testing to the viral particles on target cells to measure a “titer” of the virus in, e.g., infectious units per milliliter (IU/mL). For example, the titer may be assessed using a functional assay performed on the cultured tumor cell line HT1080 as described in Humbert et al. Molecular Therapy 24:1237-1246 (2016). When titer is assessed on a cultured cell line that is continually dividing, stimulation may be unnecessary, and hence the measured titer may be uninfluenced by surface engineering of the retroviral particle. Other methods for assessing the efficiency of retroviral vector systems are provided in Gaererts et al. BMC Biotechnol. 6:34 (2006).

### Payloads

The particle may be used to deliver a payload. The term “payload” refers to any molecule or combination of molecules whose delivery to a target cell is desired. Various payloads may be delivered using the particles desired herein, including but not limited to small molecules, polynucleotide and proteins. When the selected target cell is a T cell, the particles of the disclosure may be used to deliver a therapeutic agent targeting T cells, to genetically modified T cells, or to deliver a polynucleotide encoding a protein of interest to the T cell. Similarly, particles disclosed herein may be used to delivery payloads to NK cells.

The payload may be a polynucleotide comprising a polynucleotide encoding a chimeric antigen receptor (CAR). In exemplary embodiments, the payload may be a polynucleotide encoding an anti-CD20 CAR as disclosed herein.

As used herein, the term “similar” may refer to a polynucleotide or polypeptide sequence at least about 75%, at least about 80%, at least about 90%, at least about 95%, at least about 97%, at least about 98%, at least about 99%, or at least about 99.5% to a reference sequence.

The payload may comprise a polynucleotide whose sequence encodes a gene-editing system (e.g., CRISPR-Cas, meganuclease, homing endonuclease, zinc finger enzyme).

In some embodiments, the lentiviral particles of the present disclosure comprise a polynucleotide sequence encoding, in any order, on a polycistronic transcript: a promoter, a therapeutic protein (e.g. CAR), optionally a cytosolic FRB domain or a portion thereof, and optionally a synthetic cytokine polypeptide (e.g. RACR). In some embodiments, the polycistronic transcript comprises a promoter and a CAR. Illustrative promoters include, without limitation, a cytomegalovirus (CMV) promoter, a CAG promoter, an SV40 promoter, an SV40/CD43 promoter, and a MND promoter.

In some embodiments, the MND promoter comprises a nucleic acid sequence that shares at least 75%, at least 80%, at least 85%, at least 90%, at least 95%, at least 99%, or 100% identity to SEQ ID NO: 118.

(SEQ ID NO: 118)
GAACAGAGAAACAGGAGAATATGGGCCAAACAGGATATCTGTGGT
AAGCAGTTCCTGCCCCGGCTCAGGGCCAAGAACAGTTGGAACAGC
AGAATATGGGCCAAACAGGATATCTGTGGTAAGCAGTTCCTGCCC
CGGCTCAGGGCCAAGAACAGATGGTCCCCAGATGCGGTCCCGCCC
TCAGCAGTTTCTAGAGAACCATCAGATGTTTCCAGGGTGCCCCAA
GGACCTGAAATGACCCTGTGCCTTATTTGAACTAACCAATCAGTT
CGCTTCTCGCTTCTGTTCGCGCGCTTCTGCTCCCCGAGCTCTATA
TAAGCAGAGCTCGTTTAGTGAACCGTCAGATCGCTAGC

In some embodiments, the MND promoter comprises a nucleic acid sequence that shares at least 75%, at least 80%, at least 85%, at least 90%, at least 95%, at least 99%, or 100% identity to SEQ ID NO: 172.

(SEQ ID NO: 172)
AATGAAAGACCCCACCTGTAGGTTTGGCAAGCTAGGATCAAGGTC
AGGAACAGAGAGACAGCAGAATATGGGCCAAACAGGATATCTGTG
GTAAGCAGTTCCTGCCCCGGCTCAGGGCCAAGAACAGTTGGAACA
GCAGAATATGGGCCAAACAGGATATCTGTGGTAAGCAGTTCCTGC
CCCGGCTCAGGGCCAAGAACAGATGGTCCCCAGATGCGGTCCCGC
CCTCAGCAGTTTCTAGAGAACCATCAGATGTTTCCAGGGTGCCCC
AAGGACCTGAAATGACCCTGTGCCTTATTTGAACTAACCAATCAG
TTCGCTTCTCGCTTCTGTTCGCGCGCTTCTGCTCCCCGAGCTCTA
TATAAGAGCCCACAACCCCTCACTCGGC

In some embodiments, the CSF2RA signal sequence comprises a nucleic acid sequence that shares at least 75%, at least 80%, at least 85%, at least 90%, at least 95%, at least 99%, or 100% identity to SEQ ID NO: 173.

(SEQ ID NO: 173)
ATGCTGCTGCTGGTGACAAGCCTGCTGCTGTGCGAGCTGCCTCAC
CCAGCCTTTCTGCTGATCCCC

The disclosure provides a polynucleotide construct comprising a contiguous polynucleotide sequence encoding at least two synthetic receptors and methods for uses thereof. In some embodiments, the polynucleotide construct is a polycistronic construct encoding a synthetic cytokine receptor, a synthetic chimeric antigen receptor (CAR), and a freely diffusible FRB, in which the cytokine receptor is responsive to rapamycin binding. Advantageously, FRB reduces the inhibitory effects of rapamycin on mTOR in cells engineered to express the polycistronic constructs provided herein. Expression of the freely diffusible FRB can promote consistent activation and proliferation of engineered cells.

In some aspects, provided herein is a lentiviral vector comprising any one of the polycistronic constructs disclosed herein. In some aspects, provided herein is a cell comprising any of the lentiviral vectors disclosed herein.

In some aspects, provided herein is a method of transducing a cell comprising contacting a target cell with any of the polycistronic constructs disclosed herein.

In some aspects, provided herein is a method of expressing a chimeric antigen receptor and/or a synthetic cytokine receptor in a target cell. In some aspects, provided herein is a cell produced by any of the methods disclosed herein.

In some aspects, provided herein is a method of administering to a subject any of the cells disclosed herein. In some aspects, provided herein is a method of administering to a subject any of the lentiviral vectors disclosed herein.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described

Disclosed herein, in some embodiments, are lentiviral particles, comprising a polynucleotide encoding a chimeric antigen receptor that specifically binds CD20 (e.g. an anti-CD20 CAR). In some embodiments, the lentiviral particle comprises a polynucleotide encoding a free FKBP12-rapamycin binding (FRB). In some embodiments, the CD58 extracellular domain, or a functional fragment thereof, comprises a polypeptide sequence that is at least 80%, 85%, 90%, 91%, 92%, 93%, 94%, 95%, 96%, 97%, 98%, or 99% identical to the polypeptide sequence of SEQ ID NO: 10. In some embodiments, the antigen-binding fragment of an anti-CD3 antibody is an scFv domain comprising a polypeptide sequence that is at least 80%, 85%, 90%, 91%, 92%, 93%, 94%, 95%, 96%, 97%, 98%, or 99% identical to the polypeptide sequence of SEQ ID NO: 31. In some embodiments, the CD80 extracellular domain, or a functional fragment thereof, comprises a polypeptide sequence that is at least 80%, 85%, 90%, 91%, 92%, 93%, 94%, 95%, 96%, 97%, 98%, or 99% identical to the polypeptide sequence of SEQ ID NO: 12. In some embodiments, the CD86 extracellular domain, or a functional fragment thereof, comprises a polypeptide sequence that is at least 80%, 85%, 90%, 91%, 92%, 93%, 94%, 95%, 96%, 97%, 98%, or 99% identical to the polypeptide sequence of SEQ ID NO: 13. In some embodiments, the fusion molecule comprises, in N to C-terminal order, the CD58 extracellular domain, the antigen-binding fragment of an anti-CD3 antibody, and the CD86 extracellular domain. In some embodiments, the fusion molecule comprises a polypeptide sequence that is at least 80%, 85%, 90%, 91%, 92%, 93%, 94%, 95%, 96%, 97%, 98%, or 99% identical to the polypeptide sequence of SEQ ID NO: 33. In some embodiments, the fusion molecule comprises, in N to C-terminal order, the CD58 extracellular domain, the antigen-binding fragment of an anti-CD3 antibody, and the CD80 extracellular domain. In some embodiments, the viral glycoprotein (G protein) comprises a polypeptide sequence that is at least 80%, 85%, 90%, 91%, 92%, 93%, 94%, 95%, 96%, 97%, 98%, or 99% identical to the polypeptide sequence of SEQ ID NO: 74. In some embodiments, the lentivirus comprises a polycistronic construct comprising in 5′ to 3′ order: (a) a first expression cassette comprising a nucleotide sequence encoding the free FRB, (b) a second expression cassette comprising a nucleotide sequence encoding the synthetic cytokine gamma chain polypeptide, (c) a third expression cassette comprising a nucleotide sequence encoding the synthetic cytokine beta chain polypeptide, and (d) a fourth expression cassette comprising a nucleotide sequence encoding an anti-CD20 CAR; wherein each of the expression cassettes are separated by a nucleotide sequence encoding a cleavage site sequence. In some embodiments, the polynucleotide sequence encoding FRB that is at least 80%, 85%, 90%, 91%, 92%, 93%, 94%, 95%, 96%, 97%, 98%, or 99% identical to the polynucleotide sequence of SEQ ID NOs: 256, 257, or 258. The lentiviral particle of any one of embodiments 1-43, wherein the polynucleotide sequence encoding the free FRB comprises the polynucleotide sequence of SEQ ID NOs: 256, 257, or 258. In some embodiments, the free FRB polynucleotide sequence encodes a polypeptide sequence that is at least 80%, 85%, 90%, 91%, 92%, 93%, 94%, 95%, 96%, 97%, 98%, or 99% identical to the polynucleotide sequence of SEQ ID NOs: 251, 252, or 260. In some embodiments, the FRB polynucleotide sequence encodes the polypeptide sequence of SEQ ID NOs: 251, 252, or 260. In some embodiments, the polynucleotide sequence encoding a synthetic cytokine gamma chain polypeptide is at least 80%, 85%, 90%, 91%, 92%, 93%, 94%, 95%, 96%, 97%, 98%, or 99% identical to the polynucleotide sequence of SEQ ID NOs: 261, 262, or 263. In some embodiments, the polynucleotide encoding the synthetic cytokine gamma chain polypeptide comprises the polynucleotide sequence of SEQ ID NOs: 261, 262, or 263. In some embodiments, the synthetic cytokine gamma chain polypeptide comprises interleukin 2 receptor subunit γ (IL2RG) comprising a polypeptide sequence at least 80%, 85%, 90%, 91%, 92%, 93%, 94%, 95%, 96%, 97%, 98%, or 99% identical to the amino acid sequence of SEQ ID NOs: 264 or 265. In some embodiments, the IL2RG comprises the polypeptide sequence of SEQ ID NOs: 264 or 265. In some embodiments, the second expression cassette comprises a polynucleotide sequence at least 80%, 85%, 90%, 91%, 92%, 93%, 94%, 95%, 96%, 97%, 98%, or 99% identical to the polynucleotide sequence of SEQ ID NO: 266. In some embodiments, the second expression cassette comprises the polynucleotide sequence of SEQ ID NO: 266. In some embodiments, the second expression cassette encodes a polypeptide sequence at least 80%, 85%, 90%, 91%, 92%, 93%, 94%, 95%, 96%, 97%, 98%, or 99% identical to the polypeptide sequence of SEQ ID NO: 267. In some embodiments, the second expression cassette encodes a polypeptide sequence comprising the sequence of SEQ ID NO: 267. In some embodiments, the second expression cassette further comprises a polynucleotide sequence encoding FKBP12 that is at least 80%, 85%, 90%, 91%, 92%, 93%, 94%, 95%, 96%, 97%, 98%, or 99% identical to the polynucleotide sequence of SEQ ID NOs: 268 or 269. In some embodiments, the polynucleotide sequence encoding the FKBP12 comprises the polynucleotide sequence of SEQ ID NOs: 268 or 269. In some embodiments, the FKBP12 comprises a polypeptide sequence at least 80%, 85%, 90%, 91%, 92%, 93%, 94%, 95%, 96%, 97%, 98%, or 99% identical to the polypeptide sequence of SEQ ID NO: 253 In some embodiments, the FKBP12 comprises the polypeptide sequence of SEQ ID NO: 253. In some embodiments, the polynucleotide encoding the synthetic cytokine beta chain polypeptide is at least 80%, 85%, 90%, 91%, 92%, 93%, 94%, 95%, 96%, 97%, 98%, or 99% identical to the polynucleotide sequence of SEQ ID NOs: 270 or 271. In some embodiments, the polynucleotide encoding the synthetic cytokine beta chain polypeptide comprises the polynucleotide sequence of SEQ ID NOs: 270 or 271. In some embodiments, the synthetic cytokine beta chain polypeptide comprises interleukin 2 receptor subunit β (IL2RB) comprising a polypeptide sequence at least 80%, 85%, 90%, 91%, 92%, 93%, 94%, 95%, 96%, 97%, 98%, or 99% identical to the amino acid sequence of SEQ ID NOs: 272 or 273. In some embodiments, the IL2RB comprises the polypeptide sequence of SEQ ID NOs: 272 or 273. In some embodiments, the third expression cassette further comprises a polynucleotide sequence encoding FKBP12 that is at least 80%, 85%, 90%, 91%, 92%, 93%, 94%, 95%, 96%, 97%, 98%, or 99% identical to the polynucleotide sequence of SEQ ID NO: 274. In some embodiments, the polynucleotide sequence encoding the FKBP12 comprises the polynucleotide sequence of SEQ ID NO: 274. In some embodiments, the FKBP12 comprises a polypeptide sequence at least 80%, 85%, 90%, 91%, 92%, 93%, 94%, 95%, 96%, 97%, 98%, or 99% identical to the polypeptide sequence of SEQ ID NO: 275. In some embodiments, the FKBP12 comprises the polypeptide sequence of SEQ ID NO: 275. In some embodiments, the third expression cassette comprises a polynucleotide sequence at least 80%, 85%, 90%, 91%, 92%, 93%, 94%, 95%, 96%, 97%, 98%, or 99% identical to the polynucleotide sequence of SEQ ID NO: 276. In some embodiments, the third expression cassette comprises the polynucleotide sequence of SEQ ID NO: 276. In some embodiments, the third expression cassette encodes a polypeptide sequence at least 80%, 85%, 90%, 91%, 92%, 93%, 94%, 95%, 96%, 97%, 98%, or 99% identical to the polypeptide sequence of SEQ ID NO: 277. In some embodiments, the third expression cassette encodes the polypeptide sequence comprising the sequence of SEQ ID NO: 277.

Some embodiments include a FRB polypeptide, a FKBP polypeptide, a cytokine beta polypeptide, and a cytokine gamma polypeptide. In some embodiments, the FRB polypeptide and cytokine beta chain polypeptide are fused together and the FKBP polypeptide and the cytokine gamma chain polypeptide are fused together. In other embodiments, the FRB polypeptide and cytokine gamma chain polypeptide are fused together and the FKBP polypeptide and the cytokine gamma chain polypeptide are fused together.

### Polycistronic Constructs

Provided herein are polycistronic constructs encoding one or more separate proteins. In some embodiments, the polycistronic constructs comprise one, two, three, or four expression cassettes each encoding a separate protein. In some embodiments, the polycistronic constructs comprise four expression cassettes each encoding a separate protein. In some embodiments, the expression cassettes are separated by cleavable linkers.

In some embodiments, the polycistronic constructs provided herein comprise a nucleotide sequence encoding an FRB. In some embodiments, the polycistronic constructs provided herein comprise a nucleotide sequence encoding a chimeric antigen receptor (CAR). In some embodiments, the polycistronic constructs provided herein comprise a nucleotide sequence encoding a synthetic cytokine polypeptide. In some embodiments, the synthetic cytokine polypeptide comprises a synthetic cytokine gamma chain polypeptide and a synthetic cytokine beta chain polypeptide. In some embodiments, the synthetic cytokine gamma chain comprises interleukin 2 receptor subunit γ (IL-2RG). In some embodiments, the synthetic cytokine gamma chain further comprises FRB. In some embodiments, the synthetic cytokine beta chain comprises interleukin 2 receptor subunit β (IL-2RB). In some embodiments, the synthetic cytokine gamma chain comprises further FKBP12. In other embodiments, the synthetic cytokine gamma chain comprises interleukin 2 receptor subunit γ (IL-2RG). In some embodiments, the synthetic cytokine gamma chain further comprises FKBP12. In some embodiments, the synthetic cytokine beta chain comprises interleukin 2 receptor subunit β (IL-2RB). In some embodiments, the synthetic cytokine beta chain further comprises FRB.

In some embodiments, the polycistronic construct provided herein comprises nucleotide sequences encoding an FRB, a synthetic cytokine polypeptide, and a CAR.

In some embodiments, the polycistronic construct comprises a nucleotide sequence encoding FRB, a nucleotide sequence encoding a synthetic cytokine polypeptide, and a nucleotide sequence encoding a CAR. In some embodiments, the nucleotide sequence encoding the synthetic cytokine polypeptide comprises a first nucleotide sequence encoding FRB:IL-2RG and a second nucleotide sequence encoding FKBP12:IL-2RB. In some embodiments, the nucleotide sequence encoding the synthetic cytokine polypeptide comprises a first nucleotide sequence encoding FKBP12:IL-2RG and a second nucleotide sequence encoding FRB:IL-2RB.

### Cytosolic FRB

In some embodiments, an expression cassette of the polycistronic construct encodes an FRB domain. The FRB domain is an approximately 270 base pair (bp) domain from the mTOR protein kinase. It may be expressed in the cytosol as a freely diffusible soluble protein.

In some embodiments, the first expression cassette in the polycistronic construct comprises a nucleotide sequence encoding an FRB. In some embodiments, when the FRB is expressed, it is a soluble, cytoplasmic protein (termed herein “free FRB”).

In some of any embodiments, the method further including administering a non-physiological ligand to the subject. In some embodiments, the non-physiological ligand is able to bind to the synthetic cytokine receptor and induce gamma cytokine signaling in the cell. In some embodiments, the nonphysiological ligand includes rapamycin or a rapamycin analog.

In some embodiments, the nucleotide sequence encoding the FRB is at least 80%, 85%, 90%, 91%, 92%, 93%, 94%, 95%, 96%, 97%, 98%, 99% or 100% identical to the nucleotide sequence of SEQ ID NOs: 256, 257, or 258. In some embodiments, the nucleotide sequence encoding the FRB is at least 100% identical to the nucleotide sequence of SEQ ID NOs: 256, 257, or 258. In some embodiments, the nucleotide sequence encoding the FRB comprises the nucleotide sequence of SEQ ID NOs: 256, 257, or 258.

In some embodiments, the FRB comprises an amino acid sequence at least 80%, 85%, 90%, 91%, 92%, 93%, 94%, 95%, 96%, 97%, 98%, 99% or 100% identical to the amino acid sequence of SEQ ID NOs: 251, 252, or 260. In some embodiments, the FRB comprises an amino acid sequence at least 100% identical to the amino acid sequence of SEQ ID NOs: 251, 252, or 260. In some embodiments, the FRB comprises the amino acid sequence of SEQ ID NOs: 251, 252, or 260.

In some embodiments, synthetic cytokine receptor complex comprises a cytosolic polypeptide that binds to the ligand or a complex comprising the ligand.

Advantageously, the cytosolic FRB may confer resistance to the immunosuppressive effect of the non-physiological ligand (e.g., rapamycin or rapalog). For example, in cells lacking the cytosolic FRB protein, rapamycin may provide an immunosuppressive effect. Cytosolic FRB present in cells transduced by certain embodiments of the lentiviral particles disclosed herein may enable the transduced cells to be resistant to the suppressive effects of rapamycin, or analogs thereof. This may be accomplished by providing a decoy binding domain for rapamycin such that rapamycin cannot engage with mTOR and induce the natural immunosuppressive effects seen in cells lacking the exogenously provided cytosolic FRB protein.

### Synthetic Cytokine Receptor

In some embodiments, an expression cassette of the polycistronic construct encodes a synthetic cytokine receptor. The synthetic cytokine receptors of the present disclosure may comprise a synthetic gamma chain and a synthetic beta chain, each comprising a dimerization domain. The dimerization domains controllable dimerize in the present of a non-physiological ligand, thereby activating signaling the synthetic cytokine receptor. For example, in some embodiments, the synthetic cytokine receptors disclosed herein may comprise an FRB domain and an FKBP12 binding domain on separate polypeptides such that the polypeptides dimerize in the presence of rapamycin or an analog thereof.

The synthetic gamma chain polypeptide comprises a first dimerization domain, a first transmembrane domain, and an interleukin-2 receptor subunit gamma (IL-2RG) intracellular domain. The dimerization domain may be extracellular (N-terminal to the transmembrane domain) or intracellular (C-terminal to the transmembrane domain and N- or C-terminal to the IL-2G intracellular domain.

The synthetic beta chain polypeptide comprises a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, or an interleukin-21 receptor subunit beta (IL-21RB) intracellular domain. The dimerization domain may be extracellular (N-terminal to the transmembrane domain) or intracellular (C-terminal to the transmembrane domain and N- or C-terminal to the IL-2RB or IL-7RB intracellular domain).

In some embodiments, the polycistronic construct provided herein comprises one or more nucleotide sequences encoding a synthetic cytokine receptor. In some embodiments, the one or more nucleotide sequences correspond to one or more expression cassettes. In some embodiments, the polynucleotide construct provided herein comprises one expression cassette encoding IL-2RG and a second expression cassette encoding IL-2RB.

In some embodiments, the synthetic gamma chain polypeptide is encoded by a nucleic acid sequence that encodes a signal peptide. In some embodiments, the synthetic beta chain polypeptide is encoded by a nucleic acid sequence that encodes a signal peptide. A skilled artisan is readily familiar with signal peptides that can provide a signal to transport a nascent protein in the cells. Any of a variety of signal peptides can be employed.

In some embodiments, the nucleotide encoding the synthetic cytokine gamma chain polypeptide is at least 80%, 85%, 90%, 91%, 92%, 93%, 94%, 95%, 96%, 97%, 98%, 99% or 100% identical to the nucleotide sequence of SEQ ID NOs: 261, 262, or 263. In some embodiments, the nucleotide encoding the synthetic cytokine gamma chain polypeptide is at least 100% identical to the nucleotide sequence of SEQ ID NOs: 261, 262, or 263. In some embodiments, the nucleotide encoding the synthetic cytokine gamma chain polypeptide comprises the nucleotide sequence of SEQ ID NOs: 261, 262, or 263.

In some embodiments, the synthetic cytokine gamma chain polypeptide comprises interleukin 2 receptor subunit γ (IL-2RG). In some embodiments, the IL-2RG comprises an amino acid sequence at least 80%, 85%, 90%, 91%, 92%, 93%, 94%, 95%, 96%, 97%, 98%, 99% or 100% identical to the amino acid sequence of SEQ ID NOs: 264 or 265. In some embodiments, the IL-2RG comprises an amino acid sequence at least 100% identical to the amino acid sequence of SEQ ID NOs: 264 or 265. In some embodiments, the IL-2RG comprises the amino acid sequence of SEQ ID NOs: 264 or 265.

In some embodiments, the second expression cassette further comprises a nucleotide sequence encoding FRB, an FRB domain, or a functional fragment thereof. In some embodiments, the nucleotide sequence encoding the FRB is at least 80%, 85%, 90%, 91%, 92%, 93%, 94%, 95%, 96%, 97%, 98%, 99% or 100% identical to the nucleotide sequence of SEQ ID NO: 257. In some embodiments, the nucleotide sequence encoding the FRB is at least 100% identical to the nucleotide sequence of SEQ ID NO: 257. In some embodiments, the nucleotide sequence encoding the FRB comprises the nucleotide sequence of SEQ ID NO: 257.

In some embodiments, the FRB comprises an amino acid sequence at least 80%, 85%, 90%, 91%, 92%, 93%, 94%, 95%, 96%, 97%, 98%, 99% or 100% identical to the amino acid sequence of SEQ ID NO: 252. In some embodiments, the FRB comprises an amino acid sequence at least 100% identical to the amino acid sequence of SEQ ID NO: 252. In some embodiments, the FRB comprises the amino acid sequence of SEQ ID NO: 252.

In some embodiments, the second expression cassette is codon optimized.

In some embodiments, the second expression cassette comprises a nucleotide sequence at least 80%, 85%, 90%, 91%, 92%, 93%, 94%, 95%, 96%, 97%, 98%, 99% or 100% identical to the nucleotide sequence of SEQ ID NO: 266. In some embodiments, the second expression cassette comprises a nucleotide sequence at least 100% identical to the nucleotide sequence of SEQ ID NO: 266. In some embodiments, the second expression cassette comprises the nucleotide sequence of SEQ ID NO: 266.

In some embodiments, the second expression cassette encodes an amino acid sequence at least 80%, 85%, 90%, 91%, 92%, 93%, 94%, 95%, 96%, 97%, 98%, 99% or 100% identical to the amino acid sequence of SEQ ID NO: 267. In some embodiments, the second expression cassette encodes an amino acid sequence at least 100% identical to the amino acid sequence of SEQ ID NO: 267. In some embodiments, the second expression cassette encodes an amino acid sequence comprising the sequence of SEQ ID NO: 267.

In some embodiments, the second expression cassette further comprises a nucleotide sequence encoding FKBP12, a FKBP12 domain, or a functional fragment thereof. FKBP12 is also known as FKBP1A or FK506 binding protein. In some embodiments, the nucleotide sequence encoding the FKBP12 is at least 80%, 85%, 90%, 91%, 92%, 93%, 94%, 95%, 96%, 97%, 98%, 99% or 100% identical to the nucleotide sequence of SEQ ID NOs: 268 or 269. In some embodiments, the nucleotide sequence encoding the FKBP12 is at least 100% identical to the nucleotide sequence of SEQ ID NOs: 268 or 269. In some embodiments, the nucleotide sequence encoding the FKBP12 comprises the nucleotide sequence of SEQ ID NOs: 268 or 269.

In some embodiments, the FKBP12 comprises an amino acid sequence at least 80%, 85%, 90%, 91%, 92%, 93%, 94%, 95%, 96%, 97%, 98%, 99% or 100% identical to the amino acid sequence of SEQ ID NO: 253. In some embodiments, the FKBP12 comprises an amino acid sequence at least 100% identical to the amino acid sequence of SEQ ID NO: 253. In some embodiments, the FKBP12 comprises the amino acid sequence of SEQ ID NO: 253.

In some embodiments, the nucleotide encoding the synthetic cytokine beta chain polypeptide is at least 80%, 85%, 90%, 91%, 92%, 93%, 94%, 95%, 96%, 97%, 98%, 99% or 100% identical to the nucleotide sequence of SEQ ID NOs: 270 or 271. In some embodiments, the nucleotide encoding the synthetic cytokine beta chain polypeptide is at least 100% identical to the nucleotide sequence of SEQ ID NOs: 270 or 271. In some embodiments, the nucleotide encoding the synthetic cytokine beta chain polypeptide comprises the nucleotide sequence of SEQ ID NOs: 270 or 271.

In some embodiments, the synthetic cytokine beta chain polypeptide comprises interleukin 2 receptor subunit β (IL-2RB).

In some embodiments, the IL-2RB comprises an amino acid sequence at least 80%, 85%, 90%, 91%, 92%, 93%, 94%, 95%, 96%, 97%, 98%, 99% or 100% identical to the amino acid sequence of SEQ ID NOs: 272 or 273. In some embodiments, the IL-2RB comprises an amino acid sequence at least 100% identical to the amino acid sequence of SEQ ID NOs: 272 or 273. In some embodiments, the IL-2RB comprises the amino acid sequence of SEQ ID NOs: 272 or 273.

In some embodiments, the third expression cassette further comprises a nucleotide sequence encoding FKBP12.

In some embodiments, the nucleotide sequence encoding the FKBP12 is at least 80%, 85%, 90%, 91%, 92%, 93%, 94%, 95%, 96%, 97%, 98%, 99% or 100% identical to the nucleotide sequence of SEQ ID NO: 274 In some embodiments, the nucleotide sequence encoding the FKBP12 is at least 100% identical to the nucleotide sequence of SEQ ID NO: 274. In some embodiments, the nucleotide sequence encoding the FKBP12 comprises the nucleotide sequence of SEQ ID NO: 274.

In some embodiments, the FKBP12 comprises an amino acid sequence at least 80%, 85%, 90%, 91%, 92%, 93%, 94%, 95%, 96%, 97%, 98%, 99% or 100% identical to the amino acid sequence of SEQ ID NO: 275. In some embodiments, the FKBP12 comprises an amino acid sequence at least 100% identical to the amino acid sequence of SEQ ID NO: 275. In some embodiments, the FKBP12 comprises the amino acid sequence of SEQ ID NO: 275.

In some embodiments, the third expression cassette is codon optimized.

In some embodiments, the third expression cassette comprises a nucleotide sequence at least 80%, 85%, 90%, 91%, 92%, 93%, 94%, 95%, 96%, 97%, 98%, 99% or 100% identical to the nucleotide sequence of SEQ ID NO: 276. In some embodiments, the third expression cassette comprises a nucleotide sequence at least 100% identical to the nucleotide sequence of SEQ ID NO: 276. In some embodiments, the third expression cassette comprises the nucleotide sequence of SEQ ID NO: 276.

In some embodiments, the third expression cassette encodes an amino acid sequence at least 80%, 85%, 90%, 91%, 92%, 93%, 94%, 95%, 96%, 97%, 98%, 99% or 100% identical to the amino acid sequence of SEQ ID NO: 277. In some embodiments, the third expression cassette encodes an amino acid sequence at least 100% identical to the amino acid sequence of SEQ ID NO: 277. In some embodiments, the third expression cassette encodes an amino acid sequence comprising the sequence of SEQ ID NO: 277.

In some embodiments, the third expression cassette further comprises a nucleotide sequence encoding FRB. In some embodiments, the nucleotide sequence encoding the FRB is at least 80%, 85%, 90%, 91%, 92%, 93%, 94%, 95%, 96%, 97%, 98%, 99% or 100% identical to the nucleotide sequence of SEQ ID NO: 257. In some embodiments, the nucleotide sequence encoding the FRB is at least 100% identical to the nucleotide sequence of SEQ ID NO: 275. In some embodiments, the nucleotide sequence encoding the FRB comprises the nucleotide sequence of SEQ ID NO: 257.

### Intracellular Domain

In some embodiments, the intracellular signaling domain of the first transmembrane receptor protein comprises an interleukin-2 receptor subunit gamma (IL-2RG) domain.

In some embodiments, the synthetic cytokine receptor comprises a first transmembrane receptor protein comprising an IL-2RG intracellular domain, a first dimerization domain, a second transmembrane receptor protein comprising an IL-2RB intracellular domain, and a second dimerization domain.

In some embodiments, the synthetic beta chain comprises an interleukin-2 receptor subunit beta (IL-2RB) intracellular domain. IL-2RB is also known as IL-15RB or CD122. Thus, when referred to herein, IL-2RB can also mean IL-15RB. That is, the terms are used interchangeably in the present disclosure.

In some embodiments, the synthetic cytokine receptor comprises a first transmembrane receptor protein comprising an IL-2RG intracellular domain, a first dimerization domain, a second transmembrane receptor protein comprising an IL-7RB intracellular domain, and a second dimerization domain.

In some embodiments, the synthetic beta chain comprises an interleukin-7 receptor subunit beta (IL-7RB) intracellular domain.

In some embodiments, the synthetic cytokine receptor comprises a first transmembrane receptor protein comprising an IL-2RG intracellular domain, a first dimerization domain, a second transmembrane receptor protein comprising an IL-21RB intracellular domain, and a second dimerization domain.

In some embodiments, the synthetic beta chain comprises an interleukin-21 receptor subunit beta (IL-21RB) intracellular domain.

### Dimerization Domain

The dimerization domains may be heterodimerization domains, including but not limited to FK506-Binding Protein of size 12 kD (FKBP) and a FKBP12-rapamycin binding (FRB) domain, which dimerize in the presence of rapamycin or a rapalog.

Alternatively, the first dimerization domain and the second dimerization domain may be a FK506-Binding Protein of size 12 kD (FKBP) and a calcineurin domain, which dimerize in the presence of FK506 or an analogue thereof.

In some embodiments the dimerization domains are homodimerization domains selected from:
i) FK506-Binding Protein of size 12 kD (FKBP);
ii) cyclophiliA (CypA); or
iii) gyrase B (CyrB);
   with the corresponding non-physiological ligands being, respectively
i) FK1012, AP1510, AP1903, or AP20187;
ii) cyclosporin-A (CsA); or
iii) coumermycin or analogs thereof.

In some embodiments, the first and second dimerization domains of the transmembrane receptor proteins are a FKBP domain and a cyclophilin domain.

In some embodiments, the first and second dimerization domains of the transmembrane receptor proteins are a FKBP domain and a bacterial dihydrofolate reductase (DHFR) domain.

In some embodiments, the first and second dimerization domains of the transmembrane receptor proteins are a calcineurin domain and a cyclophilin domain.

In some embodiments, the first and second dimerization domains of the transmembrane receptor proteins are PYR1-like 1 (PYL1) and abscisic acid insensitive 1 (ABI1).

### Transmembrane Domain

The transmembrane domain is the sequence of the synthetic cytokine receptor that spans the membrane. The transmembrane domain may comprise a hydrophobic alpha helix. In some embodiments, the transmembrane domain is a human protein.

In some embodiments, the TM domain and the intracellular signaling domain are from the same cytokine receptor. In some embodiments, the synthetic gamma chain polypeptide contains an IL-2RG TM domain and an IL-2RG intracellular domain. In some embodiments, the synthetic beta chain polypeptide contains an IL-2RB TM domain and an IL-2RB intracellular domain. In some embodiments, the synthetic beta chain polypeptide contains an IL-7RB TM domain and an IL-7RB intracellular domain. In some embodiments, the synthetic beta chain polypeptide contains an IL-21RB TM domain and an IL-21RB intracellular domain.

In some embodiments, one or more additional contiguous amino acids of the ectodomain directly adjacent to the TM domain of the cytokine receptor also can be included as part of the polypeptide sequence of a chain of the synthetic cytokine receptor. In some embodiments, 1-20 contiguous amino acids of the ectodomain adjacent to the TM domain of the cytokine receptor is included as part of the polypeptide sequence of a chain of the synthetic cytokine receptor. The portion of the ectodomain may be a contiguous sequence of 1, 2, 3, 4, 5, 6, 7, 8, 9, 10, 11, 12, 13, 14, 15, 16, 17, 18, 19 or 20 amino acids directly adjacent (e.g. N-terminal to) the TM sequence.

In some embodiments, the synthetic cytokine receptor is able to be bound by the non-physiological ligand rapamycin or a rapamycin analog. In some embodiments, the synthetic cytokine receptor is responsive to the non-physiological ligand rapamycin or a rapamycin analog, in which binding of the non-physiological ligand to the dimerization domains of the synthetic cytokine receptor induces cytokine receptor-mediated signaling in the cell, such as via the JAK/STAT pathway.

### Illustrative Polycistronic Constructs

In some embodiments, the polycistronic construct comprises in 5′ to 3′ order a nucleotide sequence encoding FRB, a nucleotide sequence encoding a synthetic cytokine polypeptide, and a nucleotide sequence encoding a CAR. In some embodiments, the nucleotide sequence encoding the synthetic cytokine polypeptide comprises in 5′ to 3′ order a first nucleotide sequence encoding FRB operably linked to IL-2RG and a second nucleotide sequence encoding FKBP12 operably linked to IL-2RB. In some embodiments, the nucleotide sequence encoding the synthetic cytokine polypeptide comprises in 5′ to 3′ order a first nucleotide sequence encoding FKBP12 operably linked to IL-2RG and a second nucleotide sequence encoding sFRB operably linked to IL-2RB.

In some embodiments, the lentiviral particles of the present disclosure comprise a polynucleotide sequence encoding, in 5′ to 3′ order on a polycistronic transcript:
(a) a MND promoter;
(b) a CAR;
(c) a cytosolic FRB domain or a portion thereof;
(d) a RACR cell-surface receptor; and
(e) a WPRE sequence

In some embodiments, the lentiviral particles of the present disclosure comprise a polynucleotide sequence encoding, in 5′ to 3′ order:
(a) a CAR;
(b) a cytosolic FRB domain or a portion thereof, and
(c) a RACR cell-surface receptor.

In some embodiments, the lentiviral particle comprises a nucleic acid sequence that shares at least 75%, at least 80%, at least 85%, at least 90%, at least 95%, at least 99%, or 100% identity to SEQ ID) NO: 119.

(SEQ ID NO: 119)
GAACAGAGAAACAGGAGAATATGGGCCAAACAGGATATCTGTGGT
AAGCAGTTCCTGCCCCGGCTCAGGGCCAAGAACAGTTGGAACAGC
AGAATATGGGCCAAACAGGATATCTGTGGTAAGCAGTTCCTGCCC
CGGCTCAGGGCCAAGAACAGATGGTCCCCAGATGCGGTCCCGCCC
TCAGCAGTTTCTAGAGAACCATCAGATGTTTCCAGGGTGCCCCAA
GGACCTGAAATGACCCTGTGCCTTATTTGAACTAACCAATCAGTT
CGCTTCTCGCTTCTGTTCGCGCGCTTCTGCTCCCCGAGCTCTATA
TAAGCAGAGCTCGTTTAGTGAACCGTCAGATCGCTAGC

In some embodiments, the lentiviral particle comprises a polypeptide sequence that shares at least 75%, at least 80%, at least 85%, at least 90%, at least 95%, at least 99%, or 100% identity to SEQ ID NO: 120.

(SEQ ID NO: 120)
MLLLVTSLLLCELPHPAFLLIPDIQMTQTTSSLSASLGDRVTISC
RASQDISKYLNWYQQKPDGTVKLLIYHTSRLHSGVPSRFSGSGSG
TDYSLTISNLEQEDIATYFCQQGNTLPYTFGGGTKLEITGSTSGS
GKPGSGEGSTKGEVKLQESGPGLVAPSQSLSVTCTVSGVSLPDYG
VSWIRQPPRKGLEWLGVIWGSETTYYNSALKSRLTIIKDNSKSQV
FLKMNSLQTDDTAIYYCAKHYYYGGSYAMDYWGQGTSVTVSSESK
YGPPCPPCPMFWVLVVVGGVLACYSLLVTVAFIIFWVKRGRKKLL
YIFKQPFMRPVQTTQEEDGCCRFPEEEEGGCELRVKFSRSADAPA
YQQGQNQLYNELNLGRREEYDVLDKRRGRDPEMGGKPRRKNPQEG
LYNELQKDKMAEAYSEIGMKGERRRGKGHDGLYQGLSTATKDTYD
ALHMQALPPRGSGATNFSLLKQAGDVEENPGPEMWHEGLEEASRL
YFGERNVKGMFEVLEPLHAMMERGPQTLKETSFNQAYGRDLMEAQ
EWCRKYMKSGNVKDLLQAWDLYYHVFRRISKGSGATNFSLLKQAG
DVEENPGPMPLGLLWLGLALLGALHAQAGVQVETISPGDGRTFPK
RGQTCVVHYTGMLEDGKKFDSSRDRNKPFKFMLGKQEVIRGWEEG
VAQMSVGQRAKLTISPDYAYGATGHPGIIPPHATLVFDVELLKLG
EGSNTSKENPFLFALEAVVISVGSMGLIISLLCVYFWLERTMPRI
PTLKNLEDLVTEYHGNFSAWSGVSKGLAESLQPDYSERLCLVSEI
PPKGGALGEGPGASPCNQHSPYWAPPCYTLKPETGSGATNFSLLK
QAGDVEENPGPMALPVTALLLPLALLLHAARPILWHEMWHEGLEE
ASRLYFGERNVKGMFEVLEPLHAMMERGPQTLKETSFNQAYGRDL
MEAQEWCRKYMKSGNVKDLLQAWDLYYHVFRRISKGKDTIPWLGH
LLVGLSGAFGFIILVYLLINCRNTGPWLKKVLKCNTPDPSKFFSQ
LSSEHGGDVQKWLSSPFPSSSFSPGGLAPEISPLEVLERDKVTQL
LLQQDKVPEPASLSSNHSLTSCFTNQGYFFFHLPDALEIEACQVY
FTYDPYSEEDPDEGVAGAPTGSSPQPLQPLSGEDDAYCTFPSRDD
LLLFSPSLLGGPSPPSTAPGGSGAGEERMPPSLQERVPRDWDPQP
LGPPTPGVPDLVDFQPPPELVLREAGEEVPDAGPREGVSFPWSRP
PGQGEFRALNARLPLNTDAYLSLQELQGQDPTHLV

In some embodiments, the lentiviral particle comprises a nucleic acid sequence that shares at least 75%, at least 80%, at least 85%, at least 90%, at least 95%, at least 99%, or 100% identity to SEQ ID NO: 121.

In some embodiments, the lentiviral particles of the present disclosure comprises a polynucleotide sequence encoding, in 5′ to 3′ order on a polycistronic transcript:
(a) a MND promoter;
(b) a cytosolic FRB domain or a portion thereof;
(c) a RACR cell-surface receptor;
(d) a CAR; and
(e) a WPRE sequence.

In some embodiments, the lentiviral particles of the present disclosure comprises a polynucleotide sequence encoding, in 5′ to 3′ order:
(a) a cytosolic FRB domain or a portion thereof;
(b) a RACR cell-surface receptor; and
(c) a CAR.

In some embodiments, the lentiviral particle comprises a polypeptide sequence that shares at least 75%, at least 80%, at least 85%, at least 90%, at least 95%, at least 99%, or 100% identity to SEQ ID NO: 122.

(SEQ ID NO: 122)
MEMWHEGLEEASRLYFGERNVKGMFEVLEPLHAMMERGPQTLKET
SFNQAYGRDLMEAQEWCRKYMKSGNVKDLLQAWDLYYHVFRRISK
GSGATNFSLLKQAGDVEENPGPMPLGLLWLGLALLGALHAQAGVQ
VETISPGDGRTFPKRGQTCVVHYTGMLEDGKKFDSSRDRNKPFKF
MLGKQEVIRGWEEGVAQMSVGQRAKLTISPDYAYGATGHPGIIPP
HATLVFDVELLKLGEGSNTSKENPFLFALEAVVISVGSMGLIISL
LCVYFWLERTMPRIPTLKNLEDLVTEYHGNFSAWSGVSKGLAESL
QPDYSERLCLVSEIPPKGGALGEGPGASPCNQHSPYWAPPCYTLK
PETGSGATNFSLLKQAGDVEENPGPMALPVTALLLPLALLLHAAR
PILWHEMWHEGLEEASRLYFGERNVKGMFEVLEPLHAMMERGPQT
LKETSFNQAYGRDLMEAQEWCRKYMKSGNVKDLLQAWDLYYHVFR
RISKGKDTIPWLGHLLVGLSGAFGFIILVYLLINCRNTGPWLKKV
LKCNTPDPSKFFSQLSSEHGGDVQKWLSSPFPSSSFSPGGLAPEI
SPLEVLERDKVTQLLLQQDKVPEPASLSSNHSLTSCFTNQGYFFF
HLPDALEIEACQVYFTYDPYSEEDPDEGVAGAPTGSSPQPLQPLS
GEDDAYCTFPSRDDLLLFSPSLLGGPSPPSTAPGGSGAGEERMPP
SLQERVPRDWDPQPLGPPTPGVPDLVDFQPPPELVLREAGEEVPD
AGPREGVSFPWSRPPGQGEFRALNARLPLNTDAYLSLQELQGQDP
THLVGSGATNFSLLKQAGDVEENPGPMLLLVTSLLLCELPHPAFL
LIPDIQMTQTTSSLSASLGDRVTISCRASQDISKYLNWYQQKPDG
TVKLLIYHTSRLHSGVPSRFSGSGSGTDYSLTISNLEQEDIATYF
CQQGNTLPYTFGGGTKLEITGSTSGSGKPGSGEGSTKGEVKLQES
GPGLVAPSQSLSVTCTVSGVSLPDYGVSWIRQPPRKGLEWLGVIW
GSETTYYNSALKSRLTIIKDNSKSQVFLKMNSLQTDDTAIYYCAK
HYYYGGSYAMDYWGQGTSVTVSSESKYGPPCPPCPMFWVLVVVGG
VLACYSLLVTVAFIIFWVKRGRKKLLYIFKQPFMRPVQTTQEEDG
CSCRFPEEEEGGCELRVKFSRSADAPAYQQGQNQLYNELNLGRRE
EYDVLDKRRGRDPEMGGKPRRKNPQEGLYNELQKDKMAEAYSEIG
MKGERRRGKGHDGLYQGLSTATKDTYDALHMQALPPR

In some embodiments, the lentiviral particle comprises a nucleic acid sequence that shares at least 75%, at least 80%, at least 85%, at least 90%, at least 95%, at least 99%, or 100% identity to SEQ ID NO: 123.

In some embodiments, the lentiviral particles of the present disclosure comprise a polynucleotide sequence encoding, in 5′ to 3′ order on a polycistronic transcript:
(a) a MND promoter;
(b) a cytosolic FRB domain or a portion thereof;
(c) a CAR;
(d) TGF-β DN domain or portion thereof, and
(e) a WPRE sequence.

In some embodiments, the lentiviral particles of the present disclosure comprise a polynucleotide sequence encoding, in 5′ to 3′ order:
(a) a cytosolic FRB domain or a portion thereof;
(b) a CAR; and
(c) a TGF-β DN domain or portion thereof.

In some embodiments, the lentiviral particle comprises a polypeptide sequence that shares at least 75%, at least 80%, at least 85%, at least 90%, at least 95%, at least 99%, or 100% identity to SEQ ID NO: 124.

(SEQ ID NO: 124)
MEMWHEGLEEASRLYFGERNVKGMFEVLEPLHAMMERGPQTLKET
SFNQAYGRDLMEAQEWCRKYMKSGNVKDLLQAWDLYYHVFRRISK
GSGATNFSLLKQAGDVEENPGPMLLLVTSLLLCELPHPAFLLIPD
IQMTQTTSSLSASLGDRVTISCRASQDISKYLNWYQQKPDGTVKL
LIYHTSRLHSGVPSRFSGSGSGTDYSLTISNLEQEDIATYFCQQG
NTLPYTFGGGTKLEITGSTSGSGKPGSGEGSTKGEVKLQESGPGL
VAPSQSLSVTCTVSGVSLPDYGVSWIRQPPRKGLEWLGVIWGSET
TYYNSALKSRLTIIKDNSKSQVFLKMNSLQTDDTAIYYCAKHYYY
GGSYAMDYWGQGTSVTVSSESKYGPPCPPCPMFWVLVVVGGVLAC
YSLLVTVAFIIFWVKRGRKKLLYIFKQPFMRPVQTTQEEDGCSCR
FPEEEEGGCELRVKFSRSADAPAYQQGQNQLYNELNLGRREEYDV
LDKRRGRDPEMGGKPRRKNPQEGLYNELQKDKMAEAYSEIGMKGE
RRRGKGHDGLYQGLSTATKDTYDALHMQALPPRGSGATNFSLLKQ
AGDVEENPGPMGRGLLRGLWPLHIVLWTRIASTIPPHVQKSVNND
MIVTDNNGAVKFPQLCKFCDVRFSTCDNQKSCMSNCSITSICEKP
QEVCVAVWRKNDENITLETVCHDPKLPYHDFILEDAASPKCIMKE
KKKPGETFFMCSCSSDECNDNIIFSEEYNTSNPDLLLVIFQVTGI
SLLPPLGVAISVIIIFYCYRVNRQQKRRR

In some embodiments, the lentiviral particle comprises a nucleic acid sequence that shares at least 75%, at least 80%, at least 85%, at least 90%, at least 95%, at least 99%, or 100% identity to SEQ ID NO: 125.

### (SEQ ID NO: 125)

In some embodiments, the FRB domain comprises a polypeptide sequence that shares at least 75%, at least 80%, at least 85%, at least 90%, at least 91%, at least 92%, at least 93%, at least 94%, at least 95%, at least 99%, or 100% identity to SEQ ID NO: 251.

(SEQ ID NO: 251)
MEMWHEGLEEASRLYFGERNVKGMFEVLEPLHAMMERGPQTLKET
SFNQAYGRDLMEAQEWCRKYMKSGNVKDLLQAWDLYYHVFRRISK

In some embodiments, the IL-2 Receptor gamma domain comprises a polypeptide sequence that shares at least 75%, at least 80%, at least 85%, at least 90%, at least 91%, at least 92%, at least 93%, at least 94%, at least 95%, at least 99%, or 100% identity to SEQ ID NO: 252.

(SEQ ID NO: 252)
ILWHEMWHEGLEEASRLYFGERNVKGMFEVLEPLHAMMERGPQTL
KETSFNQAYGRDLMEAQEWCRKYMKSGNVKDLLQAWDLYYHVFRR
ISK

In some embodiments, the IL-2 Receptor beta domain comprises a polypeptide sequence that shares at least 75%, at least 80%, at least 85%, at least 90%, at least 91%, at least 92%, at least 93%, at least 94%, at least 95%, at least 99%, or 100% identity to SEQ ID NO: 253.

(SEQ ID NO: 253)
GVQVETISPGDGRTFPKRGQTCVVHYTGMLEDGKKFDSSRDRNKP
FKFMLGKQEVIRGWEEGVAQMSVGQRAKLTISPDYAYGATGHPGI
IPPHATLVFDVELLKL

In some embodiments, the Rapamycin-Activated Cell-Surface Receptor (RACR) and FRB domain complex comprises a polypeptide sequence that shares at least 75%, at least 80%, at least 85%, at least 90%, at least 91%, at least 92%, at least 93%, at least 94%, at least 95%, at least 99%, or 100% identity to SEQ ID NO: 254.

(SEQ ID NO: 254)
MEMWHEGLEEASRLYFGERNVKGMFEVLEPLHAMMERGPQTLKET
SFNQAYGRDLMEAQEWCRKYMKSGNVKDLLQAWDLYYHVFRRISK
ASRRKRGSGEGRGSLLTCGDVEENPGPMPLPVTALLLPLALLLHA
ARPILWHEMWHEGLEEASRLYFGERNVKGMFEVLEPLHAMMERGP
QTLKETSFNQAYGRDLMEAQEWCRKYMKSGNVKDLLQAWDLYYHV
FRRISKGSNTSKENPFLFALEAVVISVGSMGLIISLLCVYFWLER
TMPRIPTLKNLEDLVTEYHGNFSAWSGVSKGLAESLQPDYSERLC
LVSEIPPKGGALGEGPGASPCNQHSPYWAPPCYTLKPETGSGATN
FSLLKQAGDVEENPGPMPLGLLWLGLALLGALHAQAGVQVETISP
GDGRTFPKRGQTCVVHYTGMLEDGKKFDSSRDRNKPFKFMLGKQE
VIRGWEEGVAQMSVGQRAKLTISPDYAYGATGHPGIIPPHATLVF
DVELLKLGEGKDTIPWLGHLLVGLSGAFGFIILVYLLINCRNTGP
WLKKVLKCNTPDPSKFFSQLSSEHGGDVQKWLSSPFPSSSFSPGG
LAPEISPLEVLERDKVTQLLLQQDKVPEPASLSSNHSLTSCFTNQ
GYFFFHLPDALEIEACQVYFTYDPYSEEDPDEGVAGAPTGSSPQP
LQPLSGEDDAYCTFPSRDDLLLFSPSLLGGPSPPSTAPGGSGAGE
ERMPPSLQERVPRDWDPQPLGPPTPGVPDLVDFQPPPELVLREAG
EEVPDAGPREGVSFPWSRPPGQGEFRALNARLPLNTDAYLSLQEL
QGQDPTHLV

In some embodiments, the Rapamycin-Activated Cell-Surface Receptor (RACR) and FRB domain complex and anti-CD19 CAR comprises a polypeptide sequence that shares at least 75%, at least 80%, at least 85%, at least 90%, at least 91%, at least 92%, at least 93%, at least 94%, at least 95%, at least 99%, or 100% identity to SEQ ID NO: 255.

(SEQ ID NO: 255)
MEMWHEGLEEASRLYFGERNVKGMFEVLEPLHAMMERGPQTLKET
SFNQAYGRDLMEAQEWCRKYMKSGNVKDLLQAWDLYYHVFRRISK
ASRRKRGSGEGRGSLLTCGDVEENPGPMPLPVTALLLPLALLLHA
ARPILWHEMWHEGLEEASRLYFGERNVKGMFEVLEPLHAMMERGP
QTLKETSFNQAYGRDLMEAQEWCRKYMKSGNVKDLLQAWDLYYHV
FRRISKGSNTSKENPFLFALEAVVISVGSMGLIISLLCVYFWLER
TMPRIPTLKNLEDLVTEYHGNFSAWSGVSKGLAESLQPDYSERLC
LVSEIPPKGGALGEGPGASPCNQHSPYWAPPCYTLKPETGSGATN
FSLLKQAGDVEENPGPMPLGLLWLGLALLGALHAQAGVQVETISP
GDGRTFPKRGQTCVVHYTGMLEDGKKFDSSRDRNKPFKFMLGKQE
VIRGWEEGVAQMSVGQRAKLTISPDYAYGATGHPGIIPPHATLVF
DVELLKLGEGKDTIPWLGHLLVGLSGAFGFIILVYLLINCRNTGP
WLKKVLKCNTPDPSKFFSQLSSEHGGDVQKWLSSPFPSSSFSPGG
LAPEISPLEVLERDKVTQLLLQQDKVPEPASLSSNHSLTSCFTNQ
GYFFFHLPDALEIEACQVYFTYDPYSEEDPDEGVAGAPTGSSPQP
LQPLSGEDDAYCTFPSRDDLLLFSPSLLGGPSPPSTAPGGSGAGE
ERMPPSLQERVPRDWDPQPLGPPTPGVPDLVDFQPPPELVLREAG
EEVPDAGPREGVSFPWSRPPGQGEFRALNARLPLNTDAYLSLQEL
QGQDPTHLVGSGATNFSLLKQAGDVEENPGPLLLVTSLLLCELPH
PAFLLIPDIQMTQTTSSLSASLGDRVTISCRASQDISKYLNWYQQ
KPDGTVKLLIYHTSRLHSGVPSRFSGSGSGTDYSLTISNLEQEDI
ATYFCQQGNTLPYTFGGGTKLEITGSTSGSGKPGSGEGSTKGEVK
LQESGPGLVAPSQSLSVTCTVSGVSLPDYGVSWIRQPPRKGLEWL
GVIWGSETTYYNSALKSRLTIIKDNSKSQVFLKMNSLQTDDTAIY
YCAKHYYYGGSYAMDYWGQGTSVTVSSGAVHTRGLDFACDFWVLV
VVGGVLACYSLLVTVAFIIFWVKRGRKKLLYIFKQPFMRPVQTTQ
EEDGCSCRFPEEEEGGCELRVKFSRSADAPAYQQGQNQLYNELNL
GRREEYDVLDKRRGRDPEMGGKPRRKNPQEGLYNELQKDKMAEAY
SEIGMKGERRRGKGHDGLYQGLSTATKDTYDALHMQALPPR

### Pharmaceutical Compositions and Kits

In some embodiments, the disclosure provides a pharmaceutical composition comprising a particle according to the disclosure and a pharmaceutically acceptable carrier.

In some embodiments, the disclosure provides a kit comprising the particle and instructions for use in transduction of target cells and/or treatment of a subject. The kit may include a pharmaceutically acceptable carrier and/or an injection device. The kit may further include suitable tubing for administering the particles.

Disclosed herein, in some embodiments are pharmaceutical compositions comprising a lentiviral particle that includes a polynucleotide encoding an anti-CD20 CAR, and a pharmaceutically acceptable carrier.

### Formulations

The formulations and compositions of the present disclosure may comprise a combination of any number of viral particles, and optionally one or more additional pharmaceutical agents (polypeptides, polynucleotides, compounds etc.) formulated in pharmaceutically acceptable or physiologically-acceptable compositions for administration to a cell, tissue, organ, or an animal, either alone, or in combination with one or more other modalities of therapy. In some embodiments, the one or more additional pharmaceutical agents further increases transduction efficiency of viral particles.

In some embodiments, the formulations and compositions of the present disclosure may comprise a combination of any number of viral particles, and optionally one or more nanocarriers. Illustrative nanocarriers include, but are not limited to, micelles, polymers, liposomes, and lipid nanoparticles (LNPs).

In some embodiments, the present disclosure provides compositions comprising a therapeutically-effective amount of a viral particle, as described herein, formulated together with one or more pharmaceutically acceptable carriers (additives) and/or diluents. In some embodiments, the composition further comprises other agents, such as, e.g., cytokines, growth factors, hormones, small molecules or various pharmaceutically active agents.

In some embodiments, compositions and formulations of the viral particles used in accordance with the present disclosure may be prepared for storage by mixing a viral particle having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed. In some embodiments, one or more pharmaceutically acceptable surface-active agents (surfactant), buffers, isotonicity agents, salts, amino acids, sugars, stabilizers and/or antioxidant are used in the formulation.

Suitable pharmaceutically acceptable surfactants comprise but are not limited to polyethylen-sorbitan-fatty acid esters, polyethylene-polypropylene glycols, polyoxyethylene-stearates and sodium dodecyl sulphates. Suitable buffers comprise but are not limited to histidine-buffers, citrate-buffers, succinate-buffers, acetate-buffers and phosphate-buffers.

Isotonicity agents are used to provide an isotonic formulation. An isotonic formulation is liquid, or liquid reconstituted from a solid form, e.g. a lyophilized form and denotes a solution having the same tonicity as some other solution with which it is compared, such as physiologic salt solution and the blood serum. Suitable isotonicity agents comprise but are not limited to salts, including but not limited to sodium chloride (NaCl) or potassium chloride, sugars including but not limited to glucose, sucrose, trehalose or and any component from the group of amino acids, sugars, salts and combinations thereof. In some embodiments, isotonicity agents are generally used in a total amount of about 5 mM to about 350 mM.

Non-limiting examples of salts include salts of any combinations of the cations sodium potassium, calcium or magnesium with anions chloride, phosphate, citrate, succinate, sulphate or mixtures thereof. Non-limiting examples of amino acids comprise arginine, glycine, ornithine, lysine, histidine, glutamic acid, aspartic acid, isoleucine, leucine, alanine, phenylalanine, tyrosine, tryptophan, methionine, serine, or proline. Non-limiting examples of sugars according to the invention include trehalose, sucrose, mannitol, sorbitol, lactose, glucose, mannose, maltose, galactose, fructose, sorbose, raffinose, glucosamine, N-methylglucosamine (also referred to as “meglumine”), galactosamine and neuraminic acid and combinations thereof. Non-limiting examples of stabilizer includes amino acids and sugars as described above as well as commercially available cyclodextrins and dextrans of any useful kind and molecular weight. Non-limiting examples of antioxidants include excipients such as methionine, benzylalcohol or any other excipient used to minimize oxidation.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. Except insofar as any media or agent is incompatible with the active ingredient, its use in the therapeutic compositions is contemplated. Supplementary active ingredients can also be incorporated into the compositions.

As used herein “pharmaceutically acceptable carrier” includes solvents, dispersion media, coatings, antibacterial and antifungal agents, isotonic and absorption delaying agents, and the like that are physiologically compatible, including pharmaceutically acceptable cell culture media. In some embodiments, a composition comprising a carrier is suitable for parenteral administration, e.g., intravascular (intravenous or intra-arterial), intraperitoneal or intramuscular administration. Pharmaceutically acceptable carriers may include sterile aqueous solutions or dispersions and sterile powders for the extemporaneous preparation of sterile injectable solutions or dispersion. Except insofar as any media or agent is incompatible with the transduced cells, use thereof in the pharmaceutical compositions of the present disclosure is contemplated.

The compositions may further comprise one or more polypeptides, polynucleotides, vector genomes comprising same, compounds that increase the transduction efficiency of vector genomes, formulated in pharmaceutically acceptable or physiologically-acceptable solutions for administration to a cell or an animal, either alone, or in combination with one or more other modalities of therapy. It will also be understood that, if desired, the compositions of the present disclosure may be administered in combination with other agents as well, such as, e.g., cytokines, growth factors, hormones, small molecules or various pharmaceutically active agents. There is virtually no limit to other components that may also be included in the compositions, provided that the additional agents do not adversely affect the ability of the composition to deliver the intended therapy.

The present disclosure also provides pharmaceutical compositions comprising an expression cassette or vector (e.g., therapeutic vector) disclosed herein and one or more pharmaceutically acceptable carriers, diluents or excipients. In some embodiments, the pharmaceutical composition comprises a lentiviral vector comprising an expression cassette disclosed herein, e.g., wherein the expression cassette comprises one or more polynucleotide sequences encoding one or more chimeric antigen receptor (CARs) and variants thereof.

The pharmaceutical compositions that contain the expression cassette or vector genome may be in any form that is suitable for the selected mode of administration, for example, for intraventricular, intramyocardial, intracoronary, intravenous, intra-arterial, intra-renal, intraurethral, epidural, intrathecal, intraperitoneal, or intramuscular administration. The vector genome can be administered, as sole active agent, or in combination with other active agents, in a unit administration form, as a mixture with pharmaceutical supports, to animals and human beings. In some embodiments, the pharmaceutical composition comprises cells transduced ex vivo with any of the vector genomes according to the present disclosure.

In some embodiments, the viral particle (e.g., lentiviral particle), or a pharmaceutical composition comprising that viral particle, is effective when administered systemically. For example, the viral vectors of the disclosure, in some cases, may be administered intravenously to subject (e.g., a primate, such as a non-human primate or a human). In some embodiments, the viral vectors of the disclosure are capable of inducing expression of CAR in various immune cells when administered systemically (e.g., in T-cells, dendritic cells, NK cells).

In various embodiments, the pharmaceutical compositions contain vehicles (e.g., carriers, diluents and excipients) that are pharmaceutically acceptable for a formulation capable of being injected. Illustrative excipients include a poloxamer. Formulation buffers for viral vectors may contain salts to prevent aggregation and other excipients (e.g., poloxamer) to reduce stickiness of the viral particle. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. In some embodiments, the formulation is stable for storage and use when frozen (e.g., at less than 0° C., about −60° C., or about −72° C.). In some embodiments, the formulation is a cryopreserved solution.

The pharmaceutical compositions of the present disclosure, formulation of pharmaceutically acceptable excipients and carrier solutions may be useful to those of skill in the art, such as for development of suitable dosing and treatment regimens for using the particular compositions described herein in a variety of treatment regimens, including e.g., oral, parenteral, intravenous, intranasal, intraperitoneal, and intramuscular administration and formulation.

In certain circumstances, it may be desirable to deliver the compositions disclosed herein parenterally, intravenously, intramuscularly, or intraperitoneally, for example, in U.S. Pat. Nos. 5,543,158; 5,641,515 and 5,399,363 (each incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, incorporated herein by reference in its entirety). In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Fluidity may be maintained, for example, by use of a coating, such as lecithin, by maintenance of a useful particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In some embodiments, isotonic agents, for example, sugars or sodium chloride, are added. Prolonged absorption of the injectable compositions can be brought about by the use in the compositions of agents delaying absorption, for example, aluminum monostearate and gelatin.

For parenteral administration in an aqueous solution, for example, the solution should be suitably buffered if useful or necessary and the liquid diluent first rendered isotonic with sufficient saline or glucose. These particular aqueous solutions are especially suitable for intravenous, intramuscular, subcutaneous and intraperitoneal administration. In some embodiments, the solution intended for subcutaneous administration includes hyaluronidase. In this connection, a sterile aqueous medium that can be employed may be useful. One dosage may be dissolved in 1 ml of isotonic NaCl solution and either added to 1000 ml of hypodermoclysis fluid or injected at the proposed site of infusion (see, e.g., Remington: The Science and Practice of Pharmacy, 20th Edition. Baltimore, Md.: Lippincott Williams & Wilkins, 2005). Some variation in dosage may occur depending on the condition of the subject being treated. The person responsible for administration will, in any event, determine the appropriate dose for the individual subject. In some embodiments, for human administration, preparations should meet sterility, pyrogenicity, and the general safety and purity standards set by the FDA Office of Biologics standards.

In some embodiments, the present disclosure provides formulations or compositions suitable for the delivery of viral vector systems (i.e., viral-mediated transduction) including, but not limited to, retroviral (e.g., lentiviral) vectors.

### Method of Use In Vitro or Ex Vivo

The compositions described herein such as fusion proteins or particles described may be used in vitro or ex vivo. The lentiviral particles described may be used ex vivo, in a cell manufacturing process or at a bedside as described, e.g., in Int'l Pat. Pub. No. WO 2022/072885, Int'l Pat. Pub. No. 2019/217954, Int'l Pat. Pub. No. 2020/123649, and Int'l Pat. Pub. No. 2009/072003. In some embodiments, the disclosure provides an ex vivo method of transducing target cells, comprising contacting the target cells with the particle according to the present disclosure. In some embodiments, the particles described herein may be used to transduce cells that have not been previously activated. For example, the particles described herein may be useful for transducing cells that have not been previously contacted with cell activation beads or activation reagents (e.g. Dynabeads or other reagents comprising anti-CD3 and/or anti-CD28 antibodies or binding fragments thereof). Where a method herein describes use of a lentiviral particle, use of another particle is contemplated where appropriate and feasible. Where a method herein describes use of a lentiviral particle, use of a composition or fusion molecule is also contemplated where appropriate and feasible. For example, a fusion molecule, contained on the surface of a lentiviral particle, or a pharmaceutical composition may be administered to or contacted with a cell such as an immune cell (e.g. T cell).

Non-limiting examples of cells that can be the target of the lentiviral particle described herein include T lymphocytes, dendritic cells (DC), T_{reg} cells, B cells, Natural Killer cells, and macrophages.

Disclosed herein, in some embodiments, are methods of treating a CD20+ cancer in a subject in need thereof. The method may include administering to the subject a lentiviral particle disclosed herein, such as a lentiviral particle comprising a polynucleotide encoding an anti-CD20 CAR. In some embodiments, the lentiviral particle is administered by intranodal, intravenous, or subcutaneous injection. In some embodiments, the lentiviral particle is administered by intranodal injection via an inguinal lymph node. Some methods disclosed herein include a method of treating a CD20+ cancer in a subject in need thereof, the method comprising providing immune cells of a subject, contacting the immune cells of a subject by extracorporeal incubation with the lentiviral particle of any preceding embodiment, and administering the immune cell to the subject by transfusion. In some embodiments, the subject suffers from or is at risk for a B-cell malignancy, relapsed/refractory CD20-expressing malignancy, diffuse large B-cell lymphoma (DLBCL), Burkitt's type large B-cell lymphoma (B-LBL), follicular lymphoma (FL), chronic lymphocytic leukemia (CLL), acute lymphocytic leukemia (ALL), mantle cell lymphoma (MCL), hematological malignancy, colon cancer, lung cancer, liver cancer, breast cancer, renal cancer, prostate cancer, ovarian cancer, skin cancer, melanoma, bone cancer, brain cancer, squamous cell carcinoma, leukemia, myeloma, B cell lymphoma, kidney cancer, uterine cancer, adenocarcinoma, pancreatic cancer, chronic myelogenous leukemia, glioblastoma, neuroblastoma, medulloblastoma, or sarcoma. Some embodiments include administering a non-physiological ligand. In some embodiments, the non-physiological ligand comprises rapamycin or a rapamycin analog.

### Ex-Vivo Manufacturing

In some aspects, the disclosure provides a method of delivering a nucleic acid to a cell ex vivo. In some embodiments, the disclosure provides a method of delivering a nucleic acid to an immune cell ex vivo. In some embodiments, the lentiviral particles of the disclosure activate and transduce an immune cell ex vivo. In some embodiments, delivering a nucleic acid to a cell ex vivo, as described herein, results in the generation of an engineered cell comprising one or more exogenous proteins. For example, an engineered cell comprising an anti-CD20 CAR.

In some embodiments, the disclosure provides a method of delivering a nucleic acid to a cell in an ex vivo CAR T manufacturing process. Such methods typically involve the isolation of peripheral blood mononuclear cells (PBMCs) from a patient via leukapheresis. In some embodiments, such methods involve obtaining whole blood from a patient without isolation of PBMCs and forward processing the whole blood. The PBMCs may be washed and optionally further purified via one or more selection steps to isolate particular T cell populations of interest. In some aspects, these might include CD4+ and/or CD8+ T cells. The washed and/or purified cells may be optionally activated and then transduced using a lentiviral vector. The activation step may comprise contacting the cells with an exogenous activation agent such as anti-CD3 and anti-CD28 antibodies bound to a substrate or using unbound antibodies. Illustrative activation agents include anti-CD3 and anti-CD28-presenting beads and/or soluble polymers. Advantageously, the particles of the present disclosure may not require activation prior to transduction and so the activation step may be omitted. Transduction may be accomplished by contacting the patient's PBMCs, isolated cells, or, in some cases, whole blood with the lentiviral particles described herein. After transduction, the cells may be optionally further washed and cultured until harvest. Methods of manufacturing engineered cell therapies, including CAR T cells (see e.g., Abou-el-Enein, M. et al. Blood Cancer Discov (2021), Vol 2(5): 408-422; Arcangeli, S. et al. Front. Immunol (19 Jun. 2020), Vol. 11 (1217) 1-13; Ghassemi, S. et al. Nat Biomed Eng (February 2022), Vol 6(2): 118-128; Vormittag, P. et al. Curr Opin Biotechnol (October 2018), Vol. 54: 164-181; each of which is herein incorporated by reference), may be useful. Illustrative methods of autologous CAR T manufacturing are disclosed in US Patent Publication Nos. 2019/0269727, 2016/0122782, 2021/0163893, and US 2017/0037369, each of which is incorporated herein in its entirety.

In some embodiments, the disclosure provides a method of delivering a nucleic acid to a cell in an ex-vivo closed-loop manufacturing process. In some embodiments, an ex-vivo manufacturing process is an extracorporeal process. In exemplary embodiments, the lentiviral vectors disclosed herein permit delivery of a nucleic acid to a target cell during a closed-loop process. Exemplary methods of closed-loop and/or extracorporeal processes are disclosed in US Patent Publication No. 2021/0244871 and WO2022072885, each of which are incorporated herein in their entirety. In some embodiments, the lentiviral vectors as disclosed herein may be used to transduce cells ex vivo. For example, in exemplary closed-loop manufacturing processes, cells are obtained from a subject, washed, incubated and/or contacted with lentiviral particles, optionally washed again, and infused into the subject in a closed-loop system. In such embodiments, the lentiviral particles as disclosed herein are useful even without prior activation of the cells and are capable of binding to the cells in a short incubation and/or contacting step. In some embodiments, the incubation and/or contacting step is approximately or less than one hour. In some embodiments, the incubation and/or contacting step is approximately or less than one hour, approximately or less than two hours, approximately or less than three hours, approximately or less than four hours, or approximately or less than five hours. In some embodiments, the incubation and/or contacting step is less than 12 hours or less than 24 hours. In some embodiments, a nucleic acid is delivered to a cell by transduction with a lentiviral vector such that the nucleic acid enters the cell ex-vivo. In some embodiments, a nucleic acid is delivered to a cell by contacting the lentiviral vector to the surface of the cell. In such embodiments, the nucleic acid may enter the cell ex-vivo or in vivo after the cells (complexed with the lentiviral vector) are infused back into the subject.

In some embodiments, provided herein are bedside systems and methods for performing cell-based therapies and treatments in a subject-connected, closed-loop continuous-flow manner, including cellular modifications and treatments, e.g., to produce chimeric antigen receptor T (CAR T) cells. In some embodiments of a system described herein, blood is removed from a subject, processed, customized, and returned to the subject in a closed-loop, continuous-flow manner. An arrangement of modules and units are used sequentially for separation and collection of target cells from whole blood, employing for example, leukapheresis and/or other cell enrichment techniques, optionally including cell enrichment, purification and/or washing using an elutriation device, followed by one or more cell customization procedures, e.g., to generate CAR-T cells, optionally followed by cell enrichment, purification, fractionation, and/or washing, after which the processed and modified fraction comprising CAR-T cells are returned to the subject by means of an outlet conduit. One exemplary system is manufactured by Lupagen™ and is a closed-loop, continuous-flow system. Such systems and methods are disclosed in WO2019217964, which is incorporated herein by reference in its entirety. In some embodiments, the lentiviral vectors as disclosed herein eliminate the need for an ex-vivo activation step. In such embodiments, the isolated cells could be transduced directly after leukapheresis, washing, or selection. It is contemplated that the surface engineering described herein enables the lentiviral particles disclosed herein to activate and transduce cells in a single step. In such embodiments, the lentiviral particles disclosed herein may enable a short or truncated manufacturing process, reducing the time spent in ex-vivo manufacturing by eliminating one or more unit operations (e.g. activation prior to transduction) and/or reducing the amount of time that may be necessary in post-transduction cell culture. Without wishing to be bound by theory, in some embodiments the lentiviral vectors as described herein, in particular those particles comprising a fusion multidomain protein, bind to target cells with a higher avidity than lentiviral particles not comprising a fusion multidomain protein. In such embodiments, the fusion multidomain protein may allow the described lentiviral particles to bind target cells more tightly, reducing the incubation time for transduction and increasing transduction frequency and efficiency. In some embodiments, the time to effectively bind a lentiviral particle to a target cell may be one hour or less.

It is contemplated that the present disclosure provides an ex vivo method of generating an engineered cell comprising contacting a target cell with a particle comprising a fusion molecule comprising an adhesion molecule linked to a costimulatory molecule, a fusion molecule comprising an adhesion molecule linked to an activation molecule, or a fusion molecule comprising an adhesion molecule linked to a costimulatory molecule and an activation molecule wherein the contacting step is performed for approximately one hour, for approximately two hours, approximately three hours, approximately four hours, approximately five hours, approximately six hours, approximately 12 hours, approximately 24 hours, approximately 12-24 hours (inclusive of endpoints), or longer. This method may require the contacting step to be performed in a closed-loop manufacturing or extracorporeal process as described herein. Alternatively, this method may require the contacting step to be performed in a traditional ex-vivo engineered cell manufacturing process. For example, in a perfusion incubator or a centrifuge (such as a Sepax or Rotea machine).

### Method of Use In Vivo

Disclosed herein, in some embodiments, are methods comprising administering the lentiviral particle to a subject. The administration or lentiviral particle may treat a condition of the subject. In some embodiments, the subject has cancer or is in need of cancer treatment. In some embodiments, the administration treats or alleviates symptoms of the cancer. In some embodiments, the cancer comprises CD20+ cancer cells. In some embodiments, the cancer is or includes a B-cell malignancy. In some embodiments, the subject has an autoimmune disease or is in need of treatment for an autoimmune disease. In some embodiments, the administration treats or alleviates symptoms of the autoimmune disease. In some embodiments, the autoimmune disease is caused or exacerbated by B cells, in exemplary embodiments, CD20+ cells.

In some embodiments, the lentiviral particles described herein transduce target cells in vivo. In some embodiments, the target cells are immune cells. In some embodiments, the immune cells are T cells. In some embodiments, the lentiviral particles described herein transduce T cells in vivo. In some embodiments, the lentiviral particles described herein transduce T cells in vivo generating CAR T cells. In some embodiments, the lentiviral particles described herein display a CD58-CD80-anti-CD3 scFv tri-fusion polypeptide and transduce T cells in vivo generating CAR T cells. Where a method herein describes use of a lentiviral particle, use of another particle is contemplated where feasible.

In some embodiments, the disclosure provides an in vivo method of transducing target cells in a subject in need thereof, comprising administering to the subject a particle or pharmaceutical composition of the disclosure. The particle may be administered by any appropriate method including intranodal, intravenous, or subcutaneous injection. In some embodiments, the viral particle is administered via a route selected from the group consisting of intranodal, extracorporeal, parenteral, intravenous, intramuscular, subcutaneous, intratumoral, intraperitoneal, and intralymphatic. In some embodiments, the viral particle is administered multiple times. In some embodiments, the viral particle is administered by intralymphatic injection of the viral particle. In some embodiments, the viral particle is administered by intraperitoneal injection of the viral particle. In some embodiments, the viral particle is administered by intra-nodal injection—that is, the viral particle may be administered via injection into one or more lymph nodes. In some embodiments, the lymph nodes for administration are the inguinal lymph nodes. In some embodiments, the viral particle is administered by injection of the viral particle into tumor sites (i.e. intratumoral). In some embodiments, the viral particle is administered subcutaneously. In some embodiments, the viral particle is administered systemically. In some embodiments, the viral particle is administered intravenously. In some embodiments, the viral particle is administered intra-arterially. In some embodiments, the viral particle is a lentiviral particle.

In some embodiments, the lentiviral particle is administered by intraperitoneal, subcutaneous, or intranodal injection. In some embodiments, the lentiviral particle is administered by intraperitoneal injection. In some embodiments, the lentiviral particle is administered by subcutaneous injection. In some embodiments, the lentiviral particle is administered by intranodal injection.

The present disclosure provides a method of treatment comprising administering a therapeutically effective dose of lentiviral particles to a subject in need thereof. In some embodiments, a therapeutically effective dose of the lentiviral particles described herein are administered. In some embodiments, a therapeutically effective dose comprises about 0.1×10⁶ transducing units (TUs), about 0.2×10⁶ TUs, about 0.3×10⁶ TUs, about 0.4×10⁶ TUs, about 0.5×10⁶ TUs, about 0.6×10⁶ TUs, about 0.7×10⁶ TUs, about 0.8×10⁶ TUs, about 0.9×10⁶ TUs, about 1×10⁶ TUs, about 1.2×10⁶ TUs, about 1.4×10⁶ TUs, about 1.6×10⁶ TUs, about 1.8×10⁶ TUs, about 0.1×10⁶ TUs, about 0.1×10⁶ TUs, about 0.1×10⁶ TUs, about 0.1×10⁶ TUs, about 2×10⁶ TUs, about 2.5×10⁶ TUs, about 3×10⁶ TUs, about 4×10⁶ TUs, about 5×10⁶ TUs, about 6×10⁶ TUs, about 7×10⁶ TUs, about 8×10⁶ TUs, about 9×10⁶ TUs, about 1×10⁷ TUs, about 2×10⁷ TUs, about 3×10⁷ TUs, about 4×10⁷ TUs, about 5×10⁷ TUs, about 6×10⁷ TUs, about 7×10⁷ TUs, about 8×10⁷ TUs, about 9×10⁷ TUs, about 1×10⁸ TUs, about 2×10⁸ TUs, about 3×10⁸ TUs, about 4×10⁸ TUs, about 5×10⁸ TUs, about 6×10⁸ TUs, about 7×10⁸ TUs, about 8×10⁸ TUs, about 9×10⁸ TUs, about 1×10⁹ TUs, or about 2×10⁹ TUs. In some embodiments, the therapeutically effective dose is about 1.2×10⁸ TU. In some embodiments, the therapeutically effective dose is about 3.5×10⁸ TU. In some embodiments, the therapeutically effective dose is about 1.0×10⁹ TU. In some embodiments, the therapeutically effective dose is about 3.0×10⁹ TU. In some embodiments, the therapeutically effective dose is about 9.0×10⁹ TU. In some embodiments, the therapeutically effective dose is between about 1.2×10⁸ TU and 9.0×10⁹ TU, inclusive.

Dose level may be measured in transducing units, determined by any known method. Exemplary methods include cellular culture or colony formation tests (wherein a target cell population is exposed to the particles and the number of transduced cells are counted), digital polymerase chain reaction (dPCR), or any other method known in the art.

In some embodiments, the transduced immune cells comprising the polynucleotide of the present disclosure is administered to the subject.

The disclosure provides a method of treating a malignancy in a subject, comprising administering to the subject the lentiviral particles or pharmaceutical composition of the disclosure. In some embodiments, the malignancy is a B-cell malignancy, a myeloma, or a solid tumor malignancy. The disclosure provides a method of treating diffuse large B-cell lymphoma (DLBCL), Burkitt's type large B-cell lymphoma (B-LBL), follicular lymphoma (FL), chronic lymphocytic leukemia (CLL), acute lymphocytic leukemia (ALL), mantle cell lymphoma (MCL), hematological malignancy, colon cancer, lung cancer, liver cancer, breast cancer, renal cancer, prostate cancer, ovarian cancer, skin cancer, melanoma, bone cancer, brain cancer, squamous cell carcinoma, leukemia, myeloma, B cell lymphoma, kidney cancer, uterine cancer, adenocarcinoma, pancreatic cancer, chronic myelogenous leukemia, glioblastoma, neuroblastoma, medulloblastoma, or sarcoma in a subject, comprising administering to the subject the lentiviral particles or pharmaceutical composition of the disclosure.

In some embodiments, the lentiviral particle is administered as a single injection. In some embodiments, the lentiviral particle is administered as at least 1, at least 2, at least 3, at least 4, at least 5, at least 6, at least 7, at least 8, at least 9, or at least 10 injections.

In some embodiments, the lentiviral particles disclosed herein may be used in a method to generate engineered cells in vivo. It has been observed that the lentiviral particles comprising surface engineering (e.g. a fusion protein as disclosed herein) may preferentially generate engineered central memory T cells (TCM). It has also been observed that administering lentiviral particles via one or more lymph nodes may contribute to generation of a predominately TCM engineered cell phenotype. TCM may be characterized by expression of certain surface markers, for example, TCM may be CD62L+. TCM may also be CCR7+. TCM may also be characterized as CD45RA−, CD45RO+, and/or CD27+. In some embodiments, TCM are characterized as CCR7+, CD45RA−, CD45RO+, CD62L+, and CD27+. In some embodiments, TCM are characterized as CD45RA−, CCR7+. Without wishing to be bound by theory, it is contemplated that engineered TCM may persist for a longer time in vivo and may show improved effector function compared with engineered effector memory (TEM) or similar effector cell types.

Thus, it is contemplated that the present disclosure further provides a method of generating predominately engineered TCM in vivo. Due to the presence of the fusion proteins on the surface of the engineered particles, similar observations may be found in the ex-vivo setting, so the present disclosure further provides a method of generating predominately engineered TCM ex vivo, using one or more of the methods disclosed here. For example, via extracorporeal delivery or traditional ex-vivo manufacturing.

The disclosure provides a method of treating a condition in a subject, comprising administering to the subject the lentiviral particles or pharmaceutical composition of the disclosure. In some embodiments, the condition is a B-cell malignancy, a myeloma, or a solid tumor malignancy. The disclosure provides a method of treating diffuse large B-cell lymphoma (DLBCL), Burkitt's type large B-cell lymphoma (B-LBL), follicular lymphoma (FL), chronic lymphocytic leukemia (CLL), acute lymphocytic leukemia (ALL), mantle cell lymphoma (MCL), hematological malignancy, colon cancer, lung cancer, liver cancer, breast cancer, renal cancer, prostate cancer, ovarian cancer, skin cancer, melanoma, bone cancer, brain cancer, squamous cell carcinoma, leukemia, myeloma, B cell lymphoma, kidney cancer, uterine cancer, adenocarcinoma, pancreatic cancer, chronic myelogenous leukemia, glioblastoma, neuroblastoma, medulloblastoma, or sarcoma in a subject, comprising administering to the subject the lentiviral particles or pharmaceutical composition of the disclosure.

In some embodiments, the disclosure provides a method of treating a B-cell malignancy in a subject comprising administering to the subject a lentiviral particle comprising a cocal-pseudotyped lentiviral envelope with a membrane-bound multidomain fusion protein and a transgene encoding an anti-CD20 chimeric antigen receptor (CAR) comprising an anti-CD20 scFv and 4-1BB and CD3z intracellular signaling domains. In some embodiments, the disclosure provides a method of treating a B-cell malignancy in a subject comprising administering to the subject a lentiviral particle comprising a cocal-pseudotyped lentiviral envelope with a membrane-bound multidomain fusion protein and a transgene encoding, in any order, (i) an anti-CD20 chimeric antigen receptor (CAR) comprising an anti-CD20 scFv and 4-1BB and CD3z intracellular signaling domains; (ii) an inducible T-cell proliferative signaling system (e.g. a synthetic cytokine receptor system or RACR as described herein); and, optionally, (iii) a human FRB domain.

In some embodiments, the disclosure provides a method of treating a lymphoma in a subject comprising administering to the subject a lentiviral particle comprising a cocal-pseudotyped lentiviral envelope with a membrane-bound multidomain fusion protein and a transgene encoding an anti-CD20 chimeric antigen receptor (CAR) comprising an anti-CD20 scFv and 4-1BB and CD3z intracellular signaling domains. In some embodiments, the disclosure provides a method of treating a lymphoma in a subject comprising administering to the subject a lentiviral particle comprising a cocal-pseudotyped lentiviral envelope with a membrane-bound multidomain fusion protein and a transgene encoding, in any order, (i) an anti-CD20 chimeric antigen receptor (CAR) comprising an anti-CD20 scFv and 4-1BB and CD3z intracellular signaling domains; (ii) an inducible T-cell proliferative signaling system (e.g. a synthetic cytokine receptor system or RACR as described herein); and, optionally, (iii) a human FRB domain.

In some embodiments, the condition is an autoimmune disease. The disclosure also provides a method of treating an autoimmune disease in a subject, comprising administering to the subject the lentiviral or pharmaceutical composition of the disclosure. Some examples of an autoimmune disease may include systemic lupus erythematosus, Sjögren's Syndrome, ANCA-associated vasculitis and autoimmune hemolytic anemia, rheumatoid arthritis, systemic sclerosis, multiple sclerosis, neuromyelitis optica spectrum disorder, chronic inflammatory demyelinating polyradiculoneuropathy, immune-mediated necrotizing myopathy, pemphigus vulgaris, dermatomyositis, adult-onset Still's disease, inflammatory bowle disease, type 1 diabetus mellitus, graft vs. host disease, a myasthenia gravis, multiple sclerosis, Immune dysregulation, Polyendocrinopathy Enteropathy X-linked (IPEX) or autoimmune arthritis.

In some embodiments, the disclosure provides a method of treating an autoimmune disease in a subject comprising administering to the subject a lentiviral particle comprising a cocal-pseudotyped lentiviral envelope with a membrane-bound multidomain fusion protein and a transgene encoding an anti-CD20 chimeric antigen receptor (CAR) comprising an anti-CD20 scFv and 4-1BB and CD3z intracellular signaling domains. In some embodiments, the disclosure provides a method of treating an autoimmune disease in a subject comprising administering to the subject a lentiviral particle comprising a cocal-pseudotyped lentiviral envelope with a membrane-bound multidomain fusion protein and a transgene encoding, in any order, (i) an anti-CD20 chimeric antigen receptor (CAR) comprising an anti-CD20 scFv and 4-1BB and CD3z intracellular signaling domains; (ii) an inducible T-cell proliferative signaling system (e.g. a synthetic cytokine receptor system or RACR as described herein); and, optionally, (iii) a human FRB domain.

In some embodiments, the disclosure provides a method of treating systemic lupus erythematosus (SLE) in a subject comprising administering to the subject a lentiviral particle comprising a cocal-pseudotyped lentiviral envelope with a membrane-bound multidomain fusion protein and a transgene encoding an anti-CD20 chimeric antigen receptor (CAR) comprising an anti-CD20 scFv and 4-1BB and CD3z intracellular signaling domains. In some embodiments, the disclosure provides a method of treating SLE in a subject comprising administering to the subject a lentiviral particle comprising a cocal-pseudotyped lentiviral envelope with a membrane-bound multidomain fusion protein and a transgene encoding, in any order, (i) an anti-CD20 chimeric antigen receptor (CAR) comprising an anti-CD20 scFv and 4-1BB and CD3z intracellular signaling domains; (ii) an inducible T-cell proliferative signaling system (e.g. a synthetic cytokine receptor system or RACR as described herein); and, optionally, (iii) a human FRB domain.

### Method of Making

In some embodiments, the disclosure provides a method of making a particle, comprising introducing a polynucleotide encoding a vector genome into a host cell comprising a polynucleotide encoding a fusion molecule (or fusion protein) as described herein. The fusion molecule (or fusion protein) and the vector genome are expressed by the host cell. The host cell packages the vector genome into a lentiviral particle comprising the fusion molecule (or fusion protein).

In some embodiments, the disclosure provides an in vivo method of transducing target cells in a subject in need thereof, comprising administering to the subject a particle or pharmaceutical composition of the disclosure. The particle may be administered by intranodal, intravenous, or subcutaneous injection.

Various disease or disorders may be treated using particles as disclosed herein, or pharmaceutical composition comprising them. The particles may be administered to a subject suffering from or at risk for a B-cell malignancy, relapsed/refractory malignancy, diffuse large B-cell lymphoma (DLBCL), Burkitt's type large B-cell lymphoma (B-LBL), follicular lymphoma (FL), chronic lymphocytic leukemia (CLL), acute lymphocytic leukemia (ALL), mantle cell lymphoma (MCL), hematological malignancy, colon cancer, lung cancer, liver cancer, breast cancer, renal cancer, prostate cancer, ovarian cancer, skin cancer, melanoma, bone cancer, brain cancer, squamous cell carcinoma, leukemia, myeloma, B cell lymphoma, kidney cancer, uterine cancer, adenocarcinoma, pancreatic cancer, chronic myelogenous leukemia, glioblastoma, neuroblastoma, medulloblastoma, or sarcoma.

Lentiviral particles of the present disclosure may enhance in vivo activity. Lentiviral particles of the present disclosure resist serum inactivation. Lentiviral particles of the present disclosure provide efficient targeting of activated T cells. Lentiviral particles of the present disclosure may require low physical particle per transducing unit compared to two component glycoproteins. Lentiviral particles of the present disclosure retain potential to transduce a broad range of non-T effector cells. Lentiviral particles of the present disclosure enhance particle to T cell binding. Lentiviral particles of the present disclosure enhance T cell activation. Lentiviral particles of the present disclosure enhance immune cell expansion. Lentiviral particles of the present disclosure enhance immune cell transduction. Lentiviral particles of the present disclosure enhance anti-tumor potency. Lentiviral particles of the present disclosure enhance immune cell persistence.

Some embodiments include a method of making an adhesion molecule, a costimulatory molecule, an activation molecule, or a fusion molecule. The method may include transcribing or translating a nucleic acid (such as a DNA or RNA) that encodes a protein comprising the adhesion molecule, costimulatory molecule, activation molecule, or fusion molecule.

### Kits

Disclosed herein, in some embodiments, are kits. In some embodiments, the kit includes an adhesion molecule. In some embodiments, the kit includes a costimulatory molecule. In some embodiments, the kit includes an activation molecule. In some embodiments, the kit includes a fusion molecule. In some embodiments, the kit includes a particle. In some embodiments, the kit includes a composition described herein. The kit may include instructions for use, such as instructions for use in a method herein.

### NUMBERED EMBODIMENTS

Some embodiments relate to or include any of the following:
1. A lentiviral particle, comprising, displayed on the surface of the particle:
a fusion molecule comprising:
   a) a CD58 extracellular domain, or a functional fragment thereof,
   b) a CD80 or CD86 extracellular domain, or a functional fragment thereof,
   c) an antigen-binding fragment of an anti-CD3 antibody; and a viral glycoprotein (G protein),
   wherein the lentiviral particle comprises a polynucleotide encoding a chimeric antigen receptor that specifically binds CD20.
2. The lentiviral particle of embodiment 1, wherein the lentiviral particle comprises a polynucleotide encoding a free FKBP12-rapamycin binding (FRB).
3. The lentiviral particle of any one of embodiments 1-2, wherein the lentiviral particle comprises a polynucleotide encoding a synthetic cytokine gamma chain polypeptide and a synthetic cytokine beta chain polypeptide.
4. The lentiviral particle of any one of embodiments 1-3, wherein the chimeric antigen receptor comprises a ligand-binding domain comprising an scFv domain, wherein the scFv further comprises a VL comprising the polypeptide sequence of SEQ ID NO: 176 and a VH comprising the polypeptide sequence of SEQ ID NO: 179; or wherein the VL comprises a sequences at least 80%, 85%, 90%, 91%, 92%, 93%, 94%, 95%, 96%, 97%, 98%, or 99% identical to SEQ ID NO: 176 and the VH comprises a sequences at least 80%, 85%, 90%, 91%, 92%, 93%, 94%, 95%, 96%, 97%, 98%, or 99% identical to SEQ ID NO: 179.
5. The lentiviral particle of embodiment 4, wherein the scFv comprises a spacer comprising a polypeptide sequence at least 80%, 85%, 90%, 91%, 92%, 93%, 94%, 95%, 96%, 97%, 98%, or 99% identical to the polypeptide sequence of SEQ ID NO: 278.
6. The lentiviral particle of embodiment 5, wherein the scFv spacer comprises the polypeptide sequence of SEQ ID NO: 278.
7. The lentiviral particle of any one of embodiments 4-6, wherein the scFv comprises a polypeptide sequence at least 80%, 85%, 90%, 91%, 92%, 93%, 94%, 95%, 96%, 97%, 98%, or 99% identical to the polypeptide sequence of SEQ ID NO: 174.
8. The lentiviral particle of any one of embodiments 4-7, wherein the scFv comprises the polypeptide sequence of SEQ ID NO: 174.
9. The lentiviral particle of any one of embodiments 4-8, wherein the scFv is encoded by a polynucleotide sequence at least 80%, 85%, 90%, 91%, 92%, 93%, 94%, 95%, 96%, 97%, 98%, or 99% identical to the polynucleotide sequence of SEQ ID NO: 279.
10. The lentiviral particle of any one of embodiments 4-9, wherein the scFv is encoded by the polynucleotide sequence of SEQ ID NO: 279.
11. The lentiviral particle of any one of embodiments 1-10, wherein the chimeric antigen receptor comprises a CD8 hinge domain.
12. The lentiviral particle of embodiment 11, wherein the CD8 hinge domain is encoded by a polynucleotide sequence at least 80%, 85%, 90%, 91%, 92%, 93%, 94%, 95%, 96%, 97%, 98%, or 99% identical to the polynucleotide sequence of SEQ ID NO: 182.
13. The lentiviral particle of embodiment 11, wherein the CD8 hinge domain is encoded by the polynucleotide sequence of SEQ ID NO: 182.
14. The lentiviral particle of embodiment 11, wherein the CD8 hinge domain comprises a polypeptide sequence at least 80%, 85%, 90%, 91%, 92%, 93%, 94%, 95%, 96%, 97%, 98%, or 99% identical to the polypeptide sequence of SEQ ID NO: 280.
15. The lentiviral particle of embodiment 11, wherein the CD8 hinge domain comprises the polypeptide sequence of SEQ ID NO: 280.
16. The lentiviral particle of any one of embodiments 1-15, wherein the chimeric antigen receptor comprises a CD8 transmembrane domain.
17. The lentiviral particle of embodiment 16, wherein the CD8 transmembrane domain is encoded by a polynucleotide sequence at least 80%, 85%, 90%, 91%, 92%, 93%, 94%, 95%, 96%, 97%, 98%, or 99% identical to the polynucleotide sequence of SEQ ID NO: 183.
18. The lentiviral particle of embodiment 16, wherein the CD8 transmembrane domain is encoded by the polynucleotide sequence of SEQ ID NO: 183.
19. The lentiviral particle of embodiment 16, wherein the CD8 transmembrane domain comprises a polypeptide sequence at least 80%, 85%, 90%, 91%, 92%, 93%, 94%, 95%, 96%, 97%, 98%, or 99% identical to the polypeptide sequence of SEQ ID NO: 280.
20. The lentiviral particle of embodiment 16, wherein the CD8 transmembrane domain comprises the polypeptide sequence of SEQ ID NO: 280.
21. The lentiviral particle of any one of embodiments 1-20, wherein the chimeric antigen receptor comprises a 4-1BB endodomain.
22. The lentiviral particle of embodiment 21, wherein the 4-1BB endodomain is encoded by a polynucleotide sequence at least 80%, 85%, 90%, 91%, 92%, 93%, 94%, 95%, 96%, 97%, 98%, or 99% identical to the polynucleotide sequence of SEQ ID NO: 200.
23. The lentiviral particle of embodiment 21, wherein the 4-1BB endodomain is encoded by the polynucleotide sequence of SEQ ID NO: 200.
24. The lentiviral particle of embodiment 21, wherein the 4-1BB endodomain comprises a polypeptide sequence at least 80%, 85%, 90%, 91%, 92%, 93%, 94%, 95%, 96%, 97%, 98%, or 99% identical to the polypeptide sequence of SEQ ID NO: 201.
25. The lentiviral particle of embodiment 21, wherein the 4-1BB endodomain comprises the polypeptide sequence of SEQ ID NO: 201.
26. The lentiviral particle of any one of embodiments 1-25, wherein the chimeric antigen receptor comprises a CD3ζ endodomain.
27. The lentiviral particle of embodiment 26, wherein the CD3ζ endodomain is encoded by a polynucleotide sequence at least 80%, 85%, 90%, 91%, 92%, 93%, 94%, 95%, 96%, 97%, 98%, or 99% identical to the polynucleotide sequence of SEQ ID NO: 202.
28. The lentiviral particle of embodiment 26, wherein the CD3ζ endodomain is encoded by the polynucleotide sequence of SEQ ID NO: 202.
29. The lentiviral particle of embodiment 26, wherein the CD3ζ endodomain comprises a polypeptide sequence at least 80%, 85%, 90%, 91%, 92%, 93%, 94%, 95%, 96%, 97%, 98%, or 99% identical to the polypeptide sequence of SEQ ID NO: 203.
30. The lentiviral particle of embodiment 26, wherein the CD3ζ endodomain comprises the polypeptide sequence of SEQ ID NO: 203.
31. The lentiviral particle of any one of embodiments 1-30, wherein the polynucleotide encoding the chimeric antigen receptor comprises a polynucleotide sequence at least 80%, 85%, 90%, 91%, 92%, 93%, 94%, 95%, 96%, 97%, 98%, or 99% identical to the polynucleotide sequence of SEQ ID NO: 186.
32. The lentiviral particle of any one of embodiments 1-30, wherein the polynucleotide encoding the chimeric antigen receptor comprises the polynucleotide sequence of SEQ ID NO: 186.
33. The lentiviral particle of any one of embodiments 1-30, wherein the chimeric antigen receptor comprises a polypeptide sequence at least 80%, 85%, 90%, 91%, 92%, 93%, 94%, 95%, 96%, 97%, 98%, or 99% identical to the polypeptide sequence of SEQ ID NO: 209.
34. The lentiviral particle of any one of embodiments 1-30, wherein the chimeric antigen receptor comprises the polypeptide sequence of SEQ ID NO: 209.
35. The lentiviral particle of any one of embodiments 1-34, wherein the CD58 extracellular domain, or a functional fragment thereof, comprises a polypeptide sequence that is at least 80%, 85%, 90%, 91%, 92%, 93%, 94%, 95%, 96%, 97%, 98%, or 99% identical to the polypeptide sequence of SEQ ID NO: 10.
36. The lentiviral particle of any one of embodiments 1-35, wherein the antigen-binding fragment of an anti-CD3 antibody is an scFv domain comprising a polypeptide sequence that is at least 80%, 85%, 90%, 91%, 92%, 93%, 94%, 95%, 96%, 97%, 98%, or 99% identical to the polypeptide sequence of SEQ ID NO: 31.
37. The lentiviral particle of any one of embodiments 1-36, wherein the CD80 extracellular domain, or a functional fragment thereof, comprises a polypeptide sequence that is at least 80%, 85%, 90%, 91%, 92%, 93%, 94%, 95%, 96%, 97%, 98%, or 99% identical to the polypeptide sequence of SEQ ID NO: 12.
38. The lentiviral particle of any one of embodiments 1-37, wherein the CD86 extracellular domain, or a functional fragment thereof, comprises a polypeptide sequence that is at least 80%, 85%, 90%, 91%, 92%, 93%, 94%, 95%, 96%, 97%, 98%, or 99% identical to the polypeptide sequence of SEQ ID NO: 13.
39. The lentiviral particle of any one of embodiments 1-38, wherein the fusion molecule comprises, in N to C-terminal order, the CD58 extracellular domain, the antigen-binding fragment of an anti-CD3 antibody, and the CD86 extracellular domain.
40. The lentiviral particle of any one of embodiments 1-37, wherein the fusion molecule comprises a polypeptide sequence that is at least 80%, 85%, 90%, 91%, 92%, 93%, 94%, 95%, 96%, 97%, 98%, or 99% identical to the polypeptide sequence of SEQ ID NO: 33.
41. The lentiviral particle of any one of embodiments 1-37, wherein the fusion molecule comprises, in N to C-terminal order, the CD58 extracellular domain, the antigen-binding fragment of an anti-CD3 antibody, and the CD80 extracellular domain.
42. The lentiviral particle of any one of embodiments 1-41, wherein the viral glycoprotein (G protein) comprises a polypeptide sequence that is at least 80%, 85%, 90%, 91%, 92%, 93%, 94%, 95%, 96%, 97%, 98%, or 99% identical to the polypeptide sequence of SEQ ID NO: 74.
43. The lentiviral particle of any one of embodiments 1-42, further comprising a polycistronic construct comprising in 5′ to 3′ order:
   a. a first expression cassette comprising a nucleotide sequence encoding the free FRB,
   b. a second expression cassette comprising a nucleotide sequence encoding the synthetic cytokine gamma chain polypeptide,
   c. a third expression cassette comprising a nucleotide sequence encoding the synthetic cytokine beta chain polypeptide, and
   d. a fourth expression cassette comprising a nucleotide sequence encoding the chimeric antigen receptor (CAR);
wherein each of the expression cassettes are separated by a nucleotide sequence encoding a cleavage site sequence.
44. The lentiviral particle of any one of embodiments 1-43, wherein the polynucleotide sequence encoding FRB that is at least 80%, 85%, 90%, 91%, 92%, 93%, 94%, 95%, 96%, 97%, 98%, or 99% identical to the polynucleotide sequence of SEQ ID NOs: 256, 257, or 258.
45. The lentiviral particle of any one of embodiments 1-43, wherein the polynucleotide sequence encoding the free FRB comprises the polynucleotide sequence of SEQ ID NOs: 256, 257, or 258.
46. The lentiviral particle of any one of embodiments 1-43, wherein the free FRB polynucleotide sequence encodes a polypeptide sequence that is at least 80%, 85%, 90%, 91%, 92%, 93%, 94%, 95%, 96%, 97%, 98%, or 99% identical to the polynucleotide sequence of SEQ ID NOs: 251, 252, or 260.
47. The lentiviral particle of any one of embodiments 1-43, wherein the FRB polynucleotide sequence encodes the polypeptide sequence of SEQ ID NOs: 251, 252, or 260.
48. The lentiviral particle of any one of embodiments 1-47, wherein the polynucleotide sequence encoding a synthetic cytokine gamma chain polypeptide is at least 80%, 85%, 90%, 91%, 92%, 93%, 94%, 95%, 96%, 97%, 98%, or 99% identical to the polynucleotide sequence of SEQ ID NOs: 261, 262, or 263.
49. The lentiviral particle of any one of embodiments 1-47, wherein the polynucleotide encoding the synthetic cytokine gamma chain polypeptide comprises the polynucleotide sequence of SEQ ID NOs: 261, 262, or 263.
50. The lentiviral particle of any one of embodiments 1-49, wherein the synthetic cytokine gamma chain polypeptide comprises interleukin 2 receptor subunit γ (IL2RG) comprising a polypeptide sequence at least 80%, 85%, 90%, 91%, 92%, 93%, 94%, 95%, 96%, 97%, 98%, or 99% identical to the amino acid sequence of SEQ ID NOs: 264 or 265.
51. The lentiviral particle of embodiment 50, wherein the IL2RG comprises the polypeptide sequence of SEQ ID NOs: 264 or 265.
52. The lentiviral particle of any one of embodiments 43-51, wherein the second expression cassette comprises a polynucleotide sequence at least 80%, 85%, 90%, 91%, 92%, 93%, 94%, 95%, 96%, 97%, 98%, or 99% identical to the polynucleotide sequence of SEQ ID NO: 266.
53. The lentiviral particle of any one of embodiments 43-51, wherein the second expression cassette comprises the polynucleotide sequence of SEQ ID NO: 266.
54. The lentiviral particle of any one of embodiments 43-51, wherein the second expression cassette encodes a polypeptide sequence at least 80%, 85%, 90%, 91%, 92%, 93%, 94%, 95%, 96%, 97%, 98%, or 99% identical to the polypeptide sequence of SEQ ID NO: 267.
55. The lentiviral particle of any one of embodiments 43-51, wherein the second expression cassette encodes a polypeptide sequence comprising the sequence of SEQ ID NO: 267.
56. The lentiviral particle of any one of embodiments 43-55, wherein the second expression cassette further comprises a polynucleotide sequence encoding FKBP12 that is at least 80%, 85%, 90%, 91%, 92%, 93%, 94%, 95%, 96%, 97%, 98%, or 99% identical to the polynucleotide sequence of SEQ ID NOs: 268 or 269.
57. The lentiviral particle of embodiment 56, wherein the polynucleotide sequence encoding the FKBP12 comprises the polynucleotide sequence of SEQ ID NOs: 268 or 269.
58. The lentiviral particle of any one of embodiments 56-57, wherein the FKBP12 comprises a polypeptide sequence at least 80%, 85%, 90%, 91%, 92%, 93%, 94%, 95%, 96%, 97%, 98%, or 99% identical to the polypeptide sequence of SEQ ID NO: 253
59. The lentiviral particle of any one of embodiments 56-57, wherein the FKBP12 comprises the polypeptide sequence of SEQ ID NO: 253.
60. The lentiviral particle of any one of embodiments 1-59, wherein the polynucleotide encoding the synthetic cytokine beta chain polypeptide is at least 80%, 85%, 90%, 91%, 92%, 93%, 94%, 95%, 96%, 97%, 98%, or 99% identical to the polynucleotide sequence of SEQ ID NOs: 270 or 271.
61. The lentiviral particle of any one of embodiments 1-59, wherein the polynucleotide encoding the synthetic cytokine beta chain polypeptide comprises the polynucleotide sequence of SEQ ID NOs: 270 or 271.
62. The lentiviral particle of any one of embodiments 1-61, wherein the synthetic cytokine beta chain polypeptide comprises interleukin 2 receptor subunit β (IL2RB) comprising a polypeptide sequence at least 80%, 85%, 90%, 91%, 92%, 93%, 94%, 95%, 96%, 97%, 98%, or 99% identical to the amino acid sequence of SEQ ID NOs: 272 or 273.
63. The lentiviral particle of embodiment 62, wherein the IL2RB comprises the polypeptide sequence of SEQ ID NOs: 272 or 273.
64. The lentiviral particle of any one of embodiments 43-63, wherein the third expression cassette further comprises a polynucleotide sequence encoding FKBP12 that is at least 80%, 85%, 90%, 91%, 92%, 93%, 94%, 95%, 96%, 97%, 98%, or 99% identical to the polynucleotide sequence of SEQ ID NO: 274.
65. The lentiviral particle of any one of embodiments 43-63, wherein the polynucleotide sequence encoding the FKBP12 comprises the polynucleotide sequence of SEQ ID NO: 274.
66. The lentiviral particle of any one of embodiments 43-65, wherein the FKBP12 comprises a polypeptide sequence at least 80%, 85%, 90%, 91%, 92%, 93%, 94%, 95%, 96%, 97%, 98%, or 99% identical to the polypeptide sequence of SEQ ID NO: 275.
67. The lentiviral particle of any one of embodiments 43-65, wherein the FKBP12 comprises the polypeptide sequence of SEQ ID NO: 275.
68. The lentiviral particle of any one of embodiments 43-67, wherein the third expression cassette comprises a polynucleotide sequence at least 80%, 85%, 90%, 91%, 92%, 93%, 94%, 95%, 96%, 97%, 98%, or 99% identical to the polynucleotide sequence of SEQ ID NO: 276.
69. The lentiviral particle of any one of embodiments 43-67, wherein the third expression cassette comprises the polynucleotide sequence of SEQ ID NO: 276.
70. The lentiviral particle of any one of embodiments 43-69, wherein the third expression cassette encodes a polypeptide sequence at least 80%, 85%, 90%, 91%, 92%, 93%, 94%, 95%, 96%, 97%, 98%, or 99% identical to the polypeptide sequence of SEQ ID NO: 277.
71. The lentiviral particle of any one of embodiments 43-69, wherein the third expression cassette encodes the polypeptide sequence comprising the sequence of SEQ ID NO: 277.
72. A method of treating a CD20+ cancer in a subject in need thereof, the method comprising administering to the subject the lentiviral particle of any preceding embodiment.
73. The method of embodiment 72, wherein the lentiviral particle is administered by intranodal, intravenous, or subcutaneous injection.
74. The method of embodiment 72, wherein the lentiviral particle is administered by intranodal injection via an inguinal lymph node.
75. A method of treating a CD20+ cancer in a subject in need thereof, the method comprising providing immune cells of a subject, contacting the immune cells of a subject by extracorporeal incubation with the lentiviral particle of any preceding embodiment, and administering the immune cell to the subject by transfusion.
76. The method of any one of embodiments 72-75, where the subject suffers from or is at risk for a B-cell malignancy, relapsed/refractory CD20-expressing malignancy, diffuse large B-cell lymphoma (DLBCL), Burkitt's type large B-cell lymphoma (B-LBL), follicular lymphoma (FL), chronic lymphocytic leukemia (CLL), acute lymphocytic leukemia (ALL), mantle cell lymphoma (MCL), hematological malignancy, colon cancer, lung cancer, liver cancer, breast cancer, renal cancer, prostate cancer, ovarian cancer, skin cancer, melanoma, bone cancer, brain cancer, squamous cell carcinoma, leukemia, myeloma, B cell lymphoma, kidney cancer, uterine cancer, adenocarcinoma, pancreatic cancer, chronic myelogenous leukemia, glioblastoma, neuroblastoma, medulloblastoma, or sarcoma.
77. The method of any of embodiments 72-76, further comprising administering a non-physiological ligand.
78. The method of embodiment 77, wherein the non-physiological ligand comprises rapamycin or a rapamycin analog.
79. A pharmaceutical composition comprising the lentiviral particle of any one of embodiments 1-71, and a pharmaceutically acceptable carrier.
80. A lentiviral particle, comprising, displayed on the surface of the particle:
   a fusion molecule comprising
      a) a CD58 extracellular domain, or a functional fragment thereof,
      b) an antigen-binding fragment of an anti-CD3 antibody, and
      c) a CD80 extracellular domain, or a functional fragment thereof; and a viral glycoprotein (G protein);
      wherein the lentiviral particle further comprises a polynucleotide encoding a chimeric antigen receptor that specifically binds CD20, a free FRB, a synthetic cytokine gamma chain polypeptide, and a synthetic cytokine beta chain polypeptide,
      wherein the chimeric antigen receptor comprises a ligand-binding domain comprising an scFv, a hinge domain, a transmembrane domain, a 41BB endodomain, and a CD3ζ endodomain, and
      wherein the scFv comprises a VL comprising SEQ ID NO: 176 and a VH comprising SEQ ID NO: 179, the hinge domain comprises SEQ ID NO: 280, the transmembrane domain comprises SEQ ID NO: 281, the 41BB endodomain comprises SEQ ID NO: 184, and the CD3ζ endodomain comprises SEQ ID NO: 214.
81. The lentiviral particle of embodiment 80, further comprising a polycistronic construct comprising in 5′ to 3′ order:
   a. a first expression cassette comprising a nucleotide sequence encoding the free FRB,
   b. a second expression cassette comprising a nucleotide sequence encoding the synthetic cytokine gamma chain polypeptide,
   c. a third expression cassette comprising a nucleotide sequence encoding the synthetic cytokine beta chain polypeptide, and
   d. a fourth expression cassette comprising a nucleotide sequence encoding the chimeric antigen receptor (CAR), wherein each of the expression cassettes are separated by a nucleotide sequence encoding a cleavage site sequence.
82. The lentiviral particle of embodiment 81, wherein the polynucleotide sequence encoding FRB that is at least 80%, 85%, 90%, 91%, 92%, 93%, 94%, 95%, 96%, 97%, 98%, 99%, or 100% identical to the polynucleotide sequence of SEQ ID NOs: 256, 257, or 258.
83. The lentiviral particle of any one of embodiments 81-82, wherein the free FRB polynucleotide sequence encodes a polypeptide sequence that is at least 80%, 85%, 90%, 91%, 92%, 93%, 94%, 95%, 96%, 97%, 98%, 99%, or 100% identical to the polynucleotide sequence of SEQ ID NOs: 251, 252, or 260.
84. The lentiviral particle of any one of embodiments 81-83, wherein the polynucleotide sequence encoding a synthetic cytokine gamma chain polypeptide is at least 80%, 85%, 90%, 91%, 92%, 93%, 94%, 95%, 96%, 97%, 98%, 99%, or 100% identical to the polynucleotide sequence of SEQ ID NOs: 261, 262, or 263.
85. The lentiviral particle of any one of embodiments 81-84, wherein the synthetic cytokine gamma chain polypeptide comprises interleukin 2 receptor subunit γ (IL2RG) comprising a polypeptide sequence at least 80%, 85%, 90%, 91%, 92%, 93%, 94%, 95%, 96%, 97%, 98%, 99%, or 100% identical to the amino acid sequence of SEQ ID NOs: 264 or 265.
86. The lentiviral particle of any one of embodiments 81-85, wherein the second expression cassette comprises a polynucleotide sequence at least 80%, 85%, 90%, 91%, 92%, 93%, 94%, 95%, 96%, 97%, 98%, 99%, or 100% identical to the polynucleotide sequence of SEQ ID NO: 266.
87. The lentiviral particle of any one of embodiments 81-86, wherein the second expression cassette encodes a polypeptide sequence at least 80%, 85%, 90%, 91%, 92%, 93%, 94%, 95%, 96%, 97%, 98%, 99%, or 100% identical to the polypeptide sequence of SEQ ID NO: 267.
88. The lentiviral particle of any one of embodiments 81-87, wherein the second expression cassette further comprises a polynucleotide sequence encoding FKBP12 that is at least 80%, 85%, 90%, 91%, 92%, 93%, 94%, 95%, 96%, 97%, 98%, 99%, or 100% identical to the polynucleotide sequence of SEQ ID NOs: 268 or 269.
89. The lentiviral particle of any one of embodiments 81-88, wherein the FKBP12 comprises a polypeptide sequence at least 80%, 85%, 90%, 91%, 92%, 93%, 94%, 95%, 96%, 97%, 98%, 99%, or 100% identical to the polypeptide sequence of SEQ ID NO: 253
90. The lentiviral particle of any one of embodiments 81-89, wherein the polynucleotide encoding the synthetic cytokine beta chain polypeptide is at least 80%, 85%, 90%, 91%, 92%, 93%, 94%, 95%, 96%, 97%, 98%, 99%, or 100% identical to the polynucleotide sequence of SEQ ID NOs: 270 or 271.
91. The lentiviral particle of any one of embodiments 80-90, wherein the synthetic cytokine beta chain polypeptide comprises interleukin 2 receptor subunit β (IL2RB) comprising a polypeptide sequence at least 80%, 85%, 90%, 91%, 92%, 93%, 94%, 95%, 96%, 97%, 98%, 99%, or 100% identical to the amino acid sequence of SEQ ID NOs: 272 or 273.
92. The lentiviral particle of any one of embodiments 81-91, wherein the third expression cassette further comprises a polynucleotide sequence encoding FKBP12 that is at least 80%, 85%, 90%, 91%, 92%, 93%, 94%, 95%, 96%, 97%, 98%, 99%, or 100% identical to the polynucleotide sequence of SEQ ID NO: 274.
93. The lentiviral particle of any one of embodiments 81-92, wherein the FKBP12 comprises a polypeptide sequence at least 80%, 85%, 90%, 91%, 92%, 93%, 94%, 95%, 96%, 97%, 98%, 99%, or 100% identical to the polypeptide sequence of SEQ ID NO: 275.
94. The lentiviral particle of any one of embodiments 81-93, wherein the third expression cassette comprises a polynucleotide sequence at least 80%, 85%, 90%, 91%, 92%, 93%, 94%, 95%, 96%, 97%, 98%, 99%, or 100% identical to the polynucleotide sequence of SEQ ID NO: 276.
95. The lentiviral particle of any one of embodiments 81-94, wherein the third expression cassette encodes a polypeptide sequence at least 80%, 85%, 90%, 91%, 92%, 93%, 94%, 95%, 96%, 97%, 98%, 99%, or 100% identical to the polypeptide sequence of SEQ ID NO: 277.
96. The lentiviral particle of any one of embodiments 81-95, wherein the fusion molecule comprises a polypeptide sequence at least 80%, 85%, 90%, 91%, 92%, 93%, 94%, 95%, 96%, 97%, 98%, or 99% identical to the polypeptide sequence of SEQ ID NO: 72.
97. The lentiviral particle of any one of embodiments 81-96, wherein the fusion molecule comprises the polypeptide sequence of SEQ ID NO: 72.
98. The lentiviral particle of any one of embodiments 81-96, wherein the fusion molecule comprises in 5′ to 3′ order:
   a. a CD58 extracellular domain, or a functional fragment thereof,
   b. an antigen-binding fragment of an anti-CD3 antibody, and
   c. a CD80 extracellular domain, or a functional fragment thereof.
99. The lentiviral particle of any one of embodiments 1-42, comprising a polycistronic construct comprising in 5′ to 3′ order:
   a. a first expression cassette comprising a nucleotide sequence encoding the free FRB,
   b. a second expression cassette comprising a nucleotide sequence encoding the synthetic cytokine gamma chain polypeptide,
   c. a third expression cassette comprising a nucleotide sequence encoding the synthetic cytokine beta chain polypeptide, and
   d. a fourth expression cassette comprising a nucleotide sequence encoding the chimeric antigen receptor (CAR);
wherein the CAR specifically binds CD20.
100. The lentiviral particle of embodiment 99, wherein the chimeric antigen receptor comprises a ligand-binding domain comprising an scFv comprising a VL comprising SEQ ID NO: 176 or a sequences at least 80%, 85%, 90%, 91%, 92%, 93%, 94%, 95%, 96%, 97%, 98%, or 99% identical thereto and a VH comprising SEQ ID NO: 179 or a sequences at least 80%, 85%, 90%, 91%, 92%, 93%, 94%, 95%, 96%, 97%, 98%, or 99% identical thereto.
101. The lentiviral particle of any one of embodiments 99-100, wherein the chimeric antigen receptor comprises a hinge domain comprising SEQ ID NO: 280 or a sequences at least 80%, 85%, 90%, 91%, 92%, 93%, 94%, 95%, 96%, 97%, 98%, or 99% identical thereto.
102. The lentiviral particle of any one of embodiments 99-101, wherein the chimeric antigen receptor comprises a transmembrane domain comprising SEQ ID NO: 281 or a sequences at least 80%, 85%, 90%, 91%, 92%, 93%, 94%, 95%, 96%, 97%, 98%, or 99% identical thereto.
103. The lentiviral particle of any one of embodiments 99-102, wherein the chimeric antigen receptor comprises a 41BB endodomain domain comprising SEQ ID NO: 184 or a sequences at least 80%, 85%, 90%, 91%, 92%, 93%, 94%, 95%, 96%, 97%, 98%, or 99% identical thereto.
104. The lentiviral particle of any one of embodiments 99-103, wherein the chimeric antigen receptor comprises a CD3ζ endodomain comprising SEQ ID NO: 214 or a sequences at least 80%, 85%, 90%, 91%, 92%, 93%, 94%, 95%, 96%, 97%, 98%, or 99% identical thereto.
105. The lentiviral particle of any one of embodiments 99-104, wherein the fourth expression cassette encodes a polypeptide comprising SEQ ID NO: 209 or a sequences at least 80%, 85%, 90%, 91%, 92%, 93%, 94%, 95%, 96%, 97%, 98%, or 99% identical thereto.
106. The lentiviral particle of any one of embodiments 99-105, wherein the fourth expression cassette comprises a polynucleotide sequence at least 80%, 85%, 90%, 91%, 92%, 93%, 94%, 95%, 96%, 97%, 98%, or 99% identical to the polynucleotide sequence of SEQ ID NO: 186.
107. The lentiviral particle of any one of embodiments 99-106, wherein the fourth expression cassette comprises the polynucleotide sequence of SEQ ID NO: 186.
108. A lentiviral particle, comprising, displayed on the surface of the particle:
   a fusion molecule comprising
      a) a CD58 extracellular domain, or a functional fragment thereof,
      b) an antigen-binding fragment of an anti-CD3 antibody, and
      c) a CD80 extracellular domain, or a functional fragment thereof; and a viral glycoprotein (G protein);
      wherein the lentiviral particle further comprises a polynucleotide encoding a chimeric antigen receptor that specifically binds CD20,
      wherein the chimeric antigen receptor comprises a ligand-binding domain comprising an scFv, a hinge domain, a transmembrane domain, a 41BB endodomain, and a CD3ζ endodomain, and
      wherein the scFv comprises a VL comprising SEQ ID NO: 176 and a VH comprising SEQ ID NO: 179, the hinge domain comprises SEQ ID NO: 280, the transmembrane domain comprises SEQ ID NO: 281, the 41BB endodomain comprises SEQ ID NO: 184, and the CD3ζ endodomain comprises SEQ ID NO: 214.

### EXAMPLES

The following Examples describe how embodiments of the invention may be made, evaluated, and used. The Examples are intended to be illustrative and non-limiting.

### Example 1

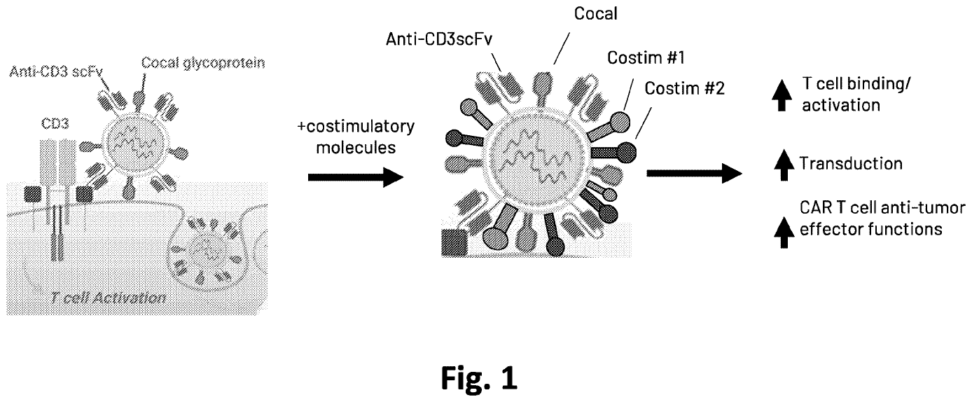

This Example shows some impacts of incorporating a costimulatory molecule such as CD80, and/or an adhesion protein such as CD58, onto the surface of a lentiviral particle. A schematic of some such lentiviral particles is provided in FIG. 1.

### Virus Production

1.2×10⁶ 293T cells were seeded into TC-treated 6-well plates in a total volume of 2.5 ml Complete DMEM media per well. 24 hours later, cells were transfected at room temperature.

The following DNA was added to 500 μl serum free OptiMEM™ media: 2 μg transfer plasmid, 1 μg Gag/pol plasmid, 1 μg REV plasmid, and 1 μg envelope plasmid. 15 μl (15 μg) PEI was then added to the media/DNA mix. The solution was then mixed well and incubated at room temperature for 20 minutes. The media/DNA/PEI mix was then added to 2.5 ml fresh Complete DMEM media. Seeding media in 293T-containing well was removed and replaced with fresh media containing the transfection reagents and placed in a 37° C. humidified incubator. 48 hours later, the supernatant was collected and filtered through a 0.45 μm PVDF filter. The virus-containing supernatant was concentrated using an Amicon-Ultra 15 100K column and centrifuged at 3000×g for 30 minutes at 4° C. The virus was then stored at 4° C. until use.

### 293T Transduction Titers

1×10⁵ 293T cells were seeded into TC-treated 12 well plates in 1 ml Complete DMEM media. 24 hours later, empty wells were counted 3× to calculate titer. Then virus was added to wells in amounts of: 2 ul, 1 ul, 0.5 ul, 0.2 ul, 0.1 ul, or 0.05 ul virus per well. Virus was diluted 1:100 before adding to 293T cells. 3 days later, 293T cells were harvested for analysis by flow cytometry. Media was removed, and cells were washed in PBS. Cells were then washed in Trypsin and incubate for ~3-5 minutes in a 37° C. incubator. Cells were resuspended in 1 ml FACS buffer and ~100-200 ul were added to a 96 well V bottom plate. Flow cytometry analysis was performed for mCherry expression.

### 293T Titer Calculation

TU/ml=(# of cells at time of transduction x % mCherry+×100)/(vector volume in ul×1000)

Engineered particles packaging an anti-CD19 CAR containing either a CD3scFV alone or a CD3scFV+CD80, CD3scFV+CD58, or CD3scFV+CD80+CD58 were added to PBMCs from 2-3 donors.

### Example 2

This Example shows that incorporation of a costimulatory molecule and/or adhesion molecule on a lentiviral particle enhanced transduction of PBMCs by lentiviral particles as generated in Example 1.

### Virus Production

All solutions used were the same as those described in Example 1. 28×10⁶ 293T cells were seeded into 16× T175 flasks (8× per vector) with 28e6 293T cells each in a total volume of 25 ml Complete DMEM media. 24 hours later, cells were transfected. Virus was produced as described in Example 1. All viruses included a Cocal envelope protein.

List of Virus preps made for study:
1. CD3scfv only
2. CD3scfv+CD58
3. CD3scfv+CD80

### PBMC Transduction and Staining for Flow Cytometry

50×10⁶ PBMCs were thawed, diluted to 2×10⁶ cells/ml in complete media (e.g. RPMI or Optimem). IL-2 was added to a final concentration of 50 IU/ml.

500 μl (1e6 cells) were added to the wells of a Non TC-treated 48 well plate. Vector was added to the wells at MOI=10, 5, and 2 based on the SupT1 ddPCR titer and the plates were placed in 37° C. incubator.

After 3 days, vector was washed out and replaced with 500 μl fresh RPMI media+IL-2 (50 IU/ml). Cells were mixed and 100-300 μl were added to wells in a 96 well V-bottom plate for activation flow cytometry analysis. Cells were then washed with 200 μl FACS buffer. The cell pellets were resuspended in 50-100 μl PBS containing LiveDead Stain (1:1000) and incubate at 4° C. for 20 min followed by another wash in 200 μl FACS buffer. Cells were resuspended in 50 μl of FACS buffer+surface stain cocktail, incubated for 30 min at 4° C., washed in 200 μl FACS buffer.

### Results and Conclusions

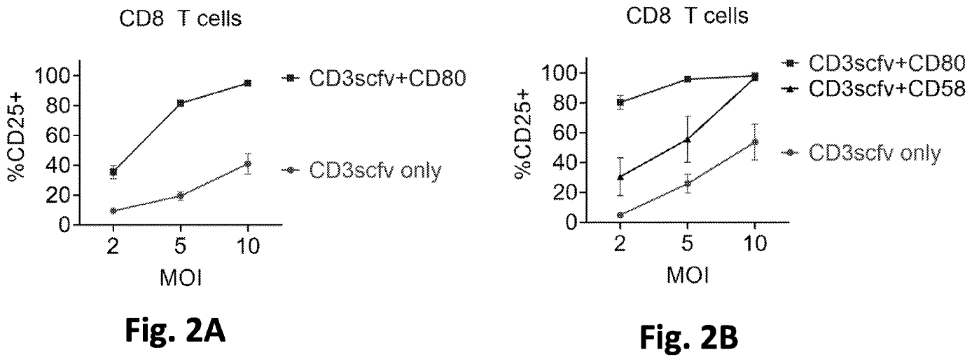

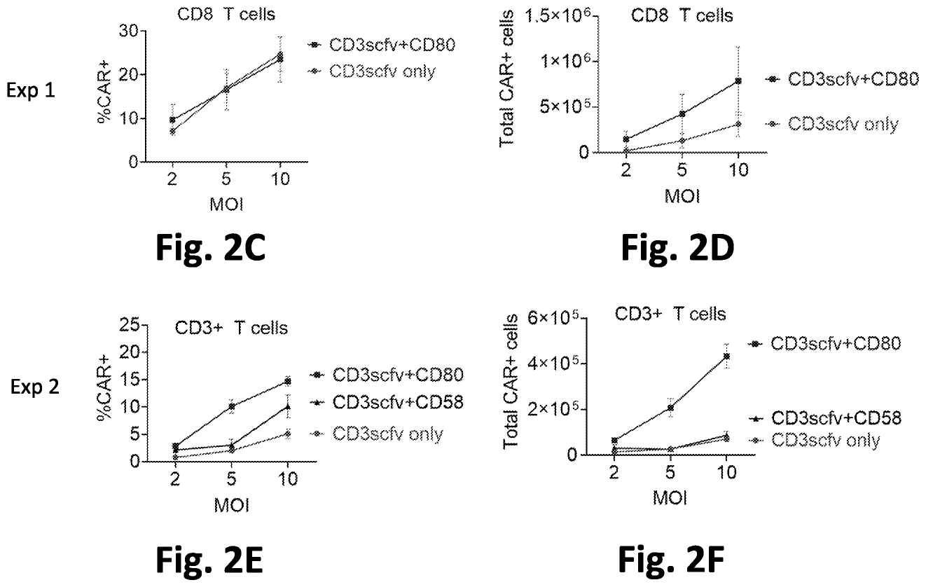

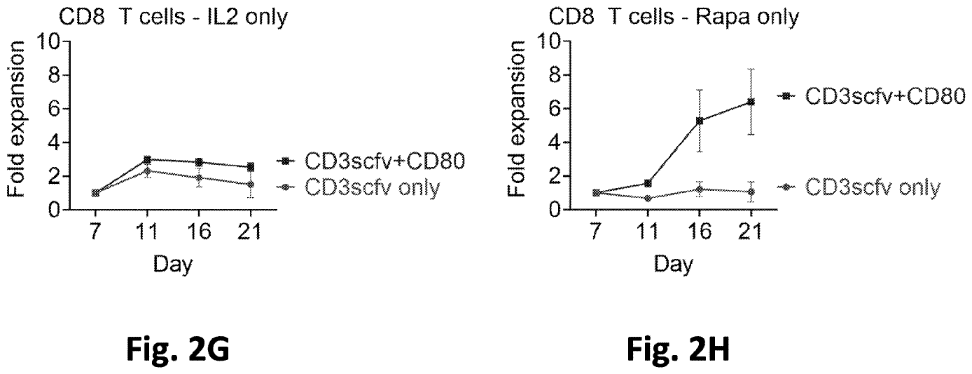

To assess if lentiviral particles with costimulatory molecules could better activate human T cells, the vector particles were added to human PBMCs at several MOI's. 3 days later, the virus was removed and the cells were given fresh media and analyzed for the activation marker CD25. CD3scfv+CD58 and CD3scfv+CD80 particles potently activated CD8 T cells compared to CD3scfv only (FIG. 2A and FIG. 2B). Furthermore, CD25 upregulation was dose-dependent (FIG. 2A and FIG. 2B). CD3scfv only lentiviral particles induced minimal levels of CD25 compared to the particles with CD80 or CD58 (FIG. 2A and FIG. 2B).

To examine transduction, 6 total days after vector addition, samples were analyzed for anti-CD19 CAR expression. Mirroring the CD25 expression on day 3, CD3scfv+CD58 and CD3scfv+CD80 particles were capable of transducing unstimulated PBMCs while CD3scfv only particles transduced unstimulated PBMCs to a lesser extent (FIGS. 2C-2F). Furthermore, transduction occurred in a dose-dependent manner for both CD3 and CD8 T cells (FIGS. 2C-2F). The data show that CD3scfv+CD58 and CD3scfv+CD80 particles efficiently activate and transduce unstimulated PBMCs in vitro compared to CD3scfv only. Importantly, the enhanced particles results in increased numbers of CAR+ T cells (FIGS. 2D-2F).

To determine if adding costimulatory molecules to particles enhances Rapa-mediated expansion of CAR+ cells in vitro, the fold expansion of CD8 T cells was determined using CD3scfv+CD80 particles compared to CD3scfv only particles. PMBCs were cultures in either IL-2 only media or Rapamycin-only media. The addition of the costimulatory molecule did not affect the fold expansion when cultured with IL-2 only (FIG. 2G) but the costimulatory molecule induced a dramatic expansion when cultured with Rapamycin-media (FIG. 2H). These results demonstrate that adding costimulatory molecules to particles enhanced Rapamycin-mediated expansion of CAR+ cells in vitro.

This study demonstrated the ability of the CD3scfv+costimulatory molecules envelope construct to deliver payloads consisting of an anti-CD19 CAR to unstimulated PBMCs in vitro. The CD3scfv+CD58 and CD3scfv+CD80 particles induced activation of T cells as measured by CD25 expression and this activation correlated with transduction as measured by % of T cells expressing the anti-CD19 CAR and total CAR+ T cells. Furthermore, activation and transduction occurred in a dose-dependent manner. Costimulatory molecules also enhance Rapamycin-mediated expansion of CAR+ cells in vitro. This data further supports the use CD3scfv+CD58 and CD3scfv+CD80 particles to deliver CAR payloads to unstimulated PBMCs in vitro and in vivo.

### Example 3

This Example shows that a combination of a costimulatory molecule (CD80 in this case), and an adhesion protein (CD58 in this case) further enhanced T cell activation and transduction. Particles having both molecules were generated. These particles were examined for their ability to activate and transduce unstimulated human PBMCs compared to particles only having anti-CD3scFv.

### Virus Production

All solutions used were the same as those described in Example 1. 28×10⁶ 293T cells were seeded into 16× T175 flasks (8× per vector) with 28×10⁶ 293T cells each in a total volume of 25 ml Complete DMEM media. 24 hours later, cells were transfected. Virus was produced as described in Example 1.

### List of Virus Preps Made for Study:

1. CD3scfv only
2. CD3scfv+CD58
3. CD3scfv+CD8
4. CD3scfv+CD80+CD58

### PBMC Transduction and Analysis

PBMCs were transduced and analyzed for expression as described in Example 2.

Supernatant cytokine analysis was measured by Meso Scale Discovery (MSD) 3 days after transduction.

Total K562.CD19, Raji, and Nalm6 tumor cells were tracked over time on an IncuCyte® for up to 15 days.

### Results and Conclusions

To assess whether lentiviral particles with costimulatory molecules and adhesion molecules enhance T cell activation and transduction, the lentiviral particles were added to human PBMCs at several MOI's. 3 days later, the virus was removed and the cells were given fresh media and analyzed for the activation marker CD25. CD3scfv+CD80+CD58 particles potently activated CD8 T cells compared to CD3scfv+CD58, CD3scfv+CD80, and CD3scfv only (FIG. 3A and FIG. 3B). Furthermore, CD25 upregulation was dose-dependent and CD3scfv+CD80+CD58 particles activated CD8 T cells at a much lower dose (FIG. 3A and FIG. 3B). CD3scfv+CD58, CD3scfv+CD80, and CD3scfv only lentiviral particles induced minimal levels of CD25 compared to the CD3scfv+CD80+CD58 particles (FIG. 3A and FIG. 3B).

To further characterize T cell activation, 3 total days after vector addition, samples were analyzed for cytokine expression. Similar to CD25 expression, CD3scfv+CD80 and CD3scfv+CD80+CD58 particles were capable of inducing IFN-γ production unstimulated PBMCs at lower doses whereas CD3scfv+CD58 and CD3scfv only particles transduced unstimulated PBMCs to a lesser extent (FIG. 3C). Furthermore, CD3scfv+CD80+CD58 particles induced robust IL-2 and TNF-α whereas CD3scfv+CD58, CD3scfv+CD80, and CD3scfv only did not (FIG. 3D and FIG. 3E). The data show that CD3scfv+CD80+CD58 particles efficiently induce cytokine production in unstimulated PBMCs in vitro compared to CD3scfv+CD58, CD3scfv+CD80, and CD3scfv only.

To examine the role of CD80 and CD58 on transduction, 3 total days after vector addition, samples were analyzed for anti-CD19 CAR expression with CD3scfv+CD80 and CD3scfv+CD58 mixed particles (FIG. 3F and FIG. 3G) or CD3scfv+CD80+CD58 on the same particle (FIG. 3H and FIG. 3I) compared to CD3scfv+CD58, CD3scfv+CD80, and CD3scfv only. CD3scfv+CD80 and CD3scfv+CD58 mixed particles or CD3scfv+CD80+CD58 on the same particle were both capable of transducing unstimulated PBMCs to a greater extent than CD3scfv+CD58, CD3scfv+CD80, and CD3scfv only (FIG. 3F, FIG. 3G, FIG. 3H and FIG. 3I). Furthermore, transduction occurred in a dose-dependent manner for both CD3 and CD8 T cells (FIG. 3F, FIG. 3G, FIG. 3H and FIG. 3I). The data show that both CD58 and CD80 either in mixed particles or on the same particle better activate and transduce unstimulated PBMCs in vitro compared to CD3scfv+CD58, CD3scfv+CD80, and CD3scfv only.

To determine whether lentiviral particles with costimulatory and/or adhesion molecules have enhanced particle binding to T cells, the particles were cultured with PBMCs for 6 hours and then were analyzed for particle-associated molecules on T cells (Cocal, CD80, and CD58). Both CD3scfv+CD58 and CD3scfv+CD80+CD58 increased Cocal staining (FIG. 3J) and only CD3scfv+CD80+CD58 demonstrated high stating for CD80 (FIG. 3K) and CD58 (FIG. 3L). The data show that the combination of CD3scfv+CD80+CD58 enhances particle binding to T cells.

To determine whether different T cell subtypes are generated by the lentiviral particles, PBMCs cultured with the lentiviral particles were profiled and gated on viable, CD3+ and CD8+. The cells were further analyzed by flow and principal component analysis was done based on parameters listed CCR7, CD45R, CD45RA, CD27, CD25, CAR+, total cells, CD4, and CD8. The analysis revealed that 3 main clusters of differentiation are produced by the different particles (FIG. 3M).

Next, T cell subtypes generated by the particles was profiled. The cells were assessed using CD45RA and CCR7 markers 7 days post transduction at an MOI of 10. Naïve T cells are CD45RA+CCR7+, effector T cells (T_{eff}) are CD45RA−CCR7−, central memory T cells (T_{cm}) are CD45RA−CCR7+, and terminally differentiated effector memory T cells (Tₑₘᵣₐ) are CD45RA+CCR7−. In a first experiment, CD3scfv only particles produced a majority of T_{eff} cells whereas CD3scfv+CD80 particles produced a majority of T_{cm} cells (FIG. 3N). In a second experiment, CD3scfv only particles produced both T_{eff} and T_{cm} cells, CD3scfv+CD80 particles produced a majority of T_{cm} cells, CD3scfv+CD58 particles produced a majority of T_{cm} cells, and CD3scfv+CD80+CD58 produced a majority of T_{cm} cells (FIG. 3O). The data show that both the addition of CD80 and/or CD58 to the particle consistently produces a T_{cm} cell phenotype. CD45RA-CCR7+ T_{cm} cells are thought to have increased longevity and proliferative capacity and correlate with better antitumor responses in vivo.

To assess the ant-tumor efficacy of CAR T cells generated using lentiviral particles expressing costimulatory and/or adhesion molecules, PBMCs were transduced and cultured with tumor cells. Specifically, particles comprising a nucleotide sequence encoding an anti-CD19 CAR were added to PBMCs at an MOI of 10, along with tumor cells (K562.CD19 or Raji cells) at PBMC:Tumor ratio of 5:1 and put directly on an Incucyte. Tumor cell killing was measured over time. The highest killing was observed with particles composed of at least CD80 in addition to CD3scfv (FIG. 4A and FIG. 4B). In a subsequent experiment, tumor cell killing was measured 7 days after transduction with an MOI of 10. The total number of CAR+ cells were calculated and incubated with either K562.CD19 or Raji cells at E:T ratios of 0.5 and 1, respectively. CAR T cells generated using a mixture of individual particles with CD80 or CD58. Similarly, the highest killing was observed with particles composed of at least CD80 in addition to CD3scfv, including CD80+CD58 (FIG. 4C and FIG. 4D). An additional experiment determined the effect for CAR T cells generated with a single lentiviral particle having both CD80 and CD58. Tumor cell killing was measured 7 days after transduction at an MOI 10. The total number of CAR+ cells were calculated and incubated with either K562.CD19 or Nalm6 cells at E:T ratios of 1:1, respectively. The CD80+CD58 dual particle provided the highest cytotoxic function (FIG. 4E and FIG. 4F).

This study demonstrated the CD3scfv+CD80+CD58 particles induced the highest differentiation of T cells and the highest cytokine production at the lowest MOI. CD3scfv+CD80+CD58 particles further had the highest T cell binding. Furthermore, this study demonstrated that CD3scfv+CD80+CD58 particles provided the highest cytolytic function in vitro.

### Example 4

This Example shows tumor control by in vivo transduction of T cells with a lentiviral particle with CD3scfv or CD3scfv+CD80. The lentiviral particle contains a polynucleotide encoding an anti-CD19 CAR. The lentiviral particle was delivered via intravenous injection into NSG MHCI/II KO mice. The mice used in the study were immune-compromised and contain engrafted human T cells and circulating human B cells.

### Study Design

### Virus Preparation, Animal Strain, Cell Lines

11 female NSG MHCI/II KO mice (Jackson laboratory) were and housed following institutional guidelines (Fred Hutchinson Cancer Research Center).

### Study Protocol

11 female NSG MHCI/II KO mice were acclimated for one week after receipt. At day −7, blood from all mice was collected for flow cytometry analysis to quantify degree of humanization. Mice were randomized according to their total human CD3 levels into the treatment groups described in Table 5.

TABLE 5
Study Treatment Groups

| | | | Administration | Virus Dose |
|---|---|---|---|---|
| Group | N | Virus type | route* | (Titre Unit) |
| 1 | 6 | CD3scfv | IV | 50 Million TU |
| | | | | Day 0 |
| 2 | 5 | CD3scfv + CD80 | IV | 50 Million TU |
| | | | | Day 0 |

### Study Timeline

At study Day 0 (SD0), 20×10⁶ PBMCs were injected into the intraperitoneal cavity. Mice were then dosed with virus particles according to the table above, followed with a challenge of 5×10⁵ luciferase+ Nalm6 tumor cells intravenously. Tumor burden was measured for the duration of the study. At SD11, blood was collected and CAR T cells were measured.

At SD75, survivor mice were rechallenged with 5×10⁶ Nalm6 cells.

### Results and Conclusions

On Day 11 of the study, blood was collected from both groups. The level of CAR T cells in the blood was higher in the CD3scfv+CD80 particle-treated group compared to the CD3scfv particle-treated group (FIG. 5A). The CD3scfv+CD80 particle-treated group was also able to decrease tumor burden during the initial challenge and subsequent rechallenge compared to the CD3scfv particle-treated group (FIG. 5B and FIG. 5C).

In summary, when delivered intravenously, CD3scfv and CD3scfv+CD80 engineered lentivirus particles successfully transduced T cells in vivo. While both groups decreased tumor burden after initial challenge and subsequent rechallenge, particles with costimulatory molecule CD80 provided greater anti-tumor efficacy and anti-tumor immune response.

### Example 5

This Example shows transduction with engineered lentiviral particles as described herein to transduce T cells in a short incubation period. Without wishing to be bound by theory, this study provides proof of concept support that the engineered particles described may be useful in an extracorporeal intravenous system.

PBMCs from 3 healthy donors were thawed and cultured with vector particles containing an anti-CD19 CAR-mCherry payload pseudotyped with either CD3scfv+cocal or CD3scfv+CD80+CD58+Cocal, generally as described in Example 2. After the indicated time point, cells were washed in serum-free media containing IL-2, human ab serum, HEPES, and glutamine. Cells were then plated in 1 ml serum-free media with IL-2 in a 24 well non-TC-treated plate. 3 days later cells were harvested and CD25 expression was measured by flow cytometry on viable T cells (FIG. 6A). The remaining cells were washed and re-plated in 1 ml fresh media containing IL-2. 4 days later (Day 7 after transduction) viable T cells were analyzed by flow cytometry for CAR surface expression (FIG. 6B). % CAR was measured by staining for anti-CD19 mAb and mCherry expression.

As shown in FIGS. 6A-6B, vector particles comprising activation, costimulation, and adhesion molecules (e.g. CD3scFv+CD80+CD58 particles) efficiently transduced T cells after short incubation periods to a greater extent than particles comprising a CD3scFv without costimulation and adhesion components. These results indicate that vector particles as described herein may enable T cell transduction during short incubation periods ex vivo, for example in a closed-loop and/or extracorporeal system.

### Example 6

This Example shows the transduction potential of lentiviral particles comprising a mutated (blinded) envelope protein. Envelope proteins, such as VSV-G or Cocal, can be mutated such that they cannot bind the LDL receptor. These modifications may enhance the specificity of lentiviral particles and reduce or eliminate off-target transduction.

SupT1 cells were cultured with vector particles containing an anti-CD19 CAR-mCherry payload, generated generally as described in Example 2. Specifically, 0.02 uL of concentrated particles were added to 3.75×10⁴ SupT1 cells and assessed for CAR expression 3 days later. The cells were cultured in the following conditions:

TABLE 6

| Condition | Particle Description |
|---|---|
| αCD3/CD58/CD80; no VSVG | CD3scFv + CD80 + CD58; no envelope |
| VSVG | VSV-G only |
| VSVG (R354Q) | VSV-G (R354Q) only |
| VSVG (K47Q) | VSV-G (K47Q) only |
| αCD3/CD58/CD80; VSVG | CD3scFv + CD80 + CD58; VSV-G |
| αCD3/CD58/CD80; VSVG (R354Q) | CD3scFv + CD80 + CD58; VSV-G (R354Q) |
| αCD3/CD58/CD80; VSVG (K47Q) | CD3scFv + CD80 + CD58; VSV-G (K47Q) |

As shown in the top row of FIG. 7A lentiviral particles comprising the blinded VSV-G mutant envelopes alone (without CD3scFv+CD80+CD58) exhibited greatly reduced transduction of SupT1 cells compared with a non-blinded VSV-G control. The bottom row depicted in FIG. 7A shows that the addition of activation, costimulation, and adhesion molecules in particles comprising blinded VSV-G mutant envelope proteins resulted in increased transduction.

Wishing to confirm the results seen using a T cell line, the experiment was replicated using PBMCs. On day 0, PBMCs from 2 donors were thawed and 2×10\hat{ }6 cells were placed in wells of a 24-well plate. Vector particles containing an anti-CD19 CAR-mCherry payload, generated generally as described in Example 2, were added to each cell-containing well according to the following chart.

TABLE 7

| Viral Particle Surface Engineering | Particle Description |
|---|---|
| αCD3 + CD58 + CD80; no VSVG | Anti-CD3scFv + CD80 + CD58; no envelope |
| VSVG | VSV-G only |
| VSVG (R354Q) | VSV-G (R354Q) only |
| VSVG (K47Q) | VSV-G (K47Q) only |
| αCD3 + CD58 + CD80; VSVG | Anti-CD3scFv + CD80 + CD58; VSV-G |
| αCD3 + CD58 + CD80; VSVG (R354Q) | Anti-CD3scFv + CD80 + CD58; VSV-G (R354Q) |
| αCD3 + CD58 + CD80; VSVG (K47Q) | Anti-CD3scFv + CD80 + CD58; VSV-G (K47Q) |

On day 3, the media was changed, and cells were re-plated with fresh media and samples were taken for assessment of transduction via flow cytometry. As shown in FIGS. 7B-7C, lentiviral particles comprising blinded VSV-G envelopes resulted in reduced transduction compared with the non-blinded VSV-G control in both CD4 (FIG. 7B) and CD8 (FIG. 7C) T cells. In addition, the addition of CD3scFv+CD80+CD58 to lentiviral particles resulted in increased transduction compared to lentiviral particles without CD3scFv+CD80+CD58. In addition, lentiviral particles comprising CD3scFv+CD80+CD58 without VSV-G also exhibited poor transduction.

On day 5, additional samples were taken for assessment of transduction via flow cytometry. Expression of CAR on day 5 was similar to expression on day 3 (data not shown).

The results of this study support the hypothesis that lentiviral particles comprising a blinded envelope protein and activation, costimulation, and adhesion molecules are capable of transducing primary T cells.

### Example 7

This Example shows expansion of non-transduced T cells after administration of a lentiviral particle with CD3scfv or CD3scfv+CD80+CD58. The lentiviral particle contains a polynucleotide encoding an anti-CD19 CAR. The lentiviral particle was delivered via intravenous injection into mice.

### Study Design

Mice were acclimated for one week after receipt. At day −7, blood from all mice was collected for flow cytometry analysis to quantify degree of humanization. Mice were randomized according to their total human CD3 levels into the treatment groups described in the table below.

TABLE 8

| | Administration | Virus Dose |
|---|---|---|
| Virus type | route | (Titre Unit) |
| CD3scfv | IV | 100 Million TU |
| CD3scfv | IV | 50 Million TU |
| CD3scfv + CD58 + CD80 | IV | 100 Million TU |
| CD3scfv + CD58 + CD80 | IV | 50 Million TU |
| CD3scfv + CD58 + CD80 | IV | 25 Million TU |

### Study Timeline

At study Day 0 (SD0) mice were then dosed with virus particles according to the table above. At SD11, blood was collected, and CAR negative T cells were measured.

### Results and Conclusions

On Day 11 of the study, blood was collected from both groups. The level of CAR negative T cells in the blood was higher in the CD3scfv+CD58+CD80 particle-treated group and was dose-dependent compared to the CD3scfv particle-treated group (FIG. 8). These results indicate, when delivered intravenously, CD3scfv+CD58+CD80 engineered lentivirus particles appears to activate and expand even non-transduced T cells in vivo. Without wishing to be bound by theory, this activation of non-transduced T cells may enable a lower dose of engineered lentiviral particles as non-transduced cells may exhibit anti-tumor activity.

### Example 8

The Examples shows that combination of a costimulatory molecules CD80, anti-CD3scFv, and an adhesion protein CD58 expressed as a single fusion polypeptide (FIG. 9A) further enhances T cell activation and transduction. Particles having the fusion polypeptide were generated. These particles were examined for their ability to activate and transduce unstimulated human PBMCs compared to particles expressing the three proteins separately and compared to particles expressing a CD80/CD58 fusion polypeptide (FIG. 9B) and expressing anti-CD3scFv separately.

### Virus Production

All solutions used were the same as those described in Example 1. Virus was produced as described in Example 1.

List of Virus preps made for study:
1. α-CD3scfv+CD80+CD58 expressed as a single fusion polypeptide (#498 triple fusion)
2. CD80+CD58 expressed as a single fusion polypeptide and α-CD3scfv expressed separately (#455 dual fusion)
3. α-CD3scfv+CD80+CD58 expressed separately “Separate”

### PBMC Transduction and Analysis

Three healthy PBMC donors were incubated with the indicated lentiviral particles described above at a multiplicity if infection (MOI) of 10 in 0.9% Sodium Chloride buffer at a concentration of 20e6 cells/ml in a total volume of 100 ul in a 96 well U-bottom plate. After 1 hour of incubation, the cells were washed, stained with antibodies, and analyzed by flow cytometry. The percentage of cells with bound Cocal and Cocal geometric mean fluorescence intensity (gMFI) are shown in FIG. 10. Cells analyzed by flow cytometry were gated on viable, CD3+, CD14−, CD56−, CD8+, Cocal+ cells. Similar data was seen for CD4+ T cells (data not shown).

### Results and Conclusions

To assess whether lentiviral particles with costimulatory molecules and adhesion molecules α-CD3scfv+CD80+CD58 expressed as a single fusion polypeptide enhance T cell activation and transduction, the lentiviral particles were added to PBMCs from three healthy PBMC donors at several MOI's and at 2E6 cells/ml in RPMI media. 3 days later, the virus was removed and the cells were washed, given fresh media, and analyzed for the activation marker CD25 by flow cytometry. Cell were gated on viable, CD3+, CD4+ or CD8+ cells. α-CD3scfv+CD80+CD58 expressed as a single fusion polypeptide “#498” particles potently activated CD4+ (FIG. 11A and FIG. 11C) and CD8+ (FIG. 11B and FIG. 11D) T cells. Furthermore, the triple fusion “#498” particles activated CD4+ (FIG. 11A and FIG. 11C) and CD8+ (FIG. 11B and FIG. 11D) T cells through displayed CD25 upregulation at a much lower dose as compared to “#455” the dual fusion and “Separate” lentiviral particles.

To further characterize T cell activation, lentiviral particles were added to PBMCs from three healthy PBMC donors at several MOI's and at 2E6 cells/ml in RPMI media. 3 days later, supernatant was harvested and cytokines were measured using V-PLEX™ Proinflammatory Panel 1 Human Kit. Similar to CD25 expression, the triple fusion “#498” particles were capable of inducing more T cell activation-associated cytokines including IFN-γ, IL-2, and TNF-α as compared to “#455” the dual fusion and “Separate” particles. Higher IFN-γ production in unstimulated PBMCs was observed at lower doses (FIG. 12A). Furthermore, the triple fusion “#498” particles induced robust IL-2 and TNF-α production as compared to “Fusion-455” and “Separate” particles (FIG. 12B and FIG. 12C). The data show that the triple fusion “#498” particles efficiently induce cytokine production in unstimulated PBMCs in vitro compared to “#455” the dual fusion and “Separate” particles.

T cell subtypes generated by the particles were profiled. The cells were assessed using CCR7, CD27, CD28, and CD57 markers. Lentiviral particles were added to PBMCs from three healthy PBMC donors at several MOI's and at 2E6 cells/ml in RPMI media. 7 days post transduction, cells were washed and CAR surface marker expression was analyzed by flow cytometry. Cells were gated on viable, CD3+, CD4+ or CD8+, CAR+ cells.

Non-terminally differentiated memory T cells are CCR7+CD27+CD28+. Triple fusion-containing particles “#498” produced a greater percentage of CCR7+CD27+CD28+ memory-like CAR+ T cells as compared to dual fusion “#455” and “Separate” particles (FIG. 13A and FIG. 13C).

CCR7+CD27+CD28+ memory-like CAR+ T cells are thought to have increased longevity and proliferative capacity and correlate with better antitumor responses in vivo. Triple fusion particles “#498” produced a smaller percentage of senescence marker CD57 at an MOI of 2 as compared to dual fusion “#455” and “Separate” particles (FIG. 13B and FIG. 13D).

### Example 9

This Example shows T cell activation and IFNγ production following in vivo transduction of T cells by a lentiviral particle displaying #498 triple fusion polypeptide as compared to #455 dual fusion and “Separate” particles as described above. The lentiviral particle contains a polynucleotide encoding an anti-CD19 CAR.

On study Day −4 NSG MHC I/I dKO mice were injected via tail vein injection with 2.5E5 Nalm6 cells expressing firefly luciferase (ffluc) (FIG. 14A). 3 days later (study Day −1), mice were imaged via bioluminescence imaging and randomized to study arms according to tumor burden (total flux), the same day all mice were humanized by injecting 20E6 human PBMCs intraperitoneally in 100 μl of 1× sterile PBS. The mice used in the study were immune-compromised and contain engrafted human T cells and circulating human B cells.

The next day (study Day 0), mice were treated via intraperitoneal injection with different doses of lentiviral particles displaying:
1. α-CD3scfv+CD80+CD58 expressed as a single fusion polypeptide “#498” triple fusion;
2. CD80+CD58 expressed as a single fusion polypeptide and α-CD3scfv expressed separately “#455” dual fusion; or
3. α-CD3scfv+CD80+CD58 expressed separately “Separate”.

A control study arm treated mice with 1×PBS (Neg) via intraperitoneal injection. Mice were then weighted two times a week throughout the study to monitor body weight change and imaged weekly to monitor tumor burden. Mice were bleed on study Days 4, 11, 18, 25, and 32 to perform flow cytometry analysis. Study day 4 activation markers CD25 (FIG. 14B) and CD71 (FIG. 14C) on CD3+ T cells were analyzed. 4 days after lentiviral particle treatment (study Day 4) serum was collected from blood and IFNγ levels in the serum were measured using V-PLEX™ proinflammatory panel 1 human kit (Mesoscale Discovery) (FIG. 14D).

Apheresis blood was washed on the Lupagen™ machine and incubated with lentiviral particles at an MOI of 2 for 1 hour in saline. The lentiviral particle contains a polynucleotide encoding an anti-CD19-mCherry transgene.

The particle-bound cells were then washed of unbound particles to generate the “Final” material. Particle-bound cells were assessed by staining for Cocal on various cell populations (CD4+ T cells, CD8+ T cells, NK T cells, NK cells, CD56+NK cells, monocytes, B cells, and other MFI) and analyzing by flow cytometery. Cocal geometric mean fluorescence intensity are shown (FIG. 15A-15C). The strongest binding was observed with particles displaying the triple fusion “#498” as compared to particles displaying the dual fusion “#455”.

### Example 10

This Examples shows in vivo antitumor responses to lentiviral particles displaying a costimulatory and adhesion molecule fusion protein using the Lupagen™ System.

On study Day −4 NSG MHC I/II DKO mice were injected via tail vein injection with 2.5E5 Nalm6 cells expressing GFP/firefly luciferase (ffluc). 3 days later (study Day −1), mice were imaged via Bioluminescence imaging and randomized to study arms according to tumor burden (total flux). On study Days 0 and 1, mice were injected with PBMCs from 2 different donors either after Lupagen™ wash or after incubation with lentiviral particles comprising “#455” dual fusion or triple fusion “#498” polypeptides in the lentiviral particle surface. On study Day 8 and every week through the study, mice were imaged via Bioluminescence imaging using the IVIS™ spectrum system to analyze tumor burden (total flux) (FIGS. 16C and 16D). Serial weekly blood draws were collected to perform flow cytometry analysis and assess CAR T cell expansion and persistence (FIGS. 16A and 16B). Disease progression was monitored by Bioluminescence imaging once a week after d-Luciferin subcutaneous injection using the IVIS™ imaging system (FIG. 16E).

### Example 11

This Example shows screening of lentiviral particles displaying variations of CD58 and CD80 dual-fusion polypeptides and screening of lentiviral particles displaying variations of CD58, CD80, and anti-CD3 scFv tri-fusion polypeptides.

Cryopreserved human PBMCs from normal donors were obtained from AllCells™. Human PBMCs were cultured in T cell growth (TCGM) media (RPMI1640+5% HuAB serum+1× GlutaMax+HEPES). For lentiviral transduction, virus was added to the PBMC cells for 3 days. Stimulation and lentiviral infection were then terminated by washing and re-seeding PBMCs in fresh TCGM media.

To analyze T cell activation, about 0.1×10⁶ cells were pelleted after the 3-day production period following lentiviral transduction described above. Cells were then analyzed by flow cytometry as follows. Cells were resuspended in Fixable Viability Dye eFluor 780 in PBS for 10 minutes, then washed with Cell Staining Buffer. T cell activation was measured by detection of hCD25 marker using an anti-CD25-PE/Cy7 antibody diluted 1:100 in Cell Staining Buffer.

To measure CAR expression levels and transduction efficiencies, about 0.1×10⁶ cells were pelleted after a 7-day production period following lentiviral transduction. Cells were then analyzed by flow cytometry as follows. Cells were resuspended in Fixable Viability Dye eFluor 780 in PBS for 10 minutes, then washed with Cell Staining Buffer. FMC63 CAR surface expression was detected using an anti-ID-FITC antibody diluted 1:100 in Cell Staining Buffer. Cells were pelleted after a 20 minute incubation in the dark, followed by 2× wash with Cell Staining Buffer. All flow cytometric analysis was done on an Attune™ NxT Flow Cytometer and analyzed with FlowJo™.

Day 7 transduced primary T cells expressing FMC63 CAR were counted, resuspended to a cell density equal to 0.4×10⁶ CAR+ cells/ml in cell-assay media (RPMI1640+10% FBS). 100 μl volume of CAR+ cells (40,000 CAR+ cells) were added to a flat-bottom 96 well plate containing 10,000 Nalm6 cells and incubated at 37° C. for effector to target ratio of 4:1. CAR+ cells are serially diluted in cell-assay media prior to plating to achieve lower effector to target ratios. Killing of target cells was analyzed in an IncuCyte™ Live Cell Analysis System. Each well was imaged every 6 hours and the number of Nalm6 cells was quantified to assess the kinetics of T cell cytotoxicity. After 24 hours, supernatant from each well was collected for cytokine measurements according to manufacturer's protocol. Nalm6 target cell lysis was tracked over >4 days. The Nalm6 target cell line was stably labeled with nuclear mKate2 by lentiviral transduction with IncuCyte™ NucLight Red Lentivirus Reagent.

### Results

Healthy donor PBMCs were transduced with lentiviruses carrying a FMC63 CAR transgene and displaying various surface engineered dual-fusion proteins at MOI 2 and 5. Early activation is determined based on hCD25 staining on Day 3 (FIG. 17), and CAR expression level was measured by staining with an anti-FMC63 antibody conjugated to FITC (FIG. 18). CAR-T cells were challenged with Nalm6-NIR (FIG. 19A-19D) to compare killing kinetics and target-dependent cytokine production levels (FIG. 20). Pro-inflammatory cytokine concentrations in co-culture supernatant after 24 hours of assay setup, in combination of killing kinetics indicate that CAR-T cells comprising the #455 dual fusion construct and separately expressed anti-CD3 scFv performed the best in functional assay.

With very low MOI transduction (MOI=0.5 and 1), lentiviral particles produced using anti-CD3 scFv and dual-fusion plasmids promoted T cell activation on Day 3 (FIG. 21), enhanced transduction efficiency and increased CAR expression on Day 7 (FIG. 22).

Tri-fusion protein “#498” surface engineered particles were compared against dual-fusion “#455” particles in PBMC transduction using both high and low MOI (MOI=1 and 10). Lentiviral particles produced using tri-fusion versions #479, #496, and #498 enhanced early T cell activation in PBMCs (FIG. 23). #496 and #498 outperformed in transduction efficiency and CAR expression on Day 7 (FIG. 24). In this experiment, #498 had the most significant effect on CAR+ T cell expansion (FIG. 25).

### Example 12

This Example shows T cell activation and transduction with lentiviral particles displaying a CD58, CD80, and anti-CD3 scFv tri-fusion polypeptide.

Human PBMCs from 3 normal donors were cultured in T cell growth (TCGM) media (RPMI1640+5% HuAB serum+1× GlutaMax+HEPES). For lentiviral transduction, lentiviral particles were added to the PBMC cells.

To analyze T cell activation, cells were pelleted after 3 days and then analyzed by flow cytometry. T cell activation was measured by detection of hCD25 marker using an anti-CD25-PE/Cy7 antibody diluted 1:100 in Cell Staining Buffer. To measure CAR expression levels and transduction efficiencies, cells were pelleted after a 7-day production period following lentiviral transduction. Cells were then analyzed by flow cytometry. Anti-CD19 CAR surface expression was detected and all flow cytometric analysis was done on an Attune™ NxT Flow Cytometer and analyzed with FlowJo™. Day 7 transduced primary T cells expressing anti-CD19 CAR were counted, resuspended, and added to Nalm6 tumor cells. Killing of target Nalm6 cells was analyzed in an IncuCyte™ Live Cell Analysis System. Each well was imaged every 6 hours and the number of Nalm6 cells was quantified to assess the kinetics of T cell cytotoxicity. After 24 hours, supernatant from each well was collected for cytokine measurements according to manufacturer's protocol.

Healthy donor PBMCs (from three donors) were contacted for less than one hour with lentiviruses carrying an anti-CD19 CAR transgene and displaying surface engineered tri-fusion proteins at MOI 2 (FIG. 28A). Consistent and efficient binding of T cells to engineered lentiviral particles was observed and measured by percentage of CD3+ T cells positively staining for Cocal (FIG. 28B). Selective T cell binding was observed in a Cocal staining peak shift for CD3+ T cells relative to CD3− T cells (FIG. 28C). Activation was determined based on hCD25 staining on Day 3 (FIG. 28D), and CAR expression level was measured (FIG. 28E). The engineered lentiviral particles demonstrated robust avidity and selectivity for T cell binding following short duration (<1 hour) culture. Transduced PBMCs were cultured with Nalm6 tumor cells. Specifically, anti-CD19 CAR+ T cells were serial-stimulated with Nalm6 tumor cells every 2-3 days. Total Nalm6 tumor cells were measured over time using an IncuCyte® providing a measurement of tumor cell killing over time (FIG. 29). This assay measures the ability of the CAR T cells to expand and kill multiple tumor cells over time and showed that anti-CD19 CAR T cells generated with lentivirus particles displaying a CD58, CD80, and anti-CD3 scFv tri-fusion protein demonstrated serial killing in vitro.

In a study of hematologic malignancy in a tumor xenograft model, on Day −4, 2.5×10′ Nalm6 cells were intravenously injected into NSG MHCI/II KO mice. At study Day −1, 20×10⁶ PBMCs were injected into the intraperitoneal cavity. At study Day 0, mice were dosed with virus particles displaying a CD58, CD80, and anti-CD3 scFv tri-fusion protein (FIG. 30A).

Four days after lentiviral particle administration, cells were harvested and expression of activation markers CD25 (FIG. 30B) and CD71 (FIG. 30C) and cytokine IFN-γ production (FIG. 30D) were measured by flow cytometry on viable CD3+ T cells in the blood. CAR T cell expansion was analyzed at doses of 10 Million and 50 Million transducing units (TU). On Day 11, total anti-CD19 CAR+ T cells found in the blood were analyzed by flow cytometry for CAR surface expression (FIG. 30E). Tumor burden was assessed as total flux and measured for the duration of the study using an In vivo Imaging system (IVIS®) (FIG. 30F).

### Example 13

This Example analyzed transduction of T cells by a lentiviral particle displaying either a dual-fusion (#455) construct or a triple fusion (#498) construct. FIGS. 3211-32I show total tumor burden (Total flux) over the course of 28 days of the study in the blood of mice injected with PBMCs from Donor 1 (FIG. 32H) or Donor 2 (FIG. 32I) after Lupagen™ incubation with untreated PBMC control, lentiviral particles displaying either a dual “#455” or triple “#498” fusion construct. The data show that extracorporeal incubation of PBMCs with the lentiviral particles described herein generates potent antitumor responses in vivo. Lentiviral particles displaying a CD58, CD80, and anti-CD3 scFv tri-fusion “#498” polypeptide showed enhanced antitumor activity in Donor 2 with a lower cell dose (Donor 2-15e6 cells were injected; Donor 1-25e6 cells were injected).

On study Day 49 (rechallenge Day 0) the mice were injected via tail vein injection with an additional 2.5E5 Nalm6 cells expressing firefly luciferase (ffluc) to assess clearance of tumor re-challenge (FIG. 33A). Tumor burden was assessed as total flux and measured for the duration of the rechallenge study for Donor 1 (D1) and Donor 2 (D2) using an In vivo Imaging system (IVIS®) (FIG. 33C). FIG. 33B shows the tumor burden in NSG MHCI/II KO mice after administration of T cells produced via extracorporeal incubation of PBMCs from Donor 1 (D1) or Donor 2 (D2) incubated with lentiviral particles displaying either a dual fusion “#455” or triple fusion “#498” construct following tumor cell rechallenge at Day 49. Lentiviral particles displaying a CD58, CD80, and anti-CD3 scFv tri-fusion “#498” polypeptide generated anti-CD19 CAR T cells, which showed persistence following primary tumor clearance and protection against tumor rechallenge in vivo.

### Example 14

This Example shows T cell transduction and activation with viral particles displaying a CD58, CD80, and anti-CD3 scFv tri-fusion polypeptide.

Human PBMCs from 3 normal donors were cultured in T cell growth (TCGM) media (RPMI1640+5% HuAB serum+1× GlutaMax+HEPES). For lentiviral transduction, viral particles were added to the PBMC cells.

Healthy donor PBMCs (from three donors) were contacted for one hour with engineered lentiviruses carrying an anti-FITC CAR transgene and displaying multi-domain fusion (MDF) surface proteins (FIG. 34A). Consistent and efficient binding of T cells to the engineered lentiviral particles was observed and measured by percentage of CD3+ T cells positively staining for Cocal (FIG. 34B). Lentiviral particles displaying MDF (multidomain fusion) proteins showed a significant increase in particle-bound cells after just one hour of incubation. Selective T cell binding was observed in increased % Cocal (FIG. 34C) and geometric mean fluorescent intensity (gMFI) Cocal (FIG. 34D) for CD4+ and CD8+ T cells relative to B cells, monocytes, and NK cells. The lentiviruses engineered to display MDF surface proteins showed selective binding to T cells over NK cells, B cells, or monocytes in a dose dependent manner. Activation was determined by % CD25 staining on Day 3 (FIG. 34F) and % CAR expression level on Day 7 post-PBMC transduction (FIG. 34G). The engineered lentiviral particles demonstrated robust transduction and activation of T cells in vitro in a dose-dependent manner.

### Example 15

Example 15 describes the efficacy of lymph node administration of engineered viral particles in non-human primates. T cell transduction and activation with viral particles surface engineered to display a CD58 and CD80 di-fusion polypeptide and anti-CD3 scFv showed in vivo CAR T cell generation. The viral particles were engineered to display an anti-CD3 scFv that binds NHP CD3 and a payload comprising a human-specific anti-CD20 CAR which cross-reacts with NHP CD20 (FIG. 35A).

TABLE 9
Abbreviations

| | |
|---|---|
| CAR | Chimeric antigen receptor |
| CBC | Complete blood count |
| CRS | Cytokine release syndrome |
| CRP | C-reactive protein |
| gDNA | Genomic DNA |
| ICANS | Immune effector cell-associated neurotoxicity syndrome |
| IL-6 | Interleukin 6 |
| IV | Intravenous |
| LN | Lymph node |
| LNGFR | Low-affinity nerve growth factor receptor |
| NHP | Non-human primate |
| PBMCs | Peripheral blood mononuclear cells |
| qPCR | Quantitative PCR |

The objectives of the study included analyzing the ability of engineered viral particles to transduce T cells and generate functional CAR T cells in vivo in a large animal model. Another objective of the study was to assess lymph node injection as a viable route of administration for engineered viral particles. Another objective of the study was to analyze the function of the generated CAR T cells on B cell depletion as well the persistence of the CAR T cells and/or B cell depletion.

In the present NHP study, the subjects of the study were Pig-tailed macaques (Macaca nemestrina) that were either male or female, a minimum size of 3.5-5 kg, no age restriction (other than to ensure sufficient size). Three animals were studied. A summary of the animals treated is shown in FIG. 35C.

Engineered viral particles were generated with surface molecules that are cross-reactive with pig-tailed macaque (Human CD58-Human CD80 fusion polypeptide and an NHP-specific anti-CD3 scFv) (FIG. 35A) and αCD20-CAR and low-affinity nerve growth factor receptor (LNGFR) molecule payload (FIG. 35B).

The particles were dosed in transducing units per kg (TU/kg) as determined by ddPCR titration on SupT1 cells. FIG. 36 shows the study design and timeline for the study.

Vector formulations were maintained at −80° C. until the day of animal treatment. Vector was kept on ice for transport to the animal facility and equilibrated at room temperature for approximately 5-15 minutes prior to lymph node injection in macaques using a single-use sterile needle and 1 ml syringe. Vector was injected within 2 hours of preparation.

Animals were individually housed (i) during the initial pre-study period in which the animals were acclimated to the jacket and tether and (ii) for at least 4 weeks following engineered viral particle injection. Animals had ad libitum access to water and food was only restricted prior to sedation or anesthesia.

### Study Procedure

Blood was drawn pre-study for isolation of (i) PBMCs, (ii) serum, and (iii) gDNA. Serum and gDNA were isolated and stored at −80° C. for later analyses. PBMCs were cryopreserved, analyzed by a pre-injection flow cytometry panel and transduced with the same engineered viral particles that were injected in vivo.

In addition, in the two-week period prior to lymph node injection, up to two blood draws were taken to determine baseline values for the assays below.

### Day 0:

Prophylactic diphenhydramine (IV injection, one dose)
Surgical placement of central catheter (if on tether)
Surgical placement of telemetry device to monitor body temperature
Optional: blood draw
Ultrasound-guided Lymph Node injection of engineered viral particles

Animals with a central intravenous catheter implanted received a continuous IV antibiotic infusion via the catheter to prevent septicemia.

Animal Monitoring and Blood Collection: Blood was drawn throughout the study with biweekly or triweekly blood draws planned for the first 3-4 weeks of the experiment and weekly thereafter. The number of draws will depend on assay results and the clinical condition of each animal. Blood draw schedule may be adjusted dependent on maximum blood draw allowance of 10 ml/kg body weight every two weeks.

Animals were observed daily for general health, appetite, activity level, responsiveness, and fecal production. Body temperature was measured continually using a telemetry device. Animals were monitored for clinical signs of cytokine release syndrome (CRS) and immune effector cell-associated neurotoxicity syndrome (ICANS) based on an established set of criteria. Clinical signs of CRS or ICANS were confirmed by CBCs, serum chemistries, serum IL-6, serum ferritin and serum CRP levels. Animals treated with engineered viral particle product in the present study did not display any clinical signs of CRS.

Study Endpoint: The animals were monitored and have blood drawn periodically following injection of engineered viral particles.

### Analytical Assays:

A. Cell Surface Flow Cytometry
B. Serum IL-6
C. Serum ferritin
D. Serum CRP

### Safety Efficacy Criteria:

Safety and Efficacy was assessed in each animal using a combination of analytics comparing results pre- and post-injection.
A. Depletion of B Cells (Flow Cytometry). The fraction of starting CD20+ cells were plotted longitudinally starting with pre-injection samples and extending throughout the study.
B. Clinical Symptoms of CRS. The serum concentration of IL-6, ferritin, and CRP were plotted longitudinally starting with pre-injection samples and extending throughout the study.
   This study is a pilot however, despite the small study size, meaningful trends in regards to B cell depletion in vivo were detected.

### Safety Criteria:

Safety was assessed using a combination of analytics.
A. CRS Monitoring
   a. Body temperature
   b. CBCs
   c. Serum IL-6
   d. Serum ferritin
   e. Serum CRP
   f. Serum chemistries (kidney (creatinine, BUN, total protein, electrolytes) and liver (AST, ALT, ALP, total bilirubin, albumin) function or damage)
B. Clinical and Physical Assessments—including general health, appetite, activity level, responsiveness, hydration, and fecal production
C. Necropsy and Histopathology on a subset of animals
   a. Gross findings
   b. Histopathology of tissue sections
   c. qPCR of tissue sections
   d. RNA-ISH of tissue positive by qPCR.

### Results

Depletion of B Cells was analyzed throughout the study in the fraction of starting CD20+ cells. In two of the three animals studied, B cell reduction was observed throughout the course of the study (FIG. 37). In vivo transduction with the engineered viral particles was well-tolerated in the animals as seen in minor increases in inflammatory markers following injection (FIG. 38). The safety data confirm none of the animals showed clinical symptoms of Cytokine release syndrome (CRS). Thus, this study supports a finding that a viral particle comprising a dual-fusion polypeptide along with a separately expressed anti-CD3 targeting protein can effectively transduce and generate functional CAR T cells in vivo via an intranodal pathway administration.

### Example 16

Example 16 describes in vivo T cell transduction and activation with viral particles surface engineered to display a CD58, CD80, and anti-CD3 scFv tri-fusion polypeptide. The methods depict successful in vivo CAR T cell generation. This non-human primate (NHP) study was conducted in M. nemestrina and showed generation of anti-CD20 CAR T cells and was well tolerated in all animals. The viral particles were engineered to display an anti-CD3 scFv that binds NHP CD3 and a payload comprising a human-specific anti-CD20 CAR which cross-reacts with NHP CD20 (FIG. 39A). Study Design and study methods are in large part the same as those described in Example 15.

The objectives of the study included analyzing the ability of engineered viral particles to transduce T cells and generate functional CAR T cells in a large animal model. Another objective of the study was to assess lymph node injection as a viable route of administration for engineered viral particles.

In the preliminary experiment described in Example 14 above, healthy donor PBMCs were contacted for one hour with engineered lentiviruses displaying CD58-anti-CD3 scFv-CD80 multi-domain fusion (MDF) surface proteins (FIG. 34A). Lentiviral particles displaying MDF (multidomain fusion) proteins showed a significant increase in particle-bound cells after just one hour of incubation (FIG. 34B). Selective T cell binding was observed in increased % Cocal (FIG. 34C) and geometric mean fluorescent intensity (gMFI) Cocal (FIG. 34D) for CD4+ and CD8+ T cells relative to B cells, monocytes, and NK cells. Engineered lentiviruses displaying MDF surface proteins 7 days after the PBMCs were contacted with the engineered particles displaying CD58, CD80, and anti-CD3 scFv MDF polypeptide showed an increased yield of CAR T cells (FIG. 34H and FIG. 34I).

In the present NHP study, the subjects of the study were Pig-tailed macaques (Macaca nemestrina) that were either male of female, a minimum size of 3.5-5 kg, no age restriction (other than to ensure sufficient size). Six animals were studied: 4 test article treated+2 control (PBS or empty particle) treated. Pigtailed macaques are well suited for lentiviral studies due to TRIM5α allele that permits efficient transduction. TRIM5α in rhesus and cynomolgus macaques restricts retroviral transduction. A summary of the aminals treated is shown in FIG. 41B.

Engineered viral particles were generated with surface molecules that are cross-reactive with pig-tailed macaque (Human CD58-NHP-specific anti-CD3 scFv-Human CD80 multi-domain fusion (MDF) polypeptide) (FIG. 39A) and αCD20-CAR-Flag payload (FIG. 39B). The particles showed efficient transduction of pig-tailed macaque PBMCs in vitro at MOI=0.2 (FIG. 40).

The particles were dosed in transducing units per kg (TU/kg) as determined by ddPCR titration on SupT1 cells. The payload was designed to express a Flag-tagged αCD20-CAR since there is no available antibody that recognizes the αCD20-CAR.

FIG. 41A shows the study design and timeline for the study.

Vector formulations were maintained at −80° C. until the day of animal treatment. Vector was kept on ice for transport to the animal facility and equilibrated at room temperature for approximately 5-15 minutes prior to lymph node injection in macaques using a single-use sterile needle and 1 ml syringe. Vector was injected within 2 hours of preparation. Control animals had an equivalent volume of vehicle (PBS) or equivalent number of surface engineered particles that did not contain a viral payload (empty particle) injected into the lymph node.

Animals were individually housed (i) during the initial pre-study period in which the animals were acclimated to the jacket and tether and (ii) for at least 4 weeks following engineered viral particle injection. Animals had ad libitum access to water and food was only restricted prior to sedation or anesthesia.

### Study Procedure

Blood was drawn pre-study for isolation of (i) PBMCs, (ii) serum, and (iii) gDNA. Serum and gDNA were isolated and stored at −80° C. for later analyses. PBMCs were cryopreserved, analyzed by a pre-injection flow cytometry panel and transduced with the same engineered viral particles that were injected in vivo.

Criteria for exclusion from study include:
(i) inability of engineered viral particles to activate T cells,
(ii) inability of engineered viral particles to transduce T cells, and
(iii) inability of CAR expressing T cells to kill target cells and produce cytokines.

Pre-Study Blood Draws: In the two-week period prior to lymph node injection, up to two blood draws were taken to determine baseline values for the assays below.

### Day 0:

Prophylactic diphenhydramine (IV injection, one dose)
Surgical placement of central catheter (if on tether)
Surgical placement of telemetry device to monitor body temperature
Optional: blood draw
Ultrasound-guided LN injection of engineered viral particles

Animals with a central intravenous catheter implanted received a continuous IV antibiotic infusion via the catheter to prevent septicemia.

Animal Monitoring and Blood Collection: Blood was drawn throughout the study with biweekly or triweekly blood draws planned for the first 3-4 weeks of the experiment and weekly thereafter. The number of draws will depend on assay results and the clinical condition of each animal. Blood draw schedule may be adjusted dependent on maximum blood draw allowance of 10 ml/kg body weight every two weeks.

Animals were observed daily for general health, appetite, activity level, responsiveness, and fecal production. Body temperature was measured continually using a telemetry device. Animals were monitored for clinical signs of cytokine release syndrome (CRS) and immune effector cell-associated neurotoxicity syndrome (ICANS) based on an established set of criteria. Clinical signs of CRS or ICANS will be confirmed by CBCs, serum chemistries, serum IL-6, serum ferritin and serum CRP levels. Animals treated with engineered viral particle product in the present study did not display any clinical signs of CRS.

Study Endpoint: The animals were monitored and have blood drawn periodically following injection of engineered viral particles.

### Analytical Assays:

A. Cell Surface Flow Cytometry
   a. NHP PBMC Screening Panel

TABLE 10

| Antigen | Clone | Fluorphore |
|---|---|---|
| CD3 | SP34-2 | PerCP-Cy5.5 |
| CD4 | OKT4 | BV605 |
| CD8 | SK1 | PE-Cy7 |
| CD45 | D058-1283 | AF700 |
| CD20 | 2H7 | AF488 |
| CD22 | RFB-4 | PE |
| CD25 | BC96 | BV421 |
| Flag | L5 | APC |
| Live/Dead | n/a | Zombie NIR |

B. qPCR Assay on gDNA extracted from peripheral blood to detect transduced cells
C. MSD Assay to measure cytokines using serum isolated from peripheral blood
D. CBC
E. Serum chemistries
F. Serum IL-6
G. Serum ferritin
H. Serum CRP

### Efficacy Criteria:

Efficacy was assessed in each animal using a combination of analytics comparing results pre- and post-injection.
a. Depletion of B Cells (Flow Cytometry). The number of B cells per μl present were plotted longitudinally starting with pre-injection samples and extending throughout the study.
b. Cytokine Release (MSD Assay). The serum concentration of each cytokine were plotted longitudinally starting with pre-injection samples and extending throughout the study.
c. Detection of CAR T Cells in Peripheral Blood (Flow Cytometry, qPCR). The number of Flag-expressing T cells (Flow Cytometry) present were plotted longitudinally starting with pre-injection samples and extending throughout the study.

With a total of 4 animals treated significant trends in regards to CAR T cell generation and B cell depletion in vivo were detected.

### Safety Criteria:

Safety will be assessed using a combination of analytics.
a. CRS Monitoring
   i. Body temperature
   ii. CBCs
   iii. Serum IL-6
   iv. Serum ferritin
   v. Serum CRP
   vi. Serum chemistries (kidney (creatinine, BUN, total protein, electrolytes) and liver (AST, ALT, ALP, total bilirubin, albumin) function or damage)

Clinical and Physical Assessments: including general health, appetite, activity level, responsiveness, hydration, and fecal production

### Necropsy and Histopathology on a Subset of Animals:

a. Gross findings
b. Histopathology of tissue sections
c. qPCR of tissue sections
d. RNA-ISH of tissue positive by qPCR.

### Results

Intranodal injection of engineered viral particles generated functional CAR T cells in Animal #1 (FIG. 42). Flow cytometry analysis showed a recurring expansion of the CAR T cells as well as persistence and the emergence of a population of memory CAR T cells at Day 35 (FIG. 42). CAR T cell kinetics were analyzed through flow cytometry analysis of cell staining with the activation marker CD25 (FIG. 43). CAR T cell activation peaked on Day 7 and Day 49 and such kinetics are consistent with antigen-engagement and B cell eradication. The presence of CD25_CAR T cells at Day 49 indicates antigen-driven expansion from a persistent memory T cell population. The generation of functional CAR T cells was further evidenced by sustained B cell aplasia for approximately 70 days (FIG. 44). The depletion of B cells coincides with the increase in CAR+ T cells (FIG. 45 and FIG. 61A). In vivo transduction with the engineered viral particles was well-tolerated in Animal #1. FIG. 46A shows a timeline of observed clinical symptoms. Increase in inflammatory markers and the onset of clinical symptoms of mild CRS (FIG. 46B peaks) and neurotoxicity (FIG. 46B first peak) coincided with CAR T cell expansion (FIG. 46B).

Animal #1 responded rapidly to invention with a single dose of each medication. In summary, the data show that intranodal administration of engineered viral particles was well-tolerated in Animal #1. The engineered viral particles show potent in vivo CAR T cell generating activity as demonstrated by flow cytometric detection of CAR T cells and sustained B cell depletion. Biodistribution of CAR T cells was also assessed in Animal #1 with the greatest number of CAR T cells observed in the injected lymph node and a lymph node downstream of the injected node (FIG. 61B). This data indicates that off-tissue transduction was not observed.

A dose de-escalation study was performed with Animal #2 wherein the animal was dosed with a half-log lower dose (FIG. 47). Animal #2 did not show symptoms of CRS: no fever, decreased appetite, or decreased activity.

Animal #3 showed symptoms of mild CRS: fever, decreased appetite, and decreased activity on Day 3. Animal #3 responded rapidly to invention with Tocilizumab and Anakinra. Expansion of CAR+ T cells on Day 7 appeared concomitantly with B cell aplasia (FIG. 48). B cell aplasia was complete and persistent rough at least day 50 of the study.

Animal #4 did not show symptoms of CRS or ICAN. Animal #4 was prophylactically treated with Anakinra between Day 7 and Day 10 (FIG. 49). Similar to Animals #1 and #3, Animal #4 exhibited peak expansion of CAR+ T cells around Day 10 with corresponding complete and persistent B cell aplasia through at least Day 50 of the study. While a CAR T memory response was not reflected in Animal #4's data, it is contemplated that the sample collection timeline missed the memory CAR T expansion. A memory CAR T response likely did occur in this animal, given the suppression of B cells after the burst observed around day 42 (FIG. 61A).

In summary, viral particles displaying multi-domain fusion (MDF) protein surface engineering showed potent in vivo anti-CD20 CAR T cell generation. Consistent generation of anti-CD20 CAR T cells that drive persistent B cell aplasia was observed in three out of three animals at the full dose (Animals 1, 3, and 4 of this Example). Typically, circulating CAR T cells are not detectable by industry standard flow cytometry. However, surprisingly, substantial circulating CAR-T cells generated by the viral particles described were also detectable by an industry standard flow cytometry assay. Further, none of the animals in the studies underwent lymphodepletion or supportive cytokine treatment prior to administration. The engineered viral particles were well-tolerated and toxicity associated with particle administration was either mild or not observed during peak CAR T expansion in most animals studied.

In conclusion, these findings demonstrate in vivo CAR T cell generation was achieved with CAR+ T cell expansion peaking around Day 10 of the study. The generation of functional CAR T cells was further evidenced B cell aplasia which coincided with the increase in CAR+ T cells beginning at Day 7 of the study. Thus, this study supports a finding that a viral particle comprising a tri-fusion polypeptide comprising binding domains from CD58 and CD80, and an anti-CD3 binding domain, can effectively transduce and generate functional CAR T cells in vivo via an intranodal route of administration.

### Example 17. In Vivo Engineered CAR-T Cells for Oncology: Nonclinical Safety and Biodistribution

This Example demonstrates the biodistribution and preliminary safety of the engineered particles of the present disclosure when delivered via either intranodal (IN) or intravenous (IV) routes of administration (ROAs).

In vivo biodistribution was assessed in canines and humanized mice (FIG. 52A-52B). FIG. 52A: CD34-NCG mice were treated IV or IP (a surrogate ROA as IN is not feasible in this model) with viral particles encoding a CAR and RACR payload with CD58+CD3 scFv+CD80 trifusion polypeptide and Cocal glycoprotein surface engineering. There were no adverse safety events during the in-life portion and mice were sacrificed at 1 week to assess biodistribution. FIG. 52B: Canines were treated with a maximal IN dose of viral particles encoding an eGFP payload, or a 10-fold higher dose IV. Animals were sacrificed at 1 week or 8 weeks to assess biodistribution. There were no test article related adverse findings after a full non-GLP tox assessment (in life, histopathology, clin chem & hematology).

Viral particle transduction was dose-dependent and highest in spleen and liver following IP or IV dosing of CD34-NCG mice (FIG. 53A-53B). Viral particle biodistribution was measured using a qPCR assay performed on genomic DNA extracted from tissues 1 week post IP or IV administration of engineered viral particle or vehicle control. N=6-7; bars are group median and dots individual animals. In FIG. 53A, for each tissue type, the left bar represents vehicle data, the middle bar represents low dose intraperitoneal data, and the right bar represents high dose intraperitoneal data. In FIG. 53B, for each tissue type, the left bar represents vehicle data, and the right bar represents high dose intraperitoneal intravenous data.

Viral particle transduction events in CD34-NCG mice were predominantly detected in immune cells (hCD3+ or mCD68+). A multiplex RNA ISH (RNAscope) assay was performed to determine transduced cell type in qPCR positive tissues. Across all tissues, transduction was predominantly detected in mouse CD68+ phagocytic cells. In the spleen, transduction of human T cells was also detected but dependent on surface engineering with the trifusion protein.

Quantifiable viral particle transduction was observed in lymphoid tissues following IN or IV administration to canines (FIG. 54). The figure depicts transduction 1 week after viral particle IN or IV administration as measured using a qPCR assay on genomic DNA extracted from tissues. Samples below LOD are depicted as “1” for graphing; samples below LLOQ depicted at LOD. N=2-3; bars are group median and dots individual animals. Following IN or IV administration of viral particles to canines, transduction of immune cells was observed.

Nonclinical studies in two species demonstrated a favorable viral particle safety and biodistribution profiles following multiple ROAs. Administration of engineered viral particles to CD34-NCG mice by IP (surrogate IN) or IV injection showed transduction of immune cells. Human T cell transduction improved with trifusion surface engineering. Following IN or IV administration to canines at high doses, quantifiable transduction was observed in lymphoid tissues. No observable in-life toxicity was observed following high viral particle doses in humanized mice or beagles. These findings support the usefulness of the engineered viral particles disclosed herein and related embodiments herein to generate CAR T cells in vivo, which may be useful for generation of CAR T cells without the need for ex vivo cell therapy manufacturing or lymphodepletion.

### Example 18. Functional Study of Engineered Particles in Lentiviral Vector Transduction

Engineered lentiviral particles comprising two different fusion proteins were evaluated.

The lentiviral particles comprise two variations of a CD58-CD3 scFv-CD80 trifusion polypeptide and Cocal glycoprotein surface engineering. Version 1 of the fusion polypeptide comprised, in order, a CD58 binding domain, an anti-CD3 scFv, and a CD80 full length polypeptide. Version 2 comprised, in order, an anti-CD3 scFv, a CD58 binding domain, and the full length CD80 polypeptide.

TABLE 11

| | Surface | Payload | | Viral Titer |
|---|---|---|---|---|
| VPL | Plasmids | Plasmid | Description | (TU/ml) |
| 341 | V1 | Anti-CD19 CAR | V1 | 4.67E+08 |
| 343 | V2 | Anti-CD19 CAR | V2 | 3.56E+08 |
| 349 | Cocal only | Anti-CD19 CAR | Cocal only | 6.83E+08 |

As shown in FIGS. 55A-55B, both versions of the fusion polypeptide effectively transduced target cells and resulted in CAR-T positive cells (FIG. 55A-55B).

### Example 19

This Example shows the incorporation of a costimulatory molecule on a lentiviral particle enhances transduction of PBMCs by lentiviral particles as generated in Example 1.

### Virus Production

All solutions used were the same as those described in Example 1. 293T cells were seeded into T175 flasks with Complete DMEM media. 24 hours later, cells were transfected. Virus was produced as described in Example 1. All viruses included a Cocal envelope protein.

List of Virus preps made for study (viruses contained an anti-CD19 CAR payload):
1. CD3scfv only
2. CD3scfv, CD80, and CD58 proteins separately expressed (“Tri protein”)

### PBMC Transduction and Staining for Flow Cytometry

50×10⁶ PBMCs were thawed, diluted to 2×10⁶ cells/ml in complete media (e.g. RPMI or Optimem). IL-2 was added to a final concentration of 50 IU/ml.

500 μl (1e6 cells) were added to the wells of a Non-TC-treated 48 well plate. Vector was added to the wells at MOI=10, 5, and 2 based on the SupT1 ddPCR titer and the plates were placed in 37° C. incubator.

After 3 days, vector was washed out and replaced with 500 μl fresh RPMI media+IL-2 (50 IU/ml). Cells were mixed and 100-300 μl were added to wells in a 96 well V-bottom plate for activation flow cytometry analysis. Cells were then washed with 200 μl FACS buffer. The cell pellets were resuspended in 50-100 μl PBS containing LiveDead Stain (1:1000) and incubate at 4° C. for 20 min followed by another wash in 200 μl FACS buffer. Cells were resuspended in 50 μl of FACS buffer+surface stain cocktail, incubated for 30 min at 4° C., washed in 200 μl FACS buffer.

### Results and Conclusions

To assess if lentiviral particles with separately expressed costimulatory and adhesion molecules can better activate human T cells, the vector particles were added to human PBMCs at several MOI's. 3 days later, the virus was removed and the cells were given fresh media and analyzed for the activation marker CD25. Tri protein particles potently activated CD8 T cells compared to CD3scfv only (FIG. 56A). Furthermore, CD25 upregulation was dose-dependent (FIG. 56A). CD3scfv only lentiviral particles induced minimal levels of CD25 compared to the Tri protein particles (FIG. 56A). To determine whether lentiviral particles with costimulatory and/or adhesion molecules have enhanced particle binding to T cells, the particles were cultured with PBMCs for 6 hours and then were analyzed for particle-associated molecules on T cells (Cocal). The separate expression of CD58, CD80, and an anti-CD3 scFv increased Cocal staining (FIG. 56B)

To examine transduction, 7 total days after transduction, samples were analyzed for anti-CD19 CAR expression. Tri protein particles were capable of transducing unstimulated PBMCs while CD3scfv only particles transduced unstimulated PBMCs to a lesser extent (FIG. 56C). Furthermore, transduction occurred in a dose-dependent manner for both CD4+ and CD8+ T cells (FIG. 56C). The data show that Tri protein particles efficiently activate and transduce unstimulated PBMCs in vitro compared to CD3scfv only. Importantly, the enhanced particles result in increased numbers of CAR+ T cells (FIG. 56C, right panels: Total CAR+ cells).

To further characterize T cell activation, 3 total days after vector addition, samples were analyzed for cytokine expression. Tri protein particles were capable of inducing IFN-γ production in unstimulated PBMCs at lower doses whereas CD3scfv only particles transduced unstimulated PBMCs to a lesser extent (FIG. 56D). Furthermore, Tri protein particles induced robust IL-2 and TNF-α whereas CD3scfv only did not (FIG. 56D). The data show that Tri protein particles efficiently induce cytokine production in unstimulated PBMCs in vitro compared to CD3scfv only.

Transduced PBMCs were then cultured with Nalm6 tumor cells. Specifically, anti-CD19 CAR+ T cells were serial-stimulated with Nalm6 tumor cells every 2-3 days. Total Nalm6 tumor cells were measured over time using an IncuCyte® providing a measurement of tumor cell killing over time (FIG. 56E). This assay measures the ability of the CAR T cells to expand and kill multiple tumor cells over time and showed that anti-CD19 CAR T cells generated with lentiviral particles displaying “Tri protein” demonstrated serial killing in vitro as compared to particles displaying CD3scfv only.

To determine whether lentiviral particles with costimulatory and/or adhesion molecules have enhanced particle binding to T cells, the particles were cultured with PBMCs for 6 hours and then were analyzed for particle-associated molecules on T cells (Cocal, anti-CD3scFv, CD80, and CD58). Both Tri protein and Fusion particles demonstrated high stating for anti-CD3scFv, CD80, and CD58, and only Fusion particles demonstrated high staining for CD3scfv+CD80+CD58 (data not shown). The data show that the fusion of CD58v+CD3 scFv+CD80 enhances particle binding to T cells. Samples were then analyzed for cytokine expression. Tri protein particles were capable of inducing IFN-γ production in unstimulated PBMCs whereas CD3scfv only particles transduced unstimulated PBMCs to a lesser extent (FIG. 56F). Furthermore, Tri protein particles induced robust IL-2 and TNF-α production whereas CD3scfv only did not (FIG. 56F). The data show that Tri protein surface engineered particles efficiently induce cytokine production in unstimulated PBMCs as compared to CD3scfv only.

To determine whether different T cell subtypes are generated by the lentiviral particles, PBMCs cultured with the lentiviral particles were profiled and gated on viable, CD4+ and CD8+. The cells were further analyzed by flow cytometry and analysis was done based on the parameters CCR7+ and CD27+ (FIG. 56G). Tri protein particles showed an increased population of CCR7+CD27+ T cells as compared to CD3 scFv only. CCR7+CD27+CD28+ memory-like CAR+ T cells are thought to have increased longevity and proliferative capacity and correlate with better antitumor responses in vivo.

### Example 20

This Example shows T cell activation and IFNγ production following in vivo transduction of T cells by a lentiviral particle displaying CD58, CD80, and an anti-CD3 scFv separately expressed as compared to CD3scfv only. The lentiviral particles contained a polynucleotide encoding an anti-CD19 CAR.

On study Day −4 NSG MHC I/I dKO mice were injected via tail vein injection with 2.5E5 Nalm6 cells expressing firefly luciferase (ffluc) (FIG. 57A). 3 days later (study Day −1), mice were imaged via bioluminescence imaging and randomized to study arms according to tumor burden (total flux), the same day all mice were humanized by injecting 20E6 human PBMCs intraperitoneally in 100 μl of 1× sterile PBS. The mice used in the study were immune-compromised and contain engrafted human T cells and circulating human B cells.

The next day (study Day 0), mice were treated via intraperitoneal injection with different doses of lentiviral particles displaying:
1. CD58, CD80, and an α-CD3 scFv separately expressed (“Tri protein”);
2. α-CD3scfv only.

A control study arm treated mice with 1×PBS (Neg) via intraperitoneal injection. Mice were then weighted two times a week throughout the study to monitor body weight change and imaged weekly to monitor tumor burden. Mice were bleed on study Days 4, 11, 18, 25, and 32 to perform flow cytometry analysis. Study day 4 activation markers CD25 (FIG. 57B) and CD71 on T cells were analyzed. A blood draw on Day 11 was collected to perform flow cytometry analysis and assess CAR T cell expansion and persistence (FIG. 57C, top panel) and CAR expression level was measured by staining with an anti-FMC63 antibody (FIG. 57C, bottom panel). On study Day 6 and every week through the study, mice were imaged via Bioluminescence imaging using the IVIS™ spectrum system to analyze tumor burden (total flux) (FIG. 57D).

### Example 21

This Example shows the incorporation of a costimulatory molecule and an adhesion molecule on a lentiviral particle enhances transduction of PBMCs by lentiviral particles as generated in Example 1.

### Virus Production

All solutions used were the same as those described in Example 1. 28×10⁶ 293T cells were seeded into 16× T175 flasks (8× per vector) with 28e6 293T cells each in a total volume of 25 ml Complete DMEM media. 24 hours later, cells were transfected. Virus was produced as described in Example 1. All viruses included a Cocal envelope protein and an anti-CD19 CAR payload.

List of Virus preps made for study:
1. CD3scfv, CD80, and CD58 separately expressed (“Tri protein”)
2. CD58+CD3scFv+CD80 expressed as a fusion protein (“Fusion”)

### Results and Conclusions

To assess if lentiviral particles with costimulatory molecules can better activate human T cells, the vector particles were added to human PBMCs at several MOI's. CD58+CD3 scFv+CD80 fusion particles potently activated CD4+ and CD8+ T cells compared to the Tri protein particles (FIG. 58B). Furthermore, CD25 upregulation was dose-dependent (FIG. 58B). To determine whether lentiviral particles with a singular fusion protein comprising costimulatory and/or adhesion molecules have enhanced particle binding to T cells, the particles were cultured with PBMCs and then were analyzed for particle-associated molecules on T cells (Cocal). The fusion particles resulted in increased Cocal staining (FIG. 58A).

To examine transduction, 7 total days after transduction, samples were analyzed for anti-CD19 CAR expression. CD58+CD3 scFv+CD80 fusion particles were capable of transducing unstimulated PBMCs at lower doses while Tri protein particles transduced unstimulated PBMCs to a lesser extent (FIG. 58C). Furthermore, transduction occurred in a dose-dependent manner for both CD4+ and CD8+ T cells (FIG. 58C). The data show that CD58+CD3 scFv+CD80 fusion particles efficiently activate and transduce unstimulated PBMCs in vitro compared to Tri protein particles.

To further characterize T cell activation, 3 total days after vector addition, samples were analyzed for cytokine expression. CD58+CD3 scFv+CD80 fusion particles induced robust IL-2 and TNF-α production as compared to Tri protein particles (FIG. 58D). The data show that CD58+CD3 scFv+CD80 fusion particles efficiently induce cytokine production in unstimulated PBMCs in vitro compared to Tri protein displaying particles.

Transduced PBMCs were then cultured with Nalm6 tumor cells. Specifically, anti-CD19 CAR+ T cells were serial-stimulated with Nalm6 tumor cells every 2-3 days. Total Nalm6 tumor cells were measured over time using an IncuCyte® providing a measurement of tumor cell killing over time. This showed that CAR T cells generated with lentiviral particles displaying a CD58+CD3 scFv+CD80 fusion protein demonstrated improved serial killing in vitro as compared to Tri protein displaying particles. In particular, CAR T cells generated with the fusion particles (Fusion) were able to continuously control tumor cells for at least 35 days. CAR T cells generated with the separately expressed proteins (Tri protein) exhibited slow tumor outgrowth beginning around day 15.

To determine whether different T cell subtypes are generated by the lentiviral particles, PBMCs cultured with the lentiviral particles were profiled and gated on viable, CD4+ and CD8+. The cells were further analyzed by flow cytometry based on parameters CCR7+ and CD27+. At most MOIs tested, both the tri protein and fusion particles were capable of generating high levels of CCR7+ and CD27+ CAR+ cells.

### Example 22

This Example shows T cell activation and IFNγ production following in vivo transduction of T cells by a lentiviral particle displaying the CD58+CD3 scFv+CD80 fusion as compared to particles comprising CD58, CD3 scFv, and CD80 separately expressed. The lentiviral particle contains a polynucleotide encoding an anti-CD19 CAR.

On study Day −4 NSG MHC I/II dKO mice were injected via tail vein injection with 2.5E5 Nalm6 cells expressing firefly luciferase (ffluc). 3 days later (study Day −1), mice were imaged via bioluminescence imaging and randomized to study arms according to tumor burden (total flux), the same day all mice were humanized by injecting 20E6 human PBMCs intraperitoneally in 100 μl of 1× sterile PBS. The mice used in the study were immune-compromised and contain engrafted human T cells and circulating human B cells.

The next day (study Day 0), mice were treated via intraperitoneal injection with different doses of lentiviral particles displaying:
1. CD3scfv, CD80, and CD58 separately expressed (“Tri protein”)
2. CD58+CD3scFv+CD80 expressed as a fusion protein (“Fusion”)

A control study arm treated mice with 1×PBS (Vehicle) via intraperitoneal injection. Mice were then weighted two times a week throughout the study to monitor body weight change and imaged weekly to monitor tumor burden. Mice were bleed on study Days 4, 11, 18, 25, and 32 to perform flow cytometry analysis. Study day 4 activation markers CD25 (FIG. 59A) and CD71 on T cells were analyzed. A blood draw on Day 11 was collected to perform flow cytometry analysis and assess CAR T cell expansion and persistence (FIG. 59B, top panel) and CAR expression level was measured by staining with an anti-FMC63 antibody (FIG. 59B, bottom panel). On study Day 6 and every week through the study, mice were imaged via Bioluminescence imaging using the IVIS™ spectrum system to analyze tumor burden (total flux) (FIG. 59C). As shown in FIG. 59C, tumor growth was better controlled in both of the fusion particle cohorts, with the higher dose exhibiting more robust tumor control. Overall % survival of the mice was analyzed over the course of the study. Lentiviral particles displaying CD58+CD3 scFv+CD80 fusion particles transduced at 50E6 TU showed increased overall survival in mice as compared to lentiviral Tri protein displaying particles.

The section headings used herein are for organizational purposes only and are not to be construed as limiting the subject matter described. Specifically, features described in one section may be combined with features in any other section of the description.

Unless otherwise defined, all terms (including technical and scientific terms) used herein have the same meaning as commonly understood by one of ordinary skill in the art to which this disclosure belongs.

The singular forms “a”, “an”, and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (or).

All publications and patents mentioned herein are hereby incorporated by reference in their entirety as if each individual publication or patent was specifically and individually indicated to be incorporated by reference. In case of conflict, the present application, including any definitions herein, controls. However, mention of any reference, article, publication, patent, patent publication, and patent application cited herein is not, and should not be taken as an acknowledgment, or any form of suggestion, that they constitute valid prior art or form part of the common general knowledge in any country in the world.

While illustrative embodiments have been described and depicted, it will be appreciated that various changes can be made to these illustrative embodiments without departing from the spirit and scope of the invention.

1. A lentiviral particle, comprising:
a polynucleotide encoding a chimeric antigen receptor (CAR) that specifically binds cluster of differentiation-20 (CD20),
wherein the CAR comprises a VL or VH sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 176 or 179; and
wherein the lentiviral particle comprises any of the following:
a heterologous viral glycoprotein (G protein),
a surface displayed polypeptide selected from: an anti-CD3 antibody or antigen-binding fragment, a CD28 ligand, or a 4-1BB ligand, or
a fusion molecule comprising: a CD58 extracellular domain, or a functional fragment thereof, a CD80 or CD86 extracellular domain, or a functional fragment thereof, and an anti-CD3 antibody or antigen-binding fragment thereof.

2. The lentiviral particle of claim 1, comprising a heterologous viral glycoprotein (G protein).

3. The lentiviral particle of claim 2, wherein the viral G protein comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 74.

4. The lentiviral particle of claim 1, comprising a surface displayed polypeptide selected from: an anti-CD3 antibody or antigen-binding fragment, a CD28 ligand, or a 4-1BB ligand.

5. The lentiviral particle of claim 1, comprising a fusion molecule comprising:
a CD58 extracellular domain, or a functional fragment thereof,
a CD80 or CD86 extracellular domain, or a functional fragment thereof, and
an anti-CD3 antibody or antigen-binding fragment thereof.

6. The lentiviral particle of claim 5, wherein the CD58 extracellular domain comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 10.

7. The lentiviral particle of claim 5, wherein the CD80 extracellular domain comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 12.

8. The lentiviral particle of claim 5, wherein the CD86 extracellular domain comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 13.

9. The lentiviral particle of claim 5, wherein the anti-CD3 antigen-binding fragment comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 31.

10. The lentiviral particle of claim 1, wherein the VL comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 176.

11. The lentiviral particle of claim 1, wherein the VL is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 175.

12. The lentiviral particle of claim 1, wherein the VH comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 179.

13. The lentiviral particle of claim 1, wherein the VH is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 178.

14. The lentiviral particle of claim 1, wherein the CAR comprises a CD8 hinge and transmembrane domain sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 212.

15. The lentiviral particle of claim 14, wherein the CD8 hinge and transmembrane domain are encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 181.

16. The lentiviral particle of claim 1, wherein the CAR comprises a 4-1BB sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 213.

17. The lentiviral particle of claim 16, wherein the 4-1BB is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 184.

18. The lentiviral particle of claim 1, wherein the CAR comprises a CD3zeta sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 214.

19. The lentiviral particle of claim 18, wherein the CD3zeta is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 185.

20. The lentiviral particle of claim 1, wherein the lentiviral particle comprises a polynucleotide encoding a free FKBP12-rapamycin binding (FRB) domain polypeptide.

21. The lentiviral particle of claim 20, wherein the free FRB polypeptide comprises a sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 251, 252, or 260.

22. The lentiviral particle of claim 20, wherein the free FRB polypeptide is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 256, 257, or 258.

23. The lentiviral particle of claim 1, wherein the lentiviral particle comprises a polynucleotide encoding a FRB polypeptide.

24. The lentiviral particle of claim 23, wherein the FRB polypeptide comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 252.

25. The lentiviral particle of claim 23, wherein the FRB polypeptide is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 257.

26. The lentiviral particle of claim 1, wherein the lentiviral particle comprises a polynucleotide encoding a cytokine gamma chain polypeptide.

27. The lentiviral particle of claim 26, wherein the cytokine gamma chain polypeptide comprises a polypeptide sequence at least 80%, at least 85%, at least 90%, at least 91%, at least 92%, at least 93%, at least 94%, at least 95%, at least 96%, at least 97%, at least 98%, at least 99%, or 100% identical to SEQ ID NO: 264 or 265.

28. The lentiviral particle of claim 26, wherein the cytokine gamma chain polypeptide is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 261, 262, or 263.

29. The lentiviral particle of claim 1, wherein the lentivirus comprises a fused FRB and the cytokine gamma chain polypeptide.

30. The lentiviral particle of claim 1, wherein the lentiviral particle comprises a polynucleotide encoding a FKBP polypeptide.

31. The lentiviral particle of claim 30, wherein the FKBP polypeptide comprises a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 253.

32. The lentiviral particle of claim 30, wherein the FKBP polypeptide is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: SEQ ID NO: 268.

33. The lentiviral particle of claim 1, wherein the lentiviral particle comprises a polynucleotide encoding a cytokine beta chain polypeptide.

34. The lentiviral particle of claim 33, wherein the cytokine beta chain polypeptide comprises a polypeptide sequence at least 80%, at least 85%, at least 90%, at least 91%, at least 92%, at least 93%, at least 94%, at least 95%, at least 96%, at least 97%, at least 98%, at least 99%, or 100% identical to SEQ ID NO: 272 or 273.

35. The lentiviral particle of claim 33, wherein the cytokine beta chain polypeptide is encoded by a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 270 or 271.

36. The lentiviral particle of claim 1, wherein the lentiviral particle comprises a fused FKBP and cytokine beta chain polypeptide.

37. A method, comprising administering the lentiviral particle of any one of the previous claims to a subject.

38. The method of claim 37, wherein the subject has cancer or is in need of cancer treatment.

39. The method of claim 38, wherein the administration treats the cancer.

40. The method of claim 38, wherein the cancer comprises CD20+ cancer cells.

41. The method of claim 37, wherein the subject has an autoimmune disease or is in need of autoimmune disease treatment.

42. The method of claim 41, wherein the administration treats the autoimmune disease.

43. The method of claim 41, wherein the autoimmune disease is caused by or exacerbated by B cells.

44. The method of claim 43, wherein the B cells are CD20+.
